(19) United States
(12) Patent Application Publication
(10) Pub. No.: US 2006/0229268 A1
(43) Pub. Date: Oct. 12, 2006

(54) Small interference RNA (siRNA) molecules for modulating superoxide dismutase (SOD)

(75) Inventors: Daniel Benjamin, Englishtown, NJ (US); Sean Scott, San Francisco, CA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ALSGEN, LLC, San Francisco, CA (US)

(21) Appl. No.: 11/303,452

(22) Filed: Dec. 16, 2005

Related U.S. Application Data
(60) Provisional application No. 60/636,752, filed on Dec. 16, 2004.

(51) Int. Cl. A61K 48/00

(52) U.S. Cl. 514/44

(57) ABSTRACT

The invention pertains to using double stranded ribonucleic acid molecules such as small interfering RNA (siRNA) molecules to target an SOD gene to interfere with gene expression and SOD protein production. Method are disclosed for inhibiting expression of a target protein in a subject with a neurological disorder by introducing a small interference ribonucleic acid (siRNA) molecule into the subject with the neurological disorder, such as amyotrophic lateral sclerosis (ALS).

### PRIORITY

This application claims priority from U.S. Provisional Application No. 60/636,752 filed Dec. 16, 2004, the contents of which is incorporated herein by reference in its entirety.

### BACKGROUND OF THE INVENTION

Amyotrophic lateral sclerosis (ALS) is the most commonly diagnosed progressive motor neuron disease. The disease is characterized by degeneration of motor neurons in the cortex, brainstem and spinal cord (Principles of Internal Medicine, 1991 McGraw-Hill, Inc., New York; Tandan et al. (1985) Ann. Neurol, 18:271-280, 419-431). The cause of the disease is unknown and ALS may only be diagnosed when the patient begins to experience asymmetric limb weakness and fatigue, localized fasciculation in the upper limbs and/or spasticity in the legs which typifies onset. There is increasing evidence that there is a genetic component to at least some incidences of ALS.

In almost all instances, sporadic ALS and autosomal dominant familial ALS (FALS) are clinically similar (Mulder et al. (1986) Neurology, 36:511-517). It has been shown that in some but not all FALS pedigrees the disease is linked to a genetic defect on chromosome 21q (Siddique et al., (1991) New Engl. J. Med., 324:1381-1384).

In particular, mutations in the SOD-1 gene which is localized on chromosome 21q, appear to be associated with the familial form of ALS. The deleterious effects of various mutations on SOD-1 are most likely mediated through a gain of toxic function rather than a loss of SOD-1 activity (Al-Chalabi and Leigh, (2000) Curr. Opin. Neurol., 13, 397-405; Alisky et al. (2000) Hum. Gene Ther., 11, 2315-2329). While the toxicity is unclear, there exists evidence to suggest that elimination of the protein itself will ameliorate the toxicity.

In the last few years, advances in nucleic acid chemistry and gene transfer have inspired new approaches to engineer specific interference with gene expression and protein production. For antisense strategies, stochiometric amounts of single-stranded nucleic acid complementary to the messenger RNA (mRNA) for the gene of interest are introduced into the cell. Some difficulties with antisense-based approaches relate to delivery, stability, and dose requirements. In general, cells do not have an uptake mechanism for single-stranded nucleic acids, hence uptake of unmodified single-stranded material is extremely inefficient. While waiting for uptake into cells, the single-stranded material is also subject to degradation.

A need exists to develop therapies that can alter the course of neurodegenerative diseases or prolong the survival time of patients with such diseases. In particular, a need exists to reduce the SOD-1 protein produced in the brain and spinal cord of ALS patients. Preventing the formation of wild type or mutant SOD-1 protein may stop disease progression and allow for amelioration of ALS symptoms.

### SUMMARY OF THE INVENTION

The invention pertains to nucleic acid chemistry and gene transfer to engineer specific interference with gene expression and protein production. In particular, the invention relates to using double stranded ribonucleic acid molecules such as small interfering RNA (siRNA) molecules to target an SOD gene to interfere with gene expression and SOD protein production. The invention relies on generating a small number of siRNA molecules that are able to interfere with SOD gene expression and SOD protein production irrelevant of any particular mutation in the SOD gene.

Although antisense strategies have been used to silence genes, the difficulties associated with antisense technology relating to delivery, stability, dose requirements and degradation, limit the use of this technology. An alternative approach is to use small interfering RNA (siRNA) molecules. With siRNA interference, a small double-stranded RNA can be used to cleave and destroy its cognate RNA, thus inhibiting the expression of the gene and the protein it encodes. The siRNA works by first assembling into an RNA-induced silencing complex (RISC), and then activating the complex by unwinding its RNA strands. The unwound RNA strands subsequently guide the complex to the complementary RNA molecules, where the complex cleaves and destroys the cognate RNA, which results in the RNA interference.

Accordingly, in one aspect the invention pertains to a method of inhibiting expression of a target protein in a subject with a neurological disorder by introducing a small interference ribonucleic acid (siRNA) molecule into the subject with the neurological disorder. The siRNA comprises a first strand and a second strand hybridized together, and at least one strand of the siRNA is complementary to the nucleotide sequence of a target gene encoding the target protein. The siRNA interacts with an RNA induced silencing complex (RISC) to activate and direct the RISC to the target gene. The destruction of the gene product is promoted, e.g., by cleavage of the mRNA sequence. This in turn prevents transcription and translation of the gene into its corresponding protein, thereby inhibiting expression of the target protein.

The method of the invention can be used to ameliorate any neurological disorder such as Amyotrophic Lateral Sclerosis (ALS), multiple sclerosis, Down's syndrome, Huntington's disease, Parkinson's disease, Spinocerebellar ataxia, Spinomuscular atrophy, Creutzfeldt-Jakob disease, Gerstmann-Straussler-Scheinker disease, and Alzheimer's disease.

The small interfering RNA can be about 15 to about 25 bases in length, preferably about 19 to about 23 bases in length. The small interfering RNA can either be an unmodified small interfering RNA or a modified RNA molecule, for example, modified to be a locked base molecule.

The siRNA are designed to target a gene that encodes a target protein. The target gene can be any gene in the disease causing pathway. For example, the target gene can be the SOD gene and the target protein can be the SOD protein. Preferably, the target gene is the SOD-1 gene, SOD-2 gene, and SOD-3 gene, and the target protein is the SOD-1 protein, SOD-2 protein and the SOD-3 protein, respectively. The SOD-1 gene can be a wild type gene or a mutant gene with at least one mutation. Likewise, the SOD-1 protein can be a wild type protein or a mutant protein with at least one mutation.

The methods of the present invention can be used to substantially inhibit expression of a target gene. As used herein, the term substantially inhibit is intended to mean inhibition of the target gene by at least 10%, more preferably about 20%, more preferably about 30%, 40%, 50%, 60%, 70%, 80%, 90% and 100%. Likewise, the expression of the target protein can be inhibited by at least 10%, more preferably about 20%, more preferably about 30%, 40%, 50%, 60%, 70%, 80%, 90% and 100%.

In another aspect, the invention pertains to a method of inhibiting expression of a SOD-1 protein in a subject with a neurological disorder by introducing a small interference ribonucleic acid (siRNA) molecule into the subject with the neurological disorder. The siRNA comprises a first strand and a second strand hybridized together, where at least one strand of the siRNA is complementary to the nucleotide sequence of an SOD-1 gene encoding the SOD-1 protein. The siRNA interacts with an RNA induced silencing complex (RISC) to activate and direct the RISC to the SOD-1 gene. Destruction of the SOD-1 gene product is promoted, thereby substantially inhibiting expression of the SOD-1 protein.

In yet another aspect, the invention pertains to a method of ameliorating amyotrophic lateral sclerosis (ALS) in subject by introducing a small interference ribonucleic acid (siRNA) molecule into the subject with the ALS, where the siRNA comprises a first strand and a second strand hybridized together. In some embodiment, ALS is familial ALS, which has been linked to SOD1 mutations. At least one strand of the siRNA is complementary to a nucleotide sequence of wild type SOD-1 gene. The siRNA interacts with an RNA induced silencing complex (RISC) to activate and direct the RISC to the wild type SOD-1 gene. Destruction of the wild type SOD-1 gene product is promoted to inhibit expression of the wild type SOD-1 protein, thereby ameliorating ALS in the subject. In another embodiments, ALS is sporadic ALS. The target genes in sporadic ALS can be identified by performing gene expression profiling on both the mouse and human sporadic patients to identify differentially expressed genes that are common to both. If genes can be found that are altered in both the mouse and human, those genes can be targeted using the methods of this invention.

In some embodiments, at least one strand of the small interfering RNA is complementary to an exon region of a SOD gene. For example, at least one strand of the small interfering RNA is complementary to the region of Exon 3 of the wild type SOD-1 gene. The small interfering RNA is about 15-25 bases in length, preferably about 19 bases in length. The small interfering RNA can be an unmodified small interfering RNA or a modified RNA molecule. The SOD-1 gene can be inhibited by at least 10%, more preferably about 20%, more preferably about 30%, 40%, 50%, 60%, 70%, 80%, 90% and 100%. Likewise, the expression of the SOD-1 protein can e inhibited by at least 10%, more preferably about 20%, more preferably about 30%, 40%, 50%, 60%, 70%, 80%, 90% and 100%. The SOD-1 gene can be the wild type SOD-1 gene or a mutant SOD-1 gene with at least one mutation. The SOD-1 protein can be the wild type SOD-1 protein or a mutant SOD-1 protein with at least one mutation.

In another aspect, the invention discloses methods of assessing the ability of an unmodified siRNA sequence to enter the cell. This selection method facilitates the selection of siRNA sequences that exhibit greater potency by virtue of improved access to the cytosolic site of action. The method of identifying a siRNA molecule useful for treating neurological disorders comprises incubating mammalian cells capable of expressing a target gene in the presence of dsRNA test compound in the absence and presence of a transfection reagent; incubating mammalian cells in the presence of a control nucleic acid compound, in the absence and presence of a transfection reagent; assaying the incubated mammalian cells for target gene expression; comparing the expression levels of the target gene. The siRNA molecule is useful for treating neurological disorders when the expression level in the presence of the dsRNA and in the absence of the transfection reagent is substantially modified when compared to the control levels (i.e., the siRNA molecule in the presence of the transfection agent, the control nucleic acid in the presences and absence of the transfection reagent). The assaying step can further include assaying for protein activity. The target gene can be a SOD gene, i.e., SOD-1. The method allows for selection of siRNA sequences with improved cell permeability and ability to reach and contact their intracellular target based on their ability to modify target gene expression in cultured cells without the use of transfection reagents. Identified siRNA sequences can then be evaluated further in vivo for their ability to modify the function and/or expression level of a target gene.

### BRIEF DESCRIPTION OF THE FIGURES

FIG. 1 is a bar chart depicting the effect of incubation with various siRNA on SOD-1 protein expression in HeLa cells;

FIG. 2 is a bar chart depicting decreased SOD levels in spinal cord of mice following intrathecal delivery of siRNA.

### DETAILED DESCRIPTION OF THE INVENTION

The practice of the present invention employs, unless otherwise indicated, conventional methods of microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, Vol. I & II (B. N. Fields and D. M Knipe, eds.))

So that the invention is more clearly understood, the following terms are defined:

The phrase “double-stranded ribonucleic acid molecule” or “dsRNA” as used herein refers to any RNA molecule, fragment or segment containing two strands forming an RNA duplex, notwithstanding the presence of single stranded overhangs of unpaired nucleotides. Further, as used herein, a double-stranded ribonucleic acid molecule includes single stranded RNA molecules forming functional stem-loop structures, such as small temporal RNAs, short hairpin RNAs and microRNAs, thereby forming the structural equivalent of an RNA duplex with single strand overhangs. The RNA molecule of the present invention may be isolated, purified, native or recombinant, and may be modified by the addition, deletion, substitution and/or alteration of one or more nucleotides, including non-naturally occurring nucleotides, also including those added at 5′ and/or 3′ ends to increase nuclease resistance.

The double-stranded ribonucleic acid molecule may be any one of a number of non-coding RNAs (i.e., RNA which is not mRNA, tRNA or rRNA), including, preferably, a small interfering RNA, but may also comprise a small temporal RNA, small nuclear RNA, small nucleolar RNA, short hairpin RNA or a microRNA having either a double-stranded structure or a stem loop configuration comprising an RNA duplex with or without single strand overhangs. The double-stranded RNA molecule may be very large, comprising thousands of nucleotides, or preferably in the case of siRNA protocols involving mammalian cells, may be small, in the range of about 15 to about 25 nucleotides, preferably in the range of about 15 to about 19 nucleotides.

The phrase “small interfering RNA” or “siRNA” as used herein, refers to a double stranded RNA duplex of any length, with or without single strand overhangs, wherein at least one strand, putatively the antisense strand, is homologous to the target mRNA to be degraded. The difference between antisense and double stranded small interfering molecules is that an antisense molecule is a single stranded oligonucleotide which is complementary to a section of the target RNA and must hybridize or bind to it in a 1:1 ratio in order to cause its degradation. In contrast, siRNA provides a substrate for the RNA-induced silencing complex (RISC), and unlike antisense, is inactive until incorporated into this macromolecular complex. This RISC complex is then guided by the unwound siRNA to its target gene. Once the target gene is located, it is destroyed by cleaving the target gene into small pieces, and thereby preventing its expression.

In a preferred embodiment, the siRNA of the present invention comprises a double-stranded RNA duplex of at least about 15, or preferably at least about 19, nucleotides with no overhanging nucleotides. In another embodiment, the siRNA of the present invention has nucleotide overhangs. For example, the siRNA may have two nucleotide overhangs, thus the siRNA will comprise a 21 nucleotide sense strand and a 21 nucleotide antisense strand paired so as to have a 19 nucleotide duplex region. The number of nucleotides in the overhang can be in the range of about 1 to about 6 homologous nucleotide overhangs at each of the 5′ and 3′ ends, preferably, about 2-4, more preferably, about 3 homologous nucleotide overhangs at each of the 5′ and 3′ ends. The nucleotides overhang can be modified, for example to increase nuclease resistance. For example, the 3′ overhang can comprise 2′ deoxynucleotides, e.g., TT, for improved nuclease resistance.

The term “homology” or “identity” as used herein refers to the percentage of likeness between nucleic acid molecules. To determine the homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. (48):444-453) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In another example, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In yet another example, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty.

The phrase “homologous” particularly refers to a nucleotide sequence that has at least 80% sequence identity, preferably at least 90% sequence identity, more preferably at least 95% sequence identity, and even more preferably at least 98% and 99% sequence identity, to a portion of mRNA transcribed from the target gene, e.g., the SOD-1 gene. The most preferred embodiment of the invention comprises a siRNA having 100% sequence identity with the target mRNA, the e.g., SOD-1 protein. Specifically, the small interfering RNA must be of sufficient homology to guide the RNA-induced silencing complex (RISC) to the target mRNA for degradation. Limited mutations in siRNA relative to the target mRNA are also within the scope of the invention.

The term “complement” refers to a nucleotide sequence which is complementary to an indicated sequence and which is able to hybridize to the indicated sequences.

In a preferred embodiment of the invention, at least a portion of one strand of the double-stranded ribonucleic acid molecule (i.e., the antisense strand) homologous to a portion of mRNA transcribed from the SOD-1 gene, preferably the human SOD-1 gene, and most preferably to exon 3 of the human SOD-1 gene. The double-stranded ribonucleic acid can be a small interfering RNA molecule selected from the siRNAs shown in Tables 1, 2, and 3.

Also included within the present invention are sequence variants of the polynucleic acids as selected from any of the nucleotide sequences as given in any of the given SEQ ID numbers or listed in Tables 1-3 with sequence variants containing either deletion and/or insertions of one or more nucleotides, especially insertions or deletions of 1 or more codons, mainly at the extremities of oligonucleotides (either 3′ or 5′), or substitutions of some non-essential nucleotides by others (including modified nucleotides an/or inosine). Other preferred variant polynucleic acids of the present invention include sequences which are redundant as a result of the degeneracy of the genetic code.

Particularly preferred variant polynucleic acids of the present invention include also sequences which hybridize under stringent conditions with any of the polynucleic acid sequences of the present invention. Particularly, sequences which show a high degree of homology (similarity) to any of the polynucleic acids of the invention as described above. Particularly sequences which are at least 80%, 85%, 90%, 95% or more homologous to said polynucleic acid sequences of the invention. Preferably said sequences will have less than 20%, 15%, 10%, or 5% variation of the original nucleotides of said polynucleic acid sequence.

Polynucleic acid sequences according to the present invention which are homologous to the sequences as represented by a SEQ ID NO can be characterized and isolated according to any of the techniques known in the art, such as amplification by means of sequence-specific primers, hybridization with sequence-specific probes under more or less stringent conditions, serological screening methods or via the LiPA typing system.

The term “inhibit” or “inhibiting” as used herein refers to a measurable reduction of expression of a target gene or a target protein. The term also refers to a measurable reduction in the activity of a target protein. Preferably a reduction in expression is at least about 10%. More preferably the reduction of expression is about 20%, 30%, 40%, 50%, 60%, 80%, 90% and even more preferably, about 100%.

The phrase “a disorder associated with SOD activity” or “a disease associated with SOD activity” as used herein refers to any disease state associated with the expression of SOD protein (e.g., SOD-1, SOD-2, SOD-3, and the like). In particular, this phrase refers to the gain of toxic function associated with SOD protein production. The SOD protein can be a wild type SOD protein or a mutant SOD protein and can be derived from a wild type SOD gene or an SOD gene with at least one mutation.

The phrase “a disorder associated with SOD-1 activity” or “a disease associated with SOD-1 activity” as used herein refers to any disease state associated with the expression of SOD-1 protein, for example, ALS. In particular, this phrase refers to the gain of toxic function associated with SOD-1 protein production. The SOD-1 protein can be a wild type SOD-1 protein or a mutant SOD-1 protein and can be derived from a wild type SOD-1 gene or an SOD-1 gene with at least one mutation.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The terms “neurological disorder” and “neurodegenerative disorder,” “neuromuscular disorder,” as used interchangeably herein refer to an impairment or absence of a normal neurological function or presence of an abnormal neurological function in a subject. For example, neurological disorders can be the result of disease, injury, and/or aging. As used herein, neurological disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many neurological disorders including, for example, Amyotrophic Lateral Sclerosis (ALS), multiple sclerosis, Down's syndrome, Huntington's disease, Parkinson's disease, Spinocerebellar ataxia, Spinomuscular atrophy, Creutzfeldt-Jakob disease, Gerstmann-Straussler-Scheinker disease, and Alzheimer's disease.

“Amyotrophic lateral sclerosis” or “ALS” are terms understood in the art and as used herein to denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

The term “therapeutically effective amount” refers to an amount effective, at dosages and for periods of time necessary, to achieve the desired therapeutic result. A therapeutically effective amount of the siRNA molecule of the present invention may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The term “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

I. RNA Interference

In one aspect, the invention pertains to using a double stranded RNA molecule to interfere with gene expression and protein production. Antisense technology is the most commonly cited approach for achieving post-transcriptional gene silencing. However, RNA interference with double stranded RNA (dsRNA) molecules has numerous advantages over antisense technology. For example, cellular uptake of unmodified antisense nucleic acid is very inefficient, therefore a large amount of antisense nucleic acid needs to be synthesized and applied in order to achieve and maintain a sufficient concentration in the target cells, which is usually at or above the level of the endogenous target mRNA. Therefore, a successful antisense strategy requires the introduction of large amounts of single-stranded antisense nucleic acid (DNA or RNA) into cells. In contrast, the cellular uptake of double-stranded RNA is more efficient, thereby permitting RNA interference to occur with much smaller amounts of dsRNA.

When double-stranded RNA (dsRNA) is introduced into a cell, it has the ability to silence the expression of a homologous gene within the cell, i.e., “interfere” with gene expression. In 1998, Fire et al. demonstrated the efficacy of RNA interference by injecting the gut of C. elegans with a dsRNA that had been prepared in vitro (Fire, et al. (1998) Nature, 391, 806-811). The injection of dsRNA into C. elegans resulted in loss of expression of the homologous target gene, not only throughout the worm, but also in its progeny.

The difference between antisense and double stranded small interfering molecules is that antisense molecule is a single stranded oligonucleotide which is complementary to a section of the target RNA and must hybridize or bind to it in a 1:1 ratio in order to cause it's degradation. In contrast, siRNA provides a substrate for the RNA-induced silencing complex (RISC), and unlike antisense, is inactive until incorporated into this macromolecular complex.

More specifically, in eukaryotes, the current model of the RNA interference mechanism involves both an initiation and an effector step. In the initiation step, a processing enzyme cleaves the introduced dsRNA into small interfering RNAs of 21-23 nucleotides. In the effector step, each siRNA is incorporated into an RNA induced silencing complex (“RISC”), comprising a helicase, an exonucleolytic nuclease, and an endonucleolytic nuclease. The siRNA, now incorporated into the RISC, serves as a guide molecule, directing the RISC to the homologous mRNA transcript for degradation (Hammond, et al., “Post-transcriptional gene silencing by double-stranded RNA,” Nature Rev. Gen., 2, 110-119). The RISC complex is led to the intended mRNA by the incorporated siRNA molecule and catalyzes the cleavage of multiple copies of the mRNA, whereas the antisense sequence is destroyed after mediating the cleavage a single mRNA molecule. Double stranded small interfering molecules have the advantage of being more stable than single stranded RNA, and being more effective at inhibition at lower concentrations than single stranded RNA. In addition, siRNA does not require the use of viral vectors.

Other double stranded RNA molecules are also included within the scope of the invention. A growing number of RNAs do not function as messenger RNAs, transfer RNAs or ribosomal RNAs. These so-called “non-coding” RNAs describe a wide variety of RNAs of incredibly diverse function, ranging from the purely structural to the purely regulatory (Riddihough, (2002) Science, 296, 1259). The non-coding RNA that has generated the most interest, however, is the “small interfering RNA” or “siRNA” associated with the phenomenon of RNA interference (“RNAi”). Other representative non-coding RNAs include small nuclear RNAs, involved in the splicing of pre-mRNAs in eukaryotes (Will et al., (2001) Curr. Opin. Cell Biol., 13, 290), small nucleolar RNAs, which direct 2′-O-ribose methylation and pseudouridylation of rRNA and tRNA (Kiss, (2001) EMBO J, 20, 3617) and “micro-RNAs” (“miRNAs”), very small RNAs of approximately 22 nucleotides in length which appear to be involved in various aspects of mRNA regulation and degradation. Two miRNAs characterized in some detail are the “small temporal RNAs” (“stRNAs”) lin4 and let7, which control developmental timing in the nematode worm C. elegans and repress the translation of their target genes by binding to the 3′ untranslated regions of their mRNAs (Riddihough, (2002) Supra); Ruvkun, (2001) Science, 294, 797; Grosshans, et al., (2002) J. Cell. Biol. 156, 17). Also known are the short hairpin RNAs (“shRNAs”), patterned from endogenously encoded triggers of the RNA interference pathway (Paddison, et al., (2002) Genes and Dev., 16:948-958).

In the present invention, siRNA are introduced into the cell rather than large dsRNA molecules, thus circumventing the initiation step of the mechanism. Although composed of two structural elements that resemble oligonucleotides used in antisense gene inhibition, the siRNA molecule has clear structural distinctions from the former. A siRNA molecule is composed of two complementary strands of RNA that must be hybridized with one another. There must be base-pair overhangs at each end of the molecule. Although the two oligonucleotides used for siRNA are the same length as those used for antisense, they will not be incorporated into the RISC complex unless they form this RNA duplex.

Where the siRNA of the present invention is delivered to a cell for the purposes of inhibiting expression of a target gene within the cell, at least one strand of the small interfering RNA is homologous to a portion of mRNA transcribed from the target gene, e.g., wild type SOD-1. In a preferred embodiment, the siRNA strand is at least 85% homologous to a portion of mRNA transcribed from the target gene. Preferably, the siRNA strand is 90% homologous, more preferably is 95% homologous, and even more preferably, is 98% and 99% homologous to a portion of mRNA transcribed from the target gene, e.g., wild type SOD-1. In the most preferred embodiment, at least one strand of the siRNA is 100% homologous to a portion of mRNA transcribed from the target gene, e.g., wild type SOD-1.

In one embodiment, at least one siRNA molecule can be delivered to the cell, for example an siRNA molecule associated with a region of the SOD-1 gene, e.g., the exon 3 region of the SOD-1 gene. In another embodiment, a plurality of siRNA molecules can be delivered to the cell, for example, a plurality of siRNA molecules associated with one region of the SOD-1 gene, e.g., exon 3 region. In another embodiment, the plurality of siRNA molecules can be associated with different regions of the SOD-1 gene, for example, exon-1, and exon-3; or exon-2, exon-3, and exon-4; or exon-1, exon-2, exon-4, and exon-4, and so forth. Thus, it will be appreciated that the scope of the invention covers any combination of siRNA molecules that can target and interfere with one or more desired regions of the SOD-1 gene.

The target gene may be an endogenous gene in relation to the cell, as in the case of a regulatory gene or a gene coding for a native protein, or it may be heterologous in relation to the cell, as in the case of a viral or bacterial gene, transposon, or transgene. In either case, uninhibited expression of the target gene may result in a disease or a condition. To inhibit expression of the target gene, the cell is contacted with the siRNA in an amount sufficient to inhibit expression of the target gene, e.g., wild type SOD-1.

The cell receiving the siRNA of the present invention may be isolated, within a tissue, or within an organism. It may be an animal cell, a plant cell, a fungal cell, a protozoan, or a bacterium. An animal cell may be derived from vertebrates or invertebrates, but in a preferred embodiment of the invention, the cell is derived from a mammal, such as a rodent or a primate, and even more preferably, is derived from a human. The cell may be of any type, including neural cells, neuronal cells, epithelial cells, endothelial cells, muscle cells or nerve cells. Representative cell types include, but are not limited to, microglia, myoblasts, fibroblasts, astrocytes, neurons, oligodendrocytes, macrophages, myotubes, lymphocytes, NIH3T3 cells, PC12 cells, and neuroblastoma cells. Such delivery may be accomplished either in vitro or in vivo by standard techniques.

The siRNA can be obtained by chemical synthesis or by DNA-vector based RNA interference technology. Custom siRNAs can be generated on order from Dharmacon Research, Inc., Lafayette, Colo. Other sources for custom siRNA preparation include Xeragon Oligonucleotides, Huntsville, Ala. and Ambion of Austin, Tex. Alternatively, siRNAs can be chemically synthesized using ribonucleoside phosphoramidites and a DNA/RNA synthesizer. In the present invention, the siRNA molecules were chemically synthesized using the Invitrogen commercially available technique with ribonucleoside phosphoramidites and a DNA/RNA synthesizer.

Using DNA vector based siRNA technology, a small DNA insert (about 70 bp) encoding a short hairpin RNA targeting the gene of interest is cloned into a commercially available vector. The insert-containing vector can be transfected into the cell, and it expresses the short hairpin RNA. The hairpin RNA is rapidly processed by the cellular machinery into 19-22 nt double stranded RNA (siRNA). The following is a list of commercially available GenScript siRNA expression vectors: U6 like promoter: pRNA-U6.1/Neo, pRNA-U6.1/Hygro, pRNA-U6.1/Zeo, pRNAT-U6.1/Neo (with GFP marker), pRNAT-U6.1/Hygro (with GFP marker). H1 like promoter: pRNA-H1.1/Neo, pRNA-H1.1/Hygro, pRNA-H1.1/Zeo, pRNAT-H1.1/Neo (with GFP marker), pRNAT-H1.1/Hygro (with GFP marker).

To improve hybridization, locked bases, which differ from native RNA bases in that they contain a 2′-O, 4′-C methylene bridge, can be used. By chemically modifying the siRNA, enhanced hybridization and improved biostability, can be achieved. The siRNA can be chemically modified at either or both the 5′ and 3′ end bases to increase stability, hybridization, and cellular uptake. The molecules can be modified using the locked base technology described by Proligo in U.S. Pat. No. 6,794,499 and U.S. Pat. No. 6,670,461, incorporated herein by reference.

The siRNA can be chemically modified, for example, by N-type modification to produce a linked nucleic acid (LNA). LNA is a synthetic nucleic acid analogue, incorporating “internally bridged” nucleoside analogues. Synthesis of LNA, and properties thereof, have been described by a number of authors: Nielsen et al, (1997) J. Chem. Soc. Perkin Trans. 1, 3423); Koshkin et al, (1998) Tetrahedron Letters 39, 4381; Singh & Wengel (1998) Chem. Commun. 1247; and Singh et al, (1998) Chem. Commun. 455. LNA exhibits greater thermal stability when paired with DNA, than do conventional DNA/DNA heteroduplexes.

A sugar engineered into an N-type (RNA-like) pucker usually conveys an increase in helical thermostability when hybridized with complementary RNA (Freier et al. (1997) Nucleic Acids Res. 25, 4429-4443). Prominent examples of such N-type nucleic acid analogues are 2′-O-alkylated RNA (Manoharan (1999) Biochim. Biophys. Acta 1489, 117-130), 2′F-RNA (Kawasaki et al. (1993) J. Med. Chem. 36, 831-841), phosphoramidates (Gryaznov (1999) Biochim. Biophys. Acta 1489, 131-140), HNA (Hendrix et al. (1997) Chem. Eur. J. 3, 1513-1520), and LNA (Koshkin, et al. (1998) Tetrahedron 54, 3607-3630; Obika, et al. (1998) Tetrahedron Lett. 39, 5401-5404; Wengel, (1999) Acc. Chem. Res. 32,301-310; and Petersen (2003) Trends Biotechnol. 21, 74-81).

In LNA, the furanose conformation is chemically locked in an N-type (C3′-endo) conformation by the introduction of a 2′-O,4′-C methylene linkage. LNAs have shown high thermal affinities when hybridized with either DNA (Tm=1-8° C. per modification) (Koshkin et al. (1998) Tetrahedron 54, 3607-3630; Obika, et al. (1998) Tetrahedron Lett. 39, 5401-5404; Wengel. (1999) Acc. Chem. Res. 32, 301-310; Petersen, et al. (2003) Trends Biotechnol. 21, 74-81; Kvaern, et al. (2000) J. Org. Chem. 65, 5167-5176; and Braasch, et al. (2001) Chem. Biol. 8, 1-7), RNA (Tm=2-10° C. per modification) (Braasch, (2001) Chem. Biol. 8, 1-7; Bondensgaard, et al. (2000) Chem. Eur. J. 6, 2687-2695; and Kurreck et al. (2002) Nucleic Acids Res. 30, 1911-1918) or LNA (Tm>5° C. per modification) (Koshkin, (1998) J. Am. Chem. Soc. 120, 13252-13253).

II. SOD and SOD Mutations

The invention pertains to eliminating the SOD-1 protein, particularly wild type SOD-1 protein in cells by causing the degradation of the mRNA encoding SOD-1 protein using dsRNA, interference, specifically with siRNA molecules. The siRNA generated will target the human wild type SOD-1 mRNA in regions that do not contain mutations. This strategy allows the silencing of the bulk of familial mutations without designing individual molecules for each mutation. While the target of the siRNA will be the wild type SOD-1 protein, sequences that target mutations in SOD-1 are also within the scope of the invention.

The SOD-1 gene is localized to chromosome 21q22.1. SOD-1 sequences are disclosed in PCT publication WO 94/19493 are oligonucleotide sequences encoding SOD-1 and generally claimed is the use of an antisense DNA homolog of a gene encoding SOD-1 in either mutant and wild-type forms in the preparation of a medicament for treating a patient with a disease (Brown et al., 1994).

The nucleic acid sequence of human SOD-1 gene can be found at Genbank accession no. NM_{—}000454. The nucleotide sequence of human SOD-1 is also presented in SEQ ID NO: 1. the underlined regions are the exon regions. The corresponding SOD-1 protein sequence is presented in SEQ ID NO: 2. The siRNA molecules were designed around exon 3 of the SOD-1 gene. The entire sequence of exon 3 is disclosed in SEQ ID NO: 3. siRNA molecules that can be used to inhibit the SOD-1 gene are disclosed in Table 1, and preferred siRNA molecules that inhibit expression of the SOD-1 gene are described in the Examples section.

The siRNA molecules are all sequences are listed in the 5′-3′ direction, with the sense sequence of the pair listed first. All sequences were rigorously tested for similarity with known human mRNAs in GeneBank using the Blast algorithm for short, nearly exact matches. Examples of some preferred sequences are shown in the Examples section. These and other siRNA sequences can readily be made using the methods and sequences disclosed in the invention.

RNA interference with siRNA produces a measurable reduction of expression of a target gene or a target protein. Preferably a reduction in expression is at least about 10%. More preferably the reduction of expression is about 20%, 30%, 40%, 50%, 60%, 80%, 90% and even more preferably, about 100%.

III. Delivery of Double Stranded RNA

Previous methods of delivering double stranded RNA primarily involve transfection (for general transfection protocols, see Elbashir, et al., (2001) Nature, 411, 494-498; Elbashir, et al., (2001b) Genes & Dev., 15, 188-200). The efficiency of transfection depends on cell type, passage number and the confluency of the cells. The time and the manner of formation of dsRNA are also critical. One example of transfection of siRNA molecules includes using U6 and CMV promoters in any suitable transfection vector.

Yet another method of delivering double stranded molecules to a cell involves using cell-penetration enhancing peptides conjugated to the double stranded molecules. The membrane shuttling proteins such as the Drosophila homeobox protein Antennapedia, the HIV-1 transcriptional factor TAT and VP22 from HSV-1 can be conjugated to the siRNA molecule to increase its cellular uptake and thus efficacy.

Other techniques for dsRNA uptake include electroporation, injection, liposome-facilitated transport, and microinjection. Although direct microinjection of dsRNA into cells is generally considered to be the most effective means known for inducing RNA interference, the characteristics of this technique severely limit its practical utility. In particular, direct microinjection can only be performed in vitro, which limits its application to gene therapy. Furthermore, only one cell at a time can be microinjected, which limits the technique's efficiency. As a means of introducing dsRNA into cells, electroporation is also relatively impractical because it is not possible in vivo. Finally, while dsRNA can be introduced into cells using liposome-facilitated transportation or passive uptake. The siRNA sequences can be assessed for their ability to inhibit gene expression in cultured cells in the absence of transfection reagent. In a preferred embodiment, the siRNA is delivered intraspinally without a gene therapy vector. Delivery of siRNA molecules can also be accomplished by passive cellular uptake in vivo (see United States Patent Application 20040248174).

It is also possible to introduce dsRNA indirectly into cells, by transforming the cells with expression vectors containing DNA coding for dsRNA (See, e.g., U.S. Pat. No. 6,278,039, U.S. published application 2002/0006664, WO 99/32619, WO 01/29058, WO 01/68836, and WO 01/96584). Cells transformed with the dsRNA-encoding expression vector will then produce dsRNA in vivo.

Another delivery method involves delivering naked siRNA molecules directly into the central nervous system of the subject. This can be accomplished by using a ventricular Omaya reservoir spinal catheter (e.g., portacath). Alternatively, cirect delivery of the siRNA molecules can be accomplished by using continuous spinal infusion using pump technologies (e.g., for Medtronic pump). For continuous spinal infusion, the lumbar catheterization protocol can be conducted by initially preparing a catheter using for example, polyethylene tubing (PE10) with outer diameter of about 0.6 mm, and a total tubing length of about 4.5 cm. A thin tungsten wire (e.g., with a diameter of about 0.12 mm) can be inserted into the PE10 tube as a guide wire. One end of the tubing can be stretched so the outer diameter shrinks. A triple knot can be made with silk suture at each end of the tubing in order to provide anchor points for the tubing after catheter implantation. An ALZET pump can be filled and primed with at least one siRNA molecule formulated in a delivery vehicle such as saline, dextrose, artificial cerebrospinal fluid, and the like. The siRNA can be delivered at a rate of about 6 μl/day. It will be appreciated that the volume of the siRNA formulation, and the rate at which it is delivered will depend on the size and weight of the subject. An adapter tube can be made using 0.69 mmID tubing cut to approximately 5 mm.

To implant the catheter, the subject, e.g., mice can be anesthetized with ketamine/domitor combination IP injection. The mice can be injected with Buprenex as a pain medication. A 2 cm longitudinal skin incision can be made above vertebrae L5 and L6. While holding the mouse's pelvic girdle firmly, a hole can be made in the muscle at the L5 and L6 junction using a 23 gauge needle. The needle can be gently pressed and spun through the muscle tissue. The catheter with metal wire inside can be pushed into the side of the L5-6 process initially at a 70 degree angle from the vertebral column. The angle can be flattened once resistance is reached until the catheter and wire is about 20-30 degrees from the vertebral column. The catheter with the wire can be pushed through the intervertebral space and dura until the sign of dura penetration (tail flick and/or hind limb quiver) occurs. At this point the guide wire is withdrawn in order to protect the spinal cord from damage. The catheter is then fed into the vertebral space until the silk suture knot rests adjacent to the hole in the muscle. A knot is tied through the fascia that rests superficially to the lumbar muscle so that the knot anchors the original silk catheter knot into its place. This keeps the catheter in place. The ALZET pump is attached to the catheter tubing using an adhesive and adaptor tube. The pump is implanted in the skin pocket. The second silk knot is anchored to the fascia at the neck with a suture knot. The incision is closed and the mice are dosed with Antesedan in order to counteract the Domitor.

Where delivery is made in vivo to a living organism, administration may be by any procedure known in the art, including but not limited to, oral, parenteral, intraspinal, intracisternal, subdural, rectal, intradermal, transdermal, intramuscular, or topical administration. To facilitate delivery, the dsRNA may be formulated in various compositions with a pharmaceutically acceptable carrier, excipient or diluent. “Pharmaceutically acceptable” means the carrier, excipient or diluent of choice does not adversely affect the biological activity of the dsRNA, or the recipient of the composition.

Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water.

For oral administration, the composition may be presented as capsules or tablets, powders, granules or a suspension. The composition may be further presented in convenient unit dosage form, and may be prepared using a controlled-release formulation, buffering agents and/or enteric coatings.

For parenteral administration (i.e., subcutaneous, intravenous, or intramuscular administration), the dsRNA may be dissolved or suspended in a sterile aqueous or non-aqueous isotonic solution, containing one or more of the carriers, excipients or diluents noted above. Such formulations may be prepared by dissolving a composition containing the dsRNA in sterile water containing physiologically compatible substances such as sodium chloride, glycine, and the like, and having a buffered pH compatible with physiological conditions to produce an aqueous solution. Alternatively, a composition containing the dsRNA may be dissolved in non-aqueous isotonic solutions of polyethylene glycol, propylene glycol, ethanol, corn oil, cottonseed oil, peanut oil, etc.

The dsRNA may be administered by formulation with any suitable carrier that is solid at room temperature but dissolves at body temperature. Such carriers include cocoa butter, synthetic mono-, di-, or tri-glycerides, fatty acids, polyethylene glycols, glycerinated gelatin, hydrogenated vegetable oils, and the like.

Intradermal administration of the dsRNA, i.e., administration via injectable preparation, may be accomplished by suspending or dissolving the dsRNA in a non-toxic parenterally acceptable diluent or solvent, e.g., as a solution in 1,3-butanediol, water, Ringer's solution, and isotonic sodium chloride solution. Occasionally, sterile fixed oils or fatty acids are employed as a solvent or suspending medium.

For transdermal or topical administration, the dsRNA may be combined with compounds that act to increase the permeability of the skin and allow passage of the dsRNA into the bloodstream. Such enhancers include propylene glycol, polyethylene glycol, isopropanol, ethanol, oleic acid, N-methylpyrrolidone, and the like. Delivery of such compositions may be via transdermal patch or iontophoresis device.

Specific formulations of compounds for therapeutic treatment are discussed in Hoover, J. E., Remington's Pharmaceutical Sciences (Easton, Pa.: Mack Publishing Co., 1975) and Liberman, H. A., and Lachman, L., Eds., Pharmaceutical Dosage Forms (New York, N.Y.: Marcel Decker Publishers, 1980).

The quantity of dsRNA administered to tissue or to a subject should be an amount that is effective to inhibit expression of the target gene within the tissue or subject, and are readily determined by the practitioner skilled in the art. Specific dosage will depend further upon the dsRNA, e.g., siRNA used, the target gene to be inhibited and the cell type having target gene expression. Quantities will be adjusted for the body weight of the subject and the particular disease or condition being targeted.

A stable cell line with a specific gene knocked-out can be established, and its phenotype can be studied. A knock-out mouse line can be established using transgenic dsRNA, e.g., siRNA method (Kunach et al. (2003) Nature Biotechnology 21:559-561). dsRNA can be inserted into a vector with an inducible promoter to study its effect. The dsRNA can be delivered by using for example, a viral vector (Shen et al. (2003) FEBS Lett 539(1-3):111-114; and Barton et al. (2002) Proc Natl Acad Sci USA 99(23):14943-14945) and used for gene therapy purpose.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Designing siRNA

Targets for siRNA were designed for wild type SOD-1 mRNA. A general strategy for designing siRNA targets comprises beginning at the start codon for exon 3 of SOD-1 and then scanning the length of exon 3. The potential target site can then be compared to the appropriate genome database, so that any target sequences that have significant homology to non-target genes can be discarded. Multiple target sequences along the length of the gene should be located, so that target sequences are derived from the 3′, 5′ and medial portions of the mRNA of exon 3. Negative control siRNAs can be generated using the same nucleotide composition as the subject siRNA, but scrambled and checked so as to lack sequence homology to any genes of the cells being transfected (Elbashir et al. (2001) Nature, 411, 494-498; Ambion siRNA Design Protocol, at www.ambion.com).

In the present invention, generated target sequences were 19 bases long, beginning with start codon of exon 3 (SEQ ID NO: 3). Exon 3 was selected as a target gene for siRNA molecules because it is the stretch of SOD1 mRNA on exon 3 that harbors the fewest (practically zero) disease-associated mutations. This is important because there are numerous different disease-associated SOD-1 mutations on the SOD-1 gene. However, the present invention eliminates the need for separate siRNA molecules for each mutation by creating one siRNA molecule that inhibits SOD-1 gene expression and protein production. The method of the invention is therefore suitable for all FALS patients with various different mutations, regardless of their particular mutation.

The siRNAs were chemically synthesized using ribonucleoside phosphoramidites and a DNA/RNA synthesizer.

TABLE 1

siRNA molecules that can be used to
inhibit the SOD-1 gene.

| | |
|---|---|
| SEQ ID NO: 4 | 5′-UUAAUCCUCUAUCCAGAAA-3′ (sense) |
| SEQ ID NO: 5 | 5′-UUUCUCCAUAGAGGAUUAA-3′ (antisense) |
| SEQ ID NO: 6 | 5′-GUGCAGGUCCUCACUUUAA-3′ (sense) |
| SEQ ID NO: 7 | 5′-UUAAAGUGAGGACCUGCAC-3′ (antisense) |
| SEQ ID NO: 8 | 5′-AGUGCAGGUCCUCACUUUA-3′ (sense) |
| SEQ ID NO: 9 | 5′-UAAAGUGAGGACCUGCACU-3′ (antisense) |
| SEQ ID NO: 10 | 5′-UCCUCACUUUAAUCCUCUA-3′ (sense) |
| SEQ ID NO: 11 | 5′-UAGAGGAUUAAAGUGAGGA-3′ (antisense) |
| SEQ ID NO: 12 | 5′-AAUACAGCAGGCUGUACCA-3′ (sense) |
| SEQ ID NO: 13 | 5′-UGGUACAGCCUGCUGUAUU-3′ (antisense) |
| SEQ ID NO: 14 | 5′-GCAGGUCCUCACUUUAAUC-3′ (sense) |
| SEQ ID NO: 15 | 5′-GAUUAAAGUGAGGACCUGC-3′ (antisense) |
| SEQ ID NO: 16 | 5′-CCUCACUUUAAUCCUCUAU-3′ (sense) |
| SEQ ID NO: 17 | 5′-AUAGAGGAUUAAAGUGAGG-3′ (antisense) |
| SEQ ID NO: 18 | 5′-UCACUUUAAUCCUCUAUCC-3′ (sense) |
| SEQ ID NO: 19 | 5′-GGAUAGAGGAUUAAAGUGA-3′ (antisense) |
| SEQ ID NO: 20 | 5′-CACUUUAAUCCUCUAUCCA-3′ (sense) |
| SEQ ID NO: 21 | 5′-UGGAUAGAGGAUUAAAGUG-3′ (antisense) |
| SEQ ID NO: 22 | 5′-CUUUAAUCCUCUAUCCAGA-3 (sense) |
| SEQ ID NO: 23 | 5′-UCUGGAUAGACCAUUAAAG-3′ (antisense) |
| SEQ ID NO: 24 | 5′-UUUAAUCCUCUAUCCAGAA-3′ (sense) |
| SEQ ID NO: 25 | 5′-UUCUGGAUAGAGGAUUAAA-3′ (antisense) |
| SEQ ID NO: 26 | 5′-AAUCCUCUAUCCAGAAAAC-3′ (sense) |
| SEQ ID NO: 27 | 5′-GUUUUCUGGAUAGAGGAUU-3′ (antisense) |
| SEQ ID NO: 28 | 5′-AUCCUCUAUCCAGAAAACA-3′ (sense) |
| SEQ ID NO: 29 | 5′-UGUUUUCAGGAUAGAGGAU-3′ (antisense) |
| SEQ ID NO: 30 | 5′-CCAGUGCAGGUCCUCACUU-3′ (sense) |
| SEQ ID NO: 31 | 5′-AAGUGAGGACCUGCACUGG-3′ (antisense) |
| SEQ ID NO: 32 | 5′-GCUUAAAGGAAUUGACAAA-3′ (sense) |
| SEQ ID NO: 33 | 5′-UUUGUCAAUUCCUUUAAGC-3′ (antisense) |

### Example 2

### Testing siRNA In Vitro

To quantify the effect the inhibition of expression in vitro, attenuation of gene function was assessed by the measurement of mRNA using typical real time fluorescence detection technologies, and by the measurement of immunoreactivity using an enzyme linked immunosorbent assay (ELISA; Bender Medsystems MST222).

Briefly, HeLa cells (ATCC) were plated into 96 well microtiter plates at a density of 4000 cells/well and allowed 12 hours to attach. Following an initial 12 hour incubation, annealed duplex RNA was added to each well at concentrations from 20 nM through 10 uM, in the presence and absence of lipid transfection reagents. Cultures were assayed following 24-72 h of RNA treatment. Control sequences with the same base composition but different orders of nucleotides were tested in parallel fashion.

The data showed that siRNA targeted to SOD-1 could decrease SOD-1 expression. Cultured hippocampal neurons were treated with various concentrations of siRNA targeted to SOD-1, and assayed for SOD activity.

Since transfection reagents are generally used in cell-based studies using siRNA to allow the siRNA to enter the cell, but are not usually compatible with most in vivo applications, a protocol was developed to quantify the cell penetration of unmodified and modified siRNA sequences. Sequences, from 18 to 30 base-pairs long were added in concentrations from 10 micromolar to 10 picomolar to cell cultures. Cells can be HeLa, human embryonic kidney HEK-293 cells, or any neuroblastoma, glial, microglial, lymphocyte, or other mammalian cell line or primary cell, maintained in serum-free medium. Following addition of the siRNA sequences to the medium, cells are assayed at time points ranging from 24 to 168 hours for target protein levels (via ELISA or Western, or dot immunoblot), target mRNA levels, or target enzyme activity. Minimal effective concentrations of the siRNA and their IC₅₀ values for inhibition of SOD1 expression are then used to rank efficacy and cell penetration.

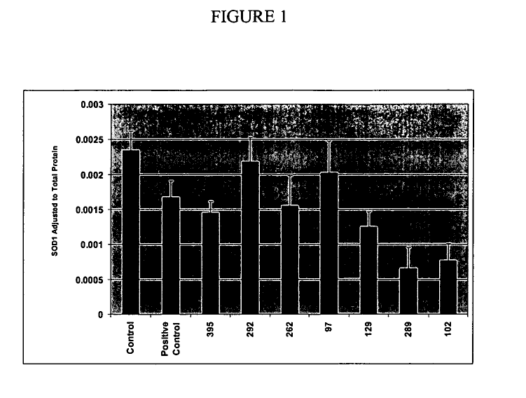

Over 1000 siRNA duplexes have been designed using rational and computer assisted design tools (see Table 3). The siRNA sequences listed in Table 3 are part of the present invention. Several very potent sequences were identified as shown by FIG. 1. FIG. 1 depicts a bar graphs showing the decease in SOD-1 protein following incubation of HeLa cells with various siRNA listed in Table 2 (n=5, p<0.005). Candidate siRNA molecules that show greater than a 10% reduction in SOD-1 protein in vitro are tested in vivo.

TABLE 2
Sequences of siRNAs tested in Example 2.

| | | |
|---|---|---|
| SEQ ID NO. 34 | 395Forward | GUGGAAAUGAAGAAAGUACAAAG |
| SEQ ID NO. 35 | 395Reverse | CUUUGUACUUUCUUCAUUUCCAC |
| SEQ ID NO. 36 | 292Forward | GCCGAUGUGUCUAUUGAAGAUUC |
| SEQ ID NO. 37 | 292Reverse | GAAUCUUCAAUAGACACAUCGGC |
| SEQ ID NO. 38 | 262Forward | GGCAAUGUGACUGCUGACAAAGA |
| SEQ ID NO. 39 | 262Reverse | UCUUUGUCAGCAGUCACAUUGCC |
| SEQ ID NO. 40 | 97Forward | AAGGUGUGGGGAAGCAUUAAAGG |
| SEQ ID NO. 41 | 97Reverse | CCUUUAAUGCUUCCCCACACCUU |
| SEQ ID NO. 42 | 129Forward | AGGCCUGCAUGGAUUCCAUGUUC |
| SEQ ID NO. 43 | 129Reverse | GAACAUGGAAUCCAUGCAGGCCU |
| SEQ ID NO. 44 | 289Forward | GUGGCCGAUGUGUCUAUUGAAGA |
| SEQ ID NO. 45 | 289Reverse | UCUUCAAUAGACACAUCGGCCAC |
| SEQ ID NO. 46 | 102Forward | GUGGGGAAGCAUUAAAGGACUGA |
| SEQ ID NO. 47 | 102Reverse | UCAGUCCUUUAAUGCUUCCCCAC |

### Example 3

### Testing siRNA In Vivo: siRNA Knockdown of Mouse SOD1 mRNA

To quantify the effect the inhibition of expression in vitro, the siRNA molecules was introduced into the SOD-93A murine model (GTC Biotherapeutics, Inc., Framingham, Mass.) for ALS, and the life expectancy measured. The inhibition of RNA expression was monitored by isolated blood samples from a mouse pre- and post introduction of the siRNA molecule using standard RT-PCR techniques. The expression of the SOD-1 protein was determined using ELISA, Western blot techniques, or TaqMan quantitative PCR.

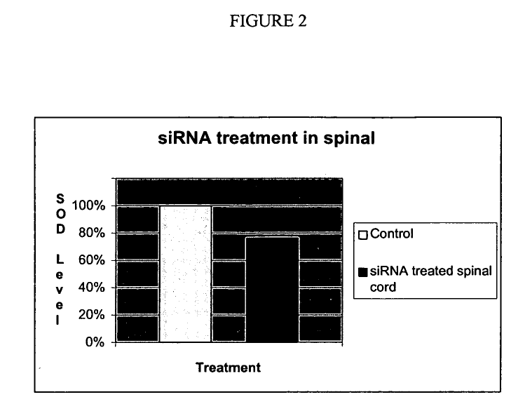

In vivo experiments were conducted with siRNA molecules that show a significant reduction (i.e., greater than 10%, preferably greater than 20%, most preferably greater than 50%) of SOD1 levels. siRNA molecules that were showed a 50% reduction in vitro at a concentration of about 50 nM siRNA were tested in vivo. This concentration is low enough that therapeutically relevant drug levels should be achievable in the spinal cord. Animal testing demonstrates about 25% knockdown in the spinal cord via intrathecal delivery of 50 nM siRNA sequence 289 (SEQ ID NO. 44 and 45) (See FIG. 2). These experiments were repeated in triplicate and demonstrate statistically significant results (p<0.05). Similar in vivo experiments can be performed using alternative routes of deliver (i.e., oral, parenteral, intraspinal, intracisternal, subdural, rectal, intradermal, transdermal, intramuscular, or topical administration). This experiment demonstrates that the methods of the invention can be used effectively in vivo.

TABLE 3

siRNA sequences of SOD-1 designed using
rational and computer assisted design tools.

| | |
|---|---|
| Sense Strand (5′-3′) | Lower Strand (3′-5′) |

| | |
|---|---|
| UCAAGCCUGUGAAUAAAAA | AGUUCGGACACUUAUUUUU |
| UCAUGAGUUUGGAGAUAAU | AGUACUCAAACCUCUAUUA |
| UUAAUCCUCUAUCCAGAAA | AAUUAGGAGAUAGGUCUUU |
| CAAUGUGACUGCUGACAAA | GUUACACUGACGACUGUUU |
| UAAUUGGGAUCGCCCAAUA | AUUAACCCUAGCGGGUUAU |
| GUAGAAAUGUAUCCUGAUA | CAUCUUUACAUAGGACUAU |
| GUAGUGAGAAACUGAUUUA | CAUCACUCUUUGACUAAAU |
| GUAUUUUGCCAGACUUAAA | CAUAAAACGGUCUGAAUUU |
| AGAAAUGUAUCCUGAUAAA | UCUUUACAUAGGACUAUUU |
| GUAUCCUGAUAAACAUUAA | CAUAGGACUAUUUGUAAUU |
| UAAACACUGUAAUCUUAAA | AUUUGUGACAUUAGAAUUU |
| GAAGAUUUGUAUAGUUUUA | CUUCUAAACAUAUCAAAAU |
| AGAUUUGUAUAGUUUUAUA | UCUAAACAUAUCAAAAUAU |
| CGGAGGUCUGGCCUAUAAA | GCCUCCAGACCGGAUAUUU |
| UGCAGGGCAUCAUCAAUUU | ACGUCCCGUAGUAGUUAAA |
| UCAUCAAUUUCGAGCAGAA | AGUAGUUAAAGCUCGUCUU |
| GGUGUGGGGAAGCAUUAAA | CCACACCCCUUCGUAAUUU |
| UGAGUUUGGAGAUAAUACA | ACUCAAACCUCUAUUAUGU |
| GUGCAGGUCCUCACUUUAA | CACGUCCAGGAGUGAAAUU |
| UGCAGGUCCUCACUUUAAU | ACGUCCAGGAGUGAAAUUA |
| UAAUCCUCUAUCCAGAAAA | AUUAGGAGAUAGGUCUUUU |
| UGGCCGAUGUGUCUAUUGA | ACCGGCUACACAGAUAACU |
| CGAUGUGUCUAUUGAAGAU | GCUACACAGAUAACUUCUA |
| ACACUGGUGGUCCAUGAAA | UGUGACCACCAGGUACUUU |
| CACUGGUGGUCCAUGAAAA | GUGACCACCAGGUACUUUU |
| ACUGGUGGUCCAUGAAAAA | UGACCACCAGGUACUUUUU |
| GGGCAAAGGUGGAAAUGAA | CCCGUUUCCACCUUUACUU |
| CAAAGGUGGAAAUGAAGAA | GUUUCCACCUUUACUUCUU |
| GGAAAUGAAGAAAGUACAA | CCUUUACUUCUUUCAUGUU |
| UGAAGAAAGUACAAAGACA | ACUUCUUUCAUGUUUCUGU |
| UUUGGCUUGUGGUGUAAUU | AAACCGAACACCACAUUAA |
| AAUUGGGAUCGCCCAAUAA | UUAACCCUAGCGGGUUAUU |
| AUUGGGAUCGCCCAAUAAA | UAACCCUAGCGGGUUAUUU |
| CAAUAAACAUUCCCUUGGA | GUUAUUUGUAAGGGAACCU |
| GUAGUCUGAGGCCCCUUAA | CAUCAGACUCCGGGGAAUU |
| CCCCUUAACUCAUCUGUUA | GGGGAAUUGAGUAGACAAU |
| UUAUCCUGCUAGCUGUAGA | AAUAGGACGAUCGACAUCU |
| UGCUAGCUGUAGAAAUGUA | ACGAUCGACAUCUUUACAU |
| UCAGAGUUGCUUUAAAGUA | AGUCUCAACGAAAUUUCAU |
| UAAAGUACCUGUAGUGAGA | AUUUCAUGGACAUCACUCU |
| AAGUACCUGUAGUGAGAAA | UUCAUGGACAUCACUCUUU |
| GUGAGAAACUGAUUUAUGA | CACUCUUUGACUAAAUACU |
| AAAUCACAGAUGGGUAUUA | UUUAGUGUCUACCCAUAAU |
| AAUCACAGAUGGGUAUUAA | UUAGUGUCUACCCAUAAUU |
| AUCACAGAUGGGUAUUAAA | UAGUGUCUACCCAUAAUUU |
| ACAGAUGGGUAUUAAACUU | UGUCUACCCAUAAUUUGAA |
| CAUUCAAGCCUGUGAAUAA | GUAAGUUCGGACACUUAUU |
| AUUCAAGCCUGUGAAUAAA | UAAGUUCGGACACUUAUUU |
| UUCAAGCCUGUGAAUAAAA | AAGUUCGGACACUUAUUUU |
| CCCUGUAUGGCACUUAUUA | GGGACAUACCGUGAAUAAU |
| GCGGAGGUCUGGCCUAUAA | CGCCUCCAGACCGGAUAUU |
| AAACACUGUAAUCUUAAAA | UUUGUGACAUUAGAAUUUU |
| UUUUCAGAGUUGCUUUAAA | AAAAGUCUCAACGAAAUUU |
| UUUGUAUAGUUUUAUAAAA | AAACAUAUCAAAAUAUUUU |
| UUAUAAAACUCAGUUAAAA | AAUAUUUUGAGUCAAUUUU |
| AGAAUUUCUUUGUCAUUCA | UCUUAAAGAAACAGUAAGU |
| GAAUUUCUUUGUCAUUCAA | CUUAAAGAAACAGUAAGUU |
| UUAUUAUGAGGCUAUUAAA | AAUAAUACUCCGAUAAUUU |
| UUAUGAGGCUAUUAAAAGA | AAUACUCCGAUAAUUUUCU |
| AGGUCUGGCCUAUAAAGUA | UCCAGACCGGAUAUUUCAU |
| AGCGAGUUAUGGCGACGAA | UCGCUCAAUACCGCUGCUU |
| CAGUGCAGGGCAUCAUCAA | GUCACGUCCCGUAGUAGUU |
| AGGGCAUCAUCAAUUUCGA | UCCCGUAGUAGUUAAAGCU |
| UCAAUUUCGAGCAGAAGGA | AGUUAAAGCUCGUCUUCCU |
| UCGAGCAGAAGGAAAGUAA | AGCUCGUCUUCCUUUCAUU |
| CGAGCAGAAGGAAAGUAAU | GCUCGUCUUCCUUUCAUUA |
| GCAGAAGGAAAGUAAUGGA | CGUCUUCCUUUCAUUACCU |
| GAAGGAAAGUAAUGGACCA | CUUCCUUUCAUUACCUGGU |
| GAAAGUAAUGGACCAGUGA | CUUUCAUUACCUGGUCACU |
| AAAGUAAUGGACCAGUGAA | UUUCAUUACCUGGUCACUU |
| AAGGUGUGGGGAAGCAUUA | UUCCACACCCCUUCGUAAU |
| AGGUGUGGGGAAGCAUUAA | UCCACACCCCUUCGUAAUU |
| GCAUUAAAGGACUGACUGA | CGUAAUUUCCUGACUGACU |
| UGCAUGGAUUCCAUGUUCA | ACGUACCUAAGGUACAAGU |
| GCAUGGAUUCCAUGUUCAU | CGUACCUAAGGUACAAGUA |
| AUUCCAUGUUCAUGAGUUU | UUAGGUACAAGUACUCAAA |
| CCAUGUUCAUGAGUUUGGA | GGUACAAGUACUCAAACCU |
| GUUCAUGAGUUUGGAGAUA | CAAGUACUCAAACCUCUAU |
| UUCAUGAGUUUGGAGAUAA | AAGUACUCAAACCUCUAUU |
| CAUGAGUUUGGAGAUAAUA | GUACUCAAACCUCUAUUAU |
| CAGUGCAGGUCCUCACUUU | GUCACGUCCAGGAGUGAAA |
| AGUGCAGGUCCUCACUUUA | UCACGUCCAGGAGUGAAAU |
| UCCUCACUUUAAUCCUCUA | AGGAGUGAAAUUAGGAGAU |
| UGAAGAGAGGCAUGUUGGA | ACUUCUCUCCGUACAACCU |
| GGAGACUUGGGCAAUGUGA | CCUCUGAACCCGUUACACU |
| GCAAUGUGACUGCUGACAA | CGUUACACUGACGACUGUU |
| AUGUGACUGCUGACAAAGA | UACACUGACGACUGUUUCU |
| UGUGACUGCUGACAAAGAU | ACACUGACGACUGUUUCUA |
| GGCCGAUGUGUCUAUUGAA | CCGGCUACACAGAUAACUU |
| UGUGUCUAUUGAAGAUUCU | ACACAGAUAACUUCUAAGA |
| UGAAGAUUCUGUGAUCUCA | ACUUCUAAGACACUAGAGU |
| UGAUCUCACUCUCAGGAGA | ACUAGAGUGAGAGUCCUCU |
| UCAGGAGACCAUUGCAUCA | AGUCCUCUGGUAACGUAGU |
| AGGAGACCAUUGCAUCAUU | UCCUCUGGUAACGUAGUAA |
| CACACUGGUGGUCCAUGAA | GUGUGACCACCAGGUACUU |
| GGUGGUCCAUGAAAAAGCA | CCACCAGGUACUUUUUCGU |
| GCAAAGGUGGAAAUGAAGA | CGUUUCCACCUUUACUUCU |
| AAAGGUGGAAAUGAAGAAA | UUUCCACCUUUACUUCUUU |
| AGGUGGAAAUGAAGAAAGU | UCCACCUUUACUUCUUUCA |
| GAAAGUACAAAGACAGGAA | CUUUCAUGUUUCUGUCCUU |
| AAAGUACAAAGACAGGAAA | UUUCAUGUUUCUGUCCUUU |
| UCGUUUGGCUUGUGGUGUA | AGCAAACCGAACACCACAU |
| CGUUUGGCUUGUGGUGUAA | GCAAACCGAACACCACAUU |
| UGGGAUCGCCCAAUAAACA | ACCCUAGCGGGUUAUUUGU |
| GGAUCGCCCAAUAAACAUU | CCUAGCGGGUUAUUUGUAA |
| AAACAUUCCCUUGGAUGUA | UUUGUAAGGGAACCUACAU |
| ACAUUCCCUUGGAUGUAGU | UGUAAGGGAACCUACAUCA |
| UCCCUUGGAUGUAGUCUGA | AGGGAACCUACAUCAGACU |
| CUCAUCUGUUAUCCUGCUA | GAGUAGACAAUAGGACGAU |
| UAUCCUGCUAGCUGUAGAA | AUAGGACGAUCGACAUCUU |
| AAAGUGUAAUUGUGUGACU | UUUCACAUUAACACACUGA |
| GUAAUUGUGUGACUUUUUC | CAUUAACACACUGAAAAAG |
| CUUUUUCAGAGUUGCUUUA | GAAAAAGUCUCAACGAAAU |
| GUACCUGUAGUGAGAAACU | CAUGGACAUCACUCUUUGA |
| UGAUUUAUGAUCACUUGGA | ACUAAAUACUAGUGAACCU |
| GAUUUAUGAUCACUUGGAA | CUAAAUACUAGUGAACCUU |
| AUGAUCACUUGGAAGAUUU | UACUAGUGAACCUUCUAAA |
| AUCACUUGGAAGAUUUGUA | UAGUGAACCUUCUAAACAU |
| UCACUUGGAAGAUUUGUAU | AGUGAACCUUCUAAACAUA |
| CACUUGGAAGAUUUGUAUA | GUGAACCUUCUAAACAUAU |
| CUGUUUCAAUGACCUGUAU | GACAAAGUUACUGGACAUA |
| GUUUCAAUGACCUGUAUUU | CAAAGUUACUGGACAUAAA |
| UGACCUGUAUUUUGCCAGA | ACUGGACAUAAAACGGUCU |
| CUGUAUUUUGCCAGACUUA | GACAUAAAACGGUCUGAAU |
| UUUUGCCAGACUUAAAUCA | AAAACGGUCUGAAUUUAGU |
| UUGCCAGACUUAAAUCACA | AACGGUCUGAAUUUAGUGU |
| AGAUGGGUAUUAAACUUGU | UCUACCCAUAAUUUGAACA |
| UGUCAGAAUUUCUUUGUCA | ACAGUCUUAAAGAAACAGU |
| UCAUUCAAGCCUGUGAAUA | AGUAAGUUCGGACACUUAU |
| AAACCCUGUAUGGCACUUA | UUUGGGACAUACCGUGAAU |
| AACCCUGUAUGGCACUUAU | UUGGGACAUACCGUGAAUA |
| ACCCUGUAUGGCACUUAUU | UGGGACAUACCGUGAAUAA |
| CCUGUAUGGCACUUAUUAU | GGACAUACCGUGAAUAAUA |
| UGUAUGGCACUUAUUAUGA | ACAUACCGUGAAUAAUACU |
| GGCGUGGCCUAGCGAGUUA | CCGCACCGGAUCGCUCAAU |
| GCACACUGGUGGUCCAUGA | CGUGUGACCACCAGGUACU |
| AAAUGUAUCCUGAUAAACA | UUUACAUAGGACUAUUUGU |
| UAUCCUGAUAAACAUUAAA | AUAGGACUAUUUGUAAUUU |
| UAAACAUUAAACACUGUAA | AUUUGUAAUUUGUGACAUU |
| AUUAAACACUGUAAUCUUA | UAAUUUGUGACAUUAGAAU |
| UUAAACACUGUAAUCUUAA | AAUUUGUGACAUUAGAAUU |
| ACACUGUAAUCUUAAAAGU | UGUGACAUUAGAAUUUUCA |
| UAAUCUUAAAAGUGUAAUU | AUUAGAAUUUUCACAUUAA |
| UAAUUGUGUGACUUUUUCA | AUUAACACACUGAAAAAGU |
| AGAAACUGAUUUAUGAUCA | UCUUUGACUAAAUACUAGU |
| GAUUUGUAUAGUUUUAUAA | CUAAACAUAUCAAAAUAUU |
| AUUUGUAUAGUUUUAUAAA | UAAACAUAUCAAAAUAUUU |
| UUUAUAAAACUCAGUUAAA | AAAUAUUUUGAGUCAAUUU |
| AAAAUGUCUGUUUCAAUGA | UUUUACAGACAAAGUUACU |
| GUAUUAAACUUGUCAGAAU | CAUAAUUUGAACAGUCUUA |
| CUUAUUAUGAGGCUAUUAA | GAAUAAUACUCCGAUAAUU |
| UAUUAUGAGGCUAUUAkAA | AUAAUACUCCGAUAAUUUU |
| UAAAAGAAUCCAAAUUCAA | AUUUUCUUAGGUUUAAGUU |
| AAAAGAAUCCAAAUUCAAA | UUUUCUUAGGUUUAAGUUU |
| AGAAUCCAAAUUCAAACUA | UCUUAGGUUUAAGUUUGAU |
| AAUCCAAAUUCAAACUAAA | UUAGGUUUAAGUUUGAUUU |
| GGAGGUCUGGCCUAUAAAG | CCUCCAGACCGGAUAUUUC |
| GAGGUCUGGCCUAUAAAGU | CUCCAGACCGGAUAUUUCA |
| AGUGCAGGGCAUCAUCAAU | UCACGUCCCGUAGUAGUUA |
| GUGCAGGGCAUCAUCAAUU | CACGUCCCGUAGUAGUUAA |
| GCAGGGCAUCAUCAAUUUC | CGUCCCGUAGUAGUUAAAG |
| GCAUCAUCAAUUUCGAGCA | CGUAGUAGUUIMAGCUCGU |
| CAAUUUCGAGCAGAAGGAA | GUUAAAGCUCGUCUUCCUU |
| AAUUUCGAGCAGAAGGAAA | UUAAAGCUCGUCUUCCUUU |
| UUCGAGCAGAAGGAAAGUA | AAGCUCGUCUUCCUUUCAU |
| GUAAUGGACCAGUGAAGGU | CAUUACCUGGUCACUUCCA |
| GAAGGUGUGGGGAAGCAUU | CUUCCACACCCCUUCGUAA |
| GUGGGGAAGCAUUAAAGGA | CACCCCUUCGUAAUUUCCU |
| GGAAGCAUUAAAGGACUGA | CCUUCGUAAUUUCCUGACU |
| CUGAAGGCCUGCAUGGAUU | GACUUCCGGACGUACCUAA |
| AGGCCUGCAUGGAUUCCAU | UCCGGACGUACCUAAGGUA |
| CCUGCAUGGAUUCCAUGUU | GGACGUACCUAAGGUACAA |
| AUGGAUUCCAUGUUCAUGA | UACCUAAGGUACAAGUACU |
| GGAUUCCAUGUUCAUGAGU | CCUAAGGUACAAGUACUCA |
| GAUUCCAUGUUCAUGAGUU | CUAAGGUACAAGUACUCAA |
| GUUUGGAGAUAAUACAGCA | CAAACCUCUAUUAUGUCGU |
| AAUACAGCAGGCUGUACCA | UUAUGUCGUCCGACAUGGU |
| GCAGGUCCUCACUUUAAUC | CGUCCAGGAGUGAAAUUAG |
| CCUCACUUUAAUCCUCUAU | GGAGUGAAAUUAGGAGAUA |
| UCACUUUAAUCCUCUAUCC | AGUGAAAUUAGGAGAUAGG |
| CACUUUAAUCCUCUAUCCA | GUGAAAUUAGGAGAUAGGU |
| CUUUAAUCCUCUAUCCAGA | GAAAUUAGGAGAUAGGUCU |
| UUUAAUCCUCUAUCCAGAA | AAAUUAGGAGAUAGGUCUU |
| AAUCCUCUAUCCAGAAAAC | UUAGGAGAUAGGUCUUUUG |
| AUCCUCUAUCCAGAAAACA | UAGGAGAUAGGUCUUUUGU |
| GAAAACACGGUGGGCCAAA | CUUUUGUGCCACCCGGUUU |
| GUGGGCCAAAGGAUGAAGA | CACCCGGUUUCCUACUUCU |
| CAAAGGAUGAAGAGAGGCA | GUUUCCUACUUCUCUCCGU |
| GGAUGAAGAGAGGCAUGUU | CCUACUUCUCUCCGUACAA |
| AGAGGCAUGUUGGAGACUU | UCUCCGUACAACCUCUGAA |
| UUGGAGACUUGGGCAAUGU | AACCUCUGAACCCGUUACA |
| AGACUUGGGCAAUGUGACU | UCUGAACCCGUUACACUGA |
| UGGGCAAUGUGACUGCUGA | ACCCGUUACACUGACGACU |
| AAGAUGGUGUGGCCGAUGU | UUCUACCACACCGGCUACA |
| GUGUGGCCGAUGUGUCUAU | CACACCGGCUACACAGAUA |
| UGUGGCCGAUGUGUCUAUU | ACACCGGCUACACAGAUAA |
| CCGAUGUGUCUAUUGAAGA | GGCUACACAGAUAACUUCU |
| UCUAUUGAAGAUUCUGUGA | AGAUAACUUCUAAGACACU |
| CUAUUGAAGAUUCUGUGAU | GAUAACUUCUAAGACACUA |
| AAGAUUCUGUGAUCUCACU | UUCUAAGACACUAGAGUGA |
| UUCUGUGAUCUCACUCUCA | AAGACACUAGAGUGAGAGU |
| CUCUCAGGAGACCAUUGCA | GAGAGUCCUCUGGUAACGU |
| GGAGACCAUUGCAUCAUUG | CCUCUGGUAACGUAGUAAC |
| UUGCAUCAUUGGCCGCACA | AACGUAGUAACCGGCGUGU |
| UCCAUGAAAAAGCAGAUGA | AGGUACUUUUUCGUCUACU |
| AGAUGACUUGGGCAAAGGU | UCUACUGAACCCGUUUCCA |
| UGACUUGGGCAAAGGUGGA | ACUGAACCCGUUUCCACCU |
| CUUGGGCAAAGGUGGAAAU | GAACCCGUUUCCACCUUUA |
| UGGGCAAAGGUGGAAAUGA | ACCCGUUUCCACCUUUACU |
| GGUGGAAAUGAAGAAAGUA | CCACCUUUACUUCUUUCAU |
| UGGAAAUGAAGAAAGUACA | ACCUUUACUUCUUUCAUGU |
| AAAGACAGGAAACGCUGGA | UUUCUGUCCUUUGCGACCU |
| GGAAACGCUGGAAGUCGUU | CCUUUGCGACCUUCAGCAA |
| GGGAUCGCCCAAUAAACAU | CCCUAGCGGGUUAUUUGUA |
| AAUAAACAUUCCCUUGGAU | UUAUUUGUAAGGGAACCUA |
| UAAACAUUCCCUUGGAUGU | AUUUGUAAGGGAACCUACA |
| UGUAGUCUGAGGCCCCUUA | ACAUCAGACUCCGGGGAAU |
| AGUCUGAGGCCCCUUAACU | UCAGACUCCGGGGAAUUGA |
| UCUGAGGCCCCUUAACUCA | AGACUCCGGGGAAUUGAGU |
| UGAGGCCCCUUAACUCAUC | ACUCCGGGGAAUUGAGUAG |
| GAGGCCCCUUAACUCAUCU | CUCCGGGGAAUUGAGUAGA |
| CCCUUAACUCAUCUGUUAU | GGGAAUUGAGUAGACAAUA |
| UUAACUCAUCUGUUAUCCU | AAUUGAGUAGACAAUAGGA |
| UGUUAUCCUGCUAGCUGUA | ACAAUAGGACGAUCGACAU |
| AUCCUGCUAGCUGUAGAAA | UAGGACGAUCGACAUCUUU |
| UCCUGCUAGCUGUAGAAAU | AGGACGAUCGACAUCUUUA |
| CCUGCUAGCUGUAGAAAUG | GGACGAUCGACAUCUUUAC |
| GCUAGCUGUAGAAAUGUAU | CGAUCGACAUCUUUACAUA |
| CUGUAGAAAUGUAUCCUGA | GACAUCUUUACAUAGGACU |
| AGUGUAAUUGUGUGACUUU | UCACAUUAACACACUGAAA |
| GUGUAAUUGUGUGACUUUU | CACAUUAACACACUGAAAA |
| AUUGUGUGACUUUUUCAGA | UAACACACUGAAAAAGUCU |
| UGUGUGACUUUUUCAGAGU | ACACACUGAAAAAGUCUCA |
| GUGUGACUUUUUCAGAGUU | CACACUGAAAAAGUCUCAA |
| UGACUUUUUCAGAGUUGCU | ACUGAAAAAGUCUCAACGA |
| UUUCAGAGUUGCUUUAAAG | AAAGUCUCAACGAAAUUUC |
| UUCAGAGUUGCUUUAAAGU | kAGUCUCAACGAAAUUUCA |
| GAGUUGCUUUAAAGUACCU | CUCAACGAAAUUUCAUGGA |
| UUGCUUUAAAGUACCUGUA | AACGAAAUUUCAUGGACAU |
| GCUUUAAAGUACCUGUAGU | CGAAAUUUCAUGGACAUCA |
| AAAGUACCUGUAGUGAGAA | UUUCAUGGACAUCACUCUU |
| ACCUGUAGUGAGAAACUGA | UGGACAUCACUCUUUGACU |
| CUGUAGUGAGAAACUGAUU | GACAUCACUCUUUGACUAA |
| UGUAGUGAGAACUGAUUUA | CAUCACUCUUUGACUAAAA |
| GAAACUGAUUUAUGAUCAC | CUUUGACUAAAUACUAGUG |
| UUUAUGAUCACUUGGAAGA | AAAUACUAGUGAACCUUCU |
| UUAUGAUCACUUGGAAGAU | AAUACUAGUGAACCUUCUA |
| UAUGAUCACUUGGAAGAUU | AUACUAGUGAACCUUCUAA |
| UGAUCACUUGGAAGAUUUG | ACUAGUGAACCUUCUAAAC |
| CUUGGAAGAUUUGUAUAGU | GAACCUUCUAAACAUAUCA |
| AAAUGUCUGUUUCAAUGAC | UUUACAGACAAAGUUACUG |
| UCUGUUUCAAUGACCUGUA | AGACAAAGUUACUGGACAU |
| UGUUUCAAUGACCUGUAUU | ACAAAGUUACUGGACAUAA |
| UCAAUGACCUGUAUUUUGC | AGUUACUGGACAUAAAACG |
| AAUGACCUGUAUUUUGCCA | UUACUGGACAUAAAACGGU |
| ACCUGUAUUUUGCCAGACU | UGGACAUAAAACGGUCUGA |
| CCUGUAUUUUGCCAGACUU | GGACAUAAAACGGUCUGAA |
| UGUAUUUUGCCAGACUUAA | ACAUAAAACGGUCUGAAUU |
| GCCAGACUUAAAUCACAGA | CGGUCUGAAUUUAGUGUCU |
| CCAGACUUAAAUCACAGAU | GGUCUGAAUUUAGUGUCUA |
| UUAAAUCACAGAUGGGUAU | AAUUUAGUGUCUACCCAUA |
| UAAAUCACAGAUGGGUAUU | AUUUAGUGUCUACCCAUAA |
| UCACAGAUGGGUAUUAAAC | AGUGUCUACCCAUAAUUUG |
| CACAGAUGGGUAUUAAACU | GUGUCUACCCAUAAUUUGA |
| AUGGGUAUUAAACUUGUCA | UACCCAUAAUUUGAACAGU |
| GGGUAUUAAACUUGUCAGA | CCCAUAAUUUGAACAGUCU |
| CUUGUCAGAAUUUCUUUGU | GAACAGUCUUAAAGAAACA |
| GUCAGAAUUUCUUUGUCAU | CAGUCUUAAAGAAACAGUA |
| UUGUCAUUCAAGCCUGUGA | AACAGUAAGUUCGGACACU |
| UGUCAUUCAAGCCUGUGAA | ACAGUAAGUUCGGACACUU |
| GUCAUUCAAGCCUGUGAAU | CAGUAAGUUCGGACACUUA |
| GUAUGGCACUUAUUAUGAG | CAUACCGUGAAUAAUACUC |
| GCACUUAUUAUGAGGCUAU | CGUGAAUAAUACUCCGAUA |
| CACUUAUUAUGAGGCUAUU | GUGAAUAAUACUCCGAUAA |
| UGAGGCUAUUAAAAGAAUC | ACUCCGAUAAUUUUCUUAG |
| GGCUAUUAAAAGAAUCCAA | CCGAUAAUUUUCUUAGGUU |
| GGUGCUGGUUUGCGUCGUA | CCACGACCAAACGCAGCAU |
| GUCUGGGGUUUCCGUUGCA | CAGACCCCAAAGGCAACGU |
| CCAGUGCAGGGCAUCAUCA | GGUCACGUCCCGUAGUAGU |
| GAAAUGAAGAAAGUACAAA | CUUUACUUCUUUCAUGUUU |
| AAUGAAGAAAGUACAAAGA | UUACUUCUUUCAUGUUUCU |
| UAGAAAUGUAUCCUGAUAA | AUCUUUACAUAGGACUAUU |
| AAUGUAUCCUGAUAAACAU | UUACAUAGGACUAUUUGUA |
| UGUAUCCUGAUAAACAUUA | ACAUAGGACUAUUUGUAAU |
| UCCUGAUAAACAUUAAACA | AGGACUAUUUGUAAUUUGU |
| AUAAACAUUAAACACUGUA | UAUUUGUAAUUUGUGACAU |
| AAACAUUAAACACUGUAAU | UUUGUAAUUUGUGACAUUA |
| CUGUAAUCUUAAAAGUGUA | GACAUUAGAAUUUUCACAU |
| UGUAAUCUUAAAAGUGUAA | ACAUUAGAAUUUUCACAUU |
| UAAAAGUGUAAUUGUGUGA | AUUUUCACAUUAACACACU |
| UGUAAUUGUGUGACUUUUU | ACAUUAACACACUGAAAAA |
| UUUUUCAGAGUUGCUUUAA | AAAAAGUCUCAACGAAAUU |
| UGAGAAACUGAUUUAUGAU | ACUCUUUGACUAAAUACUA |
| UUGGAAGAUUUGUAUAGUU | AACCUUCUAAACAUAUCAA |
| UGGAAGAUUUGUAUAGUUU | ACCUUCUAAACAUAUCAAA |
| GGAAGAUUUGUAUAGUUUU | CCUUCUAAACAUAUCAAAA |
| UGUAUAGUUUUAUAAAACU | ACAUAUCAAAAUAUUUUGA |
| GUUUUAUAAAACUCAGUUA | CAAAAUAUUUUGAGUCAAU |
| UUUUAUAAAACUCAGUUAA | AAAAUAUUUUGAGUCAAUU |
| UAUAAAACUCAGUUAAAAU | AUAUUUUGAGUCAAUUUUA |
| UAAAACUCAGUUAAAAUGU | AUUUUGAGUCAAUUUUACA |
| AAACUCAGUUAAAAUGUCU | UUUGAGUCAAUUUUACAGA |
| UCAGUUAAAAUGUCUGUUU | AGUCAAUUUUACAGACAAA |
| AGUUAAAAUGUCUGUUUCA | UCAAUUUUACAGACAAAGU |
| GUUAAAAUGUCUGUUUCAA | CAAUUUUACAGACAAAGUU |
| UUAAAAUGUCUGUUUCAAU | AAUUUUACAGACAAAGUUA |
| UAUUUUGCCAGACUUAAAU | AUAAAACGGUCUGAAUUUA |
| UAUUAAACUUGUCAGAAUU | AUAAUUUGAACAGUCUUAA |
| AUUAAACUUGUCAGAAUUU | UAAUUUGAACAGUCUUAAA |
| UAAACUUGUCAGAAUUUCU | AUUUGAACAGUCUUAAAGA |
| AAACUUGUCAGAAUUUCUU | UUUGAACAGUCUUAAAGAA |
| UCAGAAUUUCUUUGUCAUU | AGUCUUAAAGAAACAGUAA |
| ACUUAUUAUGAGGCUAUUA | UGAAUAAUACUCCGAUAAU |
| UAUGAGGCUAUUAAAAGAA | AUACUCCGAUAAUUUUCUU |
| AUGAGGCUAUUAAAAGAAU | UACUCCGAUAAUUUUCUUA |
| CUAUUAAAAGAAUCCAAAU | GAUAAUUUUCUUAGGUUUA |
| GGUCUGGCCUAUAAAGUAG | CCAGACCGGAUAUUUCAUC |
| GUCUGGCCUAUAAAGUAGU | CAGACCGGAUAUUUCAUCA |
| UCUGGCCUAUAAAGUAGUC | AGACCGGAUAUUUCAUCAG |
| CCUAUAAAGUAGUCGCGGA | GGAUAUUUCAUCAGCGCCU |
| AUAAAGUAGUCGCGGAGAC | UAUUUCAUCAGCGCCUCUG |
| UGCUGGUUUGCGUCGUAGU | ACGACCAAACGCAGCAUCA |
| AUCAUCAAUUUCGAGCAGA | UAGUAGUUAAAGCUCGUCU |
| UUUCGAGCAGAAGGAAAGU | AAAGCUCGUCUUCCUUUCA |
| GAGCAGAAGGAAAGUAAUG | CUCGUCUUCCUUUCAUUAC |
| GGAAAGUAAUGGACCAGUG | CCUUUCAUUACCUGGUCAC |
| UGAAGGUGUGGGGAAGCAU | ACUUCCACACCCCUUCGUA |
| GUGUGGGGAAGCAUUAAAG | CACACCCCUUCGUAAUUUC |
| GGGGAAGCAUUAAAGGACU | CCCCUUCGUAAUUUCCUGA |
| AAGCAUUAAAGGACUGACU | UUCGUAAUUUCCUGACUGA |
| CAUUAAAGGACUGACUGAA | GUAAUUUCCUGACUGACUU |
| UUAAAGGACUGACUGAAGG | AAUUUCCUGACUGACUUCC |
| ACUGACUGAAGGCCUGCAU | UGACUGACUUCCGGACGUA |
| ACUGAAGGCCUGCAUGGAU | UGACUUCCGGACGUACCUA |
| UGAAGGCCUGCAUGGAUUC | ACUUCCGGACGUACCUAAG |
| AAGGCCUGCAUGGAUUCCA | UUCCGGACGUACCUAAGGU |
| GCCUGCAUGGAUUCCAUGU | CGGACGUACCUAAGGUACA |
| CUGCAUGGAUUCCAUGUUC | GACGUACCUAAGGUACAAG |
| UGGAUUCCAUGUUCAUGAG | ACCUAAGGUACAAGUACUC |
| AUGUUCAUGAGUUUGGAGA | UACAAGUACUCAAACCUCU |
| UGUUCAUGAGUUUGGAGAU | ACAAGUACUCAAACCUCUA |
| AUGAGUUUGGAGAUAAUAC | UACUCAAACCUCUAUUAUG |
| AGAUAAUACAGCAGGCUGU | UCUAUUAUGUCGUCCGACA |
| GAUAAUACAGCAGGCUGUA | CUAUUAUGUCGUCCGACAU |
| AGGUCCUCACUUUAAUCCU | UCCAGGAGUGAAAUUAGGA |
| GUCCUCACUUUAAUCCUCU | CAGGAGUGAAAUUAGGAGA |
| AGAAAACACGGUGGGCCAA | UCUUUUGUGCCACCCGGUU |
| AAAACACGGUGGGCCAAAG | UUUUGUGCCACCCGGUUUC |
| GGGCCAAAGGAUGAAGAGA | CCCGGUUUCCUACUUCUCU |
| AAGAGAGGCAUGUUGGAGA | UUCUCUCCGUACAACCUCU |
| AGAGAGGCAUGUUGGAGAC | UCUCUCCGUACAACCUCUG |
| GAGGCAUGUUGGAGACUUG | CUCCGUACAACCUCUGAAC |
| AUGUUGGAGACUUGGGCAA | UACAACCUCUGAACCCGUU |
| GGCAAUGUGACUGCUGACA | CCGUUACACUGACGACUGU |
| GACUGCUGACAAAGAUGGU | CUGACGACUGUUUCUACCA |
| ACAAAGAUGGUGUGGCCGA | UGUUUCUACCACACCGGCU |
| CAAAGAUGGUGUGGCCGAU | GUUUCUACCACACCGGCUA |
| GUGGCCGAUGUGUCUAUUG | CACCGGCUACACAGAUAAC |
| GCCGAUGUGUCUAUUGAAG | CGGCUACACAGAUAACUUC |
| GAUGUGUCUAUUGAAGAUU | CUACACAGAUAACUUCUAA |
| AUGUGUCUAUUGAAGAUUC | UACACAGAUAACUUCUAAG |
| UGUCUAUUGAAGAUUCUGU | ACAGAUAACUUCUAAGACA |
| UAUUGAAGAUUCUGUGAUC | AUAACUUCUAAGACACUAG |
| AUUGAAGAUUCUGUGAUCU | UAACUUCUAAGACACUAGA |
| GAUUCUGUGAUCUCACUCU | CUAAGACACUAGAGUGAGA |
| UGUGAUCUCACUCUCAGGA | ACACUAGAGUGAGAGUCCU |
| CUCACUCUCAGGAGACCAU | GAGUGAGAGUCCUCUGGUA |
| UCACUCUCAGGAGACCAUU | AGUGAGAGUCCUCUGGUAA |
| UCUCAGGAGACCAUUGCAU | AGAGUCCUCUGGUAACGUA |
| CAGGAGACCAUUGCAUCAU | GUCCUCUGGUAACGUAGUA |
| UGGUCCAUGAAAAAGCAGA | ACCAGGUACUUUUUCGUCU |
| GGUCCAUGAAAAAGCAGAU | CCAGGUACUUUUUCGUCUA |
| CCAUGAAAAAGCAGAUGAC | GGUACUUUUUCGUCUACUG |
| AUGAAAAAGCAGAUGACUU | UACUUUUUCGUCUACUGAA |
| UGAAAAAGCAGAUGACUUG | ACUUUUUCGUCUACUGAAC |
| AAAGCAGAUGACUUGGGCA | UUUCGUCUACUGAACCCGU |
| AAGCAGAUGACUUGGGCAA | UUCGUCUACUGAACCCGUU |
| AGCAGAUGACUUGGGCAAA | UCGUCUACUGAACCCGUUU |
| GCAGAUGACUUGGGCAAAG | CGUCUACUGAACCCGUUUC |
| GACUUGGGCAAAGGUGGAA | CUGAACCCGUUUCCACCUU |
| ACUUGGGCAAAGGUGGAAA | UGAACCCGUUUCCACCUUU |
| AGAAAGUACAAAGACAGGA | UCUUUCAUGUUUCUGUCCU |
| AAGACAGGAAACGCUGGAA | UUCUGUCCUUUGCGACCUU |
| GACAGGAAACGCUGGAAGU | CUGUCCUUUGCGACCUUCA |
| AGGAAACGCUGGAAGUCGU | UCCUUUGCGACCUUCAGCA |
| GAAACGCUGGAAGUCGUUU | CUUUGCGACCUUCAGCAAA |
| AAACGCUGGAAGUCGUUUG | UUUGCGACCUUCAGCAAAC |
| UGGAAGUCGUUUGGCUUGU | ACCUUCAGCAAACCGAACA |
| GGAAGUCGUUUGGCUUGUG | CCUUCAGCAAACCGAACAC |
| GAAGUCGUUUGGCUUGUGG | CUUCAGCAAACCGAACACC |
| GUUUGGCUUGUGGUGUAAU | CAAACCGAACACCACAUUA |
| GCUUGUGGUGUAAUUGGGA | CGAACACCACAUUAACCCU |
| CUUGUGGUGUAAUUGGGAU | GAACACCACAUUAACCCUA |
| UGUAAUUGGGAUCGCCCAA | ACAUUAACCCUAGCGGGUU |
| GUAAUUGGGAUCGCCCAAU | CAUUAACCCUAGCGGGUUA |
| UUGGGAUCGCCCAAUAAAC | AACCCUAGCGGGUUAUUUG |
| AUCGCCCAAUAAACAUUCC | UAGCGGGUUAUUUGUAAGG |
| UAGUCUGAGGCCCCUUAAC | AUCAGACUCCGGGGAAUUG |
| CUGAGGCCCCUUAACUCAU | GACUCCGGGGAAUUGAGUA |
| GGCCCCUUAACUCAUCUGU | CCGGGGAAUUGAGUAGACA |
| GCCCCUUAACUCAUCUGUU | CGGGGAAUUGAGUAGACAA |
| CCUUAACUCAUCUGUUAUC | GGAAUUGAGUAGACAAUAG |
| UAACUCAUCUGUUAUCCUG | AUUGAGUAGACAAUAGGAC |
| ACUCAUCUGUUAUCCUGCU | UGAGUAGACAAUAGGACGA |
| CUGUUAUCCUGCUAGCUGU | GACAAUAGGACGAUCGACA |
| CUGCUAGCUGUAGAAAUGU | GACGAUCGACAUCUUUACA |
| CUAGCUGUAGAAAUGUAUC | GAUCGACAUCUUUACAUAG |
| UGUAGAAAUGUAUCCUGAU | ACAUCUUUACAUAGGACUA |
| GAAAUGUAUCCUGAUAAAC | CUUUACAUAGGACUAUUUG |
| AAGUGUAAUUGUGUGACUU | UUCACAUUAACACACUGAA |
| UGUGACUUUUUCAGAGUUG | ACACUGAAAAAGUCUCAAC |
| ACUUUUUCAGAGUUGCUUU | UGAAAAAGUCUCAACGAAA |
| CAGAGUUGCUUUAAAGUAC | GUCUCAACGAAAUUUCAUG |
| AGAGUUGCUUUAAAGUACC | UCUCAACGAAAUUUCAUGG |
| UUUAAAGUACCUGUAGUGA | AAAUUUCAUGGACAUCACU |
| CCUGUAGUGAGAAACUGAU | GGACAUCACUCUUUGACUA |
| AGUGAGAAACUGAUUUAUG | UCACUCUUUGACUAAAUAC |
| ACUGAUUUAUGAUCACUUG | UGACUAAAUACUAGUGAAC |
| GAUCACUUGGAAGAUUUGU | CUAGUGAACCUUCUAAACA |
| ACUUGGAAGAUUUGUAUAG | UGAACCUUCUAAACAUAUC |
| ACUCAGUUAAAAUGUCUGU | UGAGUCAAUUUUACAGACA |
| CAGUUAAAAUGUCUGUUUC | GUCAAUUUUACAGACAAAG |
| AUGUCUGUUUCAAUGACCU | UACAGACAAAGUUACUGGA |
| UUCAAUGACCUGUAUUUUG | AAGUUACUGGACAUAAAAC |
| AUUUUGCCAGACUUAAAUC | UAAAACGGUCUGAAUUUAG |
| AGACUUAAAUCACAGAUGG | UCUGAAUUUAGUGUCUACC |
| CUUAAAUCACAGAUGGGUA | GAAUUUAGUGUCUACCCAU |
| GAUGGGUAUUAAACUUGUC | CUACCCAUAAUUUGAACAG |
| GGUAUUAAACUUGUCAGAA | CCAUAAUUUGAACAGUCUU |
| UUGUCAGAAUUUCUUUGUC | AACAGUCUUAAAGAAACAG |
| CUUUGUCAUUCAAGCCUGU | GAAACAGUAAGUUCGGACA |
| GCCUGUGAAUAAAAACCCU | CGGACACUUAUUUUUGGGA |
| UGAAUAAAAACCCUGUAUG | ACUUAUUUUUGGGACAUAC |
| GAAUAAAAACCCUGUAUGG | CUUAUUUUUGGGACAUACC |
| AAAAACCCUGUAUGGCACU | UUUUUGGGACAUACCGUGA |
| CUGUAUGGCACUUAUUAUG | GACAUACCGUGAAUAAUAC |
| UGGCACUUAUUAUGAGGCU | ACCGUGAAUAAUACUCCGA |
| GGCACUUAUUAUGAGGCUA | CCGUGAAUAAUACUCCGAU |
| AGGCUAUUAAAAGAAUCCA | UCCGAUAAUUUUCUUAGGU |
| CGCGGAGGUCUGGCCUAUA | GCGCCUCCAGACCGGAUAU |
| GGAGACGGGGUGCUGGUUU | CCUCUGCCCCACGACCAAA |
| GUAGUCUCCUGCAGCGUCU | CAUCAGAGGACGUCGCAGA |
| GCAGUCCUCGGAACCAGGA | CGUCAGGAGCCUUGGUCCU |
| AGGCCGUGUGCGUGCUGAA | UCCGGCACACGCACGACUU |
| CCAGUGAAGGUGUGGGGAA | GGUCACUUCCACACCCCUU |
| GUGAAGGUGUGGGGAAGCA | CACUUCCACACCCCUUCGU |
| AGCAGGCUGUACCAGUGCA | UCGUCCGACAUGGUCACGU |
| CCAGUGCAGGUCCUCACUU | GGUCACGUCCAGGAGUGAA |
| AACACGGUGGGCCAAAGGA | UUGUGCCACCCGGUUUCCU |
| ACACGGUGGGCCAAAGGAU | UGUGCCACCCGGUUUCCUA |
| ACGGUGGGCCAAAGGAUGA | UGCCACCCGGUUUCCUACU |
| CGGUGGGCCAAAGGAUGAA | GCCACCCGGUUUCCUACUU |
| GCCGCACACUGGUGGUCCA | CGGCGUGUGACCACCAGGU |
| AUGUAUCCUGAUAAACAUU | UACAUAGGACUAUUUGUAA |
| GAUAAACAUUAAACACUGU | CUAUUUGUAAUUUGUGACA |
| ACAUUAAACACUGUAAUCU | UGUAAUUUGUGACAUUAGA |
| CAUUAAACACUGUAAUCUU | GUAAUUUGUGACAUUAGAA |
| ACUGUAAUCUUAAAAGUGU | UGACAUUAGAAUUUUCACA |
| GUAAUCUUAAAAGUGUAAU | CAUUAGAAUUUUCACAUUA |
| AUCUUAAAAGUGUAAUUGU | UAGAAUUUUCACAUUAACA |
| UAGUGAGAAACUGAUUUAU | AUCACUCUUUGACUAAAUA |
| AMACUGAUUUAUGAUCACU | UUUGACUAAAUACUAGUGA |
| AAGAUUUGUAUAGUUUUAU | UUCUAAACAUAUCAAAAUA |
| UUGUAUAGUUUUAUAAAAC | AACAUAUCAAAAUAUUUUG |
| GUAUAGUUUUAUAAAACUC | CAUAUCAAAAUAUUUUGAG |
| UAUAGUUUUAUAAAACUCA | AUAUCAAAAUAUUUUGAGU |
| AUAAAACUCAGUUAAAAUG | UAUUUUGAGUCAAUUUUAC |
| AAAACUCAGUUAAAAUGUC | UUUUGAGUCAAUUUUACAG |
| UAAAAUGUCUGUUUCAAUG | AUUUUACAGACAAAGUUAC |
| UUUCAAUGACCUGUAUUUU | AAAGUUACUGGACAUAAAA |
| UUAAACUUGUCAGAAUUUC | AAUUUGAACAGUCUUAAAG |
| AACUUGUCAGAAUUUCUUU | UUGAACAGUCUUAAAGAAA |
| GCUAUUAAAAGAAUCCAAA | CGAUAAUUUUCUUAGGUUU |
| AAGAAUCCAAAUUCAAACU | UUCUUAGGUUUAAGUUUGA |
| UAUAAAGUAGUCGCGGAGA | AUAUUUCAUCAGCGCCUCU |
| CUGGUUUGCGUCGUAGUCU | GACCAAACGCAGCAUCAGA |
| UGGUUUGCGUCGUAGUCUC | ACCAAACGCAGCAUCAGAG |
| UUUGCGUCGUAGUCUCCUG | AAACGCAGCAUCAGAGGAC |
| UAGCGAGUUAUGGCGACGA | AUCGCUCAAUACCGCUGCU |
| CAGGGCAUCAUCAAUUUCG | GUCCCGUAGUAGUUAAAGC |
| CAUCAUCAAUUUCGAGCAG | GUAGUAGUUAAAGCUCGUC |
| AUUUCGAGCAGAAGGAAAG | UAAAGCUCGUCUUCCUUUC |
| AAGUAAUGGACCAGUGAAG | UUCAUUACCUGGUCACUUC |
| AAUGGACCAGUGAAGGUGU | UUACCUGGUCACUUCCACA |
| AUGGACCAGUGAAGGUGUG | UACCUGGUCACUUCCACAC |
| UGUGGGGAAGCAUUAAAGG | ACACCCCUUCGUAAUUUCC |
| GGGAAGCAUUAAAGGACUG | CCCUUCGUAAUUUCCUGAC |
| AUUAAAGGACUGACUGAAG | UAAUUUCCUGACUGACUUC |
| UAAAGGACUGACUGAAGGC | AUUUCCUGACUGACUUCCG |
| UCCAUGUUCAUGAGUUUGG | AGGUACAAGUACUCAAACC |
| GAGUUUGGAGAUAAUACAG | CUCAAACCUCUAUUAUGUC |
| GGAGAUAAUACAGCAGGCU | CCUCUAUUAUGUCGUCCGA |
| AUAAUACAGCAGGCUGUAC | UAUUAUGUCGUCCGACAUG |
| UAAUACAGCAGGCUGUACC | AUUAUGUCGUCCGACAUGG |
| UACAGCAGGCUGUACCAGU | AUGUCGUCCGACAUGGUCA |
| CAGGUCCUCACUUUAAUCC | GUCCAGGAGUGAAAUUAGG |
| GGUCCUCACUUUAAUCCUC | CCAGGAGUGAAAUUAGGAG |
| CUCACUUUAAUCCUCUAUC | GAGUGAAAUUAGGAGAUAG |
| UCCUCUAUCCAGAAAACAC | AGGAGAUAGGUCUUUUGUG |
| UCUAUCCAGAAAACACGGU | AGAUAGGUCUUUUGUGCCA |
| UGGGCCAAAGGAUGAAGAG | ACCCGGUUUCCUACUUCUC |
| GCCAAAGGAUGAAGAGAGG | CGGUUUCCUACUUCUCUCC |
| AAAGGAUGAAGAGAGGCAU | UUUCCUACUUCUCUCCGUA |
| AGGAUGAAGAGAGGCAUGU | UCCUACUUCUCUCCGUACA |
| GAUGAAGAGAGGCAUGUUG | CUACUUCUCUCCGUACAAC |
| GAGAGGCAUGUUGGAGACU | CUCUCCGUACAACCUCUGA |
| CAUGUUGGAGACUUGGGCA | GUACAACCUCUGAACCCGU |
| UGUUGGAGACUUGGGCAAU | ACAACCUCUGAACCCGUUA |
| GUUGGAGACUUGGGCAAUG | CAACCUCUGAACCCGUUAC |
| UGGAGACUUGGGCAAUGUG | ACCUCUGAACCCGUUACAC |
| CUUGGGCAAUGUGACUGCU | GAACCCGUUACACUGACGA |
| GUGACUGCUGACAAAGAUG | CACUGACGACUGUUUCUAC |
| UGACUGCUGACAAAGAUGG | ACUGACGACUGUUUCUACC |
| CUGCUGACAAAGAUGGUGU | GACGACUGUUUCUACCACA |
| UGACAAAGAUGGUGUGGCC | ACUGUUUCUACCACACCGG |
| UUGAAGAUUCUGUGAUCUC | AACUUCUAAGACACUAGAG |
| GAAGAUUCUGUGAUCUCAC | CUUCUAAGACACUAGAGUG |
| AGAUUCUGUGAUCUCACUC | UCUAAGACACUAGAGUGAG |
| AUUCUGUGAUCUCACUCUC | UAAGACACUAGAGUGAGAG |
| UCUGUGAUCUCACUCUCAG | AGACACUAGAGUGAGAGUC |
| UCUCACUCUCAGGAGACCA | AGAGUGAGAGUCCUCUGGU |
| CACUCUCAGGAGACCAUUG | GUGAGAGUCCUCUGGUAAC |
| ACUCUCAGGAGACCAUUGC | UGAGAGUCCUCUGGUAACG |
| GAGACCAUUGCAUCAUUGG | CUCUGGUAACGUAGUAACC |
| AGACCAUUGCAUCAUUGGC | UCUGGUAACGUAGUAACCG |
| CUGGUGGUCCAUGAAAAAG | GACCACCAGGUACUUUUUC |
| UGGUGGUCCAUGAAAAAGC | ACCACCAGGUACUUUUUCG |
| CAUGAAAAAGCAGAUGACU | GUACUUUUUCGUCUACUGA |
| AAAAAGCAGAUGACUUGGG | UUUUUCGUCUACUGAACCC |
| AAAAGCAGAUGACUUGGGC | UUUUCGUCUACUGAACCCG |
| CAGAUGACUUGGGCAAAGG | GUCUACUGAACCCGUUUCC |
| UUGGGCAAAGGUGGAAAUG | AACCCGUUUCCACCUUUAC |
| GGCAAAGGUGGAAAUGAAG | CCGUUUCCACCUUUACUUC |
| AAGGUGGAAAUGAAGAAAG | UUCCACCUUUACUUCUUUC |
| GUGGAAAUGAAGAAAGUAC | CACCUUUACUUCUUUCAUG |
| AUGAAGAAAGUACAAAGAC | UACUUCUUUCAUGUUUCUG |
| GAAGAAAGUACAAAGACAG | CUUCUUUCAUGUUUCUGUC |
| AAGUACAAAGACAGGAAAC | UUCAUGUUUCUGUCCUUUG |
| UACAAAGACAGGAAACGCU | AUGUUUCUGUCCUUUGCGA |
| AGACAGGAAACGCUGGAAG | UCUGUCCUUUGCGACCUUC |
| ACAGGAAACGCUGGAAGUC | UGUCCUUUGCGACCUUCAG |
| AACGCUGGAAGUCGUUUGG | UUGCGACCUUCAGCAAACC |
| GCUGGAAGUCGUUUGGCUU | CGACCUUCAGCAAACCGAA |
| AAGUCGUUUGGCUUGUGGU | UUCAGCAAACCGAACACCA |
| UUGGCUUGUGGUGUAAUUG | AACCGAACACCACAUUAAC |
| UGGCUUGUGGUGUAAUUGG | ACCGAACACCACAUUAACC |
| GGCUUGUGGUGUAAUUGGG | CCGAACACCACAUUAACCC |
| GUGUAAUUGGGAUCGCCCA | CACAUUAACCCUAGCGGGU |
| GAUCGCCCAAUAAACAUUC | CUAGCGGGUUAUUUGUAAG |
| CGCCCAAUAAACAUUCCCU | GCGGGUUAUUUGUAAGGGA |
| GCCCAAUAAACAUUCCCUU | CGGGUUAUUUGUAAGGGAA |
| CCAAUAAACAUUCCCUUGG | GGUUAUUUGUAAGGGAACC |
| AUAAACAUUCCCUUGGAUG | UAUUUGUAAGGGAACCUAC |
| AACAUUCCCUUGGAUGUAG | UUGUAAGGGAACCUACAUC |
| CAUUCCCUUGGAUGUAGUC | GUAAGGGAACCUACAUCAG |
| AUUCCCUUGGAUGUAGUCU | UAAGGGAACCUACAUCAGA |
| CCUUGGAUGUAGUCUGAGG | GGAACCUACAUCAGACUCC |
| AGGCCCCUUAACUCAUCUG | UCCGGGGAAUUGAGUAGAC |
| CUUAACUCAUCUGUUAUCC | GAAUUGAGUAGACAAUAGG |
| UCAUCUGUUAUCCUGCUAG | AGUAGACAAUAGGACGAUC |
| CAUCUGUUAUCCUGCUAGC | GUAGACAAUAGGACGAUCG |
| AUCUGUUAUCCUGCUAGCU | UAGACAAUAGGACGAUCGA |
| UAGCUGUAGAAAUGUAUCC | AUCGACAUCUUUACAUAGG |
| AGCUGUAGAAAUGUAUCCU | UCGACAUCUUUACAUAGGA |
| CCUGAUAAACAUUAAACAC | GGACUAUUUGUAAUUUGUG |
| CACUGUAAUCUUAAAAGUG | GUGACAUUAGAAUUUUCAC |
| AAUUGUGUGACUUUUUCAG | UUAACACACUGAAAAAGUC |
| UUGUGUGACUUUUUCAGAG | AACACACUGAAAAAGUCUC |
| GACUUUUUCAGAGUUGCUU | CUGAAAAAGUCUCAACGAA |
| GUUGCUUUAAAGUACCUGU | CAACGAAAUUUCAUGGACA |
| UGCUUUAAAGUACCUGUAG | ACGAAAUUUCAUGGACAUC |
| CUUUAAAGUACCUGUAGUG | GAAAUUUCAUGGACAUCAC |
| UUAAAGUACCUGUAGUGAG | AAUUUCAUGGACAUCACUC |
| UACCUGUAGUGAGAAACUG | AUGGACAUCACUCUUUGAC |
| GAGAAACUGAUUUAUGAUC | CUCUUUGACUAAAUACUAG |
| CUCAGUUAAAAUGUCUGUU | GAGUCAAUUUUACAGACAA |
| AAUGUCUGUUUCAAUGACC | UUACAGACAAAGUUACUGG |
| CAAUGACCUGUAUUUUGCC | GUUACUGGACAUAAAACGG |
| UUUGCCAGACUUAAAUCAC | AAACGGUCUGAAUUUAGUG |
| UGCCAGACUUAAAUCACAG | ACGGUCUGAAUUUAGUGUC |
| CAGACUUAAAUCACAGAUG | GUCUGAAUUUAGUGUCUAC |
| CAGAUGGGUAUUAAACUUG | GUCUACCCAUAAUUUGAAC |
| ACUUGUCAGAAUUUCUUUG | UGAACAGUCUUAAAGAAAC |
| CAGAAUUUCUUUGUCAUUC | GUCUUAAAGAAACAGUAAG |
| AUUUCUUUGUCAUUCAAGC | UAAAGAAACAGUAAGUUCG |
| UUCUUUGUCAUUCAAGCCU | AAGAAACAGUAAGUUCGGA |
| AAGCCUGUGAAUAAAAACC | UUCGGACACUUAUUUUUGG |
| CUGUGAAUAAAAACCCUGU | GACACUUAUUUUUGGGACA |
| UAAAAACCCUGUAUGGCAC | AUUUUUGGGACAUACCGUG |
| UAUGGCACUUAUUAUGAGG | AUACCGUGAAUAAUACUCC |
| GAGGCUAUUAAAAGAAUCC | CUCCGAUAAUUUUCUUAGG |
| GGCGCGGAGGUCUGGCCUA | CCGCGCCUCCAGACCGGAU |
| GCGCGGAGGUCUGGCCUAU | CGCGCCUCCAGACCGGAUA |
| GAGACGGGGUGCUGGUUUG | CUCUGCCCCACGACCAAAC |
| ACGGGGUGCUGGUUUGCGU | UGCCCCACGACCAAACGCA |
| UGCGUCGUAGUCUCCUGCA | ACGCAGCAUCAGAGGACGU |
| CCUGCAGCGUCUGGGGUUU | GGACGUCGCAGACCCCAAA |
| GGGUUUCCGUUGCAGUCCU | CCCAAAGGCAACGUCAGGA |
| UUCCGUUGCAGUCCUCGGA | AAGGCAACGUCAGGAGCCU |
| UCCGUUGCAGUCCUCGGAA | AGGCAACGUCAGGAGCCUU |
| GUUGCAGUCCUCGGAACCA | CAACGUCAGGAGCCUUGGU |
| AGGACCUCGGCGUGGCCUA | UCCUGGAGCCGCACCGGAU |
| CCUCGGCGUGGCCUAGCGA | GGAGCCGCACCGGAUCGCU |
| GCGUGGCCUAGCGAGUUAU | CGCACCGGAUCGCUCAAUA |
| CGUGGCCUAGCGAGUUAUG | GCACCGGAUCGCUCAAUAC |
| CGACGAAGGCCGUGUGCGU | GCUGCUUCCGGCACACGCA |
| CGAAGGCCGUGUGCGUGCU | GCUUCCGGCACACGCACGA |
| AAGGCCGUGUGCGUGCUGA | UUCCGGCACACGCACGACU |
| UGUGCGUGCUGAAGGGCGA | ACACGCACGACUUCCCGCU |
| AGGGCGACGGCCCAGUGCA | UCCCGCUGCCGGGUCACGU |
| ACGGCCCAGUGCAGGGCAU | UGCCGGGUCACGUCCCGUA |
| GGCCCAGUGCAGGGCAUCA | CCGGGUCACGUCCCGUAGU |
| GCCCAGUGCAGGGCAUCAU | CGGGUCACGUCCCGUAGUA |
| GACUGAAGGCCUGCAUGGA | CUGACUUCCGGACGUACCU |
| ACAGCAGGCUGUACCAGUG | UGUCGUCCGACAUGGUCAC |
| CUGUACCAGUGCAGGUCCU | GACAUGGUCACGUCCAGGA |
| GUACCAGUGCAGGUCCUCA | CAUGGUCACGUCCAGGAGU |
| ACCAGUGCAGGUCCUCACU | UGGUCACGUCCAGGAGUGA |
| AAACACGGUGGGCCAAAGG | UUUGUGCCACCCGGUUUCC |
| AGAUGGUGUGGCCGAUGUG | UCUACCACACCGGCUACAC |
| UGGUGUGGCCGAUGUGUCU | ACCACACCGGCUACACAGA |
| GGUGUGGCCGAUGUGUCUA | CCACACCGGCUACACAGAU |
| GCAUCAUUGGCCGCACACU | CGUAGUAACCGGCGUGUGA |
| UCAUUGGCCGCACACUGGU | AGUAACCGGCGUGUGACCA |
| AAAUGAAGAAAGUACAAAG | UUUACUUCUUUCAUGUUUC |
| CGCUGGAAGUCGUUUGGCU | GCGACCUUCAGCAAACCGA |
| AUCCUGAUAAACAUUAAAC | UAGGACUAUUUGUAAUUUG |
| CUGAUAAACAUUAAACACU | GACUAUUUGUAAUUUGUGA |
| UGAUAAACAUUAAACACUG | ACUAUUUGUAAUUUGUGAC |
| AACAUUAAACACUGUAAUC | UUGUAAUUUGUGACAUUAG |
| AACACUGUAAUCUUAAAAG | UUGUGACAUUAGAAUUUUC |
| UCUUAAAAGUGUAAUUGUG | AGAAUUUUCACAUUAACAC |
| CUUAAAAGUGUAAUUGUGU | GAAUUUUCACAUUAACACA |
| UUAAAAGUGUAAUUGUGUG | AAUUUUCACAUUAACACAC |
| AACUGAUUUAUGAUCACUU | UUGACUAAAUACUAGUGAA |
| AUAGUUUUAUAAAACUCAG | UAUCAAAAUAUUUUGAGUC |
| UAGUUUUAUAAAACUCAGU | AUCAAAAUAUUUUGAGUCA |
| AGUUUUAUAAAACUCAGUU | UCAAAAUAUUUUGAGUCAA |
| AUUAUGAGGCUAUUAAAAG | UAAUACUCCGAUAAUUUUC |
| AUUAAAAGAAUCCAAAUUC | UAAUUUUCUUAGGUUUAAG |
| AAAGAAUCCAAAUUCAAAC | UUUCUUAGGUUUAAGUUUG |
| CUAUAAAGUAGUCGCGGAG | GAUAUUUCAUCAGCGCCUC |
| UAAAGUAGUCGCGGAGACG | AUUUCAUCAGCGCCUCUGC |
| GUUUGCGUCGUAGUCUCCU | CAAACGCAGCAUCAGAGGA |
| GGGCAUCAUCAAUUUCGAG | CCCGUAGUAGUUAAAGCUC |
| CAUCAAUUUCGAGCAGAAG | GUAGUUAAAGCUCGUCUUC |
| AUCAAUUUCGAGCAGAAGG | UAGUUAAAGCUCGUCUUCC |
| AGCAGAAGGAAAGUAAUGG | UCGUCUUCCUUUCAUUACC |
| CAGAAGGAAAGUAAUGGAC | GUCUUCCUUUCAUUACCUG |
| AGAAGGAAAGUAAUGGACC | UCUUCCUUUCAUUACCUGG |
| AGGAAAGUAAUGGACCAGU | UCCUUUCAUUACCUGGUCA |
| UAAUGGACCAGUGAAGGUG | AUUACCUGGUCACUUCCAC |
| UGGGGAAGCAUUAAAGGAC | ACCCCUUCGUAAUUUCCUG |
| GAAGCAUUAAAGGACUGAC | CUUCGUAAUUUCCUGACUG |
| AGCAUUAAAGGACUGACUG | UCGUAAUUUCCUGACUGAC |
| AAGGACUGACUGAAGGCCU | UUCCUGACUGACUUCCGGA |
| CAUGGAUUCCAUGUUCAUG | GUACCUAAGGUACAAGUAC |
| UUCCAUGUUCAUGAGUUUG | AAGGUACAAGUACUCAAAC |
| CAUGUUCAUGAGUUUGGAG | GUACAAGUACUCAAACCUC |
| AGUUUGGAGAUAAUACAGC | UCAAACCUCUAUUAUGUCG |
| UUUGGAGAUAAUACAGCAG | AAACCUCUAUUAUGUCGUC |
| UGGAGAUAAUACAGCAGGC | ACCUCUAUUAUGUCGUCCG |
| AUACAGCAGGCUGUACCAG | UAUGUCGUCCGACAUGGUC |
| ACUUUAAUCCUCUAUCCAG | UGAAAUUAGGAGAUAGGUC |
| CCUCUAUCCAGAAAACACG | GGAGAUAGGUCUUUUGUGC |
| CUAUCCAGAAAACACGGUG | GAUAGGUCUUUUGUGCCAC |
| GGCCAAAGGAUGAAGAGAG | CCGGUUUCCUACUUCUCUC |
| AAGGAUGAAGAGAGGCAUG | UUCCUACUUCUCUCCGUAC |
| AUGAAGAGAGGCAUGUUGG | UACUUCUCUCCGUACAACC |
| GAAGAGAGGCAUGUUGGAG | CUUCUCUCCGUACAACCUC |
| GAGACUUGGGCAAUGUGAC | CUCUGAACCCGUUACACUG |
| ACUUGGGCAAUGUGACUGC | UGAACCCGUUACACUGACG |
| UUGGGCAAUGUGACUGCUG | AACCCGUUACACUGACGAC |
| AAUGUGACUGCUGACAAAG | UUACACUGACGACUGUUUC |
| UGCUGACAAAGAUGGUGUG | ACGACUGUUUCUACCACAC |
| AAAGAUGGUGUGGCCGAUG | UUUCUACCACACCGGCUAC |
| GUGUCUAUUGAAGAUUCUG | CACAGAUAACUUCUAAGAC |
| GUCUAUUGAAGAUUCUGUG | CAGAUAACUUCUAAGACAC |
| CUGUGAUCUCACUCUCAGG | GACACUAGAGUGAGAGUCC |
| GUGAUCUCACUCUCAGGAG | CACUAGAGUGAGAGUCCUC |
| GAUCUCACUCUCAGGAGAC | CUAGAGUGAGAGUCCUCUG |
| AUCUCACUCUCAGGAGACC | UAGAGUGAGAGUCCUCUGG |
| CUCAGGAGACCAUUGCAUC | GAGUCCUCUGGUAACGUAG |
| CAUUGCAUCAUUGGCCGCA | GUAACGUAGUAACCGGCGU |
| GUGGUCCAUGAAAAAGCAG | CACCAGGUACUUUUUCGUC |
| GUCCAUGAAAAAGCAGAUG | CAGGUACUUUUUCGUCUAC |
| GAAAAAGCAGAUGACUUGG | CUUUUUCGUCUACUGAACC |
| GAUGACUUGGGCAAAGGUG | CUACUGAACCCGUUUCCAC |
| AAGAAAGUACAAAGACAGG | UUCUUUCAUGUUUCUGUCC |
| AGUACAAAGACAGGAAACG | UCAUGUUUCUGUCCUUUGC |
| GUACAAAGACAGGAAACGC | CAUGUUUCUGUCCUUUGCG |
| ACAAAGACAGGAAACGCUG | UGUUUCUGUCCUUUGCGAC |
| CAAAGACAGGAAACGCUGG | GUUUCUGUCCUUUGCGACC |
| CUGGAAGUCGUUUGGCUUG | GACCUUCAGCAAACCGAAC |
| AGUCGUUUGGCUUGUGGUG | UCAGCAAACCGAACACCAC |
| GUCGUUUGGCUUGUGGUGU | CAGCAAACCGAACACCACA |
| UUGUGGUGUAAUUGGGAUC | AACACCACAUUAACCCUAG |
| GUGGUGUAAUUGGGAUCGC | CACCACAUUAACCCUAGCG |
| UCGCCCAAUAAACAUUCCC | AGCGGGUUAUUUGUAAGGG |
| CCCAAUAAACAUUCCCUUG | GGGUUAUUUGUAAGGGAAC |
| UUCCCUUGGAUGUAGUCUG | AAGGGAACCUACAUCAGAC |
| AUGUAGUCUGAGGCCCCUU | UACAUCAGACUCCGGGGAA |
| UCUGUUAUCCUGCUAGCUG | AGACAAUAGGACGAUCGAC |
| GUUAUCCUGCUAGCUGUAG | CAAUAGGACGAUCGACAUC |
| GCUGUAGAAAUGUAUCCUG | CGACAUCUUUACAUAGGAC |
| AAAAGUGUAAUUGUGUGAC | UUUUCACAUUAACACACUG |
| GUGACUUUUUCAGAGUUGC | CACUGAAAAAGUCUCAACG |
| AGUACCUGUAGUGAGAAAC | UCAUGGACAUCACUCUUUG |
| CUGAUUUAUGAUCACUUGG | GACUAAAUACUAGUGAACC |
| AUUUAUGAUCACUUGGAAG | UAAAUACUAGUGAACCUUC |
| AACUCAGUUAAAAUGUCUG | UUGAGUCAAUUUUACAGAC |
| UGUCUGUUUCAAUGACCUG | ACAGACAAAGUUACUGGAC |
| GUCUGUUUCAAUGACCUGU | CAGACAAAGUUACUGGACA |
| GACCUGUAUUUUGCCAGAC | CUGGACAUAAAACGGUCUG |
| GACUUAAAUCACAGAUGGG | CUGAAUUUAGUGUCUACCC |
| ACUUAAAUCACAGAUGGGU | UGAAUUUAGUGUCUACCCA |
| UGGGUAUUAAACUUGUCAG | ACCCAUAAUUUGAACAGUC |
| UUUCUUUGUCAUUCAAGCC | AAAGAAACAGUAAGUUCGG |
| UCUUUGUCAUUCAAGCCUG | AGAAACAGUAAGUUCGGAC |
| AGCCUGUGAAUAAAAACCC | UCGGACACUUAUUUUUGGG |
| UUGGGGCCAGAGUGGGCGA | AACCCCGGUCUCACCCGCU |
| AGAGUGGGCGAGGCGCGGA | UCUCACCCGCUCCGCGCCU |
| GUGGGCGAGGCGCGGAGGU | CACCCGCUCCGCGCCUCCA |
| GUAGUCGCGGAGACGGGGU | CAUCAGCGCCUCUGCCCCA |
| GCGGAGACGGGGUGCUGGU | CGCCUCUGCCCCACGACCA |
| CGGAGACGGGGUGCUGGUU | GCCUCUGCCCCACGACCAA |
| AGACGGGGUGCUGGUUUGC | UCUGCCCCACGACCAAACG |
| GGGUGCUGGUUUGCGUCGU | CCCACGACCAAACGCAGCA |
| GCUGGUUUGCGUCGUAGUC | CGACCAAACGCAGCAUCAG |
| GCGUCGUAGUCUCCUGCAG | CGCAGCAUCAGAGGACGUC |
| UCCUGCAGCGUCUGGGGUU | AGGACGUCGCAGACCCCAA |
| UGCAGCGUCUGGGGUUUCC | ACGUCGCAGACCCCAAAGG |
| CAGCGUCUGGGGUUUCCGU | GUCGCAGACCCCAAAGGCA |
| AGCGUCUGGGGUUUCCGUU | UCGCAGACCCCAAAGGCAA |
| CGUCUGGGGUUUCCGUUGC | GCAGACCCCAAAGGCAACG |
| CUGGGGUUUCCGUUGCAGU | GACCCCAAAGGCAACGUCA |
| GGUUUCCGUUGCAGUCCUC | CCAAAGGCAACGUCAGGAG |
| CCGUUGCAGUCCUCGGAAC | GGCAACGUCAGGAGCCUUG |
| GUCCUCGGAACCAGGACCU | CAGGAGCCUUGGUCCUGGA |
| GGAACCAGGACCUCGGCGU | CCUUGGUCCUGGAGCCGCA |
| ACCUCGGCGUGGCCUAGCG | UGGAGCCGCACCGGAUCGC |
| UCGGCGUGGCCUAGCGAGU | AGCCGCACCGGAUCGCUCA |
| CGGCGUGGCCUAGCGAGUU | GCCGCACCGGAUCGCUCAA |
| GCCUAGCGAGUUAUGGCGA | CGGAUCGCUCAAUACCGCU |
| CGAGUUAUGGCGACGAAGG | GCUCAAUACCGCUGCUUCC |
| UUAUGGCGACGAAGGCCGU | AAUACCGCUGCUUCCGGCA |
| GAAGGCCGUGUGCGUGCUG | CUUCCGGCACACGCACGAC |
| CCGUGUGCGUGCUGAAGGG | GGCACACGCACGACUUCCC |
| GCUGAAGGGCGACGGCCCA | CGACUUCCCGCUGCCGGGU |
| UGAAGGGCGACGGCCCAGU | ACUUCCCGCUGCCGGGUCA |
| GAAGGGCGACGGCCCAGUG | CUUCCCGCUGCCGGGUCAC |
| GACGGCCCAGUGCAGGGCA | CUGCCGGGUCACGUCCCGU |
| CCCAGUGCAGGGCAUCAUC | GGGUCACGUCCCGUAGUAG |
| GACUGACUGAAGGCCUGCA | CUGACUGACUUCCGGACGU |
| UGACUGAAGGCCUGCAUGG | ACUGACUUCCGGACGUACC |
| GAAGGCCUGCAUGGAUUCC | CUUCCGGACGUACCUAAGG |
| GCAGGCUGUACCAGUGCAG | CGUCCGACAUGGUCACGUC |
| AGGCUGUACCAGUGCAGGU | UCCGACAUGGUCACGUCCA |
| CAGAAAACACGGUGGGCCA | GUCUUUUGUGCCACCCGGU |
| GAUGGUGUGGCCGAUGUGU | CUACCACACCGGCUACACA |
| UUGGCCGCACACUGGUGGU | AACCGGCGUGUGACCACCA |
| CCGCACACUGGUGGUCCAU | GGCGUGUGACCACCAGGUA |
| GUCUGAGGCCCCUUAACUC | CAGACUCCGGGGAAUUGAG |
| AAUCUUAAAAGUGUAAUUG | UUAGAAUUUUCACAUUAAC |
| AAUUUCUUUGUCAUUCAAG | UUAAAGAAACAGUAAGUUC |
| CUGGCCUAUAAAGUAGUCG | GACCGGAUAUUUCAUCAGC |
| UGGCCUAUAAAGUAGUCGC | ACCGGAUAUUUCAUCAGCG |
| GGCCUAUAAAGUAGUCGCG | CCGGAUAUUUCAUCAGCGC |
| GGCAUCAUCAAUUUCGAGC | CCGUAGUAGUUAAAGCUCG |
| AAGGAAAGUAAUGGACCAG | UUCCUUUCAUUACCUGGUC |
| AGUAAUGGACCAGUGAAGG | UCAUUACCUGGUCACUUCC |
| AAAGGACUGACUGAAGGCC | UUUCCUGACUGACUUCCGG |
| UUGGAGAUAAUACAGCAGG | AACCUCUAUUAUGUCGUCC |
| GAGAUAAUACAGCAGGCUG | CUCUAUUAUGUCGUCCGAC |
| CUCUAUCCAGAAAACACGG | GAGAUAGGUCUUUUGUGCC |
| UAUCCAGAAAACACGGUGG | AUAGGUCUUUUGUGCCACC |
| AUCCAGAAAACACGGUGGG | UAGGUCUUUUGUGCCACCC |
| CCAAAGGAUGAAGAGAGGC | GGUUUCCUACUUCUCUCCG |
| AGGCAUGUUGGAGACUUGG | UCCGUACAACCUCUGAACC |
| GACUUGGGCAAUGUGACUG | CUGAACCCGUUACACUGAC |
| ACUGCUGACAAAGAUGGUG | UGACGACUGUUUCUACCAC |
| GCUGACAAAGAUGGUGUGG | CGACUGUUUCUACCACACC |
| GACCAUUGCAUCAUUGGCC | CUGGUAACGUAGUAACCGG |
| ACCAUUGCAUCAUUGGCCG | UGGUAACGUAGUAACCGGC |
| AUUGCAUCAUUGGCCGCAC | UAACGUAGUAACCGGCGUG |
| AUGACUUGGGCAAAGGUGG | UACUGAACCCGUUUCCACC |
| UGUGGUGUAAUUGGGAUCG | ACACCACAUUAACCCUAGC |
| UGGUGUAAUUGGGAUCGCC | ACCACAUUAACCCUAGCGG |
| CCCUUGGAUGUAGUCUGAG | GGGAACCUACAUCAGACUC |
| CUUGGAUGUAGUCUGAGGC | GAACCUACAUCAGACUCCG |
| UUGGAUGUAGUCUGAGGCC | AACCUACAUCAGACUCCGG |
| AACUCAUCUGUUAUCCUGC | UUGAGUAGACAAUAGGACG |
| AGUUGCUUUAAAGUACCUG | UCAACGAAAUUUCAUGGAC |
| AUGACCUGUAUUUUGCCAG | UACUGGACAUAAAACGGUC |
| UUUGUCAUUCAAGCCUGUG | AAACAGUAAGUUCGGACAC |
| CCUGUGAAUAAAAACCCUG | GGACACUUAUUUUUGGGAC |
| AAUAAAAACCCUGUAUGGC | UUAUUUUUGGGACAUACCG |
| AUGGCACUUAUUAUGAGGC | UACCGUGAAUAAUACUCCG |
| GGGCGAGGCGCGGAGGUCU | CCCGCUCCGCGCCUCCAGA |
| GGCGAGGCGCGGAGGUCUG | CCGCUCCGCGCCUCCAGAC |
| AGGCGCGGAGGUCUGGCCU | UCCGCGCCUCCAGACCGGA |
| GUCGCGGAGACGGGGUGCU | CAGCGCCUCUGCCCCACGA |
| GUGCUGGUUUGCGUCGUAG | CACGACCAAACGCAGCAUC |
| GGUUUGCGUCGUAGUCUCC | CCAAACGCAGCAUCAGAGG |
| GUCGUAGUCUCCUGCAGCG | CAGCAUCAGAGGACGUCGC |
| UCGUAGUCUCCUGCAGCGU | AGCAUCAGAGGACGUCGCA |
| AGUCUCCUGCAGCGUCUGG | UCAGAGGACGUCGCAGACC |
| CUCCUGCAGCGUCUGGGGU | GAGGACGUCGCAGACCCCA |
| CUGCAGCGUCUGGGGUUUC | GACGUCGCAGACCCCAAAG |
| GCAGCGUCUGGGGUUUCCG | CGUCGCAGACCCCAAAGGC |
| GCGUCUGGGGUUUCCGUUG | CGCAGACCCCAAAGGCAAC |
| UCUGGGGUUUCCGUUGCAG | AGACCCCAAAGGCAACGUC |
| UGGGGUUUCCGUUGCAGUC | ACCCCAAAGGCAACGUCAG |
| CGUUGCAGUCCUCGGAACC | GCAACGUCAGGAGCCUUGG |
| UUGCAGUCCUCGGAACCAG | AACGUCAGGAGCCUUGGUC |
| CAGUCCUCGGAACCAGGAC | GUCAGGAGCCUUGGUCCUG |
| GAACCAGGACCUCGGCGUG | CUUGGUCCUGGAGCCGCAC |
| CCAGGACCUCGGCGUGGCC | GGUCCUGGAGCCGCACCGG |
| GGACCUCGGCGUGGCCUAG | CCUGGAGCCGCACCGGAUC |
| GUGGCCUAGCGAGUUAUGG | CACCGGAUCGCUCAAUACC |
| UGGCCUAGCGAGUUAUGGC | ACCGGAUCGCUCAAUACCG |
| CCUAGCGAGUUAUGGCGAC | GGAUCGCUCAAUACCGCUG |
| CUAGCGAGUUAUGGCGACG | GAUCGCUCAAUACCGCUGC |
| GAGUUAUGGCGACGAAGGC | CUCAAUACCGCUGCUUCCG |
| AUGGCGACGAAGGCCGUGU | UACCGCUGCUUCCGGCACA |
| UGGCGACGAAGGCCGUGUG | ACCGCUGCUUCCGGCACAC |
| GGCGACGAAGGCCGUGUGC | CCGCUGCUUCCGGCACACG |
| GGCCGUGUGCGUGCUGAAG | CCGGCACACGCACGACUUC |
| GCCGUGUGCGUGCUGAAGG | CGGCACACGCACGACUUCC |
| AAGGGCGACGGCCCAGUGC | UUCCCGCUGCCGGGUCACG |
| GGGCGACGGCCCAGUGCAG | CCCGCUGCCGGGUCACGUC |
| ACCAGUGAAGGUGUGGGGA | UGGUCACUUCCACACCCCU |
| CAGUGAAGGUGUGGGGAAG | GUCACUUCCACACCCCUUC |
| AGGACUGACUGAAGGCCUG | UCCUGACUGACUUCCGGAC |
| CUGACUGAAGGCCUGCAUG | GACUGACUUCCGGACGUAC |
| GGCCUGCAUGGAUUCCAUG | CCGGACGUACCUAAGGUAC |
| CAGCAGGCUGUACCAGUGC | GUCGUCCGACAUGGUCACG |
| UACCAGUGCAGGUCCUCAC | AUGGUCACGUCCAGGAGUG |
| CACGGUGGGCCAAAGGAUG | GUGCCACCCGGUUUCCUAC |
| GGUGGGCCAAAGGAUGAAG | CCACCCGGUUUCCUACUUC |
| GGCAUGUUGGAGACUUGGG | CCGUACAACCUCUGAACCC |
| GCAUGUUGGAGACUUGGGC | CGUACAACCUCUGAACCCG |
| GGGCAAUGUGACUGCUGAC | CCCGUUACACUGACGACUG |
| AUGGUGUGGCCGAUGUGUC | UACCACACCGGCUACACAG |
| UGCAUCAUUGGCCGCACAC | ACGUAGUAACCGGCGUGUG |
| CAUCAUUGGCCGCACACUG | GUAGUAACCGGCGUGUGAC |
| AUCAUUGGCCGCACACUGG | UAGUAACCGGCGUGUGACC |
| CAUUGGCCGCACACUGGUG | GUAACCGGCGUGUGACCAC |
| GAUGUAGUCUGAGGCCCCU | CUACAUCAGACUCCGGGGA |
| CUGACAAAGAUGGUGUGGC | GACUGUUUCUACCACACCG |
| GUUUGGGGCCAGAGUGGGC | CAAACCCCGGUCUCACCCG |
| GGGCCAGAGUGGGCGAGGC | CCCGGUCUCACCCGCUCCG |
| GGCCAGAGUGGGCGAGGCG | CCGGUCUCACCCGCUCCGC |
| CCAGAGUGGGCGAGGCGCG | GGUCUCACCCGCUCCGCGC |
| GAGUGGGCGAGGCGCGGAG | CUCACCCGCUCCGCGCCUC |
| UGGGCGAGGCGCGGAGGUC | ACCCGCUCCGCGCCUCCAG |
| GCGAGGCGCGGAGGUCUGG | CGCUCCGCGCCUCCAGACC |
| CGAGGCGCGGAGGUCUGGC | GCUCCGCGCCUCCAGACCG |
| GAGGCGCGGAGGUCUGGCC | CUCCGCGCCUCCAGACCGG |
| AAAGUAGUCGCGGAGACGG | UUUCAUCAGCGCCUCUGCC |
| AAGUAGUCGCGGAGACGGG | UUCAUCAGCGCCUCUGCCC |
| UAGUCGCGGAGACGGGGUG | AUCAGCGCCUCUGCCCCAC |
| UCGCGGAGACGGGGUGCUG | AGCGCCUCUGCCCCACGAC |
| GACGGGGUGCUGGUUUGCG | CUGCCCCACGACCAAACGC |
| CGGGGUGCUGGUUUGCGUC | GCCCCACGACCAAACGCAG |
| GGGGUGCUGGUUUGCGUCG | CCCCACGACCAAACGCAGC |
| UUGCGUCGUAGUCUCCUGC | AACGCAGCAUCAGAGGACG |
| CGUCGUAGUCUCCUGCAGC | GCAGCAUCAGAGGACGUCG |
| CGUAGUCUCCUGCAGCGUC | GCAUCAGAGGACGUCGCAG |
| UAGUCUCCUGCAGCGUCUG | AUCAGAGGACGUCGCAGAC |
| UCUCCUGCAGCGUCUGGGG | AGAGGACGUCGCAGACCCC |
| GGGGUUUCCGUUGCAGUCC | CCCCAAAGGCAACGUCAGG |
| UUUCCGUUGCAGUCCUCGG | AAAGGCAACGUCAGGAGCC |
| UGCAGUCCUCGGAACCAGG | ACGUCAGGAGCCUUGGUCC |
| AGUCCUCGGAACCAGGACC | UCAGGAGCCUUGGUCCUGG |
| UCCUCGGAACCAGGACCUC | AGGAGCCUUGGUCCUGGAG |
| CUCGGAACCAGGACCUCGG | GAGCCUUGGUCCUGGAGCC |
| UCGGAACCAGGACCUCGGC | AGCCUUGGUCCUGGAGCCG |
| ACCAGGACCUCGGCGUGGC | UGGUCCUGGAGCCGCACCG |
| CAGGACCUCGGCGUGGCCU | GUCCUGGAGCCGCACCGGA |
| GACCUCGGCGUGGCCUAGC | CUGGAGCCGCACCGGAUCG |
| CUCGGCGUGGCCUAGCGAG | GAGCCGCACCGGAUCGCUC |
| GGCCUAGCGAGUUAUGGCG | CCGGAUCGCUCAAUACCGC |
| GCGAGUUAUGGCGACGAAG | CGCUCAAUACCGCUGCUUC |
| ACGAAGGCCGUGUGCGUGC | UGCUUCCGGCACACGCACG |
| CGUGCUGAAGGGCGACGGC | GCACGACUUCCCGCUGCCG |
| GCGACGGCCCAGUGCAGGG | CGCUGCCGGGUCACGUCCC |
| CGACGGCCCAGUGCAGGGC | GCUGCCGGGUCACGUCCCG |
| CGGCCCAGUGCAGGGCAUC | GCCGGGUCACGUCCCGUAG |
| UGGACCAGUGAAGGUGUGG | ACCUGGUCACUUCCACACC |
| GGACCAGUGAAGGUGUGGG | CCUGGUCACUUCCACACCC |
| GACCAGUGAAGGUGUGGGG | CUGGUCACUUCCACACCCC |
| AGUGAAGGUGUGGGGAAGC | UCACUUCCACACCCCUUCG |
| GGACUGACUGAAGGCCUGC | CCUGACUGACUUCCGGACG |
| GGCUGUACCAGUGCAGGUC | CCGACAUGGUCACGUCCAG |
| GCUGUACCAGUGCAGGUCC | CGACAUGGUCACGUCCAGG |
| UGUACCAGUGCAGGUCCUC | ACAUGGUCACGUCCAGGAG |
| CCAUUGCAUCAUUGGCCGC | GGUAACGUAGUAACCGGCG |
| AUUGGCCGCACACUGGUGG | UAACCGGCGUGUGACCACC |
| UGGCCGCACACUGGUGGUC | ACCGGCGUGUGACCACCAG |
| CGCACACUGGUGGUCCAUG | GCGUGUGACCACCAGGUAC |
| CAGGAAACGCUGGAAGUCG | GUCCUUUGCGACCUUCAGC |
| ACGCUGGAAGUCGUUUGGC | UGCGACCUUCAGCAAACCG |
| GGAUGUAGUCUGAGGCCCC | CCUACAUCAGACUCCGGGG |
| GCCUAUAAAGUAGUCGCGG | CGGAUAUUUCAUCAGCGCC |
| UGGGGCCAGAGUGGGCGAG | ACCCCGGUCUCACCCGCUC |
| GGGGCCAGAGUGGGCGAGG | CCCCGGUCUCACCCGCUCC |
| AGUGGGCGAGGCGCGGAGG | UCACCCGCUCCGCGCCUCC |
| AGUCGCGGAGACGGGGUGC | UCAGCGCCUCUGCCCCACG |
| CGCGGAGACGGGGUGCUGG | GCGCCUCUGCCCCACGACC |
| GUCUCCUGCAGCGUCUGGG | CAGAGGACGUCGCAGACCC |
| GUUUCCGUUGCAGUCCUCG | CAAAGGCAACGUCAGGAGC |
| CCUCGGAACCAGGACCUCG | GGAGCCUUGGUCCUGGAGC |
| CGGAACCAGGACCUCGGCG | GCCUUGGUCCUGGAGCCGC |
| AACCAGGACCUCGGCGUGG | UUGGUCCUGGAGCCGCACC |
| AGUUAUGGCGACGAAGGCC | UCAAUACCGCUGCUUCCGG |
| GUUAUGGCGACGAAGGCCG | CAAUACCGCUGCUUCCGGC |
| UAUGGCGACGAAGGCCGUG | AUACCGCUGCUUCCGGCAC |
| GCGACGAAGGCCGUGUGCG | CGCUGCUUCCGGCACACGC |
| GACGAAGGCCGUGUGCGUG | CUGCUUCCGGCACACGCAC |
| CGUGUGCGUGCUGAAGGGC | GCACACGCACGACUUCCCG |
| GUGUGCGUGCUGAAGGGCG | CACACGCACGACUUCCCGC |
| GUGCGUGCUGAAGGGCGAC | CACGCACGACUUCCCGCUG |
| UGCGUGCUGAAGGGCGACG | ACGCACGACUUCCCGCUGC |
| GCGUGCUGAAGGGCGACGG | CGCACGACUUCCCGCUGCC |
| UGCUGAAGGGCGACGGCCC | ACGACUUCCCGCUGCCGGG |
| CUGAAGGGCGACGGCCCAG | GACUUCCCGCUGCCGGGUC |
| GGCGACGGCCCAGUGCAGG | CCGCUGCCGGGUCACGUCC |
| CAGGCUGUACCAGUGCAGG | GUCCGACAUGGUCACGUCC |
| UCCAGAAAACACGGUGGGC | AGGUCUUUUGUGCCACCCG |
| CCAGAAAACACGGUGGGCC | GGUCUUUUGUGCCACCCGG |
| GGCCGCACACUGGUGGUCC | CCGGCGUGUGACCACCAGG |
| GGUGUAAUUGGGAUCGCCC | CCACAUUAACCCUAGCGGG |
| UGGAUGUAGUCUGAGGCCC | ACCUACAUCAGACUCCGGG |
| UUUGGGGCCAGAGUGGGCG | AAACCCCGGUCUCACCCGC |
| GCCAGAGUGGGCGAGGCGC | CGGUCUCACCCGCUCCGCG |
| CAGAGUGGGCGAGGCGCGG | GUCUCACCCGCUCCGCGCC |
| AGUAGUCGCGGAGACGGGG | UCAUCAGCGCCUCUGCCCC |
| GUGCUGAAGGGCGACGGCC | CACGACUUCCCGCUGCCGG |
| GACAAAGAUGGUGUGGCCG | CUGUUUCUACCACACCGGC |

SEQUENCES

SEQ ID NO: 1
gtaccctgtttacatcattttgccattttcgcgtactgcaaccggcgggc
cacgccgtgaaaagaaggttgttttctccacagtttcggggttctggacg
tttcccggctgcggggcggggggagtctccggcgcacgcggccccttggc
ccgccccagtcattcccggccactcgcgacccgaggctgccgcagggggc
gggctgagcgcgtgcgaggccattggtttggggccagagtgggcgaggcg
cggaggtctggcctataaagtagtcgcggagacggggtgctggcgtcgta
gtctcctgcaggtctggggtttccgttgcagtcctcggaaccaggacctc
ggcgtggcctagcgagttatggcgacgaaggccgtgtgcgtgctgaaggg
cgacggcccagtgcagggcatcatcaatttcgagcagaaggcaagggctg
ggaccgggaggcttgtgttgcgaggccgctcccgacccgctcgtcccccc
gcgaccctttgcatggacgggtcgcccgccagggctagagcagttaagca
gcttgctggaggttcactggctagaaagtggtcagcctgggattgcatgg
acggatttttccactcccaagtctggctgctttttacttcactgtgaggg
gtaaaggtaaatcagctgttttctttgttcagaaactctctccaactttg
cacttttcttaaaggaaagtaatggaccagtgaaggtgtggggaagcatt
aaaggactgactgaaggcctgcatggattccatgttcatgagtttggaga
taatacagcaggtgggtcataatttagctttfftttcttcttcttataaa
taggctgtaccagtgcaggtcctcactttaatcctctatccagaaaacac
ggtgggccaaaggatgaagagaggtaacaagatgcttaactcttgtaatc
aatggcgatacgtttctggagttcatatggtatactacttgtaaatatgt
gcctaagataattccgtgtttcccccacctttgcttttgaacttgctgac
tcatgtgaaaccctgctcccaaatgctggaatgcttttacttcctgggct
taaaggaattgacaaatgggcacttaaaacgatttggttttgtagcattt
gattgaatatagaactaatacaagtgccaaaggggaactaatacaggaaa
tgttcatgaacagtactgtcaaccactagcaaaatcaatcatcatttgat
gcttttcatataggcatgttggagacttgggcaatgtgactgctgacaaa
gatggtgtggccgatgtgtctattgaagattctgtgatctcactctcagg
agaccattgcatcattggccgcacactggtggtaagttttcataaaggat
atgcataaaacttcttctaacagtacagtcatgtatctttcactttgatt
gttagtcgcgaattctaagatccagataaactgtgtttctgctagtgatt
acttgacagcccaaagttatcttcttaaaattttttacaggtccatgaaa
aagcagatgacttgggcaaaggtggaaatgaagaaagtacaaagacagga
aacgctggaagtcgtttggcttgtggtgtaattgggatcgcccaataaac
attcccttggatgtagtctgaggccccttaaagtacctgtagtgagaaac
tgatttatgatcacttggaagatttgtatagttttataaaactcagttaa
aatgtctgtttcaatgacctgtattttgccagacttaaatcacagatggg
tattaaacttgtcagaatttctttgtcattcaagcctgtgaataaaaacc
ctgtatggcacttattatgaggctattaaaagaatccaaattcaaactaa
attagctctgatacttatttatataaacagcttcagtggaacagatttag
taatactaacagtgatagcattttattttgaaagtgttttgagaccatca
aaatgcatactttaaaacagcaggtcttttagctaaaactaacacaactc
tgcttagacaaataggctgtcctttgaagctt
SEQ ID NO: 2
ATKAVCVLKGDGPVQGIINFEQKESNGPVKVWGSIKGLTEGLHGFHVHEF
GDNTAGCTSAGPHFNPLSRKHGGPKDEERHVGDLGNVTADKDGVADVSIE
DSVISLSGDHCIIGRTLVVHEKADDLGKGGNEESTKTGNAGSRLACGVIG
IAQ
SEQ ID NO: 3
Exon3 of hSOD1
taccagtgca ggtcctcact ttaatcctct atccagaaaa
cacggtgggc caaaggatga agagaggtaa
caagatgcttaactcttgta atcaatggcg atacgtttct
ggagttcata tggtatacta cttgtaaata tgtgcctaag
ataattccgt gtttccccca cctttgcttt tgaacttgct
gactcatgtg aaaccctgct cccaaatgct ggaatgcttt
tacttcctgg gcttaaagga attgacaaat gggcacttaa
aacgatttgg ttttgtagca tttgattgaa tatagaacta
atacaagtgc caaaggggaa ctaatacagg aaatgttcat
gaacagtact gtcaaccact agcaaaatca atcatcatt

1. A method of inhibiting expression of a target protein in a subject with a neurological disorder, comprising:
introducing at least one double stranded small interfering ribonucleic acid (siRNA) molecule into the subject with the neurological disorder, wherein the siRNA comprises a first strand and a second strand hybridized together, wherein at least one strand of the siRNA is complementary to the nucleotide sequence of a target gene encoding the target protein;
allowing the siRNA to interact with an RNA induced silencing complex (RISC) to activate and direct the RISC to the target gene; and
promoting destruction of target mRNA of the target gene, thereby inhibiting expression of the target protein.

2. The method of claim 1, wherein the neurological disorder is selected from the group consisting of amyotrophic lateral sclerosis (ALS).

3. The method of claim 1, wherein the small interfering RNA is about 15 to about 25 bases in length.

4. The method of claim 1, wherein the small interfering RNA is about 19 to about 23 bases in length.

5. The method of claim 1, wherein the small interfering RNA is selected from the group consisting of an unmodified small interfering RNA and a modified RNA molecule.

6. The method of claim 1, wherein the target protein is a SOD protein.

7. The method of claim 6, wherein the SOD protein is a wild type SOD-1 protein.

8. The method of claim 6, wherein the SOD protein is a SOD-1 protein with at least one mutation.

9. The method of claim 1, wherein the target gene is an SOD gene.

10. The method of claim 9, wherein the SOD gene is a wild type SOD-1 gene.

11. The method of claim 9, wherein the SOD gene is a SOD-1 gene with at least one mutation.

12. The method of claim 1, wherein the expression of the target protein is inhibited by at least 10%.

13. The method of claim 1, wherein the step of introducing a double stranded small interfering ribonucleic acid (siRNA) further comprises producing a cDNA corresponding to the target gene from an mRNA, and producing the double stranded siRNA from the cDNA such that the siRNA sequence is identical to at least a portion of the target gene cDNA.

14. The method of claim 13, wherein the cDNA is SEQ ID No. 1.

15. The method of claim 13, wherein the cDNA is SEQ ID No. 3.

16. The method of claim 1, wherein the siRNA is selected from Table 3.

17. The method of claim 1, wherein the method further comprises selecting at least one sequence from SEQ ID Nos. 4-47.

18. The method of claim 1, wherein the siRNA comprises SEQ ID No. 44.

19. The method of claim 1, wherein the siRNA comprises SEQ ID No. 45.

20. A method of ameliorating amyotrophic lateral sclerosis (ALS) in subject, comprising:
introducing a small interference ribonucleic acid (siRNA) molecule into the subject with the ALS, wherein the siRNA comprises a first strand and a second strand hybridized together, wherein at least one strand of the siRNA is complementary to a nucleotide sequence of wild type SOD-1 gene;
allowing the siRNA to interact with an RNA induced silencing complex (RISC) to activate and direct the RISC to the wild type SOD-1 gene; and
promoting destruction of wild type SOD-1 mRNA to inhibit expression of the wild type SOD-1 protein, thereby modulating ALS in the subject.

21. The method of claim 20, wherein at least one strand of the small interfering RNA is complementary to a region of Exon 3 of the wild type SOD-1 gene.

22. The method of claim 20, wherein the small interfering RNA is about 15 to about 25 bases in length.

23. The method of claim 20, wherein the small interfering RNA is about 19 to about 23 bases in length.

24. The method of claim 20, wherein the small interfering RNA is selected from the group consisting of an unmodified small interfering RNA and a modified RNA molecule.

25. The method of claim 20, wherein the expression of the target gene is inhibited by at least about 10%.

26. An isolated polynucleic acid consisting of a sequence selected from the group consisting of the sequences listed in Table 3, and the complements thereto.

27. An isolated polynucleic acid consisting of a sequence selected from the group consisting of SEQ ID No. 44 and 45, and the complements thereto.

28. The isolated polynucleic acid of claim 27, wherein the polynucleic acid is a dsRNA molecule.

29. A method of identifying a siRNA molecule useful for treating neurological disorders, comprising:
incubating mammalian cells capable of expressing a target gene in the presence of a dsRNA test compound in the absence and presence of a transfection reagent;
assaying the incubated mammalian cells for target gene expression;
comparing the expression levels of the target gene;
wherein the siRNA molecule is useful for treating neurological disorders when the expression level in the presence of the dsRNA and in the absence of the transfection reagent is substantially modified when compared to the control level.

30. The method of claim 29, wherein the assaying step further includes assaying for protein activity.

31. The method of claim 29, wherein the target gene is SOD-1.

32. The method of claim 29, wherein the method further comprises incubating mammalian cells in the presence of a control nucleic acid compound, in the absence and presence of a transfection reagent.


SEQUENCE LISTING (1927 sequences)

SEQ ID NO: 1 - DNA, 2288 bp, Homo sapiens
gtaccctgtttacatcattttgccattttcgcgtactgcaaccggcgggccacgccgtgaaaagaaggttgttttctccacagtttcggggttctggacgtttcccggctgcggggcggggggagtctccggcgcacgcggccccttggcccgccccagtcattcccggccactcgcgacccgaggctgccgcagggggcgggctgagcgcgtgcgaggccattggtttggggccagagtgggcgaggcgcggaggtctggcctataaagtagtcgcggagacggggtgctggtttgcgtcgtagtctcctgcaggtctggggtttccgttgcagtcctcggaaccaggacctcggcgtggcctagcgagttatggcgacgaaggccgtgtgcgtgctgaagggcgacggcccagtgcagggcatcatcaatttcgagcagaaggcaagggctgggaccgggaggcttgtgttgcgaggccgctcccgacccgctcgtccccccgcgaccctttgcatggacgggtcgcccgccagggctagagcagttaagcagcttgctggaggttcactggctagaaagtggtcagcctgggattgcatggacggatttttccactcccaagtctggctgctttttacttcactgtgaggggtaaaggtaaatcagctgttttctttgttcagaaactctctccaactttgcacttttcttaaaggaaagtaatggaccagtgaaggtgtggggaagcattaaaggactgactgaaggcctgcatggattccatgttcatgagtttggagataatacagcaggtgggtcataatttagcttttttttcttcttcttataaataggctgtaccagtgcaggtcctcactttaatcctctatccagaaaacacggtgggccaaaggatgaagagaggtaacaagatgcttaactcttgtaatcaatggcgatacgtttctggagttcatatggtatactacttgtaaatatgtgcctaagataattccgtgtttcccccacctttgcttttgaacttgctgactcatgtgaaaccctgctcccaaatgctggaatgcttttacttcctgggcttaaaggaattgacaaatgggcacttaaaacgatttggttttgtagcatttgattgaatatagaactaatacaagtgccaaaggggaactaatacaggaaatgttcatgaacagtactgtcaaccactagcaaaatcaatcatcatttgatgcttttcatataggcatgttggagacttgggcaatgtgactgctgacaaagatggtgtggccgatgtgtctattgaagattctgtgatctcactctcaggagaccattgcatcattggccgcacactggtggtaagttttcataaaggatatgcataaaacttcttctaacagtacagtcatgtatctttcactttgattgttagtcgcgaattctaagatccagataaactgtgtttctgcttttaaactactaaatattagtatatctctctactaggattaatgttatttttctaatattatgaggttcttaaacatcttttgggtattgttgggaggaggtagtgattacttgacagcccaaagttatcttcttaaaattttttacaggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaattagctctgatacttatttatataaacagcttcagtggaacagatttagtaatactaacagtgatagcattttattttgaaagtgttttgagaccatcaaaatgcatactttaaaacagcaggtcttttagctaaaactaacacaactctgcttagacaaataggctgtcctttgaagctt

SEQ ID NO: 2 - protein, 153 aa, Homo sapiens
ATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 3 - DNA, 389 bp, Homo sapiens
taccagtgcaggtcctcactttaatcctctatccagaaaacacggtgggccaaaggatgaagagaggtaacaagatgcttaactcttgtaatcaatggcgatacgtttctggagttcatatggtatactacttgtaaatatgtgcctaagataattccgtgtttcccccacctttgcttttgaacttgctgactcatgtgaaaccctgctcccaaatgctggaatgcttttacttcctgggcttaaaggaattgacaaatgggcacttaaaacgatttggttttgtagcatttgattgaatatagaactaatacaagtgccaaaggggaactaatacaggaaatgttcatgaacagtactgtcaaccactagcaaaatcaatcatcatt

SEQ ID NO: 4 - RNA, 19 nt, Artificial
  SYNTHETIC CONSTRUCT
uuaauccucuauccagaaa

SEQ ID NO: 5 - RNA, 19 nt, Artificial
  SYNTHETIC CONSTRUCT
uuucuccauagaggauuaa

SEQ ID NO: 6 - RNA, 19 nt, Artificial
  SYNTHETIC CONSTRUCT
gugcagguccucacuuuaa

SEQ ID NO: 7 - RNA, 19 nt, Artificial
  SYNTHETIC CONSTRUCT
uuaaagugaggaccugcac

SEQ ID NO: 8 - RNA, 19 nt, Artificial
  SYNTHETIC CONSTRUCT
agugcagguccucacuuua

SEQ ID NO: 9 - RNA, 19 nt, Artificial
  SYNTHETIC CONSTRUCT
uaaagugaggaccugcacu

SEQ ID NO: 10 - RNA, 19 nt, Artificial
  SYNTHETIC CONSTRUCT
uccucacuuuaauccucua

SEQ ID NO: 11 - RNA, 19 nt, Artificial
  SYNTHETIC CONSTRUCT
uagaggauuaaagugagga

SEQ ID NO: 12 - RNA, 19 nt, Artificial
  SYNTHETIC CONSTRUCT
aauacagcaggcuguacca

SEQ ID NO: 13 - RNA, 19 nt, Artificial
  SYNTHETIC CONSTRUCT
ugguacagccugcuguauu

SEQ ID NO: 14 - RNA, 19 nt, Artificial
  SYNTHETIC CONSTRUCT
gcagguccucacuuuaauc

SEQ ID NO: 15 - RNA, 19 nt, Artificial
  SYNTHETIC CONSTRUCT
gauuaaagugaggaccugc

SEQ ID NO: 16 - RNA, 19 nt, Artificial
  SYNTHETIC CONSTRUCT
ccucacuuuaauccucuau

SEQ ID NO: 17 - RNA, 19 nt, Artificial
  SYNTHETIC CONSTRUCT
auagaggauuaaagugagg

SEQ ID NO: 18 - RNA, 19 nt, Artificial
  SYNTHETIC CONSTRUCT
ucacuuuaauccucuaucc

SEQ ID NO: 19 - RNA, 19 nt, Artificial
  SYNTHETIC CONSTRUCT
ggauagaggauuaaaguga

SEQ ID NO: 20 - RNA, 19 nt, Artificial
  SYNTHETIC CONSTRUCT
cacuuuaauccucuaucca

SEQ ID NO: 21 - RNA, 19 nt, Artificial
  SYNTHETIC CONSTRUCT
uggauagaggauuaaagug

SEQ ID NO: 22 - RNA, 19 nt, Artificial
  SYNTHETIC CONSTRUCT
cuuuaauccucuauccaga

SEQ ID NO: 23 - RNA, 19 nt, Artificial
  SYNTHETIC CONSTRUCT
ucuggauagaccauuaaag

SEQ ID NO: 24 - RNA, 19 nt, Artificial
  SYNTHETIC CONSTRUCT
uuuaauccucuauccagaa

SEQ ID NO: 25 - RNA, 19 nt, Artificial
  SYNTHETIC CONSTRUCT
uucuggauagaggauuaaa

SEQ ID NO: 26 - RNA, 19 nt, Artificial
  SYNTHETIC CONSTRUCT
aauccucuauccagaaaac

SEQ ID NO: 27 - RNA, 19 nt, Artificial
  SYNTHETIC CONSTRUCT
guuuucuggauagaggauu

SEQ ID NO: 28 - RNA, 19 nt, Artificial
  SYNTHETIC CONSTRUCT
auccucuauccagaaaaca

SEQ ID NO: 29 - RNA, 19 nt, Artificial
  SYNTHETIC CONSTRUCT
uguuuucaggauagaggau

SEQ ID NO: 30 - RNA, 19 nt, Artificial
  SYNTHETIC CONSTRUCT
ccagugcagguccucacuu

SEQ ID NO: 31 - RNA, 19 nt, Artificial
  SYNTHETIC CONSTRUCT
aagugaggaccugcacugg

SEQ ID NO: 32 - RNA, 19 nt, Artificial
  SYNTHETIC CONSTRUCT
gcuuaaaggaauugacaaa

SEQ ID NO: 33 - RNA, 19 nt, Artificial
  SYNTHETIC CONSTRUCT
uuugucaauuccuuuaagc

SEQ ID NO: 34 - RNA, 23 nt, Artificial
  SYNTHETIC CONSTRUCT
guggaaaugaagaaaguacaaag

SEQ ID NO: 35 - RNA, 23 nt, Artificial
  SYNTHETIC CONSTRUCT
cuuuguacuuucuucauuuccac

SEQ ID NO: 36 - RNA, 23 nt, Artificial
  SYNTHETIC CONSTRUCT
gccgaugugucuauugaagauuc

SEQ ID NO: 37 - RNA, 23 nt, Artificial
  SYNTHETIC CONSTRUCT
gaaucuucaauagacacaucggc

SEQ ID NO: 38 - RNA, 23 nt, Artificial
  SYNTHETIC CONSTRUCT
ggcaaugugacugcugacaaaga

SEQ ID NO: 39 - RNA, 23 nt, Artificial
  SYNTHETIC CONSTRUCT
ucuuugucagcagucacauugcc

SEQ ID NO: 40 - RNA, 23 nt, Artificial
  SYNTHETIC CONSTRUCT
aagguguggggaagcauuaaagg

SEQ ID NO: 41 - RNA, 23 nt, Artificial
  SYNTHETIC CONSTRUCT
ccuuuaaugcuuccccacaccuu

SEQ ID NO: 42 - RNA, 23 nt, Artificial
  SYNTHETIC CONSTRUCT
aggccugcauggauuccauguuc

SEQ ID NO: 43 - RNA, 23 nt, Artificial
  SYNTHETIC CONSTRUCT
gaacauggaauccaugcaggccu

SEQ ID NO: 44 - RNA, 23 nt, Artificial
  SYNTHETIC CONSTRUCT
guggccgaugugucuauugaaga

SEQ ID NO: 45 - RNA, 23 nt, Artificial
  SYNTHETIC CONSTRUCT
ucuucaauagacacaucggccac

SEQ ID NO: 46 - RNA, 23 nt, Artificial
  SYNTHETIC CONSTRUCT
guggggaagcauuaaaggacuga

SEQ ID NO: 47 - RNA, 23 nt, Artificial
  SYNTHETIC CONSTSRUCT
ucaguccuuuaaugcuuccccac

SEQ ID NO: 48 - RNA, 19 nt, Artificial
  SYNTHETIC CONSTRUCT
ucaagccugugaauaaaaa

SEQ ID NO: 49 - RNA, 19 nt, Artificial
  SYNTHETIC CONSTRUCT
aguucggacacuuauuuuu

SEQ ID NO: 50 - RNA, 19 nt, Artificial
  SYNTHETIC CONSTRUCT
ucaugaguuuggagauaau

SEQ ID NO: 51 - RNA, 19 nt, Artificial
  SYNTHETIC CONSTRUCT
aguacucaaaccucuauua

SEQ ID NO: 52 - RNA, 19 nt, Artificial
  SYNTHETIC CONSTRUCT
uuaauccucuauccagaaa

SEQ ID NO: 53 - RNA, 19 nt, Artificial
  SYNTHETIC CONSTRUCT
aauuaggagauaggucuuu

SEQ ID NO: 54 - RNA, 19 nt, Artificial
  SYNTHETIC CONSTRUCT
caaugugacugcugacaaa

SEQ ID NO: 55 - RNA, 19 nt, Artificial
  SYNTHETIC CONSTRUCT
guuacacugacgacuguuu

SEQ ID NO: 56 - RNA, 19 nt, Artificial
  SYNTHETIC CONSTRUCT
uaauugggaucgcccaaua

SEQ ID NO: 57 - RNA, 19 nt, Artificial
  SYNTHETIC CONSTRUCT
auuaacccuagcggguuau

SEQ ID NO: 58 - RNA, 19 nt, Artificial
  SYNTHETIC CONSTRUCT
guagaaauguauccugaua

SEQ ID NO: 59 - RNA, 19 nt, Artificial
  SYNTHETIC CONSTRUCT
caucuuuacauaggacuau

SEQ ID NO: 60 - RNA, 19 nt, Artificial
  SYNTHETIC CONSTRUCT
guagugagaaacugauuua

SEQ ID NO: 61 - RNA, 19 nt, Artificial
  SYNTHETIC CONSTRUCT
caucacucuuugacuaaau

SEQ ID NO: 62 - RNA, 19 nt, Artificial
  SYNTHETIC CONSTRUCT
guauuuugccagacuuaaa

SEQ ID NO: 63 - RNA, 19 nt, Artificial
  SYNTHETIC CONSTRUCT
cauaaaacggucugaauuu

SEQ ID NO: 64 - RNA, 19 nt, Artificial
  SYNTHETIC CONSTRUCT
agaaauguauccugauaaa

SEQ ID NO: 65 - RNA, 19 nt, Artificial
  SYNTHETIC CONSTRUCT
ucuuuacauaggacuauuu

SEQ ID NO: 66 - RNA, 19 nt, Artificial
  SYNTHETIC CONSTRUCT
guauccugauaaacauuaa

SEQ ID NO: 67 - RNA, 19 nt, Artificial
  SYNTHETIC CONSTRUCT
cauaggacuauuuguaauu

SEQ ID NO: 68 - RNA, 19 nt, Artificial
  SYNTHETIC CONSTRUCT
uaaacacuguaaucuuaaa

SEQ ID NO: 69 - RNA, 19 nt, Artificial
  SYNTHETIC CONSTRUCT
auuugugacauuagaauuu

SEQ ID NO: 70 - RNA, 19 nt, Artificial
  SYNTHETIC CONSTRUCT
gaagauuuguauaguuuua

SEQ ID NO: 71 - RNA, 19 nt, Artificial
  SYNTHETIC CONSTRUCT
cuucuaaacauaucaaaau

SEQ ID NO: 72 - RNA, 19 nt, Artificial
  SYNTHETIC CONSTRUCT
agauuuguauaguuuuaua

SEQ ID NO: 73 - RNA, 19 nt, Artificial
  SYNTHETIC CONSTRUCT
ucuaaacauaucaaaauau

SEQ ID NO: 74 - RNA, 19 nt, Artificial
  SYNTHETIC CONSTRUCT
cggaggucuggccuauaaa

SEQ ID NO: 75 - RNA, 19 nt, Artificial
  SYNTHETIC CONSTRUCT
gccuccagaccggauauuu

SEQ ID NO: 76 - RNA, 19 nt, Artificial
  SYNTHETIC CONSTRUCT
ugcagggcaucaucaauuu

SEQ ID NO: 77 - RNA, 19 nt, Artificial
  SYNTHETIC CONSTRUCT
acgucccguaguaguuaaa

SEQ ID NO: 78 - RNA, 19 nt, Artificial
  SYNTHETIC CONSTRUCT
ucaucaauuucgagcagaa

SEQ ID NO: 79 - RNA, 19 nt, Artificial
  SYNTHETIC CONSTRUCT
aguaguuaaagcucgucuu

SEQ ID NO: 80 - RNA, 19 nt, Artificial
  SYNTHETIC CONSTRUCT
gguguggggaagcauuaaa

SEQ ID NO: 81 - RNA, 19 nt, Artificial
  SYNTHETIC CONSTRUCT
ccacaccccuucguaauuu

SEQ ID NO: 82 - RNA, 19 nt, Artificial
  SYNTHETIC CONSTRUCT
ugaguuuggagauaauaca

SEQ ID NO: 83 - RNA, 19 nt, Artificial
  SYNTHETIC CONSTRUCT
acucaaaccucuauuaugu

SEQ ID NO: 84 - RNA, 19 nt, Artificial
  SYNTHETIC CONSTRUCT
gugcagguccucacuuuaa

SEQ ID NO: 85 - RNA, 19 nt, Artificial
  SYNTHETIC CONSTRUCT
cacguccaggagugaaauu

SEQ ID NO: 86 - RNA, 19 nt, Artificial
  SYNTHETIC CONSTRUCT
ugcagguccucacuuuaau

SEQ ID NO: 87 - RNA, 19 nt, Artificial
  SYNTHETIC CONSTRUCT
acguccaggagugaaauua

SEQ ID NO: 88 - RNA, 19 nt, Artificial
  SYNTHETIC CONSTRUCT
uaauccucuauccagaaaa

SEQ ID NO: 89 - RNA, 19 nt, Artificial
  SYNTHETIC CONSTRUCT
auuaggagauaggucuuuu

SEQ ID NO: 90 - RNA, 19 nt, Artificial
  SYNTHETIC CONSTRUCT
uggccgaugugucuauuga

SEQ ID NO: 91 - RNA, 19 nt, Artificial
  SYNTHETIC CONSTRUCT
accggcuacacagauaacu

SEQ ID NO: 92 - RNA, 19 nt, Artificial
  SYNTHETIC CONSTRUCT
cgaugugucuauugaagau

SEQ ID NO: 93 - RNA, 19 nt, Artificial
  SYNTHETIC CONSTRUCT
gcuacacagauaacuucua

SEQ ID NO: 94 - RNA, 19 nt, Artificial
  SYNTHETIC CONSTRUCT
acacuggugguccaugaaa

SEQ ID NO: 95 - RNA, 19 nt, Artificial
  SYNTHETIC CONSTRUCT
ugugaccaccagguacuuu

SEQ ID NO: 96 - RNA, 19 nt, Artificial
  SYNTHETIC CONSTRUCT
cacuggugguccaugaaaa

SEQ ID NO: 97 - RNA, 19 nt, Artificial
  SYNTHETIC CONSTRUCT
gugaccaccagguacuuuu

SEQ ID NO: 98 - RNA, 19 nt, Artificial
  SYNTHETIC CONSTRUCT
acuggugguccaugaaaaa

SEQ ID NO: 99 - RNA, 19 nt, Artificial
  SYNTHETIC CONSTRUCT
ugaccaccagguacuuuuu

SEQ ID NO: 100 - RNA, 19 nt, Artificial
  SYNTHETIC CONSTRUCT
gggcaaagguggaaaugaa

SEQ ID NO: 101 - RNA, 19 nt, Artificial
  SYNTHETIC CONSTRUCT
cccguuuccaccuuuacuu

SEQ ID NO: 102 - RNA, 19 nt, Artificial
  SYNTHETIC CONSTRUCT
caaagguggaaaugaagaa

SEQ ID NO: 103 - RNA, 19 nt, Artificial
  SYNTHETIC CONSTRUCT
guuuccaccuuuacuucuu

SEQ ID NO: 104 - RNA, 19 nt, Artificial
  SYNTHETIC CONSTRUCT
ggaaaugaagaaaguacaa

SEQ ID NO: 105 - RNA, 19 nt, Artificial
  SYNTHETIC CONSTRUCT
ccuuuacuucuuucauguu

SEQ ID NO: 106 - RNA, 19 nt, Artificial
  SYNTHETIC CONSTRUCT
ugaagaaaguacaaagaca

SEQ ID NO: 107 - RNA, 19 nt, Artificial
  SYNTHETIC CONSTRUCT
acuucuuucauguuucugu

SEQ ID NO: 108 - RNA, 19 nt, Artificial
  SYNTHETIC CONSTRUCT
uuuggcuugugguguaauu

SEQ ID NO: 109 - RNA, 19 nt, Artificial
  SYNTHETIC CONSTRUCT
aaaccgaacaccacauuaa

SEQ ID NO: 110 - RNA, 19 nt, Artificial
  SYNTHETIC CONSTRUCT
aauugggaucgcccaauaa

SEQ ID NO: 111 - RNA, 19 nt, Artificial
  SYNTHETIC CONSTRUCT
uuaacccuagcggguuauu

SEQ ID NO: 112 - RNA, 19 nt, Artificial
  SYNTHETIC CONSTRUCT
auugggaucgcccaauaaa

SEQ ID NO: 113 - RNA, 19 nt, Artificial
  SYNTHETIC CONSTRUCT
uaacccuagcggguuauuu

SEQ ID NO: 114 - RNA, 19 nt, Artificial
  SYNTHETIC CONSTRUCT
caauaaacauucccuugga

SEQ ID NO: 115 - RNA, 19 nt, Artificial
  SYNTHETIC CONSTRUCT
guuauuuguaagggaaccu

SEQ ID NO: 116 - RNA, 19 nt, Artificial
  SYNTHETIC CONSTRUCT
guagucugaggccccuuaa

SEQ ID NO: 117 - RNA, 19 nt, Artificial
  SYNTHETIC CONSTRUCT
caucagacuccggggaauu

SEQ ID NO: 118 - RNA, 19 nt, Artificial
  SYNTHETIC CONSTRUCT
ccccuuaacucaucuguua

SEQ ID NO: 119 - RNA, 19 nt, Artificial
  SYNTHETIC CONSTRUCT
ggggaauugaguagacaau

SEQ ID NO: 120 - RNA, 19 nt, Artificial
  SYNTHETIC CONSTRUCT
uuauccugcuagcuguaga

SEQ ID NO: 121 - RNA, 19 nt, Artificial
  SYNTHETIC CONSTRUCT
aauaggacgaucgacaucu

SEQ ID NO: 122 - RNA, 19 nt, Artificial
  SYNTHETIC CONSTRUCT
ugcuagcuguagaaaugua

SEQ ID NO: 123 - RNA, 19 nt, Artificial
  SYNTHETIC CONSTRUCT
acgaucgacaucuuuacau

SEQ ID NO: 124 - RNA, 19 nt, Artificial
  SYNTHETIC CONSTRUCT
ucagaguugcuuuaaagua

SEQ ID NO: 125 - RNA, 19 nt, Artificial
  SYNTHETIC CONSTRUCT
agucucaacgaaauuucau

SEQ ID NO: 126 - RNA, 19 nt, Artificial
  SYNTHETIC CONSTRUCT
uaaaguaccuguagugaga

SEQ ID NO: 127 - RNA, 19 nt, Artificial
  SYNTHETIC CONSTRUCT
auuucauggacaucacucu

SEQ ID NO: 128 - RNA, 19 nt, Artificial
  SYNTHETIC CONSTRUCT
aaguaccuguagugagaaa

SEQ ID NO: 129 - RNA, 19 nt, Artificial
  SYNTHETIC CONSTRUCT
uucauggacaucacucuuu

SEQ ID NO: 130 - RNA, 19 nt, Artificial
  SYNTHETIC CONSTRUCT
gugagaaacugauuuauga

SEQ ID NO: 131 - RNA, 19 nt, Artificial
  SYNTHETIC CONSTRUCT
cacucuuugacuaaauacu

SEQ ID NO: 132 - RNA, 19 nt, Artificial
  SYNTHETIC CONSTRUCT
aaaucacagauggguauua

SEQ ID NO: 133 - RNA, 19 nt, Artificial
  SYNTHETIC CONSTRUCT
uuuagugucuacccauaau

SEQ ID NO: 134 - RNA, 19 nt, Artificial
  SYNTHETIC CONSTRUCT
aaucacagauggguauuaa

SEQ ID NO: 135 - RNA, 19 nt, Artificial
  SYNTHETIC CONSTRUCT
uuagugucuacccauaauu

SEQ ID NO: 136 - RNA, 19 nt, Artificial
  SYNTHETIC CONSTRUCT
aucacagauggguauuaaa

SEQ ID NO: 137 - RNA, 19 nt, Artificial
  SYNTHETIC CONSTRUCT
uagugucuacccauaauuu

SEQ ID NO: 138 - RNA, 19 nt, Artificial
  SYNTHETIC CONSTRUCT
acagauggguauuaaacuu

SEQ ID NO: 139 - RNA, 19 nt, Artificial
  SYNTHETIC CONSTRUCT
ugucuacccauaauuugaa

SEQ ID NO: 140 - RNA, 19 nt, Artificial
  SYNTHETIC CONSTRUCT
cauucaagccugugaauaa

SEQ ID NO: 141 - RNA, 19 nt, Artificial
  SYNTHETIC CONSTRUCT
guaaguucggacacuuauu

SEQ ID NO: 142 - RNA, 19 nt, Artificial
  SYNTHETIC CONSTRUCT
auucaagccugugaauaaa

SEQ ID NO: 143 - RNA, 19 nt, Artificial
  SYNTHETIC CONSTRUCT
uaaguucggacacuuauuu

SEQ ID NO: 144 - RNA, 19 nt, Artificial
  SYNTHETIC CONSTRUCT
uucaagccugugaauaaaa

SEQ ID NO: 145 - RNA, 19 nt, Artificial
  SYNTHETIC CONSTRUCT
aaguucggacacuuauuuu

SEQ ID NO: 146 - RNA, 19 nt, Artificial
  SYNTHETIC CONSTRUCT
cccuguauggcacuuauua

SEQ ID NO: 147 - RNA, 19 nt, Artificial
  SYNTHETIC CONSTRUCT
gggacauaccgugaauaau

SEQ ID NO: 148 - RNA, 19 nt, Artificial
  SYNTHETIC CONSTRUCT
gcggaggucuggccuauaa

SEQ ID NO: 149 - RNA, 19 nt, Artificial
  SYNTHETIC CONSTRUCT
cgccuccagaccggauauu

SEQ ID NO: 150 - RNA, 19 nt, Artificial
  SYNTHETIC CONSTRUCT
aaacacuguaaucuuaaaa

SEQ ID NO: 151 - RNA, 19 nt, Artificial
  SYNTHETIC CONSTRUCT
uuugugacauuagaauuuu

SEQ ID NO: 152 - RNA, 19 nt, Artificial
  SYNTHETIC CONSTRUCT
uuuucagaguugcuuuaaa

SEQ ID NO: 153 - RNA, 19 nt, Artificial
  SYNTHETIC CONSTRUCT
aaaagucucaacgaaauuu

SEQ ID NO: 154 - RNA, 19 nt, Artificial
  SYNTHETIC CONSTRUCT
uuuguauaguuuuauaaaa

SEQ ID NO: 155 - RNA, 19 nt, Artificial
  SYNTHETIC CONSTRUCT
aaacauaucaaaauauuuu

SEQ ID NO: 156 - RNA, 19 nt, Artificial
  SYNTHETIC CONSTRUCT
uuauaaaacucaguuaaaa

SEQ ID NO: 157 - RNA, 19 nt, Artificial
  SYNTHETIC CONSTRUCT
aauauuuugagucaauuuu

SEQ ID NO: 158 - RNA, 19 nt, Artificial
  SYNTHETIC CONSTRUCT
agaauuucuuugucauuca

SEQ ID NO: 159 - RNA, 19 nt, Artificial
  SYNTHETIC CONSTRUCT
ucuuaaagaaacaguaagu

SEQ ID NO: 160 - RNA, 19 nt, Artificial
  SYNTHETIC CONSTRUCT
gaauuucuuugucauucaa

SEQ ID NO: 161 - RNA, 19 nt, Artificial
  SYNTHETIC CONSTRUCT
cuuaaagaaacaguaaguu

SEQ ID NO: 162 - RNA, 19 nt, Artificial
  SYNTHETIC CONSTRUCT
uuauuaugaggcuauuaaa

SEQ ID NO: 163 - RNA, 19 nt, Artificial
  SYNTHETIC CONSTRUCT
aauaauacuccgauaauuu

SEQ ID NO: 164 - RNA, 19 nt, Artificial
  SYNTHETIC CONSTRUCT
uuaugaggcuauuaaaaga

SEQ ID NO: 165 - RNA, 19 nt, Artificial
  SYNTHETIC CONSTRUCT
aauacuccgauaauuuucu

SEQ ID NO: 166 - RNA, 19 nt, Artificial
  SYNTHETIC CONSTRUCT
aggucuggccuauaaagua

SEQ ID NO: 167 - RNA, 19 nt, Artificial
  SYNTHETIC CONSTRUCT
uccagaccggauauuucau

SEQ ID NO: 168 - RNA, 19 nt, Artificial
  SYNTHETIC CONSTRUCT
agcgaguuauggcgacgaa

SEQ ID NO: 169 - RNA, 19 nt, Artificial
  SYNTHETIC CONSTRUCT
ucgcucaauaccgcugcuu

SEQ ID NO: 170 - RNA, 19 nt, Artificial
  SYNTHETIC CONSTRUCT
cagugcagggcaucaucaa

SEQ ID NO: 171 - RNA, 19 nt, Artificial
  SYNTHETIC CONSTRUCT
gucacgucccguaguaguu

SEQ ID NO: 172 - RNA, 19 nt, Artificial
  SYNTHETIC CONSTRUCT
agggcaucaucaauuucga

SEQ ID NO: 173 - RNA, 19 nt, Artificial
  SYNTHETIC CONSTRUCT
ucccguaguaguuaaagcu

SEQ ID NO: 174 - RNA, 19 nt, Artificial
  SYNTHETIC CONSTRUCT
ucaauuucgagcagaagga

SEQ ID NO: 175 - RNA, 19 nt, Artificial
  SYNTHETIC CONSTRUCT
aguuaaagcucgucuuccu

SEQ ID NO: 176 - RNA, 19 nt, Artificial
  SYNTHETIC CONSTRUCT
ucgagcagaaggaaaguaa

SEQ ID NO: 177 - RNA, 19 nt, Artificial
  SYNTHETIC CONSTRUCT
agcucgucuuccuuucauu

SEQ ID NO: 178 - RNA, 19 nt, Artificial
  SYNTHETIC CONSTRUCT
cgagcagaaggaaaguaau

SEQ ID NO: 179 - RNA, 19 nt, Artificial
  SYNTHETIC CONSTRUCT
gcucgucuuccuuucauua

SEQ ID NO: 180 - RNA, 19 nt, Artificial
  SYNTHETIC CONSTRUCT
gcagaaggaaaguaaugga

SEQ ID NO: 181 - RNA, 19 nt, Artificial
  SYNTHETIC CONSTRUCT
cgucuuccuuucauuaccu

SEQ ID NO: 182 - RNA, 19 nt, Artificial
  SYNTHETIC CONSTRUCT
gaaggaaaguaauggacca

SEQ ID NO: 183 - RNA, 19 nt, Artificial
  SYNTHETIC CONSTRUCT
cuuccuuucauuaccuggu

SEQ ID NO: 184 - RNA, 19 nt, Artificial
  SYNTHETIC CONSTRUCT
gaaaguaauggaccaguga

SEQ ID NO: 185 - RNA, 19 nt, Artificial
  SYNTHETIC CONSTRUCT
cuuucauuaccuggucacu

SEQ ID NO: 186 - RNA, 19 nt, Artificial
  SYNTHETIC CONSTRUCT
aaaguaauggaccagugaa

SEQ ID NO: 187 - RNA, 19 nt, Artificial
  SYNTHETIC CONSTRUCT
uuucauuaccuggucacuu

SEQ ID NO: 188 - RNA, 19 nt, Artificial
  SYNTHETIC CONSTRUCT
aagguguggggaagcauua

SEQ ID NO: 189 - RNA, 19 nt, Artificial
  SYNTHETIC CONSTRUCT
uuccacaccccuucguaau

SEQ ID NO: 190 - RNA, 19 nt, Artificial
  SYNTHETIC CONSTRUCT
agguguggggaagcauuaa

SEQ ID NO: 191 - RNA, 19 nt, Artificial
  SYNTHETIC CONSTRUCT
uccacaccccuucguaauu

SEQ ID NO: 192 - RNA, 19 nt, Artificial
  SYNTHETIC CONSTRUCT
gcauuaaaggacugacuga

SEQ ID NO: 193 - RNA, 19 nt, Artificial
  SYNTHETIC CONSTRUCT
cguaauuuccugacugacu

SEQ ID NO: 194 - RNA, 19 nt, Artificial
  SYNTHETIC CONSTRUCT
ugcauggauuccauguuca

SEQ ID NO: 195 - RNA, 19 nt, Artificial
  SYNTHETIC CONSTRUCT
acguaccuaagguacaagu

SEQ ID NO: 196 - RNA, 19 nt, Artificial
  SYNTHETIC CONSTRUCT
gcauggauuccauguucau

SEQ ID NO: 197 - RNA, 19 nt, Artificial
  SYNTHETIC CONSTRUCT
cguaccuaagguacaagua

SEQ ID NO: 198 - RNA, 19 nt, Artificial
  SYNTHETIC CONSTRUCT
auuccauguucaugaguuu

SEQ ID NO: 199 - RNA, 19 nt, Artificial
  SYNTHETIC CONSTRUCT
uaagguacaaguacucaaa

SEQ ID NO: 200 - RNA, 19 nt, Artificial
  SYNTHETIC CONSTRUCT
ccauguucaugaguuugga

SEQ ID NO: 201 - RNA, 19 nt, Artificial
  SYNTHETIC CONSTRUCT
gguacaaguacucaaaccu

SEQ ID NO: 202 - RNA, 19 nt, Artificial
  SYNTHETIC CONSTRUCT
guucaugaguuuggagaua

SEQ ID NO: 203 - RNA, 19 nt, Artificial
  SYNTHETIC CONSTRUCT
caaguacucaaaccucuau

SEQ ID NO: 204 - RNA, 19 nt, Artificial
  SYNTHETIC CONSTRUCT
uucaugaguuuggagauaa

SEQ ID NO: 205 - RNA, 19 nt, Artificial
  SYNTHETIC CONSTRUCT
aaguacucaaaccucuauu

SEQ ID NO: 206 - RNA, 19 nt, Artificial
  SYNTHETIC CONSTRUCT
caugaguuuggagauaaua

SEQ ID NO: 207 - RNA, 19 nt, Artificial
  SYNTHETIC CONSTRUCT
guacucaaaccucuauuau

SEQ ID NO: 208 - RNA, 19 nt, Artificial
  SYNTHETIC CONSTRUCTION
cagugcagguccucacuuu

SEQ ID NO: 209 - RNA, 19 nt, Artificial
  SYNTHETIC CONSTRUCT
gucacguccaggagugaaa

SEQ ID NO: 210 - RNA, 19 nt, Artificial
  SYNTHETIC CONSTRUCT
agugcagguccucacuuua

SEQ ID NO: 211 - RNA, 19 nt, Artificial
  SYNTHETIC CONSTRUCT
ucacguccaggagugaaau

SEQ ID NO: 212 - RNA, 19 nt, Artificial
  SYNTHETIC CONSTRUCT
uccucacuuuaauccucua

SEQ ID NO: 213 - RNA, 19 nt, Artificial
  SYNTHETIC CONSTRUCT
aggagugaaauuaggagau

SEQ ID NO: 214 - RNA, 19 nt, Artificial
  SYNTHETIC CONSTRUCT
ugaagagaggcauguugga

SEQ ID NO: 215 - RNA, 19 nt, Artificial
  SYNTHETIC CONSTRUCT
acuucucuccguacaaccu

SEQ ID NO: 216 - RNA, 19 nt, Artificial
  SYNTHETIC CONSTRUCT
ggagacuugggcaauguga

SEQ ID NO: 217 - RNA, 19 nt, Artificial
  SYNTHETIC CONSTRUCT
ccucugaacccguuacacu

SEQ ID NO: 218 - RNA, 19 nt, Artificial
  SYNTHETIC CONSTRUCT
gcaaugugacugcugacaa

SEQ ID NO: 219 - RNA, 19 nt, Artificial
  SYNTHETIC CONSTRUCT
cguuacacugacgacuguu

SEQ ID NO: 220 - RNA, 19 nt, Artificial
  SYNTHETIC CONSTRUCT
augugacugcugacaaaga

SEQ ID NO: 221 - RNA, 19 nt, Artificial
  SYNTHETIC CONSTRUCT
uacacugacgacuguuucu

SEQ ID NO: 222 - RNA, 19 nt, Artificial
  SYNTHETIC CONSTRUCT
ugugacugcugacaaagau

SEQ ID NO: 223 - RNA, 19 nt, Artificial
  SYNTHETIC CONSTRUCT
ugugacugcugacaaagau

SEQ ID NO: 224 - RNA, 19 nt, Artificial
  SYNTHETIC CONSTRUCT
ggccgaugugucuauugaa

SEQ ID NO: 225 - RNA, 19 nt, Artificial
  SYNTHETIC CONSTRUCT
ccggcuacacagauaacuu

SEQ ID NO: 226 - RNA, 19 nt, Artificial
  SYNTHETIC CONSTRUCT
ugugucuauugaagauucu

SEQ ID NO: 227 - RNA, 19 nt, Artificial
  SYNTHETIC CONSTRUCT
acacagauaacuucuaaga

SEQ ID NO: 228 - RNA, 19 nt, Artificial
  SYNTHETIC CONSTRUCT
ugaagauucugugaucuca

SEQ ID NO: 229 - RNA, 19 nt, Artificial
  SYNTHETIC CONSTRUCT
acuucuaagacacuagagu

SEQ ID NO: 230 - RNA, 19 nt, Artificial
  SYNTHETIC CONSTRUCT
ugaucucacucucaggaga

SEQ ID NO: 231 - RNA, 19 nt, Artificial
  SYNTHETIC CONSTRUCT
acuagagugagaguccucu

SEQ ID NO: 232 - RNA, 19 nt, Artificial
  SYNTHETIC CONSTRUCT
ucaggagaccauugcauca

SEQ ID NO: 233 - RNA, 19 nt, Artificial
  SYNTHETIC CONSTRUCT
aguccucugguaacguagu

SEQ ID NO: 234 - RNA, 19 nt, Artificial
  SYNTHETIC CONSTRUCT
aggagaccauugcaucauu

SEQ ID NO: 235 - RNA, 19 nt, Artificial
  SYNTHETIC CONSTRUCT
uccucugguaacguaguaa

SEQ ID NO: 236 - RNA, 19 nt, Artificial
  SYNTHETIC CONSTRUCT
cacacuggugguccaugaa

SEQ ID NO: 237 - RNA, 19 nt, Artificial
  SYNTHETIC CONSTRUCT
gugugaccaccagguacuu

SEQ ID NO: 238 - RNA, 19 nt, Artificial
  SYNTHETIC CONSTRUCT
ggugguccaugaaaaagca

SEQ ID NO: 239 - RNA, 19 nt, Artificial
  SYNTHETIC CONSTRUCT
ccaccagguacuuuuucgu

SEQ ID NO: 240 - RNA, 19 nt, Artificial
  SYNTHETIC CONSTRUCT
gcaaagguggaaaugaaga

SEQ ID NO: 241 - RNA, 19 nt, Artificial
  SYNTHETIC CONSTRUCT
cguuuccaccuuuacuucu

SEQ ID NO: 242 - RNA, 19 nt, Artificial
  SYNTHETIC CONSTRUCT
aaagguggaaaugaagaaa

SEQ ID NO: 243 - RNA, 19 nt, Artificial
  SYNTHETIC CONSTRUCT
uuuccaccuuuacuucuuu

SEQ ID NO: 244 - RNA, 19 nt, Artificial
  SYNTHETIC CONSTRUCT
agguggaaaugaagaaagu

SEQ ID NO: 245 - RNA, 19 nt, Artificial
  SYNTHETIC CONSTRUCT
uccaccuuuacuucuuuca

SEQ ID NO: 246 - RNA, 19 nt, Artificial
  SYNTHETIC CONSTRUCT
gaaaguacaaagacaggaa

SEQ ID NO: 247 - RNA, 19 nt, Artificial
  SYNTHETIC CONSTRUCT
cuuucauguuucuguccuu

SEQ ID NO: 248 - RNA, 19 nt, Artificial
  SYNTHETIC CONSTRUCT
aaaguacaaagacaggaaa

SEQ ID NO: 249 - RNA, 19 nt, Artificial
  SYNTHETIC CONSTRUCT
uuucauguuucuguccuuu

SEQ ID NO: 250 - RNA, 19 nt, Artificial
  SYNTHETIC CONSTRUCT
ucguuuggcuuguggugua

SEQ ID NO: 251 - RNA, 19 nt, Artificial
  SYNTHETIC CONSTRUCT
agcaaaccgaacaccacau

SEQ ID NO: 252 - RNA, 19 nt, Artificial
  SYNTHETIC CONSTRUCT
cguuuggcuugugguguaa

SEQ ID NO: 253 - RNA, 19 nt, Artificial
  SYNTHETIC CONSTRUCT
gcaaaccgaacaccacauu

SEQ ID NO: 254 - RNA, 19 nt, Artificial
  SYNTHETIC CONSTRUCT
ugggaucgcccaauaaaca

SEQ ID NO: 255 - RNA, 19 nt, Artificial
  SYNTHETIC CONSTRUCT
acccuagcggguuauuugu

SEQ ID NO: 256 - RNA, 19 nt, Artificial
  SYNTHETIC CONSTRUCT
ggaucgcccaauaaacauu

SEQ ID NO: 257 - RNA, 19 nt, Artificial
  SYNTHETIC CONSTRUCT
ccuagcggguuauuuguaa

SEQ ID NO: 258 - RNA, 19 nt, Artificial
  SYNTHETIC CONSTRUCT
aaacauucccuuggaugua

SEQ ID NO: 259 - RNA, 19 nt, Artificial
  SYNTHETIC CONSTRUCT
uuuguaagggaaccuacau

SEQ ID NO: 260 - RNA, 19 nt, Artificial
  SYNTHETIC CONSTRUCT
acauucccuuggauguagu

SEQ ID NO: 261 - RNA, 19 nt, Artificial
  SYNTHETIC CONSTRUCT
uguaagggaaccuacauca

SEQ ID NO: 262 - RNA, 19 nt, Artificial
  SYNTHETIC CONSTRUCT
ucccuuggauguagucuga

SEQ ID NO: 263 - RNA, 19 nt, Artificial
  SYNTHETIC CONSTRUCT
agggaaccuacaucagacu

SEQ ID NO: 264 - RNA, 19 nt, Artificial
  SYNTHETIC CONSTRUCT
cucaucuguuauccugcua

SEQ ID NO: 265 - RNA, 19 nt, Artificial
  SYNTHETIC CONSTRUCT
gaguagacaauaggacgau

SEQ ID NO: 266 - RNA, 19 nt, Artificial
  SYNTHETIC CONSTRUCT
uauccugcuagcuguagaa

SEQ ID NO: 267 - RNA, 19 nt, Artificial
  SYNTHETIC CONSTRUCT
auaggacgaucgacaucuu

SEQ ID NO: 268 - RNA, 19 nt, Artificial
  SYNTHETIC CONSTRUCT
aaaguguaauugugugacu

SEQ ID NO: 269 - RNA, 19 nt, Artificial
  SYNTHETIC CONSTRUCT
uuucacauuaacacacuga

SEQ ID NO: 270 - RNA, 19 nt, Artificial
  SYNTHETIC CONSTRUCT
guaauugugugacuuuuuc

SEQ ID NO: 271 - RNA, 19 nt, Artificial
  SYNTHETIC CONSTRUCT
cauuaacacacugaaaaag

SEQ ID NO: 272 - RNA, 19 nt, Artificial
  SYNTHETIC CONSTRUCT
cuuuuucagaguugcuuua

SEQ ID NO: 273 - RNA, 19 nt, Artificial
  SYNTHETIC CONSTRUCT
gaaaaagucucaacgaaau

SEQ ID NO: 274 - RNA, 19 nt, Artificial
  SYNTHETIC CONSTRUCT
guaccuguagugagaaacu

SEQ ID NO: 275 - RNA, 19 nt, Artificial
  SYNTHETIC CONSTRUCT
cauggacaucacucuuuga

SEQ ID NO: 276 - RNA, 19 nt, Artificial
  SYNTHETIC CONSTRUCT
ugauuuaugaucacuugga

SEQ ID NO: 277 - RNA, 19 nt, Artificial
  SYNTHETIC CONSTRUCT
acuaaauacuagugaaccu

SEQ ID NO: 278 - RNA, 19 nt, Artificial
  SYNTHETIC CONSTRUCT
gauuuaugaucacuuggaa

SEQ ID NO: 279 - RNA, 19 nt, Artificial
  SYNTHETIC CONSTRUCT
cuaaauacuagugaaccuu

SEQ ID NO: 280 - RNA, 19 nt, Artificial
  SYNTHETIC CONSTRUCT
augaucacuuggaagauuu

SEQ ID NO: 281 - RNA, 19 nt, Artificial
  SYNTHETIC CONSTRUCT
uacuagugaaccuucuaaa

SEQ ID NO: 282 - RNA, 19 nt, Artificial
  SYNTHETIC CONSTRUCT
aucacuuggaagauuugua

SEQ ID NO: 283 - RNA, 19 nt, Artificial
  SYNTHETIC CONSTRUCT
uagugaaccuucuaaacau

SEQ ID NO: 284 - RNA, 19 nt, Artificial
  SYNTHETIC CONSTRUCT
ucacuuggaagauuuguau

SEQ ID NO: 285 - RNA, 19 nt, Artificial
  SYNTHETIC CONSTRUCT
agugaaccuucuaaacaua

SEQ ID NO: 286 - RNA, 19 nt, Artificial
  SYNTHETIC CONSTRUCT
cacuuggaagauuuguaua

SEQ ID NO: 287 - RNA, 19 nt, Artificial
  SYNTHETIC CONSTRUCT
gugaaccuucuaaacauau

SEQ ID NO: 288 - RNA, 19 nt, Artificial
  SYNTHETIC CONSTRUCT
cuguuucaaugaccuguau

SEQ ID NO: 289 - RNA, 19 nt, Artificial
  SYNTHETIC CONSTRUCT
gacaaaguuacuggacaua

SEQ ID NO: 290 - RNA, 19 nt, Artificial
  SYNTHETIC CONSTRUCT
guuucaaugaccuguauuu

SEQ ID NO: 291 - RNA, 19 nt, Artificial
  SYNTHETIC CONSTRUCT
caaaguuacuggacauaaa

SEQ ID NO: 292 - RNA, 19 nt, Artificial
  SYNTHETIC CONSTRUCT
ugaccuguauuuugccaga

SEQ ID NO: 293 - RNA, 19 nt, Artificial
  SYNTHETIC CONSTRUCT
acuggacauaaaacggucu

SEQ ID NO: 294 - RNA, 19 nt, Artificial
  SYNTHETIC CONSTRUCT
cuguauuuugccagacuua

SEQ ID NO: 295 - RNA, 19 nt, Artificial
  SYNTHETIC CONSTRUCT
gacauaaaacggucugaau

SEQ ID NO: 296 - RNA, 19 nt, Artificial
  SYNTHETIC CONSTRUCT
uuuugccagacuuaaauca

SEQ ID NO: 297 - RNA, 19 nt, Artificial
  SYNTHETIC CONSTRUCT
aaaacggucugaauuuagu

SEQ ID NO: 298 - RNA, 19 nt, Artificial
  SYNTHETIC CONSTRUCT
uugccagacuuaaaucaca

SEQ ID NO: 299 - RNA, 19 nt, Artificial
  SYNTHETIC CONSTRUCT
aacggucugaauuuagugu

SEQ ID NO: 300 - RNA, 19 nt, Artificial
  SYNTHETIC CONSTRUCT
agauggguauuaaacuugu

SEQ ID NO: 301 - RNA, 19 nt, Artificial
  SYNTHETIC CONSTRUCT
ucuacccauaauuugaaca

SEQ ID NO: 302 - RNA, 19 nt, Artificial
  SYNTHETIC CONSTRUCT
ugucagaauuucuuuguca

SEQ ID NO: 303 - RNA, 19 nt, Artificial
  SYNTHETIC CONSTRUCT
acagucuuaaagaaacagu

SEQ ID NO: 304 - RNA, 19 nt, Artificial
  SYNTHETIC CONSTRUCT
ucauucaagccugugaaua

SEQ ID NO: 305 - RNA, 19 nt, Artificial
  SYNTHETIC CONSTRUCT
aguaaguucggacacuuau

SEQ ID NO: 306 - RNA, 19 nt, Artificial
  SYNTHETIC CONSTRUCT
aaacccuguauggcacuua

SEQ ID NO: 307 - RNA, 19 nt, Artificial
  SYNTHETIC CONSTRUCT
uuugggacauaccgugaau

SEQ ID NO: 308 - RNA, 19 nt, Artificial
  SYNTHETIC CONSTRUCT
aacccuguauggcacuuau

SEQ ID NO: 309 - RNA, 19 nt, Artificial
  SYNTHETIC CONSTRUCT
uugggacauaccgugaaua

SEQ ID NO: 310 - RNA, 19 nt, Artificial
  SYNTHETIC CONSTRUCT
acccuguauggcacuuauu

SEQ ID NO: 311 - RNA, 19 nt, Artificial
  SYNTHETIC CONSTRUCT
ugggacauaccgugaauaa

SEQ ID NO: 312 - RNA, 19 nt, Artificial
  SYNTHETIC CONSTRUCT
ccuguauggcacuuauuau

SEQ ID NO: 313 - RNA, 19 nt, Artificial
  SYNTHETIC CONSTRUCT
ggacauaccgugaauaaua

SEQ ID NO: 314 - RNA, 19 nt, Artificial
  SYNTHETIC CONSTRUCT
uguauggcacuuauuauga

SEQ ID NO: 315 - RNA, 19 nt, Artificial
  SYNTHETIC CONSTRUCT
acauaccgugaauaauacu

SEQ ID NO: 316 - RNA, 19 nt, Artificial
  SYNTHETIC CONSTRUCT
ggcguggccuagcgaguua

SEQ ID NO: 317 - RNA, 19 nt, Artificial
  SYNTHETIC CONSTRUCT
ccgcaccggaucgcucaau

SEQ ID NO: 318 - RNA, 19 nt, Artificial
  SYNTHETIC CONSTRUCT
gcacacuggugguccauga

SEQ ID NO: 319 - RNA, 19 nt, Artificial
  SYNTHETIC CONSTRUCT
cgugugaccaccagguacu

SEQ ID NO: 320 - RNA, 19 nt, Artificial
  SYNTHETIC CONSTRUCT
aaauguauccugauaaaca

SEQ ID NO: 321 - RNA, 19 nt, Artificial
  SYNTHETIC CONSTRUCT
uuuacauaggacuauuugu

SEQ ID NO: 322 - RNA, 19 nt, Artificial
  SYNTHETIC CONSTRUCT
uauccugauaaacauuaaa

SEQ ID NO: 323 - RNA, 19 nt, Artificial
  SYNTHETIC CONSTRUCT
auaggacuauuuguaauuu

SEQ ID NO: 324 - RNA, 19 nt, Artificial
  SYNTHETIC CONSTRUCT
uaaacauuaaacacuguaa

SEQ ID NO: 325 - RNA, 19 nt, Artificial
  SYNTHETIC CONSTRUCT
auuuguaauuugugacauu

SEQ ID NO: 326 - RNA, 19 nt, Artificial
  SYNTHETIC CONSTRUCT
auuaaacacuguaaucuua

SEQ ID NO: 327 - RNA, 19 nt, Artificial
  SYNTHETIC CONSTRUCT
uaauuugugacauuagaau

SEQ ID NO: 328 - RNA, 19 nt, Artificial
  SYNTHETIC CONSTRUCT
uuaaacacuguaaucuuaa

SEQ ID NO: 329 - RNA, 19 nt, Artificial
  SYNTHETIC CONSTRUCT
aauuugugacauuagaauu

SEQ ID NO: 330 - RNA, 19 nt, Artificial
  SYNTHETIC CONSTRUCT
acacuguaaucuuaaaagu

SEQ ID NO: 331 - RNA, 19 nt, Artificial
  SYNTHETIC CONSTRUCT
ugugacauuagaauuuuca

SEQ ID NO: 332 - RNA, 19 nt, Artificial
  SYNTHETIC CONSTRUCT
uaaucuuaaaaguguaauu

SEQ ID NO: 333 - RNA, 19 nt, Artificial
  SYNTHETIC CONSTRUCT
auuagaauuuucacauuaa

SEQ ID NO: 334 - RNA, 19 nt, Artificial
  SYNTHETIC CONSTRUCT
uaauugugugacuuuuuca

SEQ ID NO: 335 - RNA, 19 nt, Artificial
  SYNTHETIC CONSTRUCT
auuaacacacugaaaaagu

SEQ ID NO: 336 - RNA, 19 nt, Artificial
  SYNTHETIC CONSTRUCT
agaaacugauuuaugauca

SEQ ID NO: 337 - RNA, 19 nt, Artificial
  SYNTHETIC CONSTRUCT
ucuuugacuaaauacuagu

SEQ ID NO: 338 - RNA, 19 nt, Artificial
  SYNTHETIC CONSTRUCT
gauuuguauaguuuuauaa

SEQ ID NO: 339 - RNA, 19 nt, Artificial
  SYNTHETIC CONSTRUCT
cuaaacauaucaaaauauu

SEQ ID NO: 340 - RNA, 19 nt, Artificial
  SYNTHETIC CONSTRUCT
auuuguauaguuuuauaaa

SEQ ID NO: 341 - RNA, 19 nt, Artificial
  SYNTHETIC CONSTRUCT
uaaacauaucaaaauauuu

SEQ ID NO: 342 - RNA, 19 nt, Artificial
  SYNTHETIC CONSTRUCT
uuuauaaaacucaguuaaa

SEQ ID NO: 343 - RNA, 19 nt, Artificial
  SYNTHETIC CONSTRUCT
aaauauuuugagucaauuu

SEQ ID NO: 344 - RNA, 19 nt, Artificial
  SYNTHETIC CONSTRUCT
aaaaugucuguuucaauga

SEQ ID NO: 345 - RNA, 19 nt, Artificial
  SYNTHETIC CONSTRUCT
uuuuacagacaaaguuacu

SEQ ID NO: 346 - RNA, 19 nt, Artificial
  SYNTHETIC CONSTRUCT
guauuaaacuugucagaau

SEQ ID NO: 347 - RNA, 19 nt, Artificial
  SYNTHETIC CONSTRUCT
cauaauuugaacagucuua

SEQ ID NO: 348 - RNA, 19 nt, Artificial
  SYNTHETIC CONSTRUCT
cuuauuaugaggcuauuaa

SEQ ID NO: 349 - RNA, 19 nt, Artificial
  SYNTHETIC CONSTRUCT
gaauaauacuccgauaauu

SEQ ID NO: 350 - RNA, 19 nt, Artificial
  SYNTHETIC CONSTRUCT
uauuaugaggcuauuaaaa

SEQ ID NO: 351 - RNA, 19 nt, Artificial
  SYNTHETIC CONSTRUCT
auaauacuccgauaauuuu

SEQ ID NO: 352 - RNA, 19 nt, Artificial
  SYNTHETIC CONSTRUCT
uaaaagaauccaaauucaa

SEQ ID NO: 353 - RNA, 19 nt, Artificial
  SYNTHETIC CONSTRUCT
auuuucuuagguuuaaguu

SEQ ID NO: 354 - RNA, 19 nt, Artificial
  SYNTHETIC CONSTRUCT
aaaagaauccaaauucaaa

SEQ ID NO: 355 - RNA, 19 nt, Artificial
  SYNTHETIC CONSTRUCT
uuuucuuagguuuaaguuu

SEQ ID NO: 356 - RNA, 19 nt, Artificial
  SYNTHETIC CONSTRUCT
agaauccaaauucaaacua

SEQ ID NO: 357 - RNA, 19 nt, Artificial
  SYNTHETIC CONSTRUCT
ucuuagguuuaaguuugau

SEQ ID NO: 358 - RNA, 19 nt, Artificial
  SYNTHETIC CONSTRUCT
aauccaaauucaaacuaaa

SEQ ID NO: 359 - RNA, 19 nt, Artificial
  SYNTHETIC CONSTRUCT
uuagguuuaaguuugauuu

SEQ ID NO: 360 - RNA, 19 nt, Artificial
  SYNTHETIC CONSTRUCT
ggaggucuggccuauaaag

SEQ ID NO: 361 - RNA, 19 nt, Artificial
  SYNTHETIC CONSTRUCT
ccuccagaccggauauuuc

SEQ ID NO: 362 - RNA, 19 nt, Artificial
  SYNTHETIC CONSTRUCT
gaggucuggccuauaaagu

SEQ ID NO: 363 - RNA, 19 nt, Artificial
  SYNTHETIC CONSTRUCT
cuccagaccggauauuuca

SEQ ID NO: 364 - RNA, 19 nt, Artificial
  SYNTHETIC CONSTRUCT
agugcagggcaucaucaau

SEQ ID NO: 365 - RNA, 19 nt, Artificial
  SYNTHETIC CONSTRUCT
ucacgucccguaguaguua

SEQ ID NO: 366 - RNA, 19 nt, Artificial
  SYNTHETIC CONSTRUCT
gugcagggcaucaucaauu

SEQ ID NO: 367 - RNA, 19 nt, Artificial
  SYNTHETIC CONSTRUCT
cacgucccguaguaguuaa

SEQ ID NO: 368 - RNA, 19 nt, Artificial
  SYNTHETIC CONSTRUCT
gcagggcaucaucaauuuc

SEQ ID NO: 369 - RNA, 19 nt, Artificial
  SYNTHETIC CONSTRUCT
cgucccguaguaguuaaag

SEQ ID NO: 370 - RNA, 19 nt, Artificial
  SYNTHETIC CONSTRUCT
gcaucaucaauuucgagca

SEQ ID NO: 371 - RNA, 19 nt, Artificial
  SYNTHETIC CONSTRUCT
cguaguaguuaaagcucgu

SEQ ID NO: 372 - RNA, 19 nt, Artificial
  SYNTHETIC CONSTRUCT
caauuucgagcagaaggaa

SEQ ID NO: 373 - RNA, 19 nt, Artificial
  SYNTHETIC CONSTRUCT
guuaaagcucgucuuccuu

SEQ ID NO: 374 - RNA, 19 nt, Artificial
  SYNTHETIC CONSTRUCT
aauuucgagcagaaggaaa

SEQ ID NO: 375 - RNA, 19 nt, Artificial
  SYNTHETIC CONSTRUCT
uuaaagcucgucuuccuuu

SEQ ID NO: 376 - RNA, 19 nt, Artificial
  SYNTHETIC CONSTRUCT
uucgagcagaaggaaagua

SEQ ID NO: 377 - RNA, 19 nt, Artificial
  SYNTHETIC CONSTRUCT
aagcucgucuuccuuucau

SEQ ID NO: 378 - RNA, 19 nt, Artificial
  SYNTHETIC CONSTRUCT
guaauggaccagugaaggu

SEQ ID NO: 379 - RNA, 19 nt, Artificial
  SYNTHETIC CONSTRUCT
cauuaccuggucacuucca

SEQ ID NO: 380 - RNA, 19 nt, Artificial
  SYNTHETIC CONSTRUCT
gaagguguggggaagcauu

SEQ ID NO: 381 - RNA, 19 nt, Artificial
  SYNTHETIC CONSTRUCT
cuuccacaccccuucguaa

SEQ ID NO: 382 - RNA, 19 nt, Artificial
  SYNTHETIC CONSTRUCT
guggggaagcauuaaagga

SEQ ID NO: 383 - RNA, 19 nt, Artificial
  SYNTHETIC CONSTRUCT
caccccuucguaauuuccu

SEQ ID NO: 384 - RNA, 19 nt, Artificial
  SYNTHETIC CONSTRUCT
ggaagcauuaaaggacuga

SEQ ID NO: 385 - RNA, 19 nt, Artificial
  SYNTHETIC CONSTRUCT
ccuucguaauuuccugacu

SEQ ID NO: 386 - RNA, 19 nt, Artificial
  SYNTHETIC CONSTRUCT
cugaaggccugcauggauu

SEQ ID NO: 387 - RNA, 19 nt, Artificial
  SYNTHETIC CONSTRUCT
gacuuccggacguaccuaa

SEQ ID NO: 388 - RNA, 19 nt, Artificial
  SYNTHETIC CONSTRUCT
aggccugcauggauuccau

SEQ ID NO: 389 - RNA, 19 nt, Artificial
  SYNTHETIC CONSTRUCT
uccggacguaccuaaggua

SEQ ID NO: 390 - RNA, 19 nt, Artificial
  SYNTHETIC CONSTRUCT
ccugcauggauuccauguu

SEQ ID NO: 391 - RNA, 19 nt, Artificial
  SYNTHETIC CONSTRUCT
ggacguaccuaagguacaa

SEQ ID NO: 392 - RNA, 19 nt, Artificial
  SYNTHETIC CONSTRUCT
auggauuccauguucauga

SEQ ID NO: 393 - RNA, 19 nt, Artificial
  SYNTHETIC CONSTRUCT
uaccuaagguacaaguacu

SEQ ID NO: 394 - RNA, 19 nt, Artificial
  SYNTHETIC CONSTRUCT
ggauuccauguucaugagu

SEQ ID NO: 395 - RNA, 19 nt, Artificial
  SYNTHETIC CONSTRUCT
ccuaagguacaaguacuca

SEQ ID NO: 396 - RNA, 19 nt, Artificial
  SYNTHETIC CONSTRUCT
gauuccauguucaugaguu

SEQ ID NO: 397 - RNA, 19 nt, Artificial
  SYNTHETIC CONSTRUCT
cuaagguacaaguacucaa

SEQ ID NO: 398 - RNA, 19 nt, Artificial
  SYNTHETIC CONSTRUCT
guuuggagauaauacagca

SEQ ID NO: 399 - RNA, 19 nt, Artificial
  SYNTHETIC CONSTRUCT
caaaccucuauuaugucgu

SEQ ID NO: 400 - RNA, 19 nt, Artificial
  SYNTHETIC CONSTRUCT
aauacagcaggcuguacca

SEQ ID NO: 401 - RNA, 19 nt, Artificial
  SYNTHETIC CONSTRUCT
uuaugucguccgacauggu

SEQ ID NO: 402 - RNA, 19 nt, Artificial
  SYNTHETIC CONSTRUCT
gcagguccucacuuuaauc

SEQ ID NO: 403 - RNA, 19 nt, Artificial
  SYNTHETIC CONSTRUCT
cguccaggagugaaauuag

SEQ ID NO: 404 - RNA, 19 nt, Artificial
  SYNTHETIC CONSTRUCT
ccucacuuuaauccucuau

SEQ ID NO: 405 - RNA, 19 nt, Artificial
  SYNTHETIC CONSTRUCT
ggagugaaauuaggagaua

SEQ ID NO: 406 - RNA, 19 nt, Artificial
  SYNTHETIC CONSTRUCT
ucacuuuaauccucuaucc

SEQ ID NO: 407 - RNA, 19 nt, Artificial
  SYNTHETIC CONSTRUCT
agugaaauuaggagauagg

SEQ ID NO: 408 - RNA, 19 nt, Artificial
  SYNTHETIC CONSTRUCT
cacuuuaauccucuaucca

SEQ ID NO: 409 - RNA, 19 nt, Artificial
  SYNTHETIC CONSTRUCT
gugaaauuaggagauaggu

SEQ ID NO: 410 - RNA, 19 nt, Artificial
  SYNTHETIC CONSTRUCT
cuuuaauccucuauccaga

SEQ ID NO: 411 - RNA, 19 nt, Artificial
  SYNTHETIC CONSTRUCT
gaaauuaggagauaggucu

SEQ ID NO: 412 - RNA, 19 nt, Artificial
  SYNTHETIC CONSTRUCT
uuuaauccucuauccagaa

SEQ ID NO: 413 - RNA, 19 nt, Artificial
  SYNTHETIC CONSTRUCT
aaauuaggagauaggucuu

SEQ ID NO: 414 - RNA, 19 nt, Artificial
  SYNTHETIC CONSTRUCT
aauccucuauccagaaaac

SEQ ID NO: 415 - RNA, 19 nt, Artificial
  SYNTHETIC CONSTRUCT
uuaggagauaggucuuuug

SEQ ID NO: 416 - RNA, 19 nt, Artificial
  SYNTHETIC CONSTRUCT
auccucuauccagaaaaca

SEQ ID NO: 417 - RNA, 19 nt, Artificial
  SYNTHETIC CONSTRUCT
uaggagauaggucuuuugu

SEQ ID NO: 418 - RNA, 19 nt, Artificial
  SYNTHETIC CONSTRUCT
gaaaacacggugggccaaa

SEQ ID NO: 419 - RNA, 19 nt, Artificial
  SYNTHETIC CONSTRUCT
cuuuugugccacccgguuu

SEQ ID NO: 420 - RNA, 19 nt, Artificial
  SYNTHETIC CONSTRUCT
gugggccaaaggaugaaga

SEQ ID NO: 421 - RNA, 19 nt, Artificial
  SYNTHETIC CONSTRUCT
cacccgguuuccuacuucu

SEQ ID NO: 422 - RNA, 19 nt, Artificial
  SYNTHETIC CONSTRUCT
caaaggaugaagagaggca

SEQ ID NO: 423 - RNA, 19 nt, Artificial
  SYNTHETIC CONSTRUCT
guuuccuacuucucuccgu

SEQ ID NO: 424 - RNA, 19 nt, Artificial
  SYNTHETIC CONSTRUCT
ggaugaagagaggcauguu

SEQ ID NO: 425 - RNA, 19 nt, Artificial
  SYNTHETIC CONSTRUCT
ccuacuucucuccguacaa

SEQ ID NO: 426 - RNA, 19 nt, Artificial
  SYNTHETIC CONSTRUCT
agaggcauguuggagacuu

SEQ ID NO: 427 - RNA, 19 nt, Artificial
  SYNTHETIC CONSTRUCT
ucuccguacaaccucugaa

SEQ ID NO: 428 - RNA, 19 nt, Artificial
  SYNTHETIC CONSTRUCT
uuggagacuugggcaaugu

SEQ ID NO: 429 - RNA, 19 nt, Artificial
  SYNTHETIC CONSTRUCT
aaccucugaacccguuaca

SEQ ID NO: 430 - RNA, 19 nt, Artificial
  SYNTHETIC CONSTRUCT
agacuugggcaaugugacu

SEQ ID NO: 431 - RNA, 19 nt, Artificial
  SYNTHETIC CONSTRUCT
ucugaacccguuacacuga

SEQ ID NO: 432 - RNA, 19 nt, Artificial
  SYNTHETIC CONSTRUCT
ugggcaaugugacugcuga

SEQ ID NO: 433 - RNA, 19 nt, Artificial
  SYNTHETIC CONSTRUCT
acccguuacacugacgacu

SEQ ID NO: 434 - RNA, 19 nt, Artificial
  SYNTHETIC CONSTRUCT
aagaugguguggccgaugu

SEQ ID NO: 435 - RNA, 19 nt, Artificial
  SYNTHETIC CONSTRUCT
uucuaccacaccggcuaca

SEQ ID NO: 436 - RNA, 19 nt, Artificial
  SYNTHETIC CONSTRUCT
guguggccgaugugucuau

SEQ ID NO: 437 - RNA, 19 nt, Artificial
  SYNTHETIC CONSTRUCT
cacaccggcuacacagaua

SEQ ID NO: 438 - RNA, 19 nt, Artificial
  SYNTHETIC CONSTRUCT
uguggccgaugugucuauu

SEQ ID NO: 439 - RNA, 19 nt, Artificial
  SYNTHETIC CONSTRUCT
acaccggcuacacagauaa

SEQ ID NO: 440 - RNA, 19 nt, Artificial
  SYNTHETIC CONSTRUCT
ccgaugugucuauugaaga

SEQ ID NO: 441 - RNA, 19 nt, Artificial
  SYNTHETIC CONSTRUCT
ggcuacacagauaacuucu

SEQ ID NO: 442 - RNA, 19 nt, Artificial
  SYNTHETIC CONSTRUCT
ucuauugaagauucuguga

SEQ ID NO: 443 - RNA, 19 nt, Artificial
  SYNTHETIC CONSTRUCT
agauaacuucuaagacacu

SEQ ID NO: 444 - RNA, 19 nt, Artificial
  SYNTHETIC CONSTRUCT
cuauugaagauucugugau

SEQ ID NO: 445 - RNA, 19 nt, Artificial
  SYNTHETIC CONSTRUCT
gauaacuucuaagacacua

SEQ ID NO: 446 - RNA, 19 nt, Artificial
  SYNTHETIC CONSTRUCT
aagauucugugaucucacu

SEQ ID NO: 447 - RNA, 19 nt, Artificial
  SYNTHETIC CONSTRUCT
uucuaagacacuagaguga

SEQ ID NO: 448 - RNA, 19 nt, Artificial
  SYNTHETIC CONSTRUCT
uucugugaucucacucuca

SEQ ID NO: 449 - RNA, 19 nt, Artificial
  SYNTHETIC CONSTRUCT
aagacacuagagugagagu

SEQ ID NO: 450 - RNA, 19 nt, Artificial
  SYNTHETIC CONSTRUCT
cucucaggagaccauugca

SEQ ID NO: 451 - RNA, 19 nt, Artificial
  SYNTHETIC CONSTRUCT
gagaguccucugguaacgu

SEQ ID NO: 452 - RNA, 19 nt, Artificial
  SYNTHETIC CONSTRUCT
ggagaccauugcaucauug

SEQ ID NO: 453 - RNA, 19 nt, Artificial
  SYNTHETIC CONSTRUCT
ccucugguaacguaguaac

SEQ ID NO: 454 - RNA, 19 nt, Artificial
  SYNTHETIC CONSTRUCT
uugcaucauuggccgcaca

SEQ ID NO: 455 - RNA, 19 nt, Artificial
  SYNTHETIC CONSTRUCT
aacguaguaaccggcgugu

SEQ ID NO: 456 - RNA, 19 nt, Artificial
  SYNTHETIC CONSTRUCT
uccaugaaaaagcagauga

SEQ ID NO: 457 - RNA, 19 nt, Artificial
  SYNTHETIC CONSTRUCT
agguacuuuuucgucuacu

SEQ ID NO: 458 - RNA, 19 nt, Artificial
  SYNTHETIC CONSTRUCT
agaugacuugggcaaaggu

SEQ ID NO: 459 - RNA, 19 nt, Artificial
  SYNTHETIC CONSTRUCT
ucuacugaacccguuucca

SEQ ID NO: 460 - RNA, 19 nt, Artificial
  SYNTHETIC CONSTRUCT
ugacuugggcaaaggugga

SEQ ID NO: 461 - RNA, 19 nt, Artificial
  SYNTHETIC CONSTRUCT
acugaacccguuuccaccu

SEQ ID NO: 462 - RNA, 19 nt, Artificial
  SYNTHETIC CONSTRUCT
cuugggcaaagguggaaau

SEQ ID NO: 463 - RNA, 19 nt, Artificial
  SYNTHETIC CONSTRUCT
gaacccguuuccaccuuua

SEQ ID NO: 464 - RNA, 19 nt, Artificial
  SYNTHETIC CONSTRUCT
ugggcaaagguggaaauga

SEQ ID NO: 465 - RNA, 19 nt, Artificial
  SYNTHETIC CONSTRUCT
acccguuuccaccuuuacu

SEQ ID NO: 466 - RNA, 19 nt, Artificial
  SYNTHETIC CONSTRUCT
gguggaaaugaagaaagua

SEQ ID NO: 467 - RNA, 19 nt, Artificial
  SYNTHETIC CONSTRUCT
ccaccuuuacuucuuucau

SEQ ID NO: 468 - RNA, 19 nt, Artificial
  SYNTHETIC CONSTRUCT
uggaaaugaagaaaguaca

SEQ ID NO: 469 - RNA, 19 nt, Artificial
  SYNTHETIC CONSTRUCT
accuuuacuucuuucaugu

SEQ ID NO: 470 - RNA, 19 nt, Artificial
  SYNTHETIC CONSTRUCT
aaagacaggaaacgcugga

SEQ ID NO: 471 - RNA, 19 nt, Artificial
  SYNTHETIC CONSTRUCT
uuucuguccuuugcgaccu

SEQ ID NO: 472 - RNA, 19 nt, Artificial
  SYNTHETIC CONSTRUCT
ggaaacgcuggaagucguu

SEQ ID NO: 473 - RNA, 19 nt, Artificial
  SYNTHETIC CONSTRUCT
ccuuugcgaccuucagcaa

SEQ ID NO: 474 - RNA, 19 nt, Artificial
  SYNTHETIC CONSTRUCT
gggaucgcccaauaaacau

SEQ ID NO: 475 - RNA, 19 nt, Artificial
  SYNTHETIC CONSTRUCT
cccuagcggguuauuugua

SEQ ID NO: 476 - RNA, 19 nt, Artificial
  SYNTHETIC CONSTRUCT
aauaaacauucccuuggau

SEQ ID NO: 477 - RNA, 19 nt, Artificial
  SYNTHETIC CONSTRUCT
uuauuuguaagggaaccua

SEQ ID NO: 478 - RNA, 19 nt, Artificial
  SYNTHETIC CONSTRUCT
uaaacauucccuuggaugu

SEQ ID NO: 479 - RNA, 19 nt, Artificial
  SYNTHETIC CONSTRUCT
auuuguaagggaaccuaca

SEQ ID NO: 480 - RNA, 19 nt, Artificial
  SYNTHETIC CONSTRUCT
uguagucugaggccccuua

SEQ ID NO: 481 - RNA, 19 nt, Artificial
  SYNTHETIC CONSTRUCT
acaucagacuccggggaau

SEQ ID NO: 482 - RNA, 19 nt, Artificial
  SYNTHETIC CONSTRUCT
agucugaggccccuuaacu

SEQ ID NO: 483 - RNA, 19 nt, Artificial
  SYNTHETIC CONSTRUCT
ucagacuccggggaauuga

SEQ ID NO: 484 - RNA, 19 nt, Artificial
  SYNTHETIC CONSTRUCT
ucugaggccccuuaacuca

SEQ ID NO: 485 - RNA, 19 nt, Artificial
  SYNTHETIC CONSTRUCT
agacuccggggaauugagu

SEQ ID NO: 486 - RNA, 19 nt, Artificial
  SYNTHETIC CONSTRUCT
ugaggccccuuaacucauc

SEQ ID NO: 487 - RNA, 19 nt, Artificial
  SYNTHETIC CONSTRUCT
acuccggggaauugaguag

SEQ ID NO: 488 - RNA, 19 nt, Artificial
  SYNTHETIC CONSTRUCT
gaggccccuuaacucaucu

SEQ ID NO: 489 - RNA, 19 nt, Artificial
  SYNTHETIC CONSTRUCT
cuccggggaauugaguaga

SEQ ID NO: 490 - RNA, 19 nt, Artificial
  SYNTHETIC CONSTRUCT
cccuuaacucaucuguuau

SEQ ID NO: 491 - RNA, 19 nt, Artificial
  SYNTHETIC CONSTRUCT
gggaauugaguagacaaua

SEQ ID NO: 492 - RNA, 19 nt, Artificial
  SYNTHETIC CONSTRUCT
uuaacucaucuguuauccu

SEQ ID NO: 493 - RNA, 19 nt, Artificial
  SYNTHETIC CONSTRUCT
aauugaguagacaauagga

SEQ ID NO: 494 - RNA, 19 nt, Artificial
  SYNTHETIC CONSTRUCT
uguuauccugcuagcugua

SEQ ID NO: 495 - RNA, 19 nt, Artificial
  SYNTHETIC CONSTRUCT
acaauaggacgaucgacau

SEQ ID NO: 496 - RNA, 19 nt, Artificial
  SYNTHETIC CONSTRUCT
auccugcuagcuguagaaa

SEQ ID NO: 497 - RNA, 19 nt, Artificial
  SYNTHETIC CONSTRUCT
uaggacgaucgacaucuuu

SEQ ID NO: 498 - RNA, 19 nt, Artificial
  SYNTHETIC CONSTRUCT
uccugcuagcuguagaaau

SEQ ID NO: 499 - RNA, 19 nt, Artificial
  SYNTHETIC CONSTRUCT
aggacgaucgacaucuuua

SEQ ID NO: 500 - RNA, 19 nt, Artificial
  SYNTHETIC CONSTRUCT
ccugcuagcuguagaaaug

SEQ ID NO: 501 - RNA, 19 nt, Artificial
  SYNTHETIC CONSTRUCT
ggacgaucgacaucuuuac

SEQ ID NO: 502 - RNA, 19 nt, Artificial
  SYNTHETIC CONSTRUCT
gcuagcuguagaaauguau

SEQ ID NO: 503 - RNA, 19 nt, Artificial
  SYNTHETIC CONSTRUCT
cgaucgacaucuuuacaua

SEQ ID NO: 504 - RNA, 19 nt, Artificial
  SYNTHETIC CONSTRUCT
cuguagaaauguauccuga

SEQ ID NO: 505 - RNA, 19 nt, Artificial
  SYNTHETIC CONSTRUCT
gacaucuuuacauaggacu

SEQ ID NO: 506 - RNA, 19 nt, Artificial
  SYNTHETIC CONSTRUCT
aguguaauugugugacuuu

SEQ ID NO: 507 - RNA, 19 nt, Artificial
  SYNTHETIC CONSTRUCT
ucacauuaacacacugaaa

SEQ ID NO: 508 - RNA, 19 nt, Artificial
  SYNTHETIC CONSTRUCT
guguaauugugugacuuuu

SEQ ID NO: 509 - RNA, 19 nt, Artificial
  SYNTHETIC CONSTRUCT
cacauuaacacacugaaaa

SEQ ID NO: 510 - RNA, 19 nt, Artificial
  SYNTHETIC CONSTRUCT
auugugugacuuuuucaga

SEQ ID NO: 511 - RNA, 19 nt, Artificial
  SYNTHETIC CONSTRUCT
uaacacacugaaaaagucu

SEQ ID NO: 512 - RNA, 19 nt, Artificial
  SYNTHETIC CONSTRUCT
ugugugacuuuuucagagu

SEQ ID NO: 513 - RNA, 19 nt, Artificial
  SYNTHETIC CONSTRUCT
acacacugaaaaagucuca

SEQ ID NO: 514 - RNA, 19 nt, Artificial
  SYNTHETIC CONSTRUCT
gugugacuuuuucagaguu

SEQ ID NO: 515 - RNA, 19 nt, Artificial
  SYNTHETIC CONSTRUCT
cacacugaaaaagucucaa

SEQ ID NO: 516 - RNA, 19 nt, Artificial
  SYNTHETIC CONSTRUCT
ugacuuuuucagaguugcu

SEQ ID NO: 517 - RNA, 19 nt, Artificial
  SYNTHETIC CONSTRUCT
acugaaaaagucucaacga

SEQ ID NO: 518 - RNA, 19 nt, Artificial
  SYNTHETIC CONSTRUCT
uuucagaguugcuuuaaag

SEQ ID NO: 519 - RNA, 19 nt, Artificial
  SYNTHETIC CONSTRUCT
aaagucucaacgaaauuuc

SEQ ID NO: 520 - RNA, 19 nt, Artificial
  SYNTHETIC CONSTRUCT
uucagaguugcuuuaaagu

SEQ ID NO: 521 - RNA, 19 nt, Artificial
  SYNTHETIC CONSTRUCT
aagucucaacgaaauuuca

SEQ ID NO: 522 - RNA, 19 nt, Artificial
  SYNTHETIC CONSTRUCT
gaguugcuuuaaaguaccu

SEQ ID NO: 523 - RNA, 19 nt, Artificial
  SYNTHETIC CONSTRUCT
cucaacgaaauuucaugga

SEQ ID NO: 524 - RNA, 19 nt, Artificial
  SYNTHETIC CONSTRUCT
uugcuuuaaaguaccugua

SEQ ID NO: 525 - RNA, 19 nt, Artificial
  SYNTHETIC CONSTRUCT
aacgaaauuucauggacau

SEQ ID NO: 526 - RNA, 19 nt, Artificial
  SYNTHETIC CONSTRUCT
gcuuuaaaguaccuguagu

SEQ ID NO: 527 - RNA, 19 nt, Artificial
  SYNTHETIC CONSTRUCT
cgaaauuucauggacauca

SEQ ID NO: 528 - RNA, 19 nt, Artificial
  SYNTHETIC CONSTRUCT
aaaguaccuguagugagaa

SEQ ID NO: 529 - RNA, 19 nt, Artificial
  SYNTHETIC CONSTRUCT
uuucauggacaucacucuu

SEQ ID NO: 530 - RNA, 19 nt, Artificial
  SYNTHETIC CONSTRUCT
accuguagugagaaacuga

SEQ ID NO: 531 - RNA, 19 nt, Artificial
  SYNTHETIC CONSTRUCT
uggacaucacucuuugacu

SEQ ID NO: 532 - RNA, 19 nt, Artificial
  SYNTHETIC CONSTRUCT
cuguagugagaaacugauu

SEQ ID NO: 533 - RNA, 19 nt, Artificial
  SYNTHETIC CONSTRUCT
gacaucacucuuugacuaa

SEQ ID NO: 534 - RNA, 19 nt, Artificial
  SYNTHETIC CONSTRUCT
uguagugagaaacugauuu

SEQ ID NO: 535 - RNA, 19 nt, Artificial
  SYNTHETIC CONSTRUCT
acaucacucuuugacuaaa

SEQ ID NO: 536 - RNA, 19 nt, Artificial
  SYNTHETIC CONSTRUCT
gaaacugauuuaugaucac

SEQ ID NO: 537 - RNA, 19 nt, Artificial
  SYNTHETIC CONSTRUCT
cuuugacuaaauacuagug

SEQ ID NO: 538 - RNA, 19 nt, Artificial
  SYNTHETIC CONSTRUCT
uuuaugaucacuuggaaga

SEQ ID NO: 539 - RNA, 19 nt, Artificial
  SYNTHETIC CONSTRUCT
aaauacuagugaaccuucu

SEQ ID NO: 540 - RNA, 19 nt, Artificial
  SYNTHETIC CONSTRUCT
uuaugaucacuuggaagau

SEQ ID NO: 541 - RNA, 19 nt, Artificial
  SYNTHETIC CONSTRUCT
aauacuagugaaccuucua

SEQ ID NO: 542 - RNA, 19 nt, Artificial
  SYNTHETIC CONSTRUCT
uaugaucacuuggaagauu

SEQ ID NO: 543 - RNA, 19 nt, Artificial
  SYNTHETIC CONSTRUCT
auacuagugaaccuucuaa

SEQ ID NO: 544 - RNA, 19 nt, Artificial
  SYNTHETIC CONSTRUCT
ugaucacuuggaagauuug

SEQ ID NO: 545 - RNA, 19 nt, Artificial
  SYNTHETIC CONSTRUCT
acuagugaaccuucuaaac

SEQ ID NO: 546 - RNA, 19 nt, Artificial
  SYNTHETIC CONSTRUCT
cuuggaagauuuguauagu

SEQ ID NO: 547 - RNA, 19 nt, Artificial
  SYNTHETIC CONSTRUCT
gaaccuucuaaacauauca

SEQ ID NO: 548 - RNA, 19 nt, Artificial
  SYNTHETIC CONSTRUCT
aaaugucuguuucaaugac

SEQ ID NO: 549 - RNA, 19 nt, Artificial
  SYNTHETIC CONSTRUCT
uuuacagacaaaguuacug

SEQ ID NO: 550 - RNA, 19 nt, Artificial
  SYNTHETIC CONSTRUCT
ucuguuucaaugaccugua

SEQ ID NO: 551 - RNA, 19 nt, Artificial
  SYNTHETIC CONSTRUCT
agacaaaguuacuggacau

SEQ ID NO: 552 - RNA, 19 nt, Artificial
  SYNTHETIC CONSTRUCT
uguuucaaugaccuguauu

SEQ ID NO: 553 - RNA, 19 nt, Artificial
  SYNTHETIC CONSTRUCT
acaaaguuacuggacauaa

SEQ ID NO: 554 - RNA, 19 nt, Artificial
  SYNTHETIC CONSTRUCT
ucaaugaccuguauuuugc

SEQ ID NO: 555 - RNA, 19 nt, Artificial
  SYNTHETIC CONSTRUCT
aguuacuggacauaaaacg

SEQ ID NO: 556 - RNA, 19 nt, Artificial
  SYNTHETIC CONSTRUCT
aaugaccuguauuuugcca

SEQ ID NO: 557 - RNA, 19 nt, Artificial
  SYNTHETIC CONSTRUCT
uuacuggacauaaaacggu

SEQ ID NO: 558 - RNA, 19 nt, Artificial
  SYNTHETIC CONSTRUCT
accuguauuuugccagacu

SEQ ID NO: 559 - RNA, 19 nt, Artificial
  SYNTHETIC CONSTRUCT
uggacauaaaacggucuga

SEQ ID NO: 560 - RNA, 19 nt, Artificial
  SYNTHETIC CONSTRUCT
ccuguauuuugccagacuu

SEQ ID NO: 561 - RNA, 19 nt, Artificial
  SYNTHETIC CONSTRUCT
ggacauaaaacggucugaa

SEQ ID NO: 562 - RNA, 19 nt, Artificial
  SYNTHETIC CONSTRUCT
uguauuuugccagacuuaa

SEQ ID NO: 563 - RNA, 19 nt, Artificial
  SYNTHETIC CONSTRUCT
acauaaaacggucugaauu

SEQ ID NO: 564 - RNA, 19 nt, Artificial
  SYNTHETIC CONSTRUCT
gccagacuuaaaucacaga

SEQ ID NO: 565 - RNA, 19 nt, Artificial
  SYNTHETIC CONSTRUCT
cggucugaauuuagugucu

SEQ ID NO: 566 - RNA, 19 nt, Artificial
  SYNTHETIC CONSTRUCT
ccagacuuaaaucacagau

SEQ ID NO: 567 - RNA, 19 nt, Artificial
  SYNTHETIC CONSTRUCT
ggucugaauuuagugucua

SEQ ID NO: 568 - RNA, 19 nt, Artificial
  SYNTHETIC CONSTRUCT
uuaaaucacagauggguau

SEQ ID NO: 569 - RNA, 19 nt, Artificial
  SYNTHETIC CONSTRUCT
aauuuagugucuacccaua

SEQ ID NO: 570 - RNA, 19 nt, Artificial
  SYNTHETIC CONSTRUCT
uaaaucacagauggguauu

SEQ ID NO: 571 - RNA, 19 nt, Artificial
  SYNTHETIC CONSTRUCT
auuuagugucuacccauaa

SEQ ID NO: 572 - RNA, 19 nt, Artificial
  SYNTHETIC CONSTRUCT
ucacagauggguauuaaac

SEQ ID NO: 573 - RNA, 19 nt, Artificial
  SYNTHETIC CONSTRUCT
agugucuacccauaauuug

SEQ ID NO: 574 - RNA, 19 nt, Artificial
  SYNTHETIC CONSTRUCT
cacagauggguauuaaacu

SEQ ID NO: 575 - RNA, 19 nt, Artificial
  SYNTHETIC CONSTRUCT
gugucuacccauaauuuga

SEQ ID NO: 576 - RNA, 19 nt, Artificial
  SYNTHETIC CONSTRUCT
auggguauuaaacuuguca

SEQ ID NO: 577 - RNA, 19 nt, Artificial
  SYNTHETIC CONSTRUCT
uacccauaauuugaacagu

SEQ ID NO: 578 - RNA, 19 nt, Artificial
  SYNTHETIC CONSTRUCT
ggguauuaaacuugucaga

SEQ ID NO: 579 - RNA, 19 nt, Artificial
  SYNTHETIC CONSTRUCT
cccauaauuugaacagucu

SEQ ID NO: 580 - RNA, 19 nt, Artificial
  SYNTHETIC CONSTRUCT
cuugucagaauuucuuugu

SEQ ID NO: 581 - RNA, 19 nt, Artificial
  SYNTHETIC CONSTRUCT
gaacagucuuaaagaaaca

SEQ ID NO: 582 - RNA, 19 nt, Artificial
  SYNTHETIC CONSTRUCT
gucagaauuucuuugucau

SEQ ID NO: 583 - RNA, 19 nt, Artificial
  SYNTHETIC CONSTRUCT
cagucuuaaagaaacagua

SEQ ID NO: 584 - RNA, 19 nt, Artificial
  SYNTHETIC CONSTRUCT
uugucauucaagccuguga

SEQ ID NO: 585 - RNA, 19 nt, Artificial
  SYNTHETIC CONSTRUCT
aacaguaaguucggacacu

SEQ ID NO: 586 - RNA, 19 nt, Artificial
  SYNTHETIC CONSTRUCT
ugucauucaagccugugaa

SEQ ID NO: 587 - RNA, 19 nt, Artificial
  SYNTHETIC CONSTRUCT
acaguaaguucggacacuu

SEQ ID NO: 588 - RNA, 19 nt, Artificial
  SYNTHETIC CONSTRUCT
gucauucaagccugugaau

SEQ ID NO: 589 - RNA, 19 nt, Artificial
  SYNTHETIC CONSTRUCT
caguaaguucggacacuua

SEQ ID NO: 590 - RNA, 19 nt, Artificial
  SYNTHETIC CONSTRUCT
guauggcacuuauuaugag

SEQ ID NO: 591 - RNA, 19 nt, Artificial
  SYNTHETIC CONSTRUCT
cauaccgugaauaauacuc

SEQ ID NO: 592 - RNA, 19 nt, Artificial
  SYNTHETIC CONSTRUCT
gcacuuauuaugaggcuau

SEQ ID NO: 593 - RNA, 19 nt, Artificial
  SYNTHETIC CONSTRUCT
cgugaauaauacuccgaua

SEQ ID NO: 594 - RNA, 19 nt, Artificial
  SYNTHETIC CONSTRUCT
cacuuauuaugaggcuauu

SEQ ID NO: 595 - RNA, 19 nt, Artificial
  SYNTHETIC CONSTRUCT
gugaauaauacuccgauaa

SEQ ID NO: 596 - RNA, 19 nt, Artificial
  SYNTHETIC CONSTRUCT
ugaggcuauuaaaagaauc

SEQ ID NO: 597 - RNA, 19 nt, Artificial
  SYNTHETIC CONSTRUCT
acuccgauaauuuucuuag

SEQ ID NO: 598 - RNA, 19 nt, Artificial
  SYNTHETIC CONSTRUCT
ggcuauuaaaagaauccaa

SEQ ID NO: 599 - RNA, 19 nt, Artificial
  SYNTHETIC CONSTRUCT
ccgauaauuuucuuagguu

SEQ ID NO: 600 - RNA, 19 nt, Artificial
  SYNTHETIC CONSTRUCT
ggugcugguuugcgucgua

SEQ ID NO: 601 - RNA, 19 nt, Artificial
  SYNTHETIC CONSTRUCT
ccacgaccaaacgcagcau

SEQ ID NO: 602 - RNA, 19 nt, Artificial
  SYNTHETIC CONSTRUCT
gucugggguuuccguugca

SEQ ID NO: 603 - RNA, 19 nt, Artificial
  SYNTHETIC CONSTRUCT
cagaccccaaaggcaacgu

SEQ ID NO: 604 - RNA, 19 nt, Artificial
  SYNTHETIC CONSTRUCT
ccagugcagggcaucauca

SEQ ID NO: 605 - RNA, 19 nt, Artificial
  SYNTHETIC CONSTRUCT
ggucacgucccguaguagu

SEQ ID NO: 606 - RNA, 19 nt, Artificial
  SYNTHETIC CONSTRUCT
gaaaugaagaaaguacaaa

SEQ ID NO: 607 - RNA, 19 nt, Artificial
  SYNTHETIC CONSTRUCT
cuuuacuucuuucauguuu

SEQ ID NO: 608 - RNA, 19 nt, Artificial
  SYNTHETIC CONSTRUCT
aaugaagaaaguacaaaga

SEQ ID NO: 609 - RNA, 19 nt, Artificial
  SYNTHETIC CONSTRUCT
uuacuucuuucauguuucu

SEQ ID NO: 610 - RNA, 19 nt, Artificial
  SYNTHETIC CONSTRUCT
uagaaauguauccugauaa

SEQ ID NO: 611 - RNA, 19 nt, Artificial
  SYNTHETIC CONSTRUCT
aucuuuacauaggacuauu

SEQ ID NO: 612 - RNA, 19 nt, Artificial
  SYNTHETIC CONSTRUCT
aauguauccugauaaacau

SEQ ID NO: 613 - RNA, 19 nt, Artificial
  SYNTHETIC CONSTRUCT
uuacauaggacuauuugua

SEQ ID NO: 614 - RNA, 19 nt, Artificial
  SYNTHETIC CONSTRUCT
uguauccugauaaacauua

SEQ ID NO: 615 - RNA, 19 nt, Artificial
  SYNTHETIC CONSTRUCT
acauaggacuauuuguaau

SEQ ID NO: 616 - RNA, 19 nt, Artificial
  SYNTHETIC CONSTRUCT
uccugauaaacauuaaaca

SEQ ID NO: 617 - RNA, 19 nt, Artificial
  SYNTHETIC CONSTRUCT
aggacuauuuguaauuugu

SEQ ID NO: 618 - RNA, 19 nt, Artificial
  SYNTHETIC CONSTRUCT
auaaacauuaaacacugua

SEQ ID NO: 619 - RNA, 19 nt, Artificial
  SYNTHETIC CONSTRUCT
uauuuguaauuugugacau

SEQ ID NO: 620 - RNA, 19 nt, Artificial
  SYNTHETIC CONSTRUCT
aaacauuaaacacuguaau

SEQ ID NO: 621 - RNA, 19 nt, Artificial
  SYNTHETIC CONSTRUCT
uuuguaauuugugacauua

SEQ ID NO: 622 - RNA, 19 nt, Artificial
  SYNTHETIC CONSTRUCT
cuguaaucuuaaaagugua

SEQ ID NO: 623 - RNA, 19 nt, Artificial
  SYNTHETIC CONSTRUCT
gacauuagaauuuucacau

SEQ ID NO: 624 - RNA, 19 nt, Artificial
  SYNTHETIC CONSTRUCT
cuguaaucuuaaaagugua

SEQ ID NO: 625 - RNA, 19 nt, Artificial
  SYNTHETIC CONSTRUCT
acauuagaauuuucacauu

SEQ ID NO: 626 - RNA, 19 nt, Artificial
  SYNTHETIC CONSTRUCT
uaaaaguguaauuguguga

SEQ ID NO: 627 - RNA, 19 nt, Artificial
  SYNTHETIC CONSTRUCT
auuuucacauuaacacacu

SEQ ID NO: 628 - RNA, 19 nt, Artificial
  SYNTHETIC CONSTRUCT
uguaauugugugacuuuuu

SEQ ID NO: 629 - RNA, 19 nt, Artificial
  SYNTHETIC CONSTRUCT
acauuaacacacugaaaaa

SEQ ID NO: 630 - RNA, 19 nt, Artificial
  SYNTHETIC CONSTRUCT
uuuuucagaguugcuuuaa

SEQ ID NO: 631 - RNA, 19 nt, Artificial
  SYNTHETIC CONSTRUCT
aaaaagucucaacgaaauu

SEQ ID NO: 632 - RNA, 19 nt, Artificial
  SYNTHETIC CONSTRUCT
ugagaaacugauuuaugau

SEQ ID NO: 633 - RNA, 19 nt, Artificial
  SYNTHETIC CONSTRUCT
acucuuugacuaaauacua

SEQ ID NO: 634 - RNA, 19 nt, Artificial
  SYNTHETIC CONSTRUCT
uuggaagauuuguauaguu

SEQ ID NO: 635 - RNA, 19 nt, Artificial
  SYNTHETIC CONSTRUCT
aaccuucuaaacauaucaa

SEQ ID NO: 636 - RNA, 19 nt, Artificial
  SYNTHETIC CONSTRUCT
uggaagauuuguauaguuu

SEQ ID NO: 637 - RNA, 19 nt, Artificial
  SYNTHETIC CONSTRUCT
accuucuaaacauaucaaa

SEQ ID NO: 638 - RNA, 19 nt, Artificial
  SYNTHETIC CONSTRUCT
ggaagauuuguauaguuuu

SEQ ID NO: 639 - RNA, 19 nt, Artificial
  SYNTHETIC CONSTRUCT
ccuucuaaacauaucaaaa

SEQ ID NO: 640 - RNA, 19 nt, Artificial
  SYNTHETIC CONSTRUCT
uguauaguuuuauaaaacu

SEQ ID NO: 641 - RNA, 19 nt, Artificial
  SYNTHETIC CONSTRUCT
acauaucaaaauauuuuga

SEQ ID NO: 642 - RNA, 19 nt, Artificial
  SYNTHETIC CONSTRUCT
guuuuauaaaacucaguua

SEQ ID NO: 643 - RNA, 19 nt, Artificial
  SYNTHETIC CONSTRUCT
caaaauauuuugagucaau

SEQ ID NO: 644 - RNA, 19 nt, Artificial
  SYNTHETIC CONSTRUCT
uuuuauaaaacucaguuaa

SEQ ID NO: 645 - RNA, 19 nt, Artificial
  SYNTHETIC CONSTRUCT
aaaauauuuugagucaauu

SEQ ID NO: 646 - RNA, 19 nt, Artificial
  SYNTHETIC CONSTRUCT
uauaaaacucaguuaaaau

SEQ ID NO: 647 - RNA, 19 nt, Artificial
  SYNTHETIC CONSTRUCT
auauuuugagucaauuuua

SEQ ID NO: 648 - RNA, 19 nt, Artificial
  SYNTHETIC CONSTRUCT
uaaaacucaguuaaaaugu

SEQ ID NO: 649 - RNA, 19 nt, Artificial
  SYNTHETIC CONSTRUCT
auuuugagucaauuuuaca

SEQ ID NO: 650 - RNA, 19 nt, Artificial
  SYNTHETIC CONSTRUCT
aaacucaguuaaaaugucu

SEQ ID NO: 651 - RNA, 19 nt, Artificial
  SYNTHETIC CONSTRUCT
uuugagucaauuuuacaga

SEQ ID NO: 652 - RNA, 19 nt, Artificial
  SYNTHETIC CONSTRUCT
ucaguuaaaaugucuguuu

SEQ ID NO: 653 - RNA, 19 nt, Artificial
  SYNTHETIC CONSTRUCT
agucaauuuuacagacaaa

SEQ ID NO: 654 - RNA, 19 nt, Artificial
  SYNTHETIC CONSTRUCT
aguuaaaaugucuguuuca

SEQ ID NO: 655 - RNA, 19 nt, Artificial
  SYNTHETIC CONSTRUCT
ucaauuuuacagacaaagu

SEQ ID NO: 656 - RNA, 19 nt, Artificial
  SYNTHETIC CONSTRUCT
guuaaaaugucuguuucaa

SEQ ID NO: 657 - RNA, 19 nt, Artificial
  SYNTHETIC CONSTRUCT
caauuuuacagacaaaguu

SEQ ID NO: 658 - RNA, 19 nt, Artificial
  SYNTHETIC CONSTRUCT
uuaaaaugucuguuucaau

SEQ ID NO: 659 - RNA, 19 nt, Artificial
  SYNTHETIC CONSTRUCT
aauuuuacagacaaaguua

SEQ ID NO: 660 - RNA, 19 nt, Artificial
  SYNTHETIC CONSTRUCT
uauuuugccagacuuaaau

SEQ ID NO: 661 - RNA, 19 nt, Artificial
  SYNTHETIC CONSTRUCT
auaaaacggucugaauuua

SEQ ID NO: 662 - RNA, 19 nt, Artificial
  SYNTHETIC CONSTRUCT
uauuaaacuugucagaauu

SEQ ID NO: 663 - RNA, 19 nt, Artificial
  SYNTHETIC CONSTRUCT
auaauuugaacagucuuaa

SEQ ID NO: 664 - RNA, 19 nt, Artificial
  SYNTHETIC CONSTRUCT
auuaaacuugucagaauuu

SEQ ID NO: 665 - RNA, 19 nt, Artificial
  SYNTHETIC CONSTRUCT
uaauuugaacagucuuaaa

SEQ ID NO: 666 - RNA, 19 nt, Artificial
  SYNTHETIC CONSTRUCT
uaaacuugucagaauuucu

SEQ ID NO: 667 - RNA, 19 nt, Artificial
  SYNTHETIC CONSTRUCT
auuugaacagucuuaaaga

SEQ ID NO: 668 - RNA, 19 nt, Artificial
  SYNTHETIC CONSTRUCT
aaacuugucagaauuucuu

SEQ ID NO: 669 - RNA, 19 nt, Artificial
  SYNTHETIC CONSTRUCT
uuugaacagucuuaaagaa

SEQ ID NO: 670 - RNA, 19 nt, Artificial
  SYNTHETIC CONSTRUCT
ucagaauuucuuugucauu

SEQ ID NO: 671 - RNA, 19 nt, Artificial
  SYNTHETIC CONSTRUCT
agucuuaaagaaacaguaa

SEQ ID NO: 672 - RNA, 19 nt, Artificial
  SYNTHETIC CONSTRUCT
acuuauuaugaggcuauua

SEQ ID NO: 673 - RNA, 19 nt, Artificial
  SYNTHETIC CONSTRUCT
ugaauaauacuccgauaau

SEQ ID NO: 674 - RNA, 19 nt, Artificial
  SYNTHETIC CONSTRUCT
uaugaggcuauuaaaagaa

SEQ ID NO: 675 - RNA, 19 nt, Artificial
  SYNTHETIC CONSTRUCT
auacuccgauaauuuucuu

SEQ ID NO: 676 - RNA, 19 nt, Artificial
  SYNTHETIC CONSTRUCT
augaggcuauuaaaagaau

SEQ ID NO: 677 - RNA, 19 nt, Artificial
  SYNTHETIC CONSTRUCT
uacuccgauaauuuucuua

SEQ ID NO: 678 - RNA, 19 nt, Artificial
  SYNTHETIC CONSTRUCT
cuauuaaaagaauccaaau

SEQ ID NO: 679 - RNA, 19 nt, Artificial
  SYNTHETIC CONSTRUCT
gauaauuuucuuagguuua

SEQ ID NO: 680 - RNA, 19 nt, Artificial
  SYNTHETIC CONSTRUCT
ggucuggccuauaaaguag

SEQ ID NO: 681 - RNA, 19 nt, Artificial
  SYNTHETIC CONSTRUCT
ccagaccggauauuucauc

SEQ ID NO: 682 - RNA, 19 nt, Artificial
  SYNTHETIC CONSTRUCT
gucuggccuauaaaguagu

SEQ ID NO: 683 - RNA, 19 nt, Artificial
  SYNTHETIC CONSTRUCT
cagaccggauauuucauca

SEQ ID NO: 684 - RNA, 19 nt, Artificial
  SYNTHETIC CONSTRUCT
ucuggccuauaaaguaguc

SEQ ID NO: 685 - RNA, 19 nt, Artificial
  SYNTHETIC CONSTRUCT
agaccggauauuucaucag

SEQ ID NO: 686 - RNA, 19 nt, Artificial
  SYNTHETIC CONSTRUCT
ccuauaaaguagucgcgga

SEQ ID NO: 687 - RNA, 19 nt, Artificial
  SYNTHETIC CONSTRUCT
ggauauuucaucagcgccu

SEQ ID NO: 688 - RNA, 19 nt, Artificial
  SYNTHETIC CONSTRUCT
auaaaguagucgcggagac

SEQ ID NO: 689 - RNA, 19 nt, Artificial
  SYNTHETIC CONSTRUCT
uauuucaucagcgccucug

SEQ ID NO: 690 - RNA, 19 nt, Artificial
  SYNTHETIC CONSTRUCT
ugcugguuugcgucguagu

SEQ ID NO: 691 - RNA, 19 nt, Artificial
  SYNTHETIC CONSTRUCT
acgaccaaacgcagcauca

SEQ ID NO: 692 - RNA, 19 nt, Artificial
  SYNTHETIC CONSTRUCT
aucaucaauuucgagcaga

SEQ ID NO: 693 - RNA, 19 nt, Artificial
  SYNTHETIC CONSTRUCT
uaguaguuaaagcucgucu

SEQ ID NO: 694 - RNA, 19 nt, Artificial
  SYNTHETIC CONSTRUCT
uuucgagcagaaggaaagu

SEQ ID NO: 695 - RNA, 19 nt, Artificial
  SYNTHETIC CONSTRUCT
aaagcucgucuuccuuuca

SEQ ID NO: 696 - RNA, 19 nt, Artificial
  SYNTHETIC CONSTRUCT
gagcagaaggaaaguaaug

SEQ ID NO: 697 - RNA, 19 nt, Artificial
  SYNTHETIC CONSTRUCT
cucgucuuccuuucauuac

SEQ ID NO: 698 - RNA, 19 nt, Artificial
  SYNTHETIC CONSTRUCT
ggaaaguaauggaccagug

SEQ ID NO: 699 - RNA, 19 nt, Artificial
  SYNTHETIC CONSTRUCT
ccuuucauuaccuggucac

SEQ ID NO: 700 - RNA, 19 nt, Artificial
  SYNTHETIC CONSTRUCT
ugaagguguggggaagcau

SEQ ID NO: 701 - RNA, 19 nt, Artificial
  SYNTHETIC CONSTRUCT
acuuccacaccccuucgua

SEQ ID NO: 702 - RNA, 19 nt, Artificial
  SYNTHETIC CONSTRUCT
guguggggaagcauuaaag

SEQ ID NO: 703 - RNA, 19 nt, Artificial
  SYNTHETIC CONSTRUCT
cacaccccuucguaauuuc

SEQ ID NO: 704 - RNA, 19 nt, Artificial
  SYNTHETIC CONSTRUCT
ggggaagcauuaaaggacu

SEQ ID NO: 705 - RNA, 19 nt, Artificial
  SYNTHETIC CONSTRUCT
ccccuucguaauuuccuga

SEQ ID NO: 706 - RNA, 19 nt, Artificial
  SYNTHETIC CONSTRUCT
aagcauuaaaggacugacu

SEQ ID NO: 707 - RNA, 19 nt, Artificial
  SYNTHETIC CONSTRUCT
uucguaauuuccugacuga

SEQ ID NO: 708 - RNA, 19 nt, Artificial
  SYNTHETIC CONSTRUCT
cauuaaaggacugacugaa

SEQ ID NO: 709 - RNA, 19 nt, Artificial
  SYNTHETIC CONSTRUCT
guaauuuccugacugacuu

SEQ ID NO: 710 - RNA, 19 nt, Artificial
  SYNTHETIC CONSTRUCT
uuaaaggacugacugaagg

SEQ ID NO: 711 - RNA, 19 nt, Artificial
  SYNTHETIC CONSTRUCT
aauuuccugacugacuucc

SEQ ID NO: 712 - RNA, 19 nt, Artificial
  SYNTHETIC CONSTRUCT
acugacugaaggccugcau

SEQ ID NO: 713 - RNA, 19 nt, Artificial
  SYNTHETIC CONSTRUCT
ugacugacuuccggacgua

SEQ ID NO: 714 - RNA, 19 nt, Artificial
  SYNTHETIC CONSTRUCT
acugaaggccugcauggau

SEQ ID NO: 715 - RNA, 19 nt, Artificial
  SYNTHETIC CONSTRUCT
ugacuuccggacguaccua

SEQ ID NO: 716 - RNA, 19 nt, Artificial
  SYNTHETIC CONSTRUCT
ugaaggccugcauggauuc

SEQ ID NO: 717 - RNA, 19 nt, Artificial
  SYNTHETIC CONSTRUCT
acuuccggacguaccuaag

SEQ ID NO: 718 - RNA, 19 nt, Artificial
  SYNTHETIC CONSTRUCT
aaggccugcauggauucca

SEQ ID NO: 719 - RNA, 19 nt, Artificial
  SYNTHETIC CONSTRUCT
uuccggacguaccuaaggu

SEQ ID NO: 720 - RNA, 19 nt, Artificial
  SYNTHETIC CONSTRUCT
gccugcauggauuccaugu

SEQ ID NO: 721 - RNA, 19 nt, Artificial
  SYNTHETIC CONSTRUCT
cggacguaccuaagguaca

SEQ ID NO: 722 - RNA, 19 nt, Artificial
  SYNTHETIC CONSTRUCT
cugcauggauuccauguuc

SEQ ID NO: 723 - RNA, 19 nt, Artificial
  SYNTHETIC CONSTRUCT
gacguaccuaagguacaag

SEQ ID NO: 724 - RNA, 19 nt, Artificial
  SYNTHETIC CONSTRUCT
uggauuccauguucaugag

SEQ ID NO: 725 - RNA, 19 nt, Artificial
  SYNTHETIC CONSTRUCT
accuaagguacaaguacuc

SEQ ID NO: 726 - RNA, 19 nt, Artificial
  SYNTHETIC CONSTRUCT
auguucaugaguuuggaga

SEQ ID NO: 727 - RNA, 19 nt, Artificial
  SYNTHETIC CONSTRUCT
uacaaguacucaaaccucu

SEQ ID NO: 728 - RNA, 19 nt, Artificial
  SYNTHETIC CONSTRUCT
uguucaugaguuuggagau

SEQ ID NO: 729 - RNA, 19 nt, Artificial
  SYNTHETIC CONSTRUCT
acaaguacucaaaccucua

SEQ ID NO: 730 - RNA, 19 nt, Artificial
  SYNTHETIC CONSTRUCT
augaguuuggagauaauac

SEQ ID NO: 731 - RNA, 19 nt, Artificial
  SYNTHETIC CONSTRUCT
uacucaaaccucuauuaug

SEQ ID NO: 732 - RNA, 19 nt, Artificial
  SYNTHETIC CONSTRUCT
agauaauacagcaggcugu

SEQ ID NO: 733 - RNA, 19 nt, Artificial
  SYNTHETIC CONSTRUCT
ucuauuaugucguccgaca

SEQ ID NO: 734 - RNA, 19 nt, Artificial
  SYNTHETIC CONSTRUCT
gauaauacagcaggcugua

SEQ ID NO: 735 - RNA, 19 nt, Artificial
  SYNTHETIC CONSTRUCT
cuauuaugucguccgacau

SEQ ID NO: 736 - RNA, 19 nt, Artificial
  SYNTHETIC CONSTRUCT
agguccucacuuuaauccu

SEQ ID NO: 737 - RNA, 19 nt, Artificial
  SYNTHETIC CONSTRUCT
uccaggagugaaauuagga

SEQ ID NO: 738 - RNA, 19 nt, Artificial
  SYNTHETIC CONSTRUCT
guccucacuuuaauccucu

SEQ ID NO: 739 - RNA, 19 nt, Artificial
  SYNTHETIC CONSTRUCT
caggagugaaauuaggaga

SEQ ID NO: 740 - RNA, 19 nt, Artificial
  SYNTHETIC CONSTRUCT
agaaaacacggugggccaa

SEQ ID NO: 741 - RNA, 19 nt, Artificial
  SYNTHETIC CONSTRUCT
ucuuuugugccacccgguu

SEQ ID NO: 742 - RNA, 19 nt, Artificial
  SYNTHETIC CONSTRUCT
aaaacacggugggccaaag

SEQ ID NO: 743 - RNA, 19 nt, Artificial
  SYNTHETIC CONSTRUCT
uuuugugccacccgguuuc

SEQ ID NO: 744 - RNA, 19 nt, Artificial
  SYNTHETIC CONSTRUCT
gggccaaaggaugaagaga

SEQ ID NO: 745 - RNA, 19 nt, Artificial
  SYNTHETIC CONSTRUCT
cccgguuuccuacuucucu

SEQ ID NO: 746 - RNA, 19 nt, Artificial
  SYNTHETIC CONSTRUCT
aagagaggcauguuggaga

SEQ ID NO: 747 - RNA, 19 nt, Artificial
  SYNTHETIC CONSTRUCT
uucucuccguacaaccucu

SEQ ID NO: 748 - RNA, 19 nt, Artificial
  SYNTHETIC CONSTRUCT
agagaggcauguuggagac

SEQ ID NO: 749 - RNA, 19 nt, Artificial
  SYNTHETIC CONSTRUCT
ucucuccguacaaccucug

SEQ ID NO: 750 - RNA, 19 nt, Artificial
  SYNTHETIC CONSTRUCT
gaggcauguuggagacuug

SEQ ID NO: 751 - RNA, 19 nt, Artificial
  SYNTHETIC CONSTRUCT
cuccguacaaccucugaac

SEQ ID NO: 752 - RNA, 19 nt, Artificial
  SYNTHETIC CONSTRUCT
auguuggagacuugggcaa

SEQ ID NO: 753 - RNA, 19 nt, Artificial
  SYNTHETIC CONSTRUCT
uacaaccucugaacccguu

SEQ ID NO: 754 - RNA, 19 nt, Artificial
  SYNTHETIC CONSTRUCT
ggcaaugugacugcugaca

SEQ ID NO: 755 - RNA, 19 nt, Artificial
  SYNTHETIC CONSTRUCT
ccguuacacugacgacugu

SEQ ID NO: 756 - RNA, 19 nt, Artificial
  SYNTHETIC CONSTRUCT
gacugcugacaaagauggu

SEQ ID NO: 757 - RNA, 19 nt, Artificial
  SYNTHETIC CONSTRUCT
cugacgacuguuucuacca

SEQ ID NO: 758 - RNA, 19 nt, Artificial
  SYNTHETIC CONSTRUCT
acaaagaugguguggccga

SEQ ID NO: 759 - RNA, 19 nt, Artificial
  SYNTHETIC CONSTRUCT
uguuucuaccacaccggcu

SEQ ID NO: 760 - RNA, 19 nt, Artificial
  SYNTHETIC CONSTRUCT
caaagaugguguggccgau

SEQ ID NO: 761 - RNA, 19 nt, Artificial
  SYNTHETIC CONSTRUCT
guuucuaccacaccggcua

SEQ ID NO: 762 - RNA, 19 nt, Artificial
  SYNTHETIC CONSTRUCT
guggccgaugugucuauug

SEQ ID NO: 763 - RNA, 19 nt, Artificial
  SYNTHETIC CONSTRUCT
caccggcuacacagauaac

SEQ ID NO: 764 - RNA, 19 nt, Artificial
  SYNTHETIC CONSTRUCT
gccgaugugucuauugaag

SEQ ID NO: 765 - RNA, 19 nt, Artificial
  SYNTHETIC CONSTRUCT
cggcuacacagauaacuuc

SEQ ID NO: 766 - RNA, 19 nt, Artificial
  SYNTHETIC CONSTRUCT
gaugugucuauugaagauu

SEQ ID NO: 767 - RNA, 19 nt, Artificial
  SYNTHETIC CONSTRUCT
cuacacagauaacuucuaa

SEQ ID NO: 768 - RNA, 19 nt, Artificial
  SYNTHETIC CONSTRUCT
augugucuauugaagauuc

SEQ ID NO: 769 - RNA, 19 nt, Artificial
  SYNTHETIC CONSTRUCT
uacacagauaacuucuaag

SEQ ID NO: 770 - RNA, 19 nt, Artificial
  SYNTHETIC CONSTRUCT
ugucuauugaagauucugu

SEQ ID NO: 771 - RNA, 19 nt, Artificial
  SYNTHETIC CONSTRUCT
acagauaacuucuaagaca

SEQ ID NO: 772 - RNA, 19 nt, Artificial
  SYNTHETIC CONSTRUCT
uauugaagauucugugauc

SEQ ID NO: 773 - RNA, 19 nt, Artificial
  SYNTHETIC CONSTRUCT
auaacuucuaagacacuag

SEQ ID NO: 774 - RNA, 19 nt, Artificial
  SYNTHETIC CONSTRUCT
auugaagauucugugaucu

SEQ ID NO: 775 - RNA, 19 nt, Artificial
  SYNTHETIC CONSTRUCT
uaacuucuaagacacuaga

SEQ ID NO: 776 - RNA, 19 nt, Artificial
  SYNTHETIC CONSTRUCT
gauucugugaucucacucu

SEQ ID NO: 777 - RNA, 19 nt, Artificial
  SYNTHETIC CONSTRUCT
cuaagacacuagagugaga

SEQ ID NO: 778 - RNA, 19 nt, Artificial
  SYNTHETIC CONSTRUCT
ugugaucucacucucagga

SEQ ID NO: 779 - RNA, 19 nt, Artificial
  SYNTHETIC CONSTRUCT
acacuagagugagaguccu

SEQ ID NO: 780 - RNA, 19 nt, Artificial
  SYNTHETIC CONSTRUCT
cucacucucaggagaccau

SEQ ID NO: 781 - RNA, 19 nt, Artificial
  SYNTHETIC CONSTRUCT
gagugagaguccucuggua

SEQ ID NO: 782 - RNA, 19 nt, Artificial
  SYNTHETIC CONSTRUCT
ucacucucaggagaccauu

SEQ ID NO: 783 - RNA, 19 nt, Artificial
  SYNTHETIC CONSTRUCT
agugagaguccucugguaa

SEQ ID NO: 784 - RNA, 19 nt, Artificial
  SYNTHETIC CONSTRUCT
ucucaggagaccauugcau

SEQ ID NO: 785 - RNA, 19 nt, Artificial
  SYNTHETIC CONSTRUCT
agaguccucugguaacgua

SEQ ID NO: 786 - RNA, 19 nt, Artificial
  SYNTHETIC CONSTRUCT
caggagaccauugcaucau

SEQ ID NO: 787 - RNA, 19 nt, Artificial
  SYNTHETIC CONSTRUCT
guccucugguaacguagua

SEQ ID NO: 788 - RNA, 19 nt, Artificial
  SYNTHETIC CONSTRUCT
ugguccaugaaaaagcaga

SEQ ID NO: 789 - RNA, 19 nt, Artificial
  SYNTHETIC CONSTRUCT
accagguacuuuuucgucu

SEQ ID NO: 790 - RNA, 19 nt, Artificial
  SYNTHETIC CONSTRUCT
gguccaugaaaaagcagau

SEQ ID NO: 791 - RNA, 19 nt, Artificial
  SYNTHETIC CONSTSRUCT
ccagguacuuuuucgucua

SEQ ID NO: 792 - RNA, 19 nt, Artificial
  SYNTHETIC CONSTSRUCT
ccaugaaaaagcagaugac

SEQ ID NO: 793 - RNA, 19 nt, Artificial
  SYNTHETIC CONSTSRUCT
gguacuuuuucgucuacug

SEQ ID NO: 794 - RNA, 19 nt, Artificial
  SYNTHETIC CONSTSRUCT
augaaaaagcagaugacuu

SEQ ID NO: 795 - RNA, 19 nt, Artificial
  SYNTHETIC CONSTSRUCT
uacuuuuucgucuacugaa

SEQ ID NO: 796 - RNA, 19 nt, Artificial
  SYNTHETIC CONSTSRUCT
ugaaaaagcagaugacuug

SEQ ID NO: 797 - RNA, 19 nt, Artificial
  SYNTHETIC CONSTSRUCT
acuuuuucgucuacugaac

SEQ ID NO: 798 - RNA, 19 nt, Artificial
  SYNTHETIC CONSTSRUCT
aaagcagaugacuugggca

SEQ ID NO: 799 - RNA, 19 nt, Artificial
  SYNTHETIC CONSTSRUCT
uuucgucuacugaacccgu

SEQ ID NO: 800 - RNA, 19 nt, Artificial
  SYNTHETIC CONSTSRUCT
aagcagaugacuugggcaa

SEQ ID NO: 801 - RNA, 19 nt, Artificial
  SYNTHETIC CONSTRUCT
uucgucuacugaacccguu

SEQ ID NO: 802 - RNA, 19 nt, Artificial
  SYNTHETIC CONSTRUCT
agcagaugacuugggcaaa

SEQ ID NO: 803 - RNA, 19 nt, Artificial
  SYNTHETIC CONSTRUCT
ucgucuacugaacccguuu

SEQ ID NO: 804 - RNA, 19 nt, Artificial
  SYNTHETIC CONSTRUCT
gcagaugacuugggcaaag

SEQ ID NO: 805 - RNA, 19 nt, Artificial
  SYNTHETIC CONSTRUCT
cgucuacugaacccguuuc

SEQ ID NO: 806 - RNA, 19 nt, Artificial
  SYNTHETIC CONSTRUCT
gacuugggcaaagguggaa

SEQ ID NO: 807 - RNA, 19 nt, Artificial
  SYNTHETIC CONSTRUCT
cugaacccguuuccaccuu

SEQ ID NO: 808 - RNA, 19 nt, Artificial
  SYNTHETIC CONSTRUCT
acuugggcaaagguggaaa

SEQ ID NO: 809 - RNA, 19 nt, Artificial
  SYNTHETIC CONSTRUCT
ugaacccguuuccaccuuu

SEQ ID NO: 810 - RNA, 19 nt, Artificial
  SYNTHETIC CONSTRUCT
agaaaguacaaagacagga

SEQ ID NO: 811 - RNA, 19 nt, Artificial
  SYNTHETIC CONSTRUCT
ucuuucauguuucuguccu

SEQ ID NO: 812 - RNA, 19 nt, Artificial
  SYNTHETIC CONSTRUCT
aagacaggaaacgcuggaa

SEQ ID NO: 813 - RNA, 19 nt, Artificial
  SYNTHETIC CONSTRUCT
uucuguccuuugcgaccuu

SEQ ID NO: 814 - RNA, 19 nt, Artificial
  SYNTHETIC CONSTRUCT
gacaggaaacgcuggaagu

SEQ ID NO: 815 - RNA, 19 nt, Artificial
  SYNTHETIC CONSTRUCT
cuguccuuugcgaccuuca

SEQ ID NO: 816 - RNA, 19 nt, Artificial
  SYNTHETIC CONSTRUCT
aggaaacgcuggaagucgu

SEQ ID NO: 817 - RNA, 19 nt, Artificial
  SYNTHETIC CONSTRUCT
uccuuugcgaccuucagca

SEQ ID NO: 818 - RNA, 19 nt, Artificial
  SYNTHETIC CONSTRUCT
gaaacgcuggaagucguuu

SEQ ID NO: 819 - RNA, 19 nt, Artificial
  SYNTHETIC CONSTRUCT
cuuugcgaccuucagcaaa

SEQ ID NO: 820 - RNA, 19 nt, Artificial
  SYNTHETIC CONSTRUCT
aaacgcuggaagucguuug

SEQ ID NO: 821 - RNA, 19 nt, Artificial
  SYNTHETIC CONSTRUCT
uuugcgaccuucagcaaac

SEQ ID NO: 822 - RNA, 19 nt, Artificial
  SYNTHETIC CONSTRUCT
uggaagucguuuggcuugu

SEQ ID NO: 823 - RNA, 19 nt, Artificial
  SYNTHETIC CONSTRUCT
accuucagcaaaccgaaca

SEQ ID NO: 824 - RNA, 19 nt, Artificial
  SYNTHETIC CONSTRUCT
ggaagucguuuggcuugug

SEQ ID NO: 825 - RNA, 19 nt, Artificial
  SYNTHETIC CONSTRUCT
ccuucagcaaaccgaacac

SEQ ID NO: 826 - RNA, 19 nt, Artificial
  SYNTHETIC CONSTRUCT
gaagucguuuggcuugugg

SEQ ID NO: 827 - RNA, 19 nt, Artificial
  SYNTHETIC CONSTRUCT
cuucagcaaaccgaacacc

SEQ ID NO: 828 - RNA, 19 nt, Artificial
  SYNTHETIC CONSTRUCT
guuuggcuugugguguaau

SEQ ID NO: 829 - RNA, 19 nt, Artificial
  SYNTHETIC CONSTRUCT
caaaccgaacaccacauua

SEQ ID NO: 830 - RNA, 19 nt, Artificial
  SYNTHETIC CONSTRUCT
gcuugugguguaauuggga

SEQ ID NO: 831 - RNA, 19 nt, Artificial
  SYNTHETIC CONSTRUCT
cgaacaccacauuaacccu

SEQ ID NO: 832 - RNA, 19 nt, Artificial
  SYNTHETIC CONSTRUCT
cuugugguguaauugggau

SEQ ID NO: 833 - RNA, 19 nt, Artificial
  SYNTHETIC CONSTRUCT
gaacaccacauuaacccua

SEQ ID NO: 834 - RNA, 19 nt, Artificial
  SYNTHETIC CONSTRUCT
uguaauugggaucgcccaa

SEQ ID NO: 835 - RNA, 19 nt, Artificial
  SYNTHETIC CONSTRUCT
acauuaacccuagcggguu

SEQ ID NO: 836 - RNA, 19 nt, Artificial
  SYNTHETIC CONSTRUCT
guaauugggaucgcccaau

SEQ ID NO: 837 - RNA, 19 nt, Artificial
  SYNTHETIC CONSTRUCT
cauuaacccuagcggguua

SEQ ID NO: 838 - RNA, 19 nt, Artificial
  SYNTHETIC CONSTRUCT
uugggaucgcccaauaaac

SEQ ID NO: 839 - RNA, 19 nt, Artificial
  SYNTHETIC CONSTRUCT
aacccuagcggguuauuug

SEQ ID NO: 840 - RNA, 19 nt, Artificial
  SYNTHETIC CONSTRUCT
aucgcccaauaaacauucc

SEQ ID NO: 841 - RNA, 19 nt, Artificial
  SYNTHETIC CONSTRUCT
uagcggguuauuuguaagg

SEQ ID NO: 842 - RNA, 19 nt, Artificial
  SYNTHETIC CONSTRUCT
uagucugaggccccuuaac

SEQ ID NO: 843 - RNA, 19 nt, Artificial
  SYNTHETIC CONSTRUCT
aucagacuccggggaauug

SEQ ID NO: 844 - RNA, 19 nt, Artificial
  SYNTHETIC CONSTRUCT
cugaggccccuuaacucau

SEQ ID NO: 845 - RNA, 19 nt, Artificial
  SYNTHETIC CONSTRUCT
gacuccggggaauugagua

SEQ ID NO: 846 - RNA, 19 nt, Artificial
  SYNTHETIC CONSTRUCT
ggccccuuaacucaucugu

SEQ ID NO: 847 - RNA, 19 nt, Artificial
  SYNTHETIC CONSTRUCT
ccggggaauugaguagaca

SEQ ID NO: 848 - RNA, 19 nt, Artificial
  SYNTHETIC CONSTRUCT
gccccuuaacucaucuguu

SEQ ID NO: 849 - RNA, 19 nt, Artificial
  SYNTHETIC CONSTRUCT
cggggaauugaguagacaa

SEQ ID NO: 850 - RNA, 19 nt, Artificial
  SYNTHETIC CONSTRUCT
ccuuaacucaucuguuauc

SEQ ID NO: 851 - RNA, 19 nt, Artificial
  SYNTHETIC CONSTRUCT
ggaauugaguagacaauag

SEQ ID NO: 852 - RNA, 19 nt, Artificial
  SYNTHETIC CONSTRUCT
uaacucaucuguuauccug

SEQ ID NO: 853 - RNA, 19 nt, Artificial
  SYNTHETIC CONSTRUCT
auugaguagacaauaggac

SEQ ID NO: 854 - RNA, 19 nt, Artificial
  SYNTHETIC CONSTRUCT
acucaucuguuauccugcu

SEQ ID NO: 855 - RNA, 19 nt, Artificial
  SYNTHETIC CONSTRUCT
ugaguagacaauaggacga

SEQ ID NO: 856 - RNA, 19 nt, Artificial
  SYNTHETIC CONSTRUCT
cuguuauccugcuagcugu

SEQ ID NO: 857 - RNA, 19 nt, Artificial
  SYNTHETIC CONSTRUCT
gacaauaggacgaucgaca

SEQ ID NO: 858 - RNA, 19 nt, Artificial
  SYNTHETIC CONSTRUCT
cugcuagcuguagaaaugu

SEQ ID NO: 859 - RNA, 19 nt, Artificial
  SYNTHETIC CONSTRUCT
gacgaucgacaucuuuaca

SEQ ID NO: 860 - RNA, 19 nt, Artificial
  SYNTHETIC CONSTRUCT
cuagcuguagaaauguauc

SEQ ID NO: 861 - RNA, 19 nt, Artificial
  SYNTHETIC CONSTRUCT
gaucgacaucuuuacauag

SEQ ID NO: 862 - RNA, 19 nt, Artificial
  SYNTHETIC CONSTRUCT
uguagaaauguauccugau

SEQ ID NO: 863 - RNA, 19 nt, Artificial
  SYNTHETIC CONSTRUCT
acaucuuuacauaggacua

SEQ ID NO: 864 - RNA, 19 nt, Artificial
  SYNTHETIC CONSTRUCT
gaaauguauccugauaaac

SEQ ID NO: 865 - RNA, 19 nt, Artificial
  SYNTHETIC CONSTRUCT
cuuuacauaggacuauuug

SEQ ID NO: 866 - RNA, 19 nt, Artificial
  SYNTHETIC CONSTRUCT
aaguguaauugugugacuu

SEQ ID NO: 867 - RNA, 19 nt, Artificial
  SYNTHETIC CONSTRUCT
uucacauuaacacacugaa

SEQ ID NO: 868 - RNA, 19 nt, Artificial
  SYNTHETIC CONSTRUCT
ugugacuuuuucagaguug

SEQ ID NO: 869 - RNA, 19 nt, Artificial
  SYNTHETIC CONSTRUCT
acacugaaaaagucucaac

SEQ ID NO: 870 - RNA, 19 nt, Artificial
  SYNTHETIC CONSTRUCT
acuuuuucagaguugcuuu

SEQ ID NO: 871 - RNA, 19 nt, Artificial
  SYNTHETIC CONSTRUCT
ugaaaaagucucaacgaaa

SEQ ID NO: 872 - RNA, 19 nt, Artificial
  SYNTHETIC CONSTRUCT
cagaguugcuuuaaaguac

SEQ ID NO: 873 - RNA, 19 nt, Artificial
  SYNTHETIC CONSTRUCT
gucucaacgaaauuucaug

SEQ ID NO: 874 - RNA, 19 nt, Artificial
  SYNTHETIC CONSTRUCT
agaguugcuuuaaaguacc

SEQ ID NO: 875 - RNA, 19 nt, Artificial
  SYNTHETIC CONSTRUCT
ucucaacgaaauuucaugg

SEQ ID NO: 876 - RNA, 19 nt, Artificial
  SYNTHETIC CONSTRUCT
uuuaaaguaccuguaguga

SEQ ID NO: 877 - RNA, 19 nt, Artificial
  SYNTHETIC CONSTRUCT
aaauuucauggacaucacu

SEQ ID NO: 878 - RNA, 19 nt, Artificial
  SYNTHETIC CONSTRUCT
ccuguagugagaaacugau

SEQ ID NO: 879 - RNA, 19 nt, Artificial
  SYNTHETIC CONSTRUCT
ggacaucacucuuugacua

SEQ ID NO: 880 - RNA, 19 nt, Artificial
  SYNTHETIC CONSTRUCT
agugagaaacugauuuaug

SEQ ID NO: 881 - RNA, 19 nt, Artificial
  SYNTHETIC CONSTRUCT
ucacucuuugacuaaauac

SEQ ID NO: 882 - RNA, 19 nt, Artificial
  SYNTHETIC CONSTRUCT
acugauuuaugaucacuug

SEQ ID NO: 883 - RNA, 19 nt, Artificial
  SYNTHETIC CONSTRUCT
ugacuaaauacuagugaac

SEQ ID NO: 884 - RNA, 19 nt, Artificial
  SYNTHETIC CONSTRUCT
gaucacuuggaagauuugu

SEQ ID NO: 885 - RNA, 19 nt, Artificial
  SYNTHETIC CONSTRUCT
cuagugaaccuucuaaaca

SEQ ID NO: 886 - RNA, 19 nt, Artificial
  SYNTHETIC CONSTRUCT
acuuggaagauuuguauag

SEQ ID NO: 887 - RNA, 19 nt, Artificial
  SYNTHETIC CONSTRUCT
ugaaccuucuaaacauauc

SEQ ID NO: 888 - RNA, 19 nt, Artificial
  SYNTHETIC CONSTRUCT
acucaguuaaaaugucugu

SEQ ID NO: 889 - RNA, 19 nt, Artificial
  SYNTHETIC CONSTRUCT
ugagucaauuuuacagaca

SEQ ID NO: 890 - RNA, 19 nt, Artificial
  SYNTHETIC CONSTRUCT
caguuaaaaugucuguuuc

SEQ ID NO: 891 - RNA, 19 nt, Artificial
  SYNTHETIC CONSTRUCT
gucaauuuuacagacaaag

SEQ ID NO: 892 - RNA, 19 nt, Artificial
  SYNTHETIC CONSTRUCT
augucuguuucaaugaccu

SEQ ID NO: 893 - RNA, 19 nt, Artificial
  SYNTHETIC CONSTRUCT
uacagacaaaguuacugga

SEQ ID NO: 894 - RNA, 19 nt, Artificial
  SYNTHETIC CONSTRUCT
uucaaugaccuguauuuug

SEQ ID NO: 895 - RNA, 19 nt, Artificial
  SYNTHETIC CONSTRUCT
aaguuacuggacauaaaac

SEQ ID NO: 896 - RNA, 19 nt, Artificial
  SYNTHETIC CONSTRUCT
auuuugccagacuuaaauc

SEQ ID NO: 897 - RNA, 19 nt, Artificial
  SYNTHETIC CONSTRUCT
uaaaacggucugaauuuag

SEQ ID NO: 898 - RNA, 19 nt, Artificial
  SYNTHETIC CONSTRUCT
agacuuaaaucacagaugg

SEQ ID NO: 899 - RNA, 19 nt, Artificial
  SYNTHETIC CONSTRUCT
ucugaauuuagugucuacc

SEQ ID NO: 900 - RNA, 19 nt, Artificial
  SYNTHETIC CONSTRUCT
cuuaaaucacagaugggua

SEQ ID NO: 901 - RNA, 19 nt, Artificial
  SYNTHETIC CONSTRUCT
gaauuuagugucuacccau

SEQ ID NO: 902 - RNA, 19 nt, Artificial
  SYNTHETIC CONSTRUCT
gauggguauuaaacuuguc

SEQ ID NO: 903 - RNA, 19 nt, Artificial
  SYNTHETIC CONSTRUCT
cuacccauaauuugaacag

SEQ ID NO: 904 - RNA, 19 nt, Artificial
  SYNTHETIC CONSTRUCT
gguauuaaacuugucagaa

SEQ ID NO: 905 - RNA, 19 nt, Artificial
  SYNTHETIC CONSTRUCT
ccauaauuugaacagucuu

SEQ ID NO: 906 - RNA, 19 nt, Artificial
  SYNTHETIC CONSTRUCT
uugucagaauuucuuuguc

SEQ ID NO: 907 - RNA, 19 nt, Artificial
  SYNTHETIC CONSTRUCT
aacagucuuaaagaaacag

SEQ ID NO: 908 - RNA, 19 nt, Artificial
  SYNTHETIC CONSTRUCT
cuuugucauucaagccugu

SEQ ID NO: 909 - RNA, 19 nt, Artificial
  SYNTHETIC CONSTRUCT
gaaacaguaaguucggaca

SEQ ID NO: 910 - RNA, 19 nt, Artificial
  SYNTHETIC CONSTRUCT
gccugugaauaaaaacccu

SEQ ID NO: 911 - RNA, 19 nt, Artificial
  SYNTHETIC CONSTRUCT
cggacacuuauuuuuggga

SEQ ID NO: 912 - RNA, 19 nt, Artificial
  SYNTHETIC CONSTRUCT
ugaauaaaaacccuguaug

SEQ ID NO: 913 - RNA, 19 nt, Artificial
  SYNTHETIC CONSTRUCT
acuuauuuuugggacauac

SEQ ID NO: 914 - RNA, 19 nt, Artificial
  SYNTHETIC CONSTRUCT
gaauaaaaacccuguaugg

SEQ ID NO: 915 - RNA, 19 nt, Artificial
  SYNTHETIC CONSTRUCT
cuuauuuuugggacauacc

SEQ ID NO: 916 - RNA, 19 nt, Artificial
  SYNTHETIC CONSTRUCT
aaaaacccuguauggcacu

SEQ ID NO: 917 - RNA, 19 nt, Artificial
  SYNTHETIC CONSTRUCT
uuuuugggacauaccguga

SEQ ID NO: 918 - RNA, 19 nt, Artificial
  SYNTHETIC CONSTRUCT
cuguauggcacuuauuaug

SEQ ID NO: 919 - RNA, 19 nt, Artificial
  SYNTHETIC CONSTRUCT
gacauaccgugaauaauac

SEQ ID NO: 920 - RNA, 19 nt, Artificial
  SYNTHETIC CONSTRUCT
uggcacuuauuaugaggcu

SEQ ID NO: 921 - RNA, 19 nt, Artificial
  SYNTHETIC CONSTRUCT
accgugaauaauacuccga

SEQ ID NO: 922 - RNA, 19 nt, Artificial
  SYNTHETIC CONSTRUCT
ggcacuuauuaugaggcua

SEQ ID NO: 923 - RNA, 19 nt, Artificial
  SYNTHETIC CONSTRUCT
ccgugaauaauacuccgau

SEQ ID NO: 924 - RNA, 19 nt, Artificial
  SYNTHETIC CONSTRUCT
aggcuauuaaaagaaucca

SEQ ID NO: 925 - RNA, 19 nt, Artificial
  SYNTHETIC CONSTRUCT
uccgauaauuuucuuaggu

SEQ ID NO: 926 - RNA, 19 nt, Artificial
  SYNTHETIC CONSTRUCT
cgcggaggucuggccuaua

SEQ ID NO: 927 - RNA, 19 nt, Artificial
  SYNTHETIC CONSTRUCT
gcgccuccagaccggauau

SEQ ID NO: 928 - RNA, 19 nt, Artificial
  SYNTHETIC CONSTRUCT
ggagacggggugcugguuu

SEQ ID NO: 929 - RNA, 19 nt, Artificial
  SYNTHETIC CONSTRUCT
ccucugccccacgaccaaa

SEQ ID NO: 930 - RNA, 19 nt, Artificial
  SYNTHETIC CONSTRUCT
guagucuccugcagcgucu

SEQ ID NO: 931 - RNA, 19 nt, Artificial
  SYNTHETIC CONSTRUCT
caucagaggacgucgcaga

SEQ ID NO: 932 - RNA, 19 nt, Artificial
  SYNTHETIC CONSTRUCT
gcaguccucggaaccagga

SEQ ID NO: 933 - RNA, 19 nt, Artificial
  SYNTHETIC CONSTRUCT
cgucaggagccuugguccu

SEQ ID NO: 934 - RNA, 19 nt, Artificial
  SYNTHETIC CONSTRUCT
aggccgugugcgugcugaa

SEQ ID NO: 935 - RNA, 19 nt, Artificial
  SYNTHETIC CONSTRUCT
uccggcacacgcacgacuu

SEQ ID NO: 936 - RNA, 19 nt, Artificial
  SYNTHETIC CONSTRUCT
ccagugaagguguggggaa

SEQ ID NO: 937 - RNA, 19 nt, Artificial
  SYNTHETIC CONSTRUCT
ggucacuuccacaccccuu

SEQ ID NO: 938 - RNA, 19 nt, Artificial
  SYNTHETIC CONSTRUCT
gugaagguguggggaagca

SEQ ID NO: 939 - RNA, 19 nt, Artificial
  SYNTHETIC CONSTRUCT
cacuuccacaccccuucgu

SEQ ID NO: 940 - RNA, 19 nt, Artificial
  SYNTHETIC CONSTRUCT
agcaggcuguaccagugca

SEQ ID NO: 941 - RNA, 19 nt, Artificial
  SYNTHETIC CONSTRUCT
ucguccgacauggucacgu

SEQ ID NO: 942 - RNA, 19 nt, Artificial
  SYNTHETIC CONSTRUCT
ccagugcagguccucacuu

SEQ ID NO: 943 - RNA, 19 nt, Artificial
  SYNTHETIC CONSTRUCT
ggucacguccaggagugaa

SEQ ID NO: 944 - RNA, 19 nt, Artificial
  SYNTHETIC CONSTRUCT
aacacggugggccaaagga

SEQ ID NO: 945 - RNA, 19 nt, Artificial
  SYNTHETIC CONSTRUCT
uugugccacccgguuuccu

SEQ ID NO: 946 - RNA, 19 nt, Artificial
  SYNTHETIC CONSTRUCT
acacggugggccaaaggau

SEQ ID NO: 947 - RNA, 19 nt, Artificial
  SYNTHETIC CONSTRUCT
ugugccacccgguuuccua

SEQ ID NO: 948 - RNA, 19 nt, Artificial
  SYNTHETIC CONSTRUCT
acggugggccaaaggauga

SEQ ID NO: 949 - RNA, 19 nt, Artificial
  SYNTHETIC CONSTRUCT
ugccacccgguuuccuacu

SEQ ID NO: 950 - RNA, 19 nt, Artificial
  SYNTHETIC CONSTRUCT
cggugggccaaaggaugaa

SEQ ID NO: 951 - RNA, 19 nt, Artificial
  SYNTHETIC CONSTRUCT
gccacccgguuuccuacuu

SEQ ID NO: 952 - RNA, 19 nt, Artificial
  SYNTHETIC CONSTRUCT
gccgcacacugguggucca

SEQ ID NO: 953 - RNA, 19 nt, Artificial
  SYNTHETIC CONSTRUCT
cggcgugugaccaccaggu

SEQ ID NO: 954 - RNA, 19 nt, Artificial
  SYNTHETIC CONSTRUCT
auguauccugauaaacauu

SEQ ID NO: 955 - RNA, 19 nt, Artificial
  SYNTHETIC CONSTRUCT
uacauaggacuauuuguaa

SEQ ID NO: 956 - RNA, 19 nt, Artificial
  SYNTHETIC CONSTRUCT
gauaaacauuaaacacugu

SEQ ID NO: 957 - RNA, 19 nt, Artificial
  SYNTHETIC CONSTRUCT
cuauuuguaauuugugaca

SEQ ID NO: 958 - RNA, 19 nt, Artificial
  SYNTHETIC CONSTRUCT
acauuaaacacuguaaucu

SEQ ID NO: 959 - RNA, 19 nt, Artificial
  SYNTHETIC CONSTRUCT
uguaauuugugacauuaga

SEQ ID NO: 960 - RNA, 19 nt, Artificial
  SYNTHETIC CONSTRUCT
cauuaaacacuguaaucuu

SEQ ID NO: 961 - RNA, 19 nt, Artificial
  SYNTHETIC CONSTRUCT
guaauuugugacauuagaa

SEQ ID NO: 962 - RNA, 19 nt, Artificial
  SYNTHETIC CONSTRUCT
acuguaaucuuaaaagugu

SEQ ID NO: 963 - RNA, 19 nt, Artificial
  SYNTHETIC CONSTRUCT
ugacauuagaauuuucaca

SEQ ID NO: 964 - RNA, 19 nt, Artificial
  SYNTHETIC CONSTRUCT
guaaucuuaaaaguguaau

SEQ ID NO: 965 - RNA, 19 nt, Artificial
  SYNTHETIC CONSTRUCT
cauuagaauuuucacauua

SEQ ID NO: 966 - RNA, 19 nt, Artificial
  SYNTHETIC CONSTRUCT
aucuuaaaaguguaauugu

SEQ ID NO: 967 - RNA, 19 nt, Artificial
  SYNTHETIC CONSTRUCT
uagaauuuucacauuaaca

SEQ ID NO: 968 - RNA, 19 nt, Artificial
  SYNTHETIC CONSTRUCT
uagugagaaacugauuuau

SEQ ID NO: 969 - RNA, 19 nt, Artificial
  SYNTHETIC CONSTRUCT
aucacucuuugacuaaaua

SEQ ID NO: 970 - RNA, 19 nt, Artificial
  SYNTHETIC CONSTRUCT
aaacugauuuaugaucacu

SEQ ID NO: 971 - RNA, 19 nt, Artificial
  SYNTHETIC CONSTRUCT
uuugacuaaauacuaguga

SEQ ID NO: 972 - RNA, 19 nt, Artificial
  SYNTHETIC CONSTRUCT
aagauuuguauaguuuuau

SEQ ID NO: 973 - RNA, 19 nt, Artificial
  SYNTHETIC CONSTRUCT
uucuaaacauaucaaaaua

SEQ ID NO: 974 - RNA, 19 nt, Artificial
  SYNTHETIC CONSTRUCT
uuguauaguuuuauaaaac

SEQ ID NO: 975 - RNA, 19 nt, Artificial
  SYNTHETIC CONSTRUCT
aacauaucaaaauauuuug

SEQ ID NO: 976 - RNA, 19 nt, Artificial
  SYNTHETIC CONSTRUCT
guauaguuuuauaaaacuc

SEQ ID NO: 977 - RNA, 19 nt, Artificial
  SYNTHETIC CONSTRUCT
cauaucaaaauauuuugag

SEQ ID NO: 978 - RNA, 19 nt, Artificial
  SYNTHETIC CONSTRUCT
uauaguuuuauaaaacuca

SEQ ID NO: 979 - RNA, 19 nt, Artificial
  SYNTHETIC CONSTRUCT
auaucaaaauauuuugagu

SEQ ID NO: 980 - RNA, 19 nt, Artificial
  SYNTHETIC CONSTRUCT
auaaaacucaguuaaaaug

SEQ ID NO: 981 - RNA, 19 nt, Artificial
  SYNTHETIC CONSTRUCT
uauuuugagucaauuuuac

SEQ ID NO: 982 - RNA, 19 nt, Artificial
  SYNTHETIC CONSTRUCT
aaaacucaguuaaaauguc

SEQ ID NO: 983 - RNA, 19 nt, Artificial
  SYNTHETIC CONSTRUCT
uuuugagucaauuuuacag

SEQ ID NO: 984 - RNA, 19 nt, Artificial
  SYNTHETIC CONSTRUCT
uaaaaugucuguuucaaug

SEQ ID NO: 985 - RNA, 19 nt, Artificial
  SYNTHETIC CONSTRUCT
auuuuacagacaaaguuac

SEQ ID NO: 986 - RNA, 19 nt, Artificial
  SYNTHETIC CONSTRUCT
uuucaaugaccuguauuuu

SEQ ID NO: 987 - RNA, 19 nt, Artificial
  SYNTHETIC CONSTRUCT
aaaguuacuggacauaaaa

SEQ ID NO: 988 - RNA, 19 nt, Artificial
  SYNTHETIC CONSTRUCT
uuaaacuugucagaauuuc

SEQ ID NO: 989 - RNA, 19 nt, Artificial
  SYNTHETIC CONSTRUCT
aauuugaacagucuuaaag

SEQ ID NO: 990 - RNA, 19 nt, Artificial
  SYNTHETIC CONSTRUCT
aacuugucagaauuucuuu

SEQ ID NO: 991 - RNA, 19 nt, Artificial
  SYNTHETIC CONSTRUCT
uugaacagucuuaaagaaa

SEQ ID NO: 992 - RNA, 19 nt, Artificial
  SYNTHETIC CONSTRUCT
gcuauuaaaagaauccaaa

SEQ ID NO: 993 - RNA, 19 nt, Artificial
  SYNTHETIC CONSTRUCT
cgauaauuuucuuagguuu

SEQ ID NO: 994 - RNA, 19 nt, Artificial
  SYNTHETIC CONSTRUCT
aagaauccaaauucaaacu

SEQ ID NO: 995 - RNA, 19 nt, Artificial
  SYNTHETIC CONSTRUCT
uucuuagguuuaaguuuga

SEQ ID NO: 996 - RNA, 19 nt, Artificial
  SYNTHETIC CONSTRUCT
uauaaaguagucgcggaga

SEQ ID NO: 997 - RNA, 19 nt, Artificial
  SYNTHETIC CONSTRUCT
auauuucaucagcgccucu

SEQ ID NO: 998 - RNA, 19 nt, Artificial
  SYNTHETIC CONSTRUCT
cugguuugcgucguagucu

SEQ ID NO: 999 - RNA, 19 nt, Artificial
  SYNTHETIC CONSTRUCT
gaccaaacgcagcaucaga

SEQ ID NO: 1000 - RNA, 19 nt, Artificial
  SYNTHETIC CONSTRUCT
ugguuugcgucguagucuc

SEQ ID NO: 1001 - RNA, 19 nt, Artificial
  SYNTHETIC CONSTRUCT
accaaacgcagcaucagag

SEQ ID NO: 1002 - RNA, 19 nt, Artificial
  SYNTHETIC CONSTRUCT
uuugcgucguagucuccug

SEQ ID NO: 1003 - RNA, 19 nt, Artificial
  SYNTHETIC CONSTRUCT
aaacgcagcaucagaggac

SEQ ID NO: 1004 - RNA, 19 nt, Artificial
  SYNTHETIC CONSTRUCT
uagcgaguuauggcgacga

SEQ ID NO: 1005 - RNA, 19 nt, Artificial
  SYNTHETIC CONSTRUCT
aucgcucaauaccgcugcu

SEQ ID NO: 1006 - RNA, 19 nt, Artificial
  SYNTHETIC CONSTRUCT
cagggcaucaucaauuucg

SEQ ID NO: 1007 - RNA, 19 nt, Artificial
  SYNTHETIC CONSTRUCT
gucccguaguaguuaaagc

SEQ ID NO: 1008 - RNA, 19 nt, Artificial
  SYNTHETIC CONSTRUCT
caucaucaauuucgagcag

SEQ ID NO: 1009 - RNA, 19 nt, Artificial
  SYNTHETIC CONSTRUCT
guaguaguuaaagcucguc

SEQ ID NO: 1010 - RNA, 19 nt, Artificial
  SYNTHETIC CONSTRUCT
auuucgagcagaaggaaag

SEQ ID NO: 1011 - RNA, 19 nt, Artificial
  SYNTHETIC CONSTRUCT
uaaagcucgucuuccuuuc

SEQ ID NO: 1012 - RNA, 19 nt, Artificial
  SYNTHETIC CONSTRUCT
aaguaauggaccagugaag

SEQ ID NO: 1013 - RNA, 19 nt, Artificial
  SYNTHETIC CONSTRUCT
uucauuaccuggucacuuc

SEQ ID NO: 1014 - RNA, 19 nt, Artificial
  SYNTHETIC CONSTRUCT
aauggaccagugaaggugu

SEQ ID NO: 1015 - RNA, 19 nt, Artificial
  SYNTHETIC CONSTRUCT
uuaccuggucacuuccaca

SEQ ID NO: 1016 - RNA, 19 nt, Artificial
  SYNTHETIC CONSTRUCT
auggaccagugaaggugug

SEQ ID NO: 1017 - RNA, 19 nt, Artificial
  SYNTHETIC CONSTRUCT
uaccuggucacuuccacac

SEQ ID NO: 1018 - RNA, 19 nt, Artificial
  SYNTHETIC CONSTRUCT
uguggggaagcauuaaagg

SEQ ID NO: 1019 - RNA, 19 nt, Artificial
  SYNTHETIC CONSTRUCT
acaccccuucguaauuucc

SEQ ID NO: 1020 - RNA, 19 nt, Artificial
  SYNTHETIC CONSTRUCT
gggaagcauuaaaggacug

SEQ ID NO: 1021 - RNA, 19 nt, Artificial
  SYNTHETIC CONSTRUCT
cccuucguaauuuccugac

SEQ ID NO: 1022 - RNA, 19 nt, Artificial
  SYNTHETIC CONSTRUCT
auuaaaggacugacugaag

SEQ ID NO: 1023 - RNA, 19 nt, Artificial
  SYNTHETIC CONSTRUCT
uaauuuccugacugacuuc

SEQ ID NO: 1024 - RNA, 19 nt, Artificial
  SYNTHETIC CONSTRUCT
uaaaggacugacugaaggc

SEQ ID NO: 1025 - RNA, 19 nt, Artificial
  SYNTHETIC CONSTRUCT
auuuccugacugacuuccg

SEQ ID NO: 1026 - RNA, 19 nt, Artificial
  SYNTHETIC CONSTRUCT
uccauguucaugaguuugg

SEQ ID NO: 1027 - RNA, 19 nt, Artificial
  SYNTHETIC CONSTRUCT
agguacaaguacucaaacc

SEQ ID NO: 1028 - RNA, 19 nt, Artificial
  SYNTHETIC CONSTRUCT
gaguuuggagauaauacag

SEQ ID NO: 1029 - RNA, 19 nt, Artificial
  SYNTHETIC CONSTRUCT
cucaaaccucuauuauguc

SEQ ID NO: 1030 - RNA, 19 nt, Artificial
  SYNTHETIC CONSTRUCT
ggagauaauacagcaggcu

SEQ ID NO: 1031 - RNA, 19 nt, Artificial
  SYNTHETIC CONSTRUCT
ccucuauuaugucguccga

SEQ ID NO: 1032 - RNA, 19 nt, Artificial
  SYNTHETIC CONSTRUCT
auaauacagcaggcuguac

SEQ ID NO: 1033 - RNA, 19 nt, Artificial
  SYNTHETIC CONSTRUCT
uauuaugucguccgacaug

SEQ ID NO: 1034 - RNA, 19 nt, Artificial
  SYNTHETIC CONSTRUCT
uaauacagcaggcuguacc

SEQ ID NO: 1035 - RNA, 19 nt, Artificial
  SYNTHETIC CONSTRUCT
auuaugucguccgacaugg

SEQ ID NO: 1036 - RNA, 19 nt, Artificial
  SYNTHETIC CONSTRUCT
uacagcaggcuguaccagu

SEQ ID NO: 1037 - RNA, 19 nt, Artificial
  SYNTHETIC CONSTRUCT
augucguccgacaugguca

SEQ ID NO: 1038 - RNA, 19 nt, Artificial
  SYNTHETIC CONSTRUCT
cagguccucacuuuaaucc

SEQ ID NO: 1039 - RNA, 19 nt, Artificial
  SYNTHETIC CONSTRUCT
guccaggagugaaauuagg

SEQ ID NO: 1040 - RNA, 19 nt, Artificial
  SYNTHETIC CONSTRUCT
gguccucacuuuaauccuc

SEQ ID NO: 1041 - RNA, 19 nt, Artificial
  SYNTHETIC CONSTRUCT
ccaggagugaaauuaggag

SEQ ID NO: 1042 - RNA, 19 nt, Artificial
  SYNTHETIC CONSTRUCT
cucacuuuaauccucuauc

SEQ ID NO: 1043 - RNA, 19 nt, Artificial
  SYNTHETIC CONSTRUCT
gagugaaauuaggagauag

SEQ ID NO: 1044 - RNA, 19 nt, Artificial
  SYNTHETIC CONSTRUCT
uccucuauccagaaaacac

SEQ ID NO: 1045 - RNA, 19 nt, Artificial
  SYNTHETIC CONSTRUCT
aggagauaggucuuuugug

SEQ ID NO: 1046 - RNA, 19 nt, Artificial
  SYNTHETIC CONSTRUCT
ucuauccagaaaacacggu

SEQ ID NO: 1047 - RNA, 19 nt, Artificial
  SYNTHETIC CONSTRUCT
agauaggucuuuugugcca

SEQ ID NO: 1048 - RNA, 19 nt, Artificial
  SYNTHETIC CONSTRUCT
ugggccaaaggaugaagag

SEQ ID NO: 1049 - RNA, 19 nt, Artificial
  SYNTHETIC CONSTRUCT
acccgguuuccuacuucuc

SEQ ID NO: 1050 - RNA, 19 nt, Artificial
  SYNTHETIC CONSTRUCT
gccaaaggaugaagagagg

SEQ ID NO: 1051 - RNA, 19 nt, Artificial
  SYNTHETIC CONSTRUCT
cgguuuccuacuucucucc

SEQ ID NO: 1052 - RNA, 19 nt, Artificial
  SYNTHETIC CONSTRUCT
aaaggaugaagagaggcau

SEQ ID NO: 1053 - RNA, 19 nt, Artificial
  SYNTHETIC CONSTRUCT
uuuccuacuucucuccgua

SEQ ID NO: 1054 - RNA, 19 nt, Artificial
  SYNTHETIC CONSTRUCT
aggaugaagagaggcaugu

SEQ ID NO: 1055 - RNA, 19 nt, Artificial
  SYNTHETIC CONSTRUCT
uccuacuucucuccguaca

SEQ ID NO: 1056 - RNA, 19 nt, Artificial
  SYNTHETIC CONSTRUCT
gaugaagagaggcauguug

SEQ ID NO: 1057 - RNA, 19 nt, Artificial
  SYNTHETIC CONSTRUCT
cuacuucucuccguacaac

SEQ ID NO: 1058 - RNA, 19 nt, Artificial
  SYNTHETIC CONSTRUCT
gagaggcauguuggagacu

SEQ ID NO: 1059 - RNA, 19 nt, Artificial
  SYNTHETIC CONSTRUCT
cucuccguacaaccucuga

SEQ ID NO: 1060 - RNA, 19 nt, Artificial
  SYNTHETIC CONSTRUCT
cauguuggagacuugggca

SEQ ID NO: 1061 - RNA, 19 nt, Artificial
  SYNTHETIC CONSTRUCT
guacaaccucugaacccgu

SEQ ID NO: 1062 - RNA, 19 nt, Artificial
  SYNTHETIC CONSTRUCT
uguuggagacuugggcaau

SEQ ID NO: 1063 - RNA, 19 nt, Artificial
  SYNTHETIC CONSTRUCT
acaaccucugaacccguua

SEQ ID NO: 1064 - RNA, 19 nt, Artificial
  SYNTHETIC CONSTRUCT
guuggagacuugggcaaug

SEQ ID NO: 1065 - RNA, 19 nt, Artificial
  SYNTHETIC CONSTRUCT
caaccucugaacccguuac

SEQ ID NO: 1066 - RNA, 19 nt, Artificial
  SYNTHETIC CONSTRUCT
uggagacuugggcaaugug

SEQ ID NO: 1067 - RNA, 19 nt, Artificial
  SYNTHETIC CONSTRUCT
accucugaacccguuacac

SEQ ID NO: 1068 - RNA, 19 nt, Artificial
  SYNTHETIC CONSTRUCT
cuugggcaaugugacugcu

SEQ ID NO: 1069 - RNA, 19 nt, Artificial
  SYNTHETIC CONSTRUCT
gaacccguuacacugacga

SEQ ID NO: 1070 - RNA, 19 nt, Artificial
  SYNTHETIC CONSTRUCT
gugacugcugacaaagaug

SEQ ID NO: 1071 - RNA, 19 nt, Artificial
  SYNTHETIC CONSTRUCT
cacugacgacuguuucuac

SEQ ID NO: 1072 - RNA, 19 nt, Artificial
  SYNTHETIC CONSTRUCT
ugacugcugacaaagaugg

SEQ ID NO: 1073 - RNA, 19 nt, Artificial
  SYNTHETIC CONSTRUCT
acugacgacuguuucuacc

SEQ ID NO: 1074 - RNA, 19 nt, Artificial
  SYNTHETIC CONSTRUCT
cugcugacaaagauggugu

SEQ ID NO: 1075 - RNA, 19 nt, Artificial
  SYNTHETIC CONSTRUCT
gacgacuguuucuaccaca

SEQ ID NO: 1076 - RNA, 19 nt, Artificial
  SYNTHETIC CONSTRUCT
ugacaaagaugguguggcc

SEQ ID NO: 1077 - RNA, 19 nt, Artificial
  SYNTHETIC CONSTRUCT
acuguuucuaccacaccgg

SEQ ID NO: 1078 - RNA, 19 nt, Artificial
  SYNTHETIC CONSTRUCT
uugaagauucugugaucuc

SEQ ID NO: 1079 - RNA, 19 nt, Artificial
  SYNTHETIC CONSTRUCT
aacuucuaagacacuagag

SEQ ID NO: 1080 - RNA, 19 nt, Artificial
  SYNTHETIC CONSTRUCT
gaagauucugugaucucac

SEQ ID NO: 1081 - RNA, 19 nt, Artificial
  SYNTHETIC CONSTRUCT
cuucuaagacacuagagug

SEQ ID NO: 1082 - RNA, 19 nt, Artificial
  SYNTHETIC CONSTRUCT
agauucugugaucucacuc

SEQ ID NO: 1083 - RNA, 19 nt, Artificial
  SYNTHETIC CONSTRUCT
ucuaagacacuagagugag

SEQ ID NO: 1084 - RNA, 19 nt, Artificial
  SYNTHETIC CONSTRUCT
auucugugaucucacucuc

SEQ ID NO: 1085 - RNA, 19 nt, Artificial
  SYNTHETIC CONSTRUCT
uaagacacuagagugagag

SEQ ID NO: 1086 - RNA, 19 nt, Artificial
  SYNTHETIC CONSTRUCT
ucugugaucucacucucag

SEQ ID NO: 1087 - RNA, 19 nt, Artificial
  SYNTHETIC CONSTRUCT
agacacuagagugagaguc

SEQ ID NO: 1088 - RNA, 19 nt, Artificial
  SYNTHETIC CONSTRUCT
ucucacucucaggagacca

SEQ ID NO: 1089 - RNA, 19 nt, Artificial
  SYNTHETIC CONSTRUCT
agagugagaguccucuggu

SEQ ID NO: 1090 - RNA, 19 nt, Artificial
  SYNTHETIC CONSTRUCT
cacucucaggagaccauug

SEQ ID NO: 1091 - RNA, 19 nt, Artificial
  SYNTHETIC CONSTRUCT
gugagaguccucugguaac

SEQ ID NO: 1092 - RNA, 19 nt, Artificial
  SYNTHETIC CONSTRUCT
acucucaggagaccauugc

SEQ ID NO: 1093 - RNA, 19 nt, Artificial
  SYNTHETIC CONSTRUCT
ugagaguccucugguaacg

SEQ ID NO: 1094 - RNA, 19 nt, Artificial
  SYNTHETIC CONSTRUCT
gagaccauugcaucauugg

SEQ ID NO: 1095 - RNA, 19 nt, Artificial
  SYNTHETIC CONSTRUCT
cucugguaacguaguaacc

SEQ ID NO: 1096 - RNA, 19 nt, Artificial
  SYNTHETIC CONSTRUCT
agaccauugcaucauuggc

SEQ ID NO: 1097 - RNA, 19 nt, Artificial
  SYNTHETIC CONSTRUCT
ucugguaacguaguaaccg

SEQ ID NO: 1098 - RNA, 19 nt, Artificial
  SYNTHETIC CONSTRUCT
cuggugguccaugaaaaag

SEQ ID NO: 1099 - RNA, 19 nt, Artificial
  SYNTHETIC CONSTRUCT
gaccaccagguacuuuuuc

SEQ ID NO: 1100 - RNA, 19 nt, Artificial
  SYNTHETIC CONSTRUCT
uggugguccaugaaaaagc

SEQ ID NO: 1101 - RNA, 19 nt, Artificial
  SYNTHETIC CONSTRUCT
accaccagguacuuuuucg

SEQ ID NO: 1102 - RNA, 19 nt, Artificial
  SYNTHETIC CONSTRUCT
caugaaaaagcagaugacu

SEQ ID NO: 1103 - RNA, 19 nt, Artificial
  SYNTHETIC CONSTRUCT
guacuuuuucgucuacuga

SEQ ID NO: 1104 - RNA, 19 nt, Artificial
  SYNTHETIC CONSTRUCT
aaaaagcagaugacuuggg

SEQ ID NO: 1105 - RNA, 19 nt, Artificial
  SYNTHETIC CONSTRUCT
uuuuucgucuacugaaccc

SEQ ID NO: 1106 - RNA, 19 nt, Artificial
  SYNTHETIC CONSTRUCT
aaaagcagaugacuugggc

SEQ ID NO: 1107 - RNA, 19 nt, Artificial
  SYNTHETIC CONSTRUCT
uuuucgucuacugaacccg

SEQ ID NO: 1108 - RNA, 19 nt, Artificial
  SYNTHETIC CONSTRUCT
cagaugacuugggcaaagg

SEQ ID NO: 1109 - RNA, 19 nt, Artificial
  SYNTHETIC CONSTRUCT
gucuacugaacccguuucc

SEQ ID NO: 1110 - RNA, 19 nt, Artificial
  SYNTHETIC CONSTRUCT
uugggcaaagguggaaaug

SEQ ID NO: 1111 - RNA, 19 nt, Artificial
  SYNTHETIC CONSTRUCT
aacccguuuccaccuuuac

SEQ ID NO: 1112 - RNA, 19 nt, Artificial
  SYNTHETIC CONSTRUCT
ggcaaagguggaaaugaag

SEQ ID NO: 1113 - RNA, 19 nt, Artificial
  SYNTHETIC CONSTRUCT
ccguuuccaccuuuacuuc

SEQ ID NO: 1114 - RNA, 19 nt, Artificial
  SYNTHETIC CONSTRUCT
aagguggaaaugaagaaag

SEQ ID NO: 1115 - RNA, 19 nt, Artificial
  SYNTHETIC CONSTRUCT
uuccaccuuuacuucuuuc

SEQ ID NO: 1116 - RNA, 19 nt, Artificial
  SYNTHETIC CONSTRUCT
guggaaaugaagaaaguac

SEQ ID NO: 1117 - RNA, 19 nt, Artificial
  SYNTHETIC CONSTRUCT
caccuuuacuucuuucaug

SEQ ID NO: 1118 - RNA, 19 nt, Artificial
  SYNTHETIC CONSTRUCT
augaagaaaguacaaagac

SEQ ID NO: 1119 - RNA, 19 nt, Artificial
  SYNTHETIC CONSTRUCT
uacuucuuucauguuucug

SEQ ID NO: 1120 - RNA, 19 nt, Artificial
  SYNTHETIC CONSTRUCT
gaagaaaguacaaagacag

SEQ ID NO: 1121 - RNA, 19 nt, Artificial
  SYNTHETIC CONSTRUCT
cuucuuucauguuucuguc

SEQ ID NO: 1122 - RNA, 19 nt, Artificial
  SYNTHETIC CONSTRUCT
aaguacaaagacaggaaac

SEQ ID NO: 1123 - RNA, 19 nt, Artificial
  SYNTHETIC CONSTRUCT
uucauguuucuguccuuug

SEQ ID NO: 1124 - RNA, 19 nt, Artificial
  SYNTHETIC CONSTRUCT
uacaaagacaggaaacgcu

SEQ ID NO: 1125 - RNA, 19 nt, Artificial
  SYNTHETIC CONSTRUCT
auguuucuguccuuugcga

SEQ ID NO: 1126 - RNA, 19 nt, Artificial
  SYNTHETIC CONSTRUCT
agacaggaaacgcuggaag

SEQ ID NO: 1127 - RNA, 19 nt, Artificial
  SYNTHETIC CONSTRUCT
ucuguccuuugcgaccuuc

SEQ ID NO: 1128 - RNA, 19 nt, Artificial
  SYNTHETIC CONSTRUCT
acaggaaacgcuggaaguc

SEQ ID NO: 1129 - RNA, 19 nt, Artificial
  SYNTHETIC CONSTRUCT
uguccuuugcgaccuucag

SEQ ID NO: 1130 - RNA, 19 nt, Artificial
  SYNTHETIC CONSTRUCT
aacgcuggaagucguuugg

SEQ ID NO: 1131 - RNA, 19 nt, Artificial
  SYNTHETIC CONSTRUCT
uugcgaccuucagcaaacc

SEQ ID NO: 1132 - RNA, 19 nt, Artificial
  SYNTHETIC CONSTRUCT
gcuggaagucguuuggcuu

SEQ ID NO: 1133 - RNA, 19 nt, Artificial
  SYNTHETIC CONSTRUCT
cgaccuucagcaaaccgaa

SEQ ID NO: 1134 - RNA, 19 nt, Artificial
  SYNTHETIC CONSTRUCT
aagucguuuggcuuguggu

SEQ ID NO: 1135 - RNA, 19 nt, Artificial
  SYNTHETIC CONSTRUCT
uucagcaaaccgaacacca

SEQ ID NO: 1136 - RNA, 19 nt, Artificial
  SYNTHETIC CONSTRUCT
uuggcuugugguguaauug

SEQ ID NO: 1137 - RNA, 19 nt, Artificial
  SYNTHETIC CONSTRUCT
aaccgaacaccacauuaac

SEQ ID NO: 1138 - RNA, 19 nt, Artificial
  SYNTHETIC CONSTRUCT
uggcuugugguguaauugg

SEQ ID NO: 1139 - RNA, 19 nt, Artificial
  SYNTHETIC CONSTRUCT
accgaacaccacauuaacc

SEQ ID NO: 1140 - RNA, 19 nt, Artificial
  SYNTHETIC CONSTRUCT
ggcuugugguguaauuggg

SEQ ID NO: 1141 - RNA, 19 nt, Artificial
  SYNTHETIC CONSTRUCT
ccgaacaccacauuaaccc

SEQ ID NO: 1142 - RNA, 19 nt, Artificial
  SYNTHETIC CONSTRUCT
guguaauugggaucgccca

SEQ ID NO: 1143 - RNA, 19 nt, Artificial
  SYNTHETIC CONSTRUCT
cacauuaacccuagcgggu

SEQ ID NO: 1144 - RNA, 19 nt, Artificial
  SYNTHETIC CONSTRUCT
gaucgcccaauaaacauuc

SEQ ID NO: 1145 - RNA, 19 nt, Artificial
  SYNTHETIC CONSTRUCT
cuagcggguuauuuguaag

SEQ ID NO: 1146 - RNA, 19 nt, Artificial
  SYNTHETIC CONSTRUCT
cgcccaauaaacauucccu

SEQ ID NO: 1147 - RNA, 19 nt, Artificial
  SYNTHETIC CONSTRUCT
gcggguuauuuguaaggga

SEQ ID NO: 1148 - RNA, 19 nt, Artificial
  SYNTHETIC CONSTRUCT
gcccaauaaacauucccuu

SEQ ID NO: 1149 - RNA, 19 nt, Artificial
  SYNTHETIC CONSTRUCT
cggguuauuuguaagggaa

SEQ ID NO: 1150 - RNA, 19 nt, Artificial
  SYNTHETIC CONSTRUCT
ccaauaaacauucccuugg

SEQ ID NO: 1151 - RNA, 19 nt, Artificial
  SYNTHETIC CONSTRUCT
gguuauuuguaagggaacc

SEQ ID NO: 1152 - RNA, 19 nt, Artificial
  SYNTHETIC CONSTRUCT
auaaacauucccuuggaug

SEQ ID NO: 1153 - RNA, 19 nt, Artificial
  SYNTHETIC CONSTRUCT
uauuuguaagggaaccuac

SEQ ID NO: 1154 - RNA, 19 nt, Artificial
  SYNTHETIC CONSTRUCT
aacauucccuuggauguag

SEQ ID NO: 1155 - RNA, 19 nt, Artificial
  SYNTHETIC CONSTRUCT
uuguaagggaaccuacauc

SEQ ID NO: 1156 - RNA, 19 nt, Artificial
  SYNTHETIC CONSTRUCT
cauucccuuggauguaguc

SEQ ID NO: 1157 - RNA, 19 nt, Artificial
  SYNTHETIC CONSTRUCT
guaagggaaccuacaucag

SEQ ID NO: 1158 - RNA, 19 nt, Artificial
  SYNTHETIC CONSTRUCT
auucccuuggauguagucu

SEQ ID NO: 1159 - RNA, 19 nt, Artificial
  SYNTHETIC CONSTRUCT
uaagggaaccuacaucaga

SEQ ID NO: 1160 - RNA, 19 nt, Artificial
  SYNTHETIC CONSTRUCT
ccuuggauguagucugagg

SEQ ID NO: 1161 - RNA, 19 nt, Artificial
  SYNTHETIC CONSTRUCT
ggaaccuacaucagacucc

SEQ ID NO: 1162 - RNA, 19 nt, Artificial
  SYNTHETIC CONSTRUCT
aggccccuuaacucaucug

SEQ ID NO: 1163 - RNA, 19 nt, Artificial
  SYNTHETIC CONSTRUCT
uccggggaauugaguagac

SEQ ID NO: 1164 - RNA, 19 nt, Artificial
  SYNTHETIC CONSTRUCT
cuuaacucaucuguuaucc

SEQ ID NO: 1165 - RNA, 19 nt, Artificial
  SYNTHETIC CONSTRUCT
gaauugaguagacaauagg

SEQ ID NO: 1166 - RNA, 19 nt, Artificial
  SYNTHETIC CONSTRUCT
ucaucuguuauccugcuag

SEQ ID NO: 1167 - RNA, 19 nt, Artificial
  SYNTHETIC CONSTRUCT
aguagacaauaggacgauc

SEQ ID NO: 1168 - RNA, 19 nt, Artificial
  SYNTHETIC CONSTRUCT
caucuguuauccugcuagc

SEQ ID NO: 1169 - RNA, 19 nt, Artificial
  SYNTHETIC CONSTRUCT
guagacaauaggacgaucg

SEQ ID NO: 1170 - RNA, 19 nt, Artificial
  SYNTHETIC CONSTRUCT
aucuguuauccugcuagcu

SEQ ID NO: 1171 - RNA, 19 nt, Artificial
  SYNTHETIC CONSTRUCT
uagacaauaggacgaucga

SEQ ID NO: 1172 - RNA, 19 nt, Artificial
  SYNTHETIC CONSTRUCT
uagcuguagaaauguaucc

SEQ ID NO: 1173 - RNA, 19 nt, Artificial
  SYNTHETIC CONSTRUCT
aucgacaucuuuacauagg

SEQ ID NO: 1174 - RNA, 19 nt, Artificial
  SYNTHETIC CONSTRUCT
agcuguagaaauguauccu

SEQ ID NO: 1175 - RNA, 19 nt, Artificial
  SYNTHETIC CONSTRUCT
ucgacaucuuuacauagga

SEQ ID NO: 1176 - RNA, 19 nt, Artificial
  SYNTHETIC CONSTRUCT
ccugauaaacauuaaacac

SEQ ID NO: 1177 - RNA, 19 nt, Artificial
  SYNTHETIC CONSTRUCT
ggacuauuuguaauuugug

SEQ ID NO: 1178 - RNA, 19 nt, Artificial
  SYNTHETIC CONSTRUCT
cacuguaaucuuaaaagug

SEQ ID NO: 1179 - RNA, 19 nt, Artificial
  SYNTHETIC CONSTRUCT
gugacauuagaauuuucac

SEQ ID NO: 1180 - RNA, 19 nt, Artificial
  SYNTHETIC CONSTRUCT
aauugugugacuuuuucag

SEQ ID NO: 1181 - RNA, 19 nt, Artificial
  SYNTHETIC CONSTRUCT
uuaacacacugaaaaaguc

SEQ ID NO: 1182 - RNA, 19 nt, Artificial
  SYNTHETIC CONSTRUCT
uugugugacuuuuucagag

SEQ ID NO: 1183 - RNA, 19 nt, Artificial
  SYNTHETIC CONSTRUCT
aacacacugaaaaagucuc

SEQ ID NO: 1184 - RNA, 19 nt, Artificial
  SYNTHETIC CONSTRUCT
gacuuuuucagaguugcuu

SEQ ID NO: 1185 - RNA, 19 nt, Artificial
  SYNTHETIC CONSTRUCT
cugaaaaagucucaacgaa

SEQ ID NO: 1186 - RNA, 19 nt, Artificial
  SYNTHETIC CONSTRUCT
guugcuuuaaaguaccugu

SEQ ID NO: 1187 - RNA, 19 nt, Artificial
  SYNTHETIC CONSTRUCT
caacgaaauuucauggaca

SEQ ID NO: 1188 - RNA, 19 nt, Artificial
  SYNTHETIC CONSTRUCT
ugcuuuaaaguaccuguag

SEQ ID NO: 1189 - RNA, 19 nt, Artificial
  SYNTHETIC CONSTRUCT
acgaaauuucauggacauc

SEQ ID NO: 1190 - RNA, 19 nt, Artificial
  SYNTHETIC CONSTRUCT
cuuuaaaguaccuguagug

SEQ ID NO: 1191 - RNA, 19 nt, Artificial
  SYNTHETIC CONSTRUCT
gaaauuucauggacaucac

SEQ ID NO: 1192 - RNA, 19 nt, Artificial
  SYNTHETIC CONSTRUCT
uuaaaguaccuguagugag

SEQ ID NO: 1193 - RNA, 19 nt, Artificial
  SYNTHETIC CONSTRUCT
aauuucauggacaucacuc

SEQ ID NO: 1194 - RNA, 19 nt, Artificial
  SYNTHETIC CONSTRUCT
uaccuguagugagaaacug

SEQ ID NO: 1195 - RNA, 19 nt, Artificial
  SYNTHETIC CONSTRUCT
auggacaucacucuuugac

SEQ ID NO: 1196 - RNA, 19 nt, Artificial
  SYNTHETIC CONSTRUCT
gagaaacugauuuaugauc

SEQ ID NO: 1197 - RNA, 19 nt, Artificial
  SYNTHETIC CONSTRUCT
cucuuugacuaaauacuag

SEQ ID NO: 1198 - RNA, 19 nt, Artificial
  SYNTHETIC CONSTRUCT
cucaguuaaaaugucuguu

SEQ ID NO: 1199 - RNA, 19 nt, Artificial
  SYNTHETIC CONSTRUCT
gagucaauuuuacagacaa

SEQ ID NO: 1200 - RNA, 19 nt, Artificial
  SYNTHETIC CONSTRUCT
aaugucuguuucaaugacc

SEQ ID NO: 1201 - RNA, 19 nt, Artificial
  SYNTHETIC CONSTRUCT
uuacagacaaaguuacugg

SEQ ID NO: 1202 - RNA, 19 nt, Artificial
  SYNTHETIC CONSTRUCT
caaugaccuguauuuugcc

SEQ ID NO: 1203 - RNA, 19 nt, Artificial
  SYNTHETIC CONSTRUCT
guuacuggacauaaaacgg

SEQ ID NO: 1204 - RNA, 19 nt, Artificial
  SYNTHETIC CONSTRUCT
uuugccagacuuaaaucac

SEQ ID NO: 1205 - RNA, 19 nt, Artificial
  SYNTHETIC CONSTRUCT
aaacggucugaauuuagug

SEQ ID NO: 1206 - RNA, 19 nt, Artificial
  SYNTHETIC CONSTRUCT
ugccagacuuaaaucacag

SEQ ID NO: 1207 - RNA, 19 nt, Artificial
  SYNTHETIC CONSTRUCT
acggucugaauuuaguguc

SEQ ID NO: 1208 - RNA, 19 nt, Artificial
  SYNTHETIC CONSTRUCT
cagacuuaaaucacagaug

SEQ ID NO: 1209 - RNA, 19 nt, Artificial
  SYNTHETIC CONSTRUCT
gucugaauuuagugucuac

SEQ ID NO: 1210 - RNA, 19 nt, Artificial
  SYNTHETIC CONSTRUCT
cagauggguauuaaacuug

SEQ ID NO: 1211 - RNA, 19 nt, Artificial
  SYNTHETIC CONSTRUCT
gucuacccauaauuugaac

SEQ ID NO: 1212 - RNA, 19 nt, Artificial
  SYNTHETIC CONSTRUCT
acuugucagaauuucuuug

SEQ ID NO: 1213 - RNA, 19 nt, Artificial
  SYNTHETIC CONSTRUCT
ugaacagucuuaaagaaac

SEQ ID NO: 1214 - RNA, 19 nt, Artificial
  SYNTHETIC CONSTRUCT
cagaauuucuuugucauuc

SEQ ID NO: 1215 - RNA, 19 nt, Artificial
  SYNTHETIC CONSTRUCT
gucuuaaagaaacaguaag

SEQ ID NO: 1216 - RNA, 19 nt, Artificial
  SYNTHETIC CONSTRUCT
auuucuuugucauucaagc

SEQ ID NO: 1217 - RNA, 19 nt, Artificial
  SYNTHETIC CONSTRUCT
uaaagaaacaguaaguucg

SEQ ID NO: 1218 - RNA, 19 nt, Artificial
  SYNTHETIC CONSTRUCT
uucuuugucauucaagccu

SEQ ID NO: 1219 - RNA, 19 nt, Artificial
  SYNTHETIC CONSTRUCT
aagaaacaguaaguucgga

SEQ ID NO: 1220 - RNA, 19 nt, Artificial
  SYNTHETIC CONSTRUCT
aagccugugaauaaaaacc

SEQ ID NO: 1221 - RNA, 19 nt, Artificial
  SYNTHETIC CONSTRUCT
uucggacacuuauuuuugg

SEQ ID NO: 1222 - RNA, 19 nt, Artificial
  SYNTHETIC CONSTRUCT
cugugaauaaaaacccugu

SEQ ID NO: 1223 - RNA, 19 nt, Artificial
  SYNTHETIC CONSTRUCT
gacacuuauuuuugggaca

SEQ ID NO: 1224 - RNA, 19 nt, Artificial
  SYNTHETIC CONSTRUCT
uaaaaacccuguauggcac

SEQ ID NO: 1225 - RNA, 19 nt, Artificial
  SYNTHETIC CONSTRUCT
auuuuugggacauaccgug

SEQ ID NO: 1226 - RNA, 19 nt, Artificial
  SYNTHETIC CONSTRUCT
uauggcacuuauuaugagg

SEQ ID NO: 1227 - RNA, 19 nt, Artificial
  SYNTHETIC CONSTRUCT
auaccgugaauaauacucc

SEQ ID NO: 1228 - RNA, 19 nt, Artificial
  SYNTHETIC CONSTRUCT
gaggcuauuaaaagaaucc

SEQ ID NO: 1229 - RNA, 19 nt, Artificial
  SYNTHETIC CONSTRUCT
cuccgauaauuuucuuagg

SEQ ID NO: 1230 - RNA, 19 nt, Artificial
  SYNTHETIC CONSTRUCT
ggcgcggaggucuggccua

SEQ ID NO: 1231 - RNA, 19 nt, Artificial
  SYNTHETIC CONSTRUCT
ccgcgccuccagaccggau

SEQ ID NO: 1232 - RNA, 19 nt, Artificial
  SYNTHETIC CONSTRUCT
gcgcggaggucuggccuau

SEQ ID NO: 1233 - RNA, 19 nt, Artificial
  SYNTHETIC CONSTRUCT
cgcgccuccagaccggaua

SEQ ID NO: 1234 - RNA, 19 nt, Artificial
  SYNTHETIC CONSTRUCT
gagacggggugcugguuug

SEQ ID NO: 1235 - RNA, 19 nt, Artificial
  SYNTHETIC CONSTRUCT
cucugccccacgaccaaac

SEQ ID NO: 1236 - RNA, 19 nt, Artificial
  SYNTHETIC CONSTRUCT
acggggugcugguuugcgu

SEQ ID NO: 1237 - RNA, 19 nt, Artificial
  SYNTHETIC CONSTRUCT
ugccccacgaccaaacgca

SEQ ID NO: 1238 - RNA, 19 nt, Artificial
  SYNTHETIC CONSTRUCT
ugcgucguagucuccugca

SEQ ID NO: 1239 - RNA, 19 nt, Artificial
  SYNTHETIC CONSTRUCT
acgcagcaucagaggacgu

SEQ ID NO: 1240 - RNA, 19 nt, Artificial
  SYNTHETIC CONSTRUCT
ccugcagcgucugggguuu

SEQ ID NO: 1241 - RNA, 19 nt, Artificial
  SYNTHETIC CONSTRUCT
ggacgucgcagaccccaaa

SEQ ID NO: 1242 - RNA, 19 nt, Artificial
  SYNTHETIC CONSTRUCT
ggguuuccguugcaguccu

SEQ ID NO: 1243 - RNA, 19 nt, Artificial
  SYNTHETIC CONSTRUCT
cccaaaggcaacgucagga

SEQ ID NO: 1244 - RNA, 19 nt, Artificial
  SYNTHETIC CONSTRUCT
uuccguugcaguccucgga

SEQ ID NO: 1245 - RNA, 19 nt, Artificial
  SYNTHETIC CONSTRUCT
aaggcaacgucaggagccu

SEQ ID NO: 1246 - RNA, 19 nt, Artificial
  SYNTHETIC CONSTRUCT
uccguugcaguccucggaa

SEQ ID NO: 1247 - RNA, 19 nt, Artificial
  SYNTHETIC CONSTRUCT
aggcaacgucaggagccuu

SEQ ID NO: 1248 - RNA, 19 nt, Artificial
  SYNTHETIC CONSTRUCT
guugcaguccucggaacca

SEQ ID NO: 1249 - RNA, 19 nt, Artificial
  SYNTHETIC CONSTRUCT
caacgucaggagccuuggu

SEQ ID NO: 1250 - RNA, 19 nt, Artificial
  SYNTHETIC CONSTRUCT
aggaccucggcguggccua

SEQ ID NO: 1251 - RNA, 19 nt, Artificial
  SYNTHETIC CONSTRUCT
uccuggagccgcaccggau

SEQ ID NO: 1252 - RNA, 19 nt, Artificial
  SYNTHETIC CONSTRUCT
ccucggcguggccuagcga

SEQ ID NO: 1253 - RNA, 19 nt, Artificial
  SYNTHETIC CONSTRUCT
ggagccgcaccggaucgcu

SEQ ID NO: 1254 - RNA, 19 nt, Artificial
  SYNTHETIC CONSTRUCT
gcguggccuagcgaguuau

SEQ ID NO: 1255 - RNA, 19 nt, Artificial
  SYNTHETIC CONSTRUCT
cgcaccggaucgcucaaua

SEQ ID NO: 1256 - RNA, 19 nt, Artificial
  SYNTHETIC CONSTRUCT
cguggccuagcgaguuaug

SEQ ID NO: 1257 - RNA, 19 nt, Artificial
  SYNTHETIC CONSTRUCT
gcaccggaucgcucaauac

SEQ ID NO: 1258 - RNA, 19 nt, Artificial
  SYNTHETIC CONSTRUCT
cgacgaaggccgugugcgu

SEQ ID NO: 1259 - RNA, 19 nt, Artificial
  SYNTHETIC CONSTRUCT
gcugcuuccggcacacgca

SEQ ID NO: 1260 - RNA, 19 nt, Artificial
  SYNTHETIC CONSTRUCT
cgaaggccgugugcgugcu

SEQ ID NO: 1261 - RNA, 19 nt, Artificial
  SYNTHETIC CONSTRUCT
gcuuccggcacacgcacga

SEQ ID NO: 1262 - RNA, 19 nt, Artificial
  SYNTHETIC CONSTRUCT
aaggccgugugcgugcuga

SEQ ID NO: 1263 - RNA, 19 nt, Artificial
  SYNTHETIC CONSTRUCT
uuccggcacacgcacgacu

SEQ ID NO: 1264 - RNA, 19 nt, Artificial
  SYNTHETIC CONSTRUCT
ugugcgugcugaagggcga

SEQ ID NO: 1265 - RNA, 19 nt, Artificial
  SYNTHETIC CONSTRUCT
acacgcacgacuucccgcu

SEQ ID NO: 1266 - RNA, 19 nt, Artificial
  SYNTHETIC CONSTRUCT
agggcgacggcccagugca

SEQ ID NO: 1267 - RNA, 19 nt, Artificial
  SYNTHETIC CONSTRUCT
ucccgcugccgggucacgu

SEQ ID NO: 1268 - RNA, 19 nt, Artificial
  SYNTHETIC CONSTRUCT
acggcccagugcagggcau

SEQ ID NO: 1269 - RNA, 19 nt, Artificial
  SYNTHETIC CONSTRUCT
ugccgggucacgucccgua

SEQ ID NO: 1270 - RNA, 19 nt, Artificial
  SYNTHETIC CONSTRUCT
ggcccagugcagggcauca

SEQ ID NO: 1271 - RNA, 19 nt, Artificial
  SYNTHETIC CONSTRUCT
ccgggucacgucccguagu

SEQ ID NO: 1272 - RNA, 19 nt, Artificial
  SYNTHETIC CONSTRUCT
gcccagugcagggcaucau

SEQ ID NO: 1273 - RNA, 19 nt, Artificial
  SYNTHETIC CONSTRUCT
cgggucacgucccguagua

SEQ ID NO: 1274 - RNA, 19 nt, Artificial
  SYNTHETIC CONSTRUCT
gacugaaggccugcaugga

SEQ ID NO: 1275 - RNA, 19 nt, Artificial
  SYNTHETIC CONSTRUCT
cugacuuccggacguaccu

SEQ ID NO: 1276 - RNA, 19 nt, Artificial
  SYNTHETIC CONSTRUCT
acagcaggcuguaccagug

SEQ ID NO: 1277 - RNA, 19 nt, Artificial
  SYNTHETIC CONSTRUCT
ugucguccgacauggucac

SEQ ID NO: 1278 - RNA, 19 nt, Artificial
  SYNTHETIC CONSTRUCT
cuguaccagugcagguccu

SEQ ID NO: 1279 - RNA, 19 nt, Artificial
  SYNTHETIC CONSTRUCT
gacauggucacguccagga

SEQ ID NO: 1280 - RNA, 19 nt, Artificial
  SYNTHETIC CONSTRUCT
guaccagugcagguccuca

SEQ ID NO: 1281 - RNA, 19 nt, Artificial
  SYNTHETIC CONSTRUCT
cauggucacguccaggagu

SEQ ID NO: 1282 - RNA, 19 nt, Artificial
  SYNTHETIC CONSTRUCT
accagugcagguccucacu

SEQ ID NO: 1283 - RNA, 19 nt, Artificial
  SYNTHETIC CONSTRUCT
uggucacguccaggaguga

SEQ ID NO: 1284 - RNA, 19 nt, Artificial
  SYNTHETIC CONSTRUCT
aaacacggugggccaaagg

SEQ ID NO: 1285 - RNA, 19 nt, Artificial
  SYNTHETIC CONSTRUCT
uuugugccacccgguuucc

SEQ ID NO: 1286 - RNA, 19 nt, Artificial
  SYNTHETIC CONSTRUCT
agaugguguggccgaugug

SEQ ID NO: 1287 - RNA, 19 nt, Artificial
  SYNTHETIC CONSTRUCT
ucuaccacaccggcuacac

SEQ ID NO: 1288 - RNA, 19 nt, Artificial
  SYNTHETIC CONSTRUCT
ugguguggccgaugugucu

SEQ ID NO: 1289 - RNA, 19 nt, Artificial
  SYNTHETIC CONSTRUCT
accacaccggcuacacaga

SEQ ID NO: 1290 - RNA, 19 nt, Artificial
  SYNTHETIC CONSTRUCT
gguguggccgaugugucua

SEQ ID NO: 1291 - RNA, 19 nt, Artificial
  SYNTHETIC CONSTRUCT
ccacaccggcuacacagau

SEQ ID NO: 1292 - RNA, 19 nt, Artificial
  SYNTHETIC CONSTRUCT
gcaucauuggccgcacacu

SEQ ID NO: 1293 - RNA, 19 nt, Artificial
  SYNTHETIC CONSTRUCT
cguaguaaccggcguguga

SEQ ID NO: 1294 - RNA, 19 nt, Artificial
  SYNTHETIC CONSTRUCT
ucauuggccgcacacuggu

SEQ ID NO: 1295 - RNA, 19 nt, Artificial
  SYNTHETIC CONSTRUCT
aguaaccggcgugugacca

SEQ ID NO: 1296 - RNA, 19 nt, Artificial
  SYNTHETIC CONSTRUCT
aaaugaagaaaguacaaag

SEQ ID NO: 1297 - RNA, 19 nt, Artificial
  SYNTHETIC CONSTRUCT
uuuacuucuuucauguuuc

SEQ ID NO: 1298 - RNA, 19 nt, Artificial
  SYNTHETIC CONSTRUCT
cgcuggaagucguuuggcu

SEQ ID NO: 1299 - RNA, 19 nt, Artificial
  SYNTHETIC CONSTRUCT
gcgaccuucagcaaaccga

SEQ ID NO: 1300 - RNA, 19 nt, Artificial
  SYNTHETIC CONSTRUCT
auccugauaaacauuaaac

SEQ ID NO: 1301 - RNA, 19 nt, Artificial
  SYNTHETIC CONSTRUCT
uaggacuauuuguaauuug

SEQ ID NO: 1302 - RNA, 19 nt, Artificial
  SYNTHETIC CONSTRUCT
cugauaaacauuaaacacu

SEQ ID NO: 1303 - RNA, 19 nt, Artificial
  SYNTHETIC CONSTRUCT
gacuauuuguaauuuguga

SEQ ID NO: 1304 - RNA, 19 nt, Artificial
  SYNTHETIC CONSTRUCT
ugauaaacauuaaacacug

SEQ ID NO: 1305 - RNA, 19 nt, Artificial
  SYNTHETIC CONSTRUCT
acuauuuguaauuugugac

SEQ ID NO: 1306 - RNA, 19 nt, Artificial
  SYNTHETIC CONSTRUCT
aacauuaaacacuguaauc

SEQ ID NO: 1307 - RNA, 19 nt, Artificial
  SYNTHETIC CONSTRUCT
uuguaauuugugacauuag

SEQ ID NO: 1308 - RNA, 19 nt, Artificial
  SYNTHETIC CONSTRUCT
aacacuguaaucuuaaaag

SEQ ID NO: 1309 - RNA, 19 nt, Artificial
  SYNTHETIC CONSTRUCT
uugugacauuagaauuuuc

SEQ ID NO: 1310 - RNA, 19 nt, Artificial
  SYNTHETIC CONSTRUCT
ucuuaaaaguguaauugug

SEQ ID NO: 1311 - RNA, 19 nt, Artificial
  SYNTHETIC CONSTRUCT
agaauuuucacauuaacac

SEQ ID NO: 1312 - RNA, 19 nt, Artificial
  SYNTHETIC CONSTRUCT
cuuaaaaguguaauugugu

SEQ ID NO: 1313 - RNA, 19 nt, Artificial
  SYNTHETIC CONSTRUCT
gaauuuucacauuaacaca

SEQ ID NO: 1314 - RNA, 19 nt, Artificial
  SYNTHETIC CONSTRUCT
uuaaaaguguaauugugug

SEQ ID NO: 1315 - RNA, 19 nt, Artificial
  SYNTHETIC CONSTRUCT
aauuuucacauuaacacac

SEQ ID NO: 1316 - RNA, 19 nt, Artificial
  SYNTHETIC CONSTRUCT
aacugauuuaugaucacuu

SEQ ID NO: 1317 - RNA, 19 nt, Artificial
  SYNTHETIC CONSTRUCT
uugacuaaauacuagugaa

SEQ ID NO: 1318 - RNA, 19 nt, Artificial
  SYNTHETIC CONSTRUCT
auaguuuuauaaaacucag

SEQ ID NO: 1319 - RNA, 19 nt, Artificial
  SYNTHETIC CONSTRUCT
uaucaaaauauuuugaguc

SEQ ID NO: 1320 - RNA, 19 nt, Artificial
  SYNTHETIC CONSTRUCT
uaguuuuauaaaacucagu

SEQ ID NO: 1321 - RNA, 19 nt, Artificial
  SYNTHETIC CONSTRUCT
aucaaaauauuuugaguca

SEQ ID NO: 1322 - RNA, 19 nt, Artificial
  SYNTHETIC CONSTRUCT
aguuuuauaaaacucaguu

SEQ ID NO: 1323 - RNA, 19 nt, Artificial
  SYNTHETIC CONSTRUCT
ucaaaauauuuugagucaa

SEQ ID NO: 1324 - RNA, 19 nt, Artificial
  SYNTHETIC CONSTRUCT
auuaugaggcuauuaaaag

SEQ ID NO: 1325 - RNA, 19 nt, Artificial
  SYNTHETIC CONSTRUCT
uaauacuccgauaauuuuc

SEQ ID NO: 1326 - RNA, 19 nt, Artificial
  SYNTHETIC CONSTRUCT
auuaaaagaauccaaauuc

SEQ ID NO: 1327 - RNA, 19 nt, Artificial
  SYNTHETIC CONSTRUCT
uaauuuucuuagguuuaag

SEQ ID NO: 1328 - RNA, 19 nt, Artificial
  SYNTHETIC CONSTRUCT
aaagaauccaaauucaaac

SEQ ID NO: 1329 - RNA, 19 nt, Artificial
  SYNTHETIC CONSTRUCT
uuucuuagguuuaaguuug

SEQ ID NO: 1330 - RNA, 19 nt, Artificial
  SYNTHETIC CONSTRUCT
cuauaaaguagucgcggag

SEQ ID NO: 1331 - RNA, 19 nt, Artificial
  SYNTHETIC CONSTRUCT
gauauuucaucagcgccuc

SEQ ID NO: 1332 - RNA, 19 nt, Artificial
  SYNTHETIC CONSTRUCT
uaaaguagucgcggagacg

SEQ ID NO: 1333 - RNA, 19 nt, Artificial
  SYNTHETIC CONSTRUCT
auuucaucagcgccucugc

SEQ ID NO: 1334 - RNA, 19 nt, Artificial
  SYNTHETIC CONSTRUCT
guuugcgucguagucuccu

SEQ ID NO: 1335 - RNA, 19 nt, Artificial
  SYNTHETIC CONSTRUCT
caaacgcagcaucagagga

SEQ ID NO: 1336 - RNA, 19 nt, Artificial
  SYNTHETIC CONSTRUCT
gggcaucaucaauuucgag

SEQ ID NO: 1337 - RNA, 19 nt, Artificial
  SYNTHETIC CONSTRUCT
cccguaguaguuaaagcuc

SEQ ID NO: 1338 - RNA, 19 nt, Artificial
  SYNTHETIC CONSTRUCT
caucaauuucgagcagaag

SEQ ID NO: 1339 - RNA, 19 nt, Artificial
  SYNTHETIC CONSTRUCT
guaguuaaagcucgucuuc

SEQ ID NO: 1340 - RNA, 19 nt, Artificial
  SYNTHETIC CONSTRUCT
aucaauuucgagcagaagg

SEQ ID NO: 1341 - RNA, 19 nt, Artificial
  SYNTHETIC CONSTRUCT
uaguuaaagcucgucuucc

SEQ ID NO: 1342 - RNA, 19 nt, Artificial
  SYNTHETIC CONSTRUCT
agcagaaggaaaguaaugg

SEQ ID NO: 1343 - RNA, 19 nt, Artificial
  SYNTHETIC CONSTRUCT
ucgucuuccuuucauuacc

SEQ ID NO: 1344 - RNA, 19 nt, Artificial
  SYNTHETIC CONSTRUCT
cagaaggaaaguaauggac

SEQ ID NO: 1345 - RNA, 19 nt, Artificial
  SYNTHETIC CONSTRUCT
gucuuccuuucauuaccug

SEQ ID NO: 1346 - RNA, 19 nt, Artificial
  SYNTHETIC CONSTRUCT
agaaggaaaguaauggacc

SEQ ID NO: 1347 - RNA, 19 nt, Artificial
  SYNTHETIC CONSTRUCT
ucuuccuuucauuaccugg

SEQ ID NO: 1348 - RNA, 19 nt, Artificial
  SYNTHETIC CONSTRUCT
aggaaaguaauggaccagu

SEQ ID NO: 1349 - RNA, 19 nt, Artificial
  SYNTHETIC CONSTRUCT
uccuuucauuaccugguca

SEQ ID NO: 1350 - RNA, 19 nt, Artificial
  SYNTHETIC CONSTRUCT
uaauggaccagugaaggug

SEQ ID NO: 1351 - RNA, 19 nt, Artificial
  SYNTHETIC CONSTRUCT
auuaccuggucacuuccac

SEQ ID NO: 1352 - RNA, 19 nt, Artificial
  SYNTHETIC CONSTRUCT
uggggaagcauuaaaggac

SEQ ID NO: 1353 - RNA, 19 nt, Artificial
  SYNTHETIC CONSTRUCT
accccuucguaauuuccug

SEQ ID NO: 1354 - RNA, 19 nt, Artificial
  SYNTHETIC CONSTRUCT
gaagcauuaaaggacugac

SEQ ID NO: 1355 - RNA, 19 nt, Artificial
  SYNTHETIC CONSTRUCT
cuucguaauuuccugacug

SEQ ID NO: 1356 - RNA, 19 nt, Artificial
  SYNTHETIC CONSTRUCT
agcauuaaaggacugacug

SEQ ID NO: 1357 - RNA, 19 nt, Artificial
  SYNTHETIC CONSTRUCT
ucguaauuuccugacugac

SEQ ID NO: 1358 - RNA, 19 nt, Artificial
  SYNTHETIC CONSTRUCT
aaggacugacugaaggccu

SEQ ID NO: 1359 - RNA, 19 nt, Artificial
  SYNTHETIC CONSTRUCT
uuccugacugacuuccgga

SEQ ID NO: 1360 - RNA, 19 nt, Artificial
  SYNTHETIC CONSTRUCT
cauggauuccauguucaug

SEQ ID NO: 1361 - RNA, 19 nt, Artificial
  SYNTHETIC CONSTRUCT
guaccuaagguacaaguac

SEQ ID NO: 1362 - RNA, 19 nt, Artificial
  SYNTHETIC CONSTRUCT
uuccauguucaugaguuug

SEQ ID NO: 1363 - RNA, 19 nt, Artificial
  SYNTHETIC CONSTRUCT
aagguacaaguacucaaac

SEQ ID NO: 1364 - RNA, 19 nt, Artificial
  SYNTHETIC CONSTRUCT
cauguucaugaguuuggag

SEQ ID NO: 1365 - RNA, 19 nt, Artificial
  SYNTHETIC CONSTRUCT
guacaaguacucaaaccuc

SEQ ID NO: 1366 - RNA, 19 nt, Artificial
  SYNTHETIC CONSTRUCT
aguuuggagauaauacagc

SEQ ID NO: 1367 - RNA, 19 nt, Artificial
  SYNTHETIC CONSTRUCT
ucaaaccucuauuaugucg

SEQ ID NO: 1368 - RNA, 19 nt, Artificial
  SYNTHETIC CONSTRUCT
uuuggagauaauacagcag

SEQ ID NO: 1369 - RNA, 19 nt, Artificial
  SYNTHETIC CONSTRUCT
aaaccucuauuaugucguc

SEQ ID NO: 1370 - RNA, 19 nt, Artificial
  SYNTHETIC CONSTRUCT
uggagauaauacagcaggc

SEQ ID NO: 1371 - RNA, 19 nt, Artificial
  SYNTHETIC CONSTRUCT
accucuauuaugucguccg

SEQ ID NO: 1372 - RNA, 19 nt, Artificial
  SYNTHETIC CONSTRUCT
auacagcaggcuguaccag

SEQ ID NO: 1373 - RNA, 19 nt, Artificial
  SYNTHETIC CONSTRUCT
uaugucguccgacaugguc

SEQ ID NO: 1374 - RNA, 19 nt, Artificial
  SYNTHETIC CONSTRUCT
acuuuaauccucuauccag

SEQ ID NO: 1375 - RNA, 19 nt, Artificial
  SYNTHETIC CONSTRUCT
ugaaauuaggagauagguc

SEQ ID NO: 1376 - RNA, 19 nt, Artificial
  SYNTHETIC CONSTRUCT
ccucuauccagaaaacacg

SEQ ID NO: 1377 - RNA, 19 nt, Artificial
  SYNTHETIC CONSTRUCT
ggagauaggucuuuugugc

SEQ ID NO: 1378 - RNA, 19 nt, Artificial
  SYNTHETIC CONSTRUCT
cuauccagaaaacacggug

SEQ ID NO: 1379 - RNA, 19 nt, Artificial
  SYNTHETIC CONSTRUCT
gauaggucuuuugugccac

SEQ ID NO: 1380 - RNA, 19 nt, Artificial
  SYNTHETIC CONSTRUCT
ggccaaaggaugaagagag

SEQ ID NO: 1381 - RNA, 19 nt, Artificial
  SYNTHETIC CONSTRUCT
ccgguuuccuacuucucuc

SEQ ID NO: 1382 - RNA, 19 nt, Artificial
  SYNTHETIC CONSTRUCT
aaggaugaagagaggcaug

SEQ ID NO: 1383 - RNA, 19 nt, Artificial
  SYNTHETIC CONSTRUCT
uuccuacuucucuccguac

SEQ ID NO: 1384 - RNA, 19 nt, Artificial
  SYNTHETIC CONSTRUCT
augaagagaggcauguugg

SEQ ID NO: 1385 - RNA, 19 nt, Artificial
  SYNTHETIC CONSTRUCT
uacuucucuccguacaacc

SEQ ID NO: 1386 - RNA, 19 nt, Artificial
  SYNTHETIC CONSTRUCT
gaagagaggcauguuggag

SEQ ID NO: 1387 - RNA, 19 nt, Artificial
  SYNTHETIC CONSTRUCT
cuucucuccguacaaccuc

SEQ ID NO: 1388 - RNA, 19 nt, Artificial
  SYNTHETIC CONSTRUCT
gagacuugggcaaugugac

SEQ ID NO: 1389 - RNA, 19 nt, Artificial
  SYNTHETIC CONSTRUCT
cucugaacccguuacacug

SEQ ID NO: 1390 - RNA, 19 nt, Artificial
  SYNTHETIC CONSTRUCT
acuugggcaaugugacugc

SEQ ID NO: 1391 - RNA, 19 nt, Artificial
  SYNTHETIC CONSTRUCT
ugaacccguuacacugacg

SEQ ID NO: 1392 - RNA, 19 nt, Artificial
  SYNTHETIC CONSTRUCT
uugggcaaugugacugcug

SEQ ID NO: 1393 - RNA, 19 nt, Artificial
  SYNTHETIC CONSTRUCT
aacccguuacacugacgac

SEQ ID NO: 1394 - RNA, 19 nt, Artificial
  SYNTHETIC CONSTRUCT
aaugugacugcugacaaag

SEQ ID NO: 1395 - RNA, 19 nt, Artificial
  SYNTHETIC CONSTRUCT
uuacacugacgacuguuuc

SEQ ID NO: 1396 - RNA, 19 nt, Artificial
  SYNTHETIC CONSTRUCT
ugcugacaaagauggugug

SEQ ID NO: 1397 - RNA, 19 nt, Artificial
  SYNTHETIC CONSTRUCT
acgacuguuucuaccacac

SEQ ID NO: 1398 - RNA, 19 nt, Artificial
  SYNTHETIC CONSTRUCT
aaagaugguguggccgaug

SEQ ID NO: 1399 - RNA, 19 nt, Artificial
  SYNTHETIC CONSTRUCT
uuucuaccacaccggcuac

SEQ ID NO: 1400 - RNA, 19 nt, Artificial
  SYNTHETIC CONSTRUCT
gugucuauugaagauucug

SEQ ID NO: 1401 - RNA, 19 nt, Artificial
  SYNTHETIC CONSTRUCT
cacagauaacuucuaagac

SEQ ID NO: 1402 - RNA, 19 nt, Artificial
  SYNTHETIC CONSTRUCT
gucuauugaagauucugug

SEQ ID NO: 1403 - RNA, 19 nt, Artificial
  SYNTHETIC CONSTRUCT
cagauaacuucuaagacac

SEQ ID NO: 1404 - RNA, 19 nt, Artificial
  SYNTHETIC CONSTRUCT
cugugaucucacucucagg

SEQ ID NO: 1405 - RNA, 19 nt, Artificial
  SYNTHETIC CONSTRUCT
gacacuagagugagagucc

SEQ ID NO: 1406 - RNA, 19 nt, Artificial
  SYNTHETIC CONSTRUCT
gugaucucacucucaggag

SEQ ID NO: 1407 - RNA, 19 nt, Artificial
  SYNTHETIC CONSTRUCT
cacuagagugagaguccuc

SEQ ID NO: 1408 - RNA, 19 nt, Artificial
  SYNTHETIC CONSTRUCT
gaucucacucucaggagac

SEQ ID NO: 1409 - RNA, 19 nt, Artificial
  SYNTHETIC CONSTRUCT
cuagagugagaguccucug

SEQ ID NO: 1410 - RNA, 19 nt, Artificial
  SYNTHETIC CONSTRUCT
aucucacucucaggagacc

SEQ ID NO: 1411 - RNA, 19 nt, Artificial
  SYNTHETIC CONSTRUCT
uagagugagaguccucugg

SEQ ID NO: 1412 - RNA, 19 nt, Artificial
  SYNTHETIC CONSTRUCT
cucaggagaccauugcauc

SEQ ID NO: 1413 - RNA, 19 nt, Artificial
  SYNTHETIC CONSTRUCT
gaguccucugguaacguag

SEQ ID NO: 1414 - RNA, 19 nt, Artificial
  SYNTHETIC CONSTRUCT
cauugcaucauuggccgca

SEQ ID NO: 1415 - RNA, 19 nt, Artificial
  SYNTHETIC CONSTRUCT
guaacguaguaaccggcgu

SEQ ID NO: 1416 - RNA, 19 nt, Artificial
  SYNTHETIC CONSTRUCT
gugguccaugaaaaagcag

SEQ ID NO: 1417 - RNA, 19 nt, Artificial
  SYNTHETIC CONSTRUCT
caccagguacuuuuucguc

SEQ ID NO: 1418 - RNA, 19 nt, Artificial
  SYNTHETIC CONSTRUCT
guccaugaaaaagcagaug

SEQ ID NO: 1419 - RNA, 19 nt, Artificial
  SYNTHETIC CONSTRUCT
cagguacuuuuucgucuac

SEQ ID NO: 1420 - RNA, 19 nt, Artificial
  SYNTHETIC CONSTRUCT
gaaaaagcagaugacuugg

SEQ ID NO: 1421 - RNA, 19 nt, Artificial
  SYNTHETIC CONSTRUCT
cuuuuucgucuacugaacc

SEQ ID NO: 1422 - RNA, 19 nt, Artificial
  SYNTHETIC CONSTRUCT
gaugacuugggcaaaggug

SEQ ID NO: 1423 - RNA, 19 nt, Artificial
  SYNTHETIC CONSTRUCT
cuacugaacccguuuccac

SEQ ID NO: 1424 - RNA, 19 nt, Artificial
  SYNTHETIC CONSTRUCT
aagaaaguacaaagacagg

SEQ ID NO: 1425 - RNA, 19 nt, Artificial
  SYNTHETIC CONSTRUCT
uucuuucauguuucugucc

SEQ ID NO: 1426 - RNA, 19 nt, Artificial
  SYNTHETIC CONSTRUCT
aguacaaagacaggaaacg

SEQ ID NO: 1427 - RNA, 19 nt, Artificial
  SYNTHETIC CONSTRUCT
ucauguuucuguccuuugc

SEQ ID NO: 1428 - RNA, 19 nt, Artificial
  SYNTHETIC CONSTRUCT
guacaaagacaggaaacgc

SEQ ID NO: 1429 - RNA, 19 nt, Artificial
  SYNTHETIC CONSTRUCT
cauguuucuguccuuugcg

SEQ ID NO: 1430 - RNA, 19 nt, Artificial
  SYNTHETIC CONSTRUCT
acaaagacaggaaacgcug

SEQ ID NO: 1431 - RNA, 19 nt, Artificial
  SYNTHETIC CONSTRUCT
uguuucuguccuuugcgac

SEQ ID NO: 1432 - RNA, 19 nt, Artificial
  SYNTHETIC CONSTRUCT
caaagacaggaaacgcugg

SEQ ID NO: 1433 - RNA, 19 nt, Artificial
  SYNTHETIC CONSTRUCT
guuucuguccuuugcgacc

SEQ ID NO: 1434 - RNA, 19 nt, Artificial
  SYNTHETIC CONSTRUCT
cuggaagucguuuggcuug

SEQ ID NO: 1435 - RNA, 19 nt, Artificial
  SYNTHETIC CONSTRUCT
gaccuucagcaaaccgaac

SEQ ID NO: 1436 - RNA, 19 nt, Artificial
  SYNTHETIC CONSTRUCT
agucguuuggcuuguggug

SEQ ID NO: 1437 - RNA, 19 nt, Artificial
  SYNTHETIC CONSTRUCT
ucagcaaaccgaacaccac

SEQ ID NO: 1438 - RNA, 19 nt, Artificial
  SYNTHETIC CONSTRUCT
gucguuuggcuuguggugu

SEQ ID NO: 1439 - RNA, 19 nt, Artificial
  SYNTHETIC CONSTRUCT
cagcaaaccgaacaccaca

SEQ ID NO: 1440 - RNA, 19 nt, Artificial
  SYNTHETIC CONSTRUCT
uugugguguaauugggauc

SEQ ID NO: 1441 - RNA, 19 nt, Artificial
  SYNTHETIC CONSTRUCT
aacaccacauuaacccuag

SEQ ID NO: 1442 - RNA, 19 nt, Artificial
  SYNTHETIC CONSTRUCT
gugguguaauugggaucgc

SEQ ID NO: 1443 - RNA, 19 nt, Artificial
  SYNTHETIC CONSTRUCT
caccacauuaacccuagcg

SEQ ID NO: 1444 - RNA, 19 nt, Artificial
  SYNTHETIC CONSTRUCT
ucgcccaauaaacauuccc

SEQ ID NO: 1445 - RNA, 19 nt, Artificial
  SYNTHETIC CONSTRUCT
agcggguuauuuguaaggg

SEQ ID NO: 1446 - RNA, 19 nt, Artificial
  SYNTHETIC CONSTRUCT
cccaauaaacauucccuug

SEQ ID NO: 1447 - RNA, 19 nt, Artificial
  SYNTHETIC CONSTRUCT
ggguuauuuguaagggaac

SEQ ID NO: 1448 - RNA, 19 nt, Artificial
  SYNTHETIC CONSTRUCT
uucccuuggauguagucug

SEQ ID NO: 1449 - RNA, 19 nt, Artificial
  SYNTHETIC CONSTRUCT
aagggaaccuacaucagac

SEQ ID NO: 1450 - RNA, 19 nt, Artificial
  SYNTHETIC CONSTRUCT
auguagucugaggccccuu

SEQ ID NO: 1451 - RNA, 19 nt, Artificial
  SYNTHETIC CONSTRUCT
uacaucagacuccggggaa

SEQ ID NO: 1452 - RNA, 19 nt, Artificial
  SYNTHETIC CONSTRUCT
ucuguuauccugcuagcug

SEQ ID NO: 1453 - RNA, 19 nt, Artificial
  SYNTHETIC CONSTRUCT
agacaauaggacgaucgac

SEQ ID NO: 1454 - RNA, 19 nt, Artificial
  SYNTHETIC CONSTRUCT
guuauccugcuagcuguag

SEQ ID NO: 1455 - RNA, 19 nt, Artificial
  SYNTHETIC CONSTRUCT
caauaggacgaucgacauc

SEQ ID NO: 1456 - RNA, 19 nt, Artificial
  SYNTHETIC CONSTRUCT
gcuguagaaauguauccug

SEQ ID NO: 1457 - RNA, 19 nt, Artificial
  SYNTHETIC CONSTRUCT
cgacaucuuuacauaggac

SEQ ID NO: 1458 - RNA, 19 nt, Artificial
  SYNTHETIC CONSTRUCT
aaaaguguaauugugugac

SEQ ID NO: 1459 - RNA, 19 nt, Artificial
  SYNTHETIC CONSTRUCT
uuuucacauuaacacacug

SEQ ID NO: 1460 - RNA, 19 nt, Artificial
  SYNTHETIC CONSTRUCT
gugacuuuuucagaguugc

SEQ ID NO: 1461 - RNA, 19 nt, Artificial
  SYNTHETIC CONSTRUCT
cacugaaaaagucucaacg

SEQ ID NO: 1462 - RNA, 19 nt, Artificial
  SYNTHETIC CONSTRUCT
aguaccuguagugagaaac

SEQ ID NO: 1463 - RNA, 19 nt, Artificial
  SYNTHETIC CONSTRUCT
ucauggacaucacucuuug

SEQ ID NO: 1464 - RNA, 19 nt, Artificial
  SYNTHETIC CONSTRUCT
cugauuuaugaucacuugg

SEQ ID NO: 1465 - RNA, 19 nt, Artificial
  SYNTHETIC CONSTRUCT
gacuaaauacuagugaacc

SEQ ID NO: 1466 - RNA, 19 nt, Artificial
  SYNTHETIC CONSTRUCT
auuuaugaucacuuggaag

SEQ ID NO: 1467 - RNA, 19 nt, Artificial
  SYNTHETIC CONSTRUCT
uaaauacuagugaaccuuc

SEQ ID NO: 1468 - RNA, 19 nt, Artificial
  SYNTHETIC CONSTRUCT
aacucaguuaaaaugucug

SEQ ID NO: 1469 - RNA, 19 nt, Artificial
  SYNTHETIC CONSTRUCT
uugagucaauuuuacagac

SEQ ID NO: 1470 - RNA, 19 nt, Artificial
  SYNTHETIC CONSTRUCT
ugucuguuucaaugaccug

SEQ ID NO: 1471 - RNA, 19 nt, Artificial
  SYNTHETIC CONSTRUCT
acagacaaaguuacuggac

SEQ ID NO: 1472 - RNA, 19 nt, Artificial
  SYNTHETIC CONSTRUCT
gucuguuucaaugaccugu

SEQ ID NO: 1473 - RNA, 19 nt, Artificial
  SYNTHETIC CONSTRUCT
cagacaaaguuacuggaca

SEQ ID NO: 1474 - RNA, 19 nt, Artificial
  SYNTHETIC CONSTRUCT
gaccuguauuuugccagac

SEQ ID NO: 1475 - RNA, 19 nt, Artificial
  SYNTHETIC CONSTRUCT
cuggacauaaaacggucug

SEQ ID NO: 1476 - RNA, 19 nt, Artificial
  SYNTHETIC CONSTRUCT
gacuuaaaucacagauggg

SEQ ID NO: 1477 - RNA, 19 nt, Artificial
  SYNTHETIC CONSTRUCT
cugaauuuagugucuaccc

SEQ ID NO: 1478 - RNA, 19 nt, Artificial
  SYNTHETIC CONSTRUCT
acuuaaaucacagaugggu

SEQ ID NO: 1479 - RNA, 19 nt, Artificial
  SYNTHETIC CONSTRUCT
ugaauuuagugucuaccca

SEQ ID NO: 1480 - RNA, 19 nt, Artificial
  SYNTHETIC CONSTRUCT
uggguauuaaacuugucag

SEQ ID NO: 1481 - RNA, 19 nt, Artificial
  SYNTHETIC CONSTRUCT
acccauaauuugaacaguc

SEQ ID NO: 1482 - RNA, 19 nt, Artificial
  SYNTHETIC CONSTRUCT
uuucuuugucauucaagcc

SEQ ID NO: 1483 - RNA, 19 nt, Artificial
  SYNTHETIC CONSTRUCT
aaagaaacaguaaguucgg

SEQ ID NO: 1484 - RNA, 19 nt, Artificial
  SYNTHETIC CONSTRUCT
ucuuugucauucaagccug

SEQ ID NO: 1485 - RNA, 19 nt, Artificial
  SYNTHETIC CONSTRUCT
agaaacaguaaguucggac

SEQ ID NO: 1486 - RNA, 19 nt, Artificial
  SYNTHETIC CONSTRUCT
agccugugaauaaaaaccc

SEQ ID NO: 1487 - RNA, 19 nt, Artificial
  SYNTHETIC CONSTRUCT
ucggacacuuauuuuuggg

SEQ ID NO: 1488 - RNA, 19 nt, Artificial
  SYNTHETIC CONSTRUCT
uuggggccagagugggcga

SEQ ID NO: 1489 - RNA, 19 nt, Artificial
  SYNTHETIC CONSTRUCT
aaccccggucucacccgcu

SEQ ID NO: 1490 - RNA, 19 nt, Artificial
  SYNTHETIC CONSTRUCT
agagugggcgaggcgcgga

SEQ ID NO: 1491 - RNA, 19 nt, Artificial
  SYNTHETIC CONSTRUCT
ucucacccgcuccgcgccu

SEQ ID NO: 1492 - RNA, 19 nt, Artificial
  SYNTHETIC CONSTRUCT
gugggcgaggcgcggaggu

SEQ ID NO: 1493 - RNA, 19 nt, Artificial
  SYNTHETIC CONSTRUCT
cacccgcuccgcgccucca

SEQ ID NO: 1494 - RNA, 19 nt, Artificial
  SYNTHETIC CONSTRUCT
guagucgcggagacggggu

SEQ ID NO: 1495 - RNA, 19 nt, Artificial
  SYNTHETIC CONSTRUCT
caucagcgccucugcccca

SEQ ID NO: 1496 - RNA, 19 nt, Artificial
  SYNTHETIC CONSTRUCT
gcggagacggggugcuggu

SEQ ID NO: 1497 - RNA, 19 nt, Artificial
  SYNTHETIC CONSTRUCT
cgccucugccccacgacca

SEQ ID NO: 1498 - RNA, 19 nt, Artificial
  SYNTHETIC CONSTRUCT
cggagacggggugcugguu

SEQ ID NO: 1499 - RNA, 19 nt, Artificial
  SYNTHETIC CONSTRUCT
gccucugccccacgaccaa

SEQ ID NO: 1500 - RNA, 19 nt, Artificial
  SYNTHETIC CONSTRUCT
agacggggugcugguuugc

SEQ ID NO: 1501 - RNA, 19 nt, Artificial
  SYNTHETIC CONSTRUCT
ucugccccacgaccaaacg

SEQ ID NO: 1502 - RNA, 19 nt, Artificial
  SYNTHETIC CONSTRUCT
gggugcugguuugcgucgu

SEQ ID NO: 1503 - RNA, 19 nt, Artificial
  SYNTHETIC CONSTRUCT
cccacgaccaaacgcagca

SEQ ID NO: 1504 - RNA, 19 nt, Artificial
  SYNTHETIC CONSTRUCT
gcugguuugcgucguaguc

SEQ ID NO: 1505 - RNA, 19 nt, Artificial
  SYNTHETIC CONSTRUCT
cgaccaaacgcagcaucag

SEQ ID NO: 1506 - RNA, 19 nt, Artificial
  SYNTHETIC CONSTRUCT
gcgucguagucuccugcag

SEQ ID NO: 1507 - RNA, 19 nt, Artificial
  SYNTHETIC CONSTRUCT
cgcagcaucagaggacguc

SEQ ID NO: 1508 - RNA, 19 nt, Artificial
  SYNTHETIC CONSTRUCT
uccugcagcgucugggguu

SEQ ID NO: 1509 - RNA, 19 nt, Artificial
  SYNTHETIC CONSTRUCT
aggacgucgcagaccccaa

SEQ ID NO: 1510 - RNA, 19 nt, Artificial
  SYNTHETIC CONSTRUCT
ugcagcgucugggguuucc

SEQ ID NO: 1511 - RNA, 19 nt, Artificial
  SYNTHETIC CONSTRUCT
acgucgcagaccccaaagg

SEQ ID NO: 1512 - RNA, 19 nt, Artificial
  SYNTHETIC CONSTRUCT
cagcgucugggguuuccgu

SEQ ID NO: 1513 - RNA, 19 nt, Artificial
  SYNTHETIC CONSTRUCT
gucgcagaccccaaaggca

SEQ ID NO: 1514 - RNA, 19 nt, Artificial
  SYNTHETIC CONSTRUCT
agcgucugggguuuccguu

SEQ ID NO: 1515 - RNA, 19 nt, Artificial
  SYNTHETIC CONSTRUCT
ucgcagaccccaaaggcaa

SEQ ID NO: 1516 - RNA, 19 nt, Artificial
  SYNTHETIC CONSTRUCT
cgucugggguuuccguugc

SEQ ID NO: 1517 - RNA, 19 nt, Artificial
  SYNTHETIC CONSTRUCT
gcagaccccaaaggcaacg

SEQ ID NO: 1518 - RNA, 19 nt, Artificial
  SYNTHETIC CONSTRUCT
cugggguuuccguugcagu

SEQ ID NO: 1519 - RNA, 19 nt, Artificial
  SYNTHETIC CONSTRUCT
gaccccaaaggcaacguca

SEQ ID NO: 1520 - RNA, 19 nt, Artificial
  SYNTHETIC CONSTRUCT
gguuuccguugcaguccuc

SEQ ID NO: 1521 - RNA, 19 nt, Artificial
  SYNTHETIC CONSTRUCT
ccaaaggcaacgucaggag

SEQ ID NO: 1522 - RNA, 19 nt, Artificial
  SYNTHETIC CONSTRUCT
ccguugcaguccucggaac

SEQ ID NO: 1523 - RNA, 19 nt, Artificial
  SYNTHETIC CONSTRUCT
ggcaacgucaggagccuug

SEQ ID NO: 1524 - RNA, 19 nt, Artificial
  SYNTHETIC CONSTRUCT
guccucggaaccaggaccu

SEQ ID NO: 1525 - RNA, 19 nt, Artificial
  SYNTHETIC CONSTRUCT
caggagccuugguccugga

SEQ ID NO: 1526 - RNA, 19 nt, Artificial
  SYNTHETIC CONSTRUCT
ggaaccaggaccucggcgu

SEQ ID NO: 1527 - RNA, 19 nt, Artificial
  SYNTHETIC CONSTRUCT
ccuugguccuggagccgca

SEQ ID NO: 1528 - RNA, 19 nt, Artificial
  SYNTHETIC CONSTRUCT
accucggcguggccuagcg

SEQ ID NO: 1529 - RNA, 19 nt, Artificial
  SYNTHETIC CONSTRUCT
uggagccgcaccggaucgc

SEQ ID NO: 1530 - RNA, 19 nt, Artificial
  SYNTHETIC CONSTRUCT
ucggcguggccuagcgagu

SEQ ID NO: 1531 - RNA, 19 nt, Artificial
  SYNTHETIC CONSTRUCT
agccgcaccggaucgcuca

SEQ ID NO: 1532 - RNA, 19 nt, Artificial
  SYNTHETIC CONSTRUCT
cggcguggccuagcgaguu

SEQ ID NO: 1533 - RNA, 19 nt, Artificial
  SYNTHETIC CONSTRUCT
gccgcaccggaucgcucaa

SEQ ID NO: 1534 - RNA, 19 nt, Artificial
  SYNTHETIC CONSTRUCT
gccuagcgaguuauggcga

SEQ ID NO: 1535 - RNA, 19 nt, Artificial
  SYNTHETIC CONSTRUCT
cggaucgcucaauaccgcu

SEQ ID NO: 1536 - RNA, 19 nt, Artificial
  SYNTHETIC CONSTRUCT
cgaguuauggcgacgaagg

SEQ ID NO: 1537 - RNA, 19 nt, Artificial
  SYNTHETIC CONSTRUCT
gcucaauaccgcugcuucc

SEQ ID NO: 1538 - RNA, 19 nt, Artificial
  SYNTHETIC CONSTRUCT
uuauggcgacgaaggccgu

SEQ ID NO: 1539 - RNA, 19 nt, Artificial
  SYNTHETIC CONSTRUCT
aauaccgcugcuuccggca

SEQ ID NO: 1540 - RNA, 19 nt, Artificial
  SYNTHETIC CONSTRUCT
gaaggccgugugcgugcug

SEQ ID NO: 1541 - RNA, 19 nt, Artificial
  SYNTHETIC CONSTRUCT
cuuccggcacacgcacgac

SEQ ID NO: 1542 - RNA, 19 nt, Artificial
  SYNTHETIC CONSTRUCT
ccgugugcgugcugaaggg

SEQ ID NO: 1543 - RNA, 19 nt, Artificial
  SYNTHETIC CONSTRUCT
ggcacacgcacgacuuccc

SEQ ID NO: 1544 - RNA, 19 nt, Artificial
  SYNTHETIC CONSTRUCT
gcugaagggcgacggccca

SEQ ID NO: 1545 - RNA, 19 nt, Artificial
  SYNTHETIC CONSTRUCT
cgacuucccgcugccgggu

SEQ ID NO: 1546 - RNA, 19 nt, Artificial
  SYNTHETIC CONSTRUCT
ugaagggcgacggcccagu

SEQ ID NO: 1547 - RNA, 19 nt, Artificial
  SYNTHETIC CONSTRUCT
acuucccgcugccggguca

SEQ ID NO: 1548 - RNA, 19 nt, Artificial
  SYNTHETIC CONSTRUCT
gaagggcgacggcccagug

SEQ ID NO: 1549 - RNA, 19 nt, Artificial
  SYNTHETIC CONSTRUCT
cuucccgcugccgggucac

SEQ ID NO: 1550 - RNA, 19 nt, Artificial
  SYNTHETIC CONSTRUCT
gacggcccagugcagggca

SEQ ID NO: 1551 - RNA, 19 nt, Artificial
  SYNTHETIC CONSTRUCT
cugccgggucacgucccgu

SEQ ID NO: 1552 - RNA, 19 nt, Artificial
  SYNTHETIC CONSTRUCT
cccagugcagggcaucauc

SEQ ID NO: 1553 - RNA, 19 nt, Artificial
  SYNTHETIC CONSTRUCT
gggucacgucccguaguag

SEQ ID NO: 1554 - RNA, 19 nt, Artificial
  SYNTHETIC CONSTRUCT
gacugacugaaggccugca

SEQ ID NO: 1555 - RNA, 19 nt, Artificial
  SYNTHETIC CONSTRUCT
cugacugacuuccggacgu

SEQ ID NO: 1556 - RNA, 19 nt, Artificial
  SYNTHETIC CONSTRUCT
ugacugaaggccugcaugg

SEQ ID NO: 1557 - RNA, 19 nt, Artificial
  SYNTHETIC CONSTRUCT
acugacuuccggacguacc

SEQ ID NO: 1558 - RNA, 19 nt, Artificial
  SYNTHETIC CONSTRUCT
gaaggccugcauggauucc

SEQ ID NO: 1559 - RNA, 19 nt, Artificial
  SYNTHETIC CONSTRUCT
cuuccggacguaccuaagg

SEQ ID NO: 1560 - RNA, 19 nt, Artificial
  SYNTHETIC CONSTRUCT
gcaggcuguaccagugcag

SEQ ID NO: 1561 - RNA, 19 nt, Artificial
  SYNTHETIC CONSTRUCT
cguccgacauggucacguc

SEQ ID NO: 1562 - RNA, 19 nt, Artificial
  SYNTHETIC CONSTRUCT
aggcuguaccagugcaggu

SEQ ID NO: 1563 - RNA, 19 nt, Artificial
  SYNTHETIC CONSTRUCT
uccgacauggucacgucca

SEQ ID NO: 1564 - RNA, 19 nt, Artificial
  SYNTHETIC CONSTRUCT
cagaaaacacggugggcca

SEQ ID NO: 1565 - RNA, 19 nt, Artificial
  SYNTHETIC CONSTRUCT
gucuuuugugccacccggu

SEQ ID NO: 1566 - RNA, 19 nt, Artificial
  SYNTHETIC CONSTRUCT
gaugguguggccgaugugu

SEQ ID NO: 1567 - RNA, 19 nt, Artificial
  SYNTHETIC CONSTRUCT
cuaccacaccggcuacaca

SEQ ID NO: 1568 - RNA, 19 nt, Artificial
  SYNTHETIC CONSTRUCT
uuggccgcacacugguggu

SEQ ID NO: 1569 - RNA, 19 nt, Artificial
  SYNTHETIC CONSTRUCT
aaccggcgugugaccacca

SEQ ID NO: 1570 - RNA, 19 nt, Artificial
  SYNTHETIC CONSTRUCT
ccgcacacuggugguccau

SEQ ID NO: 1571 - RNA, 19 nt, Artificial
  SYNTHETIC CONSTRUCT
ggcgugugaccaccaggua

SEQ ID NO: 1572 - RNA, 19 nt, Artificial
  SYNTHETIC CONSTRUCT
gucugaggccccuuaacuc

SEQ ID NO: 1573 - RNA, 19 nt, Artificial
  SYNTHETIC CONSTRUCT
cagacuccggggaauugag

SEQ ID NO: 1574 - RNA, 19 nt, Artificial
  SYNTHETIC CONSTRUCT
aaucuuaaaaguguaauug

SEQ ID NO: 1575 - RNA, 19 nt, Artificial
  SYNTHETIC CONSTRUCT
uuagaauuuucacauuaac

SEQ ID NO: 1576 - RNA, 19 nt, Artificial
  SYNTHETIC CONSTRUCT
aauuucuuugucauucaag

SEQ ID NO: 1577 - RNA, 19 nt, Artificial
  SYNTHETIC CONSTRUCT
uuaaagaaacaguaaguuc

SEQ ID NO: 1578 - RNA, 19 nt, Artificial
  SYNTHETIC CONSTRUCT
cuggccuauaaaguagucg

SEQ ID NO: 1579 - RNA, 19 nt, Artificial
  SYNTHETIC CONSTRUCT
gaccggauauuucaucagc

SEQ ID NO: 1580 - RNA, 19 nt, Artificial
  SYNTHETIC CONSTRUCT
uggccuauaaaguagucgc

SEQ ID NO: 1581 - RNA, 19 nt, Artificial
  SYNTHETIC CONSTRUCT
accggauauuucaucagcg

SEQ ID NO: 1582 - RNA, 19 nt, Artificial
  SYNTHETIC CONSTRUCT
ggccuauaaaguagucgcg

SEQ ID NO: 1583 - RNA, 19 nt, Artificial
  SYNTHETIC CONSTRUCT
ccggauauuucaucagcgc

SEQ ID NO: 1584 - RNA, 19 nt, Artificial
  SYNTHETIC CONSTRUCT
ggcaucaucaauuucgagc

SEQ ID NO: 1585 - RNA, 19 nt, Artificial
  SYNTHETIC CONSTRUCT
ccguaguaguuaaagcucg

SEQ ID NO: 1586 - RNA, 19 nt, Artificial
  SYNTHETIC CONSTRUCT
aaggaaaguaauggaccag

SEQ ID NO: 1587 - RNA, 19 nt, Artificial
  SYNTHETIC CONSTRUCT
uuccuuucauuaccugguc

SEQ ID NO: 1588 - RNA, 19 nt, Artificial
  SYNTHETIC CONSTRUCT
aguaauggaccagugaagg

SEQ ID NO: 1589 - RNA, 19 nt, Artificial
  SYNTHETIC CONSTRUCT
ucauuaccuggucacuucc

SEQ ID NO: 1590 - RNA, 19 nt, Artificial
  SYNTHETIC CONSTRUCT
aaaggacugacugaaggcc

SEQ ID NO: 1591 - RNA, 19 nt, Artificial
  SYNTHETIC CONSTRUCT
uuuccugacugacuuccgg

SEQ ID NO: 1592 - RNA, 19 nt, Artificial
  SYNTHETIC CONSTRUCT
uuggagauaauacagcagg

SEQ ID NO: 1593 - RNA, 19 nt, Artificial
  SYNTHETIC CONSTRUCT
aaccucuauuaugucgucc

SEQ ID NO: 1594 - RNA, 19 nt, Artificial
  SYNTHETIC CONSTRUCT
gagauaauacagcaggcug

SEQ ID NO: 1595 - RNA, 19 nt, Artificial
  SYNTHETIC CONSTRUCT
cucuauuaugucguccgac

SEQ ID NO: 1596 - RNA, 19 nt, Artificial
  SYNTHETIC CONSTRUCT
cucuauccagaaaacacgg

SEQ ID NO: 1597 - RNA, 19 nt, Artificial
  SYNTHETIC CONSTRUCT
gagauaggucuuuugugcc

SEQ ID NO: 1598 - RNA, 19 nt, Artificial
  SYNTHETIC CONSTRUCT
uauccagaaaacacggugg

SEQ ID NO: 1599 - RNA, 19 nt, Artificial
  SYNTHETIC CONSTRUCT
auaggucuuuugugccacc

SEQ ID NO: 1600 - RNA, 19 nt, Artificial
  SYNTHETIC CONSTRUCT
auccagaaaacacgguggg

SEQ ID NO: 1601 - RNA, 19 nt, Artificial
  SYNTHETIC CONSTRUCT
uaggucuuuugugccaccc

SEQ ID NO: 1602 - RNA, 19 nt, Artificial
  SYNTHETIC CONSTRUCT
ccaaaggaugaagagaggc

SEQ ID NO: 1603 - RNA, 19 nt, Artificial
  SYNTHETIC CONSTRUCT
gguuuccuacuucucuccg

SEQ ID NO: 1604 - RNA, 19 nt, Artificial
  SYNTHETIC CONSTRUCT
aggcauguuggagacuugg

SEQ ID NO: 1605 - RNA, 19 nt, Artificial
  SYNTHETIC CONSTRUCT
uccguacaaccucugaacc

SEQ ID NO: 1606 - RNA, 19 nt, Artificial
  SYNTHETIC CONSTRUCT
gacuugggcaaugugacug

SEQ ID NO: 1607 - RNA, 19 nt, Artificial
  SYNTHETIC CONSTRUCT
cugaacccguuacacugac

SEQ ID NO: 1608 - RNA, 19 nt, Artificial
  SYNTHETIC CONSTRUCT
acugcugacaaagauggug

SEQ ID NO: 1609 - RNA, 19 nt, Artificial
  SYNTHETIC CONSTRUCT
ugacgacuguuucuaccac

SEQ ID NO: 1610 - RNA, 19 nt, Artificial
  SYNTHETIC CONSTRUCT
gcugacaaagauggugugg

SEQ ID NO: 1611 - RNA, 19 nt, Artificial
  SYNTHETIC CONSTRUCT
cgacuguuucuaccacacc

SEQ ID NO: 1612 - RNA, 19 nt, Artificial
  SYNTHETIC CONSTRUCT
gaccauugcaucauuggcc

SEQ ID NO: 1613 - RNA, 19 nt, Artificial
  SYNTHETIC CONSTRUCT
cugguaacguaguaaccgg

SEQ ID NO: 1614 - RNA, 19 nt, Artificial
  SYNTHETIC CONSTRUCT
accauugcaucauuggccg

SEQ ID NO: 1615 - RNA, 19 nt, Artificial
  SYNTHETIC CONSTRUCT
ugguaacguaguaaccggc

SEQ ID NO: 1616 - RNA, 19 nt, Artificial
  SYNTHETIC CONSTRUCT
auugcaucauuggccgcac

SEQ ID NO: 1617 - RNA, 19 nt, Artificial
  SYNTHETIC CONSTRUCT
uaacguaguaaccggcgug

SEQ ID NO: 1618 - RNA, 19 nt, Artificial
  SYNTHETIC CONSTRUCT
augacuugggcaaaggugg

SEQ ID NO: 1619 - RNA, 19 nt, Artificial
  SYNTHETIC CONSTRUCT
uacugaacccguuuccacc

SEQ ID NO: 1620 - RNA, 19 nt, Artificial
  SYNTHETIC CONSTRUCT
ugugguguaauugggaucg

SEQ ID NO: 1621 - RNA, 19 nt, Artificial
  SYNTHETIC CONSTRUCT
acaccacauuaacccuagc

SEQ ID NO: 1622 - RNA, 19 nt, Artificial
  SYNTHETIC CONSTRUCT
ugguguaauugggaucgcc

SEQ ID NO: 1623 - RNA, 19 nt, Artificial
  SYNTHETIC CONSTRUCT
accacauuaacccuagcgg

SEQ ID NO: 1624 - RNA, 19 nt, Artificial
  SYNTHETIC CONSTRUCT
cccuuggauguagucugag

SEQ ID NO: 1625 - RNA, 19 nt, Artificial
  SYNTHETIC CONSTRUCT
gggaaccuacaucagacuc

SEQ ID NO: 1626 - RNA, 19 nt, Artificial
  SYNTHETIC CONSTRUCT
cuuggauguagucugaggc

SEQ ID NO: 1627 - RNA, 19 nt, Artificial
  SYNTHETIC CONSTRUCT
gaaccuacaucagacuccg

SEQ ID NO: 1628 - RNA, 19 nt, Artificial
  SYNTHETIC CONSTRUCT
uuggauguagucugaggcc

SEQ ID NO: 1629 - RNA, 19 nt, Artificial
  SYNTHETIC CONSTRUCT
aaccuacaucagacuccgg

SEQ ID NO: 1630 - RNA, 19 nt, Artificial
  SYNTHETIC CONSTRUCT
aacucaucuguuauccugc

SEQ ID NO: 1631 - RNA, 19 nt, Artificial
  SYNTHETIC CONSTRUCT
uugaguagacaauaggacg

SEQ ID NO: 1632 - RNA, 19 nt, Artificial
  SYNTHETIC CONSTRUCT
aguugcuuuaaaguaccug

SEQ ID NO: 1633 - RNA, 19 nt, Artificial
  SYNTHETIC CONSTRUCT
ucaacgaaauuucauggac

SEQ ID NO: 1634 - RNA, 19 nt, Artificial
  SYNTHETIC CONSTRUCT
augaccuguauuuugccag

SEQ ID NO: 1635 - RNA, 19 nt, Artificial
  SYNTHETIC CONSTRUCT
uacuggacauaaaacgguc

SEQ ID NO: 1636 - RNA, 19 nt, Artificial
  SYNTHETIC CONSTRUCT
uuugucauucaagccugug

SEQ ID NO: 1637 - RNA, 19 nt, Artificial
  SYNTHETIC CONSTRUCT
aaacaguaaguucggacac

SEQ ID NO: 1638 - RNA, 19 nt, Artificial
  SYNTHETIC CONSTRUCT
ccugugaauaaaaacccug

SEQ ID NO: 1639 - RNA, 19 nt, Artificial
  SYNTHETIC CONSTRUCT
ggacacuuauuuuugggac

SEQ ID NO: 1640 - RNA, 19 nt, Artificial
  SYNTHETIC CONSTRUCT
aauaaaaacccuguauggc

SEQ ID NO: 1641 - RNA, 19 nt, Artificial
  SYNTHETIC CONSTRUCT
uuauuuuugggacauaccg

SEQ ID NO: 1642 - RNA, 19 nt, Artificial
  SYNTHETIC CONSTRUCT
auggcacuuauuaugaggc

SEQ ID NO: 1643 - RNA, 19 nt, Artificial
  SYNTHETIC CONSTRUCT
uaccgugaauaauacuccg

SEQ ID NO: 1644 - RNA, 19 nt, Artificial
  SYNTHETIC CONSTRUCT
gggcgaggcgcggaggucu

SEQ ID NO: 1645 - RNA, 19 nt, Artificial
  SYNTHETIC CONSTRUCT
cccgcuccgcgccuccaga

SEQ ID NO: 1646 - RNA, 19 nt, Artificial
  SYNTHETIC CONSTRUCT
ggcgaggcgcggaggucug

SEQ ID NO: 1647 - RNA, 19 nt, Artificial
  SYNTHETIC CONSTRUCT
ccgcuccgcgccuccagac

SEQ ID NO: 1648 - RNA, 19 nt, Artificial
  SYNTHETIC CONSTRUCT
aggcgcggaggucuggccu

SEQ ID NO: 1649 - RNA, 19 nt, Artificial
  SYNTHETIC CONSTRUCT
uccgcgccuccagaccgga

SEQ ID NO: 1650 - RNA, 19 nt, Artificial
  SYNTHETIC CONSTRUCT
gucgcggagacggggugcu

SEQ ID NO: 1651 - RNA, 19 nt, Artificial
  SYNTHETIC CONSTRUCT
cagcgccucugccccacga

SEQ ID NO: 1652 - RNA, 19 nt, Artificial
  SYNTHETIC CONSTRUCT
gugcugguuugcgucguag

SEQ ID NO: 1653 - RNA, 19 nt, Artificial
  SYNTHETIC CONSTRUCT
cacgaccaaacgcagcauc

SEQ ID NO: 1654 - RNA, 19 nt, Artificial
  SYNTHETIC CONSTRUCT
gguuugcgucguagucucc

SEQ ID NO: 1655 - RNA, 19 nt, Artificial
  SYNTHETIC CONSTRUCT
ccaaacgcagcaucagagg

SEQ ID NO: 1656 - RNA, 19 nt, Artificial
  SYNTHETIC CONSTRUCT
gucguagucuccugcagcg

SEQ ID NO: 1657 - RNA, 19 nt, Artificial
  SYNTHETIC CONSTRUCT
cagcaucagaggacgucgc

SEQ ID NO: 1658 - RNA, 19 nt, Artificial
  SYNTHETIC CONSTRUCT
ucguagucuccugcagcgu

SEQ ID NO: 1659 - RNA, 19 nt, Artificial
  SYNTHETIC CONSTRUCT
agcaucagaggacgucgca

SEQ ID NO: 1660 - RNA, 19 nt, Artificial
  SYNTHETIC CONSTRUCT
agucuccugcagcgucugg

SEQ ID NO: 1661 - RNA, 19 nt, Artificial
  SYNTHETIC CONSTRUCT
ucagaggacgucgcagacc

SEQ ID NO: 1662 - RNA, 19 nt, Artificial
  SYNTHETIC CONSTRUCT
cuccugcagcgucuggggu

SEQ ID NO: 1663 - RNA, 19 nt, Artificial
  SYNTHETIC CONSTRUCT
gaggacgucgcagacccca

SEQ ID NO: 1664 - RNA, 19 nt, Artificial
  SYNTHETIC CONSTRUCT
cugcagcgucugggguuuc

SEQ ID NO: 1665 - RNA, 19 nt, Artificial
  SYNTHETIC CONSTRUCT
gacgucgcagaccccaaag

SEQ ID NO: 1666 - RNA, 19 nt, Artificial
  SYNTHETIC CONSTRUCT
gcagcgucugggguuuccg

SEQ ID NO: 1667 - RNA, 19 nt, Artificial
  SYNTHETIC CONSTRUCT
cgucgcagaccccaaaggc

SEQ ID NO: 1668 - RNA, 19 nt, Artificial
  SYNTHETIC CONSTRUCT
gcgucugggguuuccguug

SEQ ID NO: 1669 - RNA, 19 nt, Artificial
  SYNTHETIC CONSTRUCT
cgcagaccccaaaggcaac

SEQ ID NO: 1670 - RNA, 19 nt, Artificial
  SYNTHETIC CONSTRUCT
ucugggguuuccguugcag

SEQ ID NO: 1671 - RNA, 19 nt, Artificial
  SYNTHETIC CONSTRUCT
agaccccaaaggcaacguc

SEQ ID NO: 1672 - RNA, 19 nt, Artificial
  SYNTHETIC CONSTRUCT
ugggguuuccguugcaguc

SEQ ID NO: 1673 - RNA, 19 nt, Artificial
  SYNTHETIC CONSTRUCT
accccaaaggcaacgucag

SEQ ID NO: 1674 - RNA, 19 nt, Artificial
  SYNTHETIC CONSTRUCT
cguugcaguccucggaacc

SEQ ID NO: 1675 - RNA, 19 nt, Artificial
  SYNTHETIC CONSTRUCT
gcaacgucaggagccuugg

SEQ ID NO: 1676 - RNA, 19 nt, Artificial
  SYNTHETIC CONSTRUCT
uugcaguccucggaaccag

SEQ ID NO: 1677 - RNA, 19 nt, Artificial
  SYNTHETIC CONSTRUCT
aacgucaggagccuugguc

SEQ ID NO: 1678 - RNA, 19 nt, Artificial
  SYNTHETIC CONSTRUCT
caguccucggaaccaggac

SEQ ID NO: 1679 - RNA, 19 nt, Artificial
  SYNTHETIC CONSTRUCT
gucaggagccuugguccug

SEQ ID NO: 1680 - RNA, 19 nt, Artificial
  SYNTHETIC CONSTRUCT
gaaccaggaccucggcgug

SEQ ID NO: 1681 - RNA, 19 nt, Artificial
  SYNTHETIC CONSTRUCT
cuugguccuggagccgcac

SEQ ID NO: 1682 - RNA, 19 nt, Artificial
  SYNTHETIC CONSTRUCT
ccaggaccucggcguggcc

SEQ ID NO: 1683 - RNA, 19 nt, Artificial
  SYNTHETIC CONSTRUCT
gguccuggagccgcaccgg

SEQ ID NO: 1684 - RNA, 19 nt, Artificial
  SYNTHETIC CONSTRUCT
ggaccucggcguggccuag

SEQ ID NO: 1685 - RNA, 19 nt, Artificial
  SYNTHETIC CONSTRUCT
ccuggagccgcaccggauc

SEQ ID NO: 1686 - RNA, 19 nt, Artificial
  SYNTHETIC CONSTRUCT
guggccuagcgaguuaugg

SEQ ID NO: 1687 - RNA, 19 nt, Artificial
  SYNTHETIC CONSTRUCT
caccggaucgcucaauacc

SEQ ID NO: 1688 - RNA, 19 nt, Artificial
  SYNTHETIC CONSTRUCT
uggccuagcgaguuauggc

SEQ ID NO: 1689 - RNA, 19 nt, Artificial
  SYNTHETIC CONSTRUCT
accggaucgcucaauaccg

SEQ ID NO: 1690 - RNA, 19 nt, Artificial
  SYNTHETIC CONSTRUCT
ccuagcgaguuauggcgac

SEQ ID NO: 1691 - RNA, 19 nt, Artificial
  SYNTHETIC CONSTRUCT
ggaucgcucaauaccgcug

SEQ ID NO: 1692 - RNA, 19 nt, Artificial
  SYNTHETIC CONSTRUCT
cuagcgaguuauggcgacg

SEQ ID NO: 1693 - RNA, 19 nt, Artificial
  SYNTHETIC CONSTRUCT
gaucgcucaauaccgcugc

SEQ ID NO: 1694 - RNA, 19 nt, Artificial
  SYNTHETIC CONSTRUCT
gaguuauggcgacgaaggc

SEQ ID NO: 1695 - RNA, 19 nt, Artificial
  SYNTHETIC CONSTRUCT
cucaauaccgcugcuuccg

SEQ ID NO: 1696 - RNA, 19 nt, Artificial
  SYNTHETIC CONSTRUCT
auggcgacgaaggccgugu

SEQ ID NO: 1697 - RNA, 19 nt, Artificial
  SYNTHETIC CONSTRUCT
uaccgcugcuuccggcaca

SEQ ID NO: 1698 - RNA, 19 nt, Artificial
  SYNTHETIC CONSTRUCT
uggcgacgaaggccgugug

SEQ ID NO: 1699 - RNA, 19 nt, Artificial
  SYNTHETIC CONSTRUCT
accgcugcuuccggcacac

SEQ ID NO: 1700 - RNA, 19 nt, Artificial
  SYNTHETIC CONSTRUCT
ggcgacgaaggccgugugc

SEQ ID NO: 1701 - RNA, 19 nt, Artificial
  SYNTHETIC CONSTRUCT
ccgcugcuuccggcacacg

SEQ ID NO: 1702 - RNA, 19 nt, Artificial
  SYNTHETIC CONSTRUCT
ggccgugugcgugcugaag

SEQ ID NO: 1703 - RNA, 19 nt, Artificial
  SYNTHETIC CONSTRUCT
ccggcacacgcacgacuuc

SEQ ID NO: 1704 - RNA, 19 nt, Artificial
  SYNTHETIC CONSTRUCT
gccgugugcgugcugaagg

SEQ ID NO: 1705 - RNA, 19 nt, Artificial
  SYNTHETIC CONSTRUCT
cggcacacgcacgacuucc

SEQ ID NO: 1706 - RNA, 19 nt, Artificial
  SYNTHETIC CONSTRUCT
aagggcgacggcccagugc

SEQ ID NO: 1707 - RNA, 19 nt, Artificial
  SYNTHETIC CONSTRUCT
uucccgcugccgggucacg

SEQ ID NO: 1708 - RNA, 19 nt, Artificial
  SYNTHETIC CONSTRUCT
gggcgacggcccagugcag

SEQ ID NO: 1709 - RNA, 19 nt, Artificial
  SYNTHETIC CONSTRUCT
cccgcugccgggucacguc

SEQ ID NO: 1710 - RNA, 19 nt, Artificial
  SYNTHETIC CONSTRUCT
accagugaaggugugggga

SEQ ID NO: 1711 - RNA, 19 nt, Artificial
  SYNTHETIC CONSTRUCT
uggucacuuccacaccccu

SEQ ID NO: 1712 - RNA, 19 nt, Artificial
  SYNTHETIC CONSTRUCT
cagugaagguguggggaag

SEQ ID NO: 1713 - RNA, 19 nt, Artificial
  SYNTHETIC CONSTRUCT
gucacuuccacaccccuuc

SEQ ID NO: 1714 - RNA, 19 nt, Artificial
  SYNTHETIC CONSTRUCT
aggacugacugaaggccug

SEQ ID NO: 1715 - RNA, 19 nt, Artificial
  SYNTHETIC CONSTRUCT
uccugacugacuuccggac

SEQ ID NO: 1716 - RNA, 19 nt, Artificial
  SYNTHETIC CONSTRUCT
cugacugaaggccugcaug

SEQ ID NO: 1717 - RNA, 19 nt, Artificial
  SYNTHETIC CONSTRUCT
gacugacuuccggacguac

SEQ ID NO: 1718 - RNA, 19 nt, Artificial
  SYNTHETIC CONSTRUCT
ggccugcauggauuccaug

SEQ ID NO: 1719 - RNA, 19 nt, Artificial
  SYNTHETIC CONSTRUCT
ccggacguaccuaagguac

SEQ ID NO: 1720 - RNA, 19 nt, Artificial
  SYNTHETIC CONSTRUCT
cagcaggcuguaccagugc

SEQ ID NO: 1721 - RNA, 19 nt, Artificial
  SYNTHETIC CONSTRUCT
gucguccgacauggucacg

SEQ ID NO: 1722 - RNA, 19 nt, Artificial
  SYNTHETIC CONSTRUCT
uaccagugcagguccucac

SEQ ID NO: 1723 - RNA, 19 nt, Artificial
  SYNTHETIC CONSTRUCT
auggucacguccaggagug

SEQ ID NO: 1724 - RNA, 19 nt, Artificial
  SYNTHETIC CONSTRUCT
cacggugggccaaaggaug

SEQ ID NO: 1725 - RNA, 19 nt, Artificial
  SYNTHETIC CONSTRUCT
gugccacccgguuuccuac

SEQ ID NO: 1726 - RNA, 19 nt, Artificial
  SYNTHETIC CONSTRUCT
ggugggccaaaggaugaag

SEQ ID NO: 1727 - RNA, 19 nt, Artificial
  SYNTHETIC CONSTRUCT
ccacccgguuuccuacuuc

SEQ ID NO: 1728 - RNA, 19 nt, Artificial
  SYNTHETIC CONSTRUCT
ggcauguuggagacuuggg

SEQ ID NO: 1729 - RNA, 19 nt, Artificial
  SYNTHETIC CONSTRUCT
ccguacaaccucugaaccc

SEQ ID NO: 1730 - RNA, 19 nt, Artificial
  SYNTHETIC CONSTRUCT
gcauguuggagacuugggc

SEQ ID NO: 1731 - RNA, 19 nt, Artificial
  SYNTHETIC CONSTRUCT
cguacaaccucugaacccg

SEQ ID NO: 1732 - RNA, 19 nt, Artificial
  SYNTHETIC CONSTRUCT
gggcaaugugacugcugac

SEQ ID NO: 1733 - RNA, 19 nt, Artificial
  SYNTHETIC CONSTRUCT
cccguuacacugacgacug

SEQ ID NO: 1734 - RNA, 19 nt, Artificial
  SYNTHETIC CONSTRUCT
augguguggccgauguguc

SEQ ID NO: 1735 - RNA, 19 nt, Artificial
  SYNTHETIC CONSTRUCT
uaccacaccggcuacacag

SEQ ID NO: 1736 - RNA, 19 nt, Artificial
  SYNTHETIC CONSTRUCT
ugcaucauuggccgcacac

SEQ ID NO: 1737 - RNA, 19 nt, Artificial
  SYNTHETIC CONSTRUCT
acguaguaaccggcgugug

SEQ ID NO: 1738 - RNA, 19 nt, Artificial
  SYNTHETIC CONSTRUCT
caucauuggccgcacacug

SEQ ID NO: 1739 - RNA, 19 nt, Artificial
  SYNTHETIC CONSTRUCT
guaguaaccggcgugugac

SEQ ID NO: 1740 - RNA, 19 nt, Artificial
  SYNTHETIC CONSTRUCT
aucauuggccgcacacugg

SEQ ID NO: 1741 - RNA, 19 nt, Artificial
  SYNTHETIC CONSTRUCT
uaguaaccggcgugugacc

SEQ ID NO: 1742 - RNA, 19 nt, Artificial
  SYNTHETIC CONSTRUCT
cauuggccgcacacuggug

SEQ ID NO: 1743 - RNA, 19 nt, Artificial
  SYNTHETIC CONSTRUCT
guaaccggcgugugaccac

SEQ ID NO: 1744 - RNA, 19 nt, Artificial
  SYNTHETIC CONSTRUCT
gauguagucugaggccccu

SEQ ID NO: 1745 - RNA, 19 nt, Artificial
  SYNTHETIC CONSTRUCT
cuacaucagacuccgggga

SEQ ID NO: 1746 - RNA, 19 nt, Artificial
  SYNTHETIC CONSTRUCT
cugacaaagaugguguggc

SEQ ID NO: 1747 - RNA, 19 nt, Artificial
  SYNTHETIC CONSTRUCT
gacuguuucuaccacaccg

SEQ ID NO: 1748 - RNA, 19 nt, Artificial
  SYNTHETIC CONSTRUCT
guuuggggccagagugggc

SEQ ID NO: 1749 - RNA, 19 nt, Artificial
  SYNTHETIC CONSTRUCT
caaaccccggucucacccg

SEQ ID NO: 1750 - RNA, 19 nt, Artificial
  SYNTHETIC CONSTRUCT
gggccagagugggcgaggc

SEQ ID NO: 1751 - RNA, 19 nt, Artificial
  SYNTHETIC CONSTRUCT
cccggucucacccgcuccg

SEQ ID NO: 1752 - RNA, 19 nt, Artificial
  SYNTHETIC CONSTRUCT
ggccagagugggcgaggcg

SEQ ID NO: 1753 - RNA, 19 nt, Artificial
  SYNTHETIC CONSTRUCT
ccggucucacccgcuccgc

SEQ ID NO: 1754 - RNA, 19 nt, Artificial
  SYNTHETIC CONSTRUCT
ccagagugggcgaggcgcg

SEQ ID NO: 1755 - RNA, 19 nt, Artificial
  SYNTHETIC CONSTRUCT
ggucucacccgcuccgcgc

SEQ ID NO: 1756 - RNA, 19 nt, Artificial
  SYNTHETIC CONSTRUCT
gagugggcgaggcgcggag

SEQ ID NO: 1757 - RNA, 19 nt, Artificial
  SYNTHETIC CONSTRUCT
cucacccgcuccgcgccuc

SEQ ID NO: 1758 - RNA, 19 nt, Artificial
  SYNTHETIC CONSTRUCT
ugggcgaggcgcggagguc

SEQ ID NO: 1759 - RNA, 19 nt, Artificial
  SYNTHETIC CONSTRUCT
acccgcuccgcgccuccag

SEQ ID NO: 1760 - RNA, 19 nt, Artificial
  SYNTHETIC CONSTRUCT
gcgaggcgcggaggucugg

SEQ ID NO: 1761 - RNA, 19 nt, Artificial
  SYNTHETIC CONSTRUCT
cgcuccgcgccuccagacc

SEQ ID NO: 1762 - RNA, 19 nt, Artificial
  SYNTHETIC CONSTRUCT
cgaggcgcggaggucuggc

SEQ ID NO: 1763 - RNA, 19 nt, Artificial
  SYNTHETIC CONSTRUCT
gcuccgcgccuccagaccg

SEQ ID NO: 1764 - RNA, 19 nt, Artificial
  SYNTHETIC CONSTRUCT
gaggcgcggaggucuggcc

SEQ ID NO: 1765 - RNA, 19 nt, Artificial
  SYNTHETIC CONSTRUCT
cuccgcgccuccagaccgg

SEQ ID NO: 1766 - RNA, 19 nt, Artificial
  SYNTHETIC CONSTRUCT
aaaguagucgcggagacgg

SEQ ID NO: 1767 - RNA, 19 nt, Artificial
  SYNTHETIC CONSTRUCT
uuucaucagcgccucugcc

SEQ ID NO: 1768 - RNA, 19 nt, Artificial
  SYNTHETIC CONSTRUCT
aaguagucgcggagacggg

SEQ ID NO: 1769 - RNA, 19 nt, Artificial
  SYNTHETIC CONSTRUCT
uucaucagcgccucugccc

SEQ ID NO: 1770 - RNA, 19 nt, Artificial
  SYNTHETIC CONSTRUCT
uagucgcggagacggggug

SEQ ID NO: 1771 - RNA, 19 nt, Artificial
  SYNTHETIC CONSTRUCT
aucagcgccucugccccac

SEQ ID NO: 1772 - RNA, 19 nt, Artificial
  SYNTHETIC CONSTRUCT
ucgcggagacggggugcug

SEQ ID NO: 1773 - RNA, 19 nt, Artificial
  SYNTHETIC CONSTRUCT
agcgccucugccccacgac

SEQ ID NO: 1774 - RNA, 19 nt, Artificial
  SYNTHETIC CONSTRUCT
gacggggugcugguuugcg

SEQ ID NO: 1775 - RNA, 19 nt, Artificial
  SYNTHETIC CONSTRUCT
cugccccacgaccaaacgc

SEQ ID NO: 1776 - RNA, 19 nt, Artificial
  SYNTHETIC CONSTRUCT
cggggugcugguuugcguc

SEQ ID NO: 1777 - RNA, 19 nt, Artificial
  SYNTHETIC CONSTRUCT
gccccacgaccaaacgcag

SEQ ID NO: 1778 - RNA, 19 nt, Artificial
  SYNTHETIC CONSTRUCT
ggggugcugguuugcgucg

SEQ ID NO: 1779 - RNA, 19 nt, Artificial
  SYNTHETIC CONSTRUCT
ccccacgaccaaacgcagc

SEQ ID NO: 1780 - RNA, 19 nt, Artificial
  SYNTHETIC CONSTRUCT
uugcgucguagucuccugc

SEQ ID NO: 1781 - RNA, 19 nt, Artificial
  SYNTHETIC CONSTRUCT
aacgcagcaucagaggacg

SEQ ID NO: 1782 - RNA, 19 nt, Artificial
  SYNTHETIC CONSTRUCT
cgucguagucuccugcagc

SEQ ID NO: 1783 - RNA, 19 nt, Artificial
  SYNTHETIC CONSTRUCT
gcagcaucagaggacgucg

SEQ ID NO: 1784 - RNA, 19 nt, Artificial
  SYNTHETIC CONSTRUCT
cguagucuccugcagcguc

SEQ ID NO: 1785 - RNA, 19 nt, Artificial
  SYNTHETIC CONSTRUCT
gcaucagaggacgucgcag

SEQ ID NO: 1786 - RNA, 19 nt, Artificial
  SYNTHETIC CONSTRUCT
uagucuccugcagcgucug

SEQ ID NO: 1787 - RNA, 19 nt, Artificial
  SYNTHETIC CONSTRUCT
aucagaggacgucgcagac

SEQ ID NO: 1788 - RNA, 19 nt, Artificial
  SYNTHETIC CONSTRUCT
ucuccugcagcgucugggg

SEQ ID NO: 1789 - RNA, 19 nt, Artificial
  SYNTHETIC CONSTRUCT
agaggacgucgcagacccc

SEQ ID NO: 1790 - RNA, 19 nt, Artificial
  SYNTHETIC CONSTRUCT
gggguuuccguugcagucc

SEQ ID NO: 1791 - RNA, 19 nt, Artificial
  SYNTHETIC CONSTRUCT
ccccaaaggcaacgucagg

SEQ ID NO: 1792 - RNA, 19 nt, Artificial
  SYNTHETIC CONSTRUCT
uuuccguugcaguccucgg

SEQ ID NO: 1793 - RNA, 19 nt, Artificial
  SYNTHETIC CONSTRUCT
aaaggcaacgucaggagcc

SEQ ID NO: 1794 - RNA, 19 nt, Artificial
  SYNTHETIC CONSTRUCT
ugcaguccucggaaccagg

SEQ ID NO: 1795 - RNA, 19 nt, Artificial
  SYNTHETIC CONSTRUCT
acgucaggagccuuggucc

SEQ ID NO: 1796 - RNA, 19 nt, Artificial
  SYNTHETIC CONSTRUCT
aguccucggaaccaggacc

SEQ ID NO: 1797 - RNA, 19 nt, Artificial
  SYNTHETIC CONSTRUCT
ucaggagccuugguccugg

SEQ ID NO: 1798 - RNA, 19 nt, Artificial
  SYNTHETIC CONSTRUCT
uccucggaaccaggaccuc

SEQ ID NO: 1799 - RNA, 19 nt, Artificial
  SYNTHETIC CONSTRUCT
aggagccuugguccuggag

SEQ ID NO: 1800 - RNA, 19 nt, Artificial
  SYNTHETIC CONSTRUCT
cucggaaccaggaccucgg

SEQ ID NO: 1801 - RNA, 19 nt, Artificial
  SYNTHETIC CONSTRUCT
gagccuugguccuggagcc

SEQ ID NO: 1802 - RNA, 19 nt, Artificial
  SYNTHETIC CONSTRUCT
ucggaaccaggaccucggc

SEQ ID NO: 1803 - RNA, 19 nt, Artificial
  SYNTHETIC CONSTRUCT
agccuugguccuggagccg

SEQ ID NO: 1804 - RNA, 19 nt, Artificial
  SYNTHETIC CONSTRUCT
accaggaccucggcguggc

SEQ ID NO: 1805 - RNA, 19 nt, Artificial
  SYNTHETIC CONSTRUCT
ugguccuggagccgcaccg

SEQ ID NO: 1806 - RNA, 19 nt, Artificial
  SYNTHETIC CONSTRUCT
caggaccucggcguggccu

SEQ ID NO: 1807 - RNA, 19 nt, Artificial
  SYNTHETIC CONSTRUCT
guccuggagccgcaccgga

SEQ ID NO: 1808 - RNA, 19 nt, Artificial
  SYNTHETIC CONSTRUCT
gaccucggcguggccuagc

SEQ ID NO: 1809 - RNA, 19 nt, Artificial
  SYNTHETIC CONSTRUCT
cuggagccgcaccggaucg

SEQ ID NO: 1810 - RNA, 19 nt, Artificial
  SYNTHETIC CONSTRUCT
cucggcguggccuagcgag

SEQ ID NO: 1811 - RNA, 19 nt, Artificial
  SYNTHETIC CONSTRUCT
gagccgcaccggaucgcuc

SEQ ID NO: 1812 - RNA, 19 nt, Artificial
  SYNTHETIC CONSTRUCT
ggccuagcgaguuauggcg

SEQ ID NO: 1813 - RNA, 19 nt, Artificial
  SYNTHETIC CONSTRUCT
ccggaucgcucaauaccgc

SEQ ID NO: 1814 - RNA, 19 nt, Artificial
  SYNTHETIC CONSTRUCT
gcgaguuauggcgacgaag

SEQ ID NO: 1815 - RNA, 19 nt, Artificial
  SYNTHETIC CONSTRUCT
cgcucaauaccgcugcuuc

SEQ ID NO: 1816 - RNA, 19 nt, Artificial
  SYNTHETIC CONSTRUCT
acgaaggccgugugcgugc

SEQ ID NO: 1817 - RNA, 19 nt, Artificial
  SYNTHETIC CONSTRUCT
ugcuuccggcacacgcacg

SEQ ID NO: 1818 - RNA, 19 nt, Artificial
  SYNTHETIC CONSTRUCT
cgugcugaagggcgacggc

SEQ ID NO: 1819 - RNA, 19 nt, Artificial
  SYNTHETIC CONSTRUCT
gcacgacuucccgcugccg

SEQ ID NO: 1820 - RNA, 19 nt, Artificial
  SYNTHETIC CONSTRUCT
gcgacggcccagugcaggg

SEQ ID NO: 1821 - RNA, 19 nt, Artificial
  SYNTHETIC CONSTRUCT
cgcugccgggucacguccc

SEQ ID NO: 1822 - RNA, 19 nt, Artificial
  SYNTHETIC CONSTRUCT
cgacggcccagugcagggc

SEQ ID NO: 1823 - RNA, 19 nt, Artificial
  SYNTHETIC CONSTRUCT
gcugccgggucacgucccg

SEQ ID NO: 1824 - RNA, 19 nt, Artificial
  SYNTHETIC CONSTRUCT
cggcccagugcagggcauc

SEQ ID NO: 1825 - RNA, 19 nt, Artificial
  SYNTHETIC CONSTRUCT
gccgggucacgucccguag

SEQ ID NO: 1826 - RNA, 19 nt, Artificial
  SYNTHETIC CONSTRUCT
uggaccagugaaggugugg

SEQ ID NO: 1827 - RNA, 19 nt, Artificial
  SYNTHETIC CONSTRUCT
accuggucacuuccacacc

SEQ ID NO: 1828 - RNA, 19 nt, Artificial
  SYNTHETIC CONSTRUCT
ggaccagugaagguguggg

SEQ ID NO: 1829 - RNA, 19 nt, Artificial
  SYNTHETIC CONSTRUCT
ccuggucacuuccacaccc

SEQ ID NO: 1830 - RNA, 19 nt, Artificial
  SYNTHETIC CONSTRUCT
gaccagugaaggugugggg

SEQ ID NO: 1831 - RNA, 19 nt, Artificial
  SYNTHETIC CONSTRUCT
cuggucacuuccacacccc

SEQ ID NO: 1832 - RNA, 19 nt, Artificial
  SYNTHETIC CONSTRUCT
agugaagguguggggaagc

SEQ ID NO: 1833 - RNA, 19 nt, Artificial
  SYNTHETIC CONSTRUCT
ucacuuccacaccccuucg

SEQ ID NO: 1834 - RNA, 19 nt, Artificial
  SYNTHETIC CONSTRUCT
ggacugacugaaggccugc

SEQ ID NO: 1835 - RNA, 19 nt, Artificial
  SYNTHETIC CONSTRUCT
ccugacugacuuccggacg

SEQ ID NO: 1836 - RNA, 19 nt, Artificial
  SYNTHETIC CONSTRUCT
ggcuguaccagugcagguc

SEQ ID NO: 1837 - RNA, 19 nt, Artificial
  SYNTHETIC CONSTRUCT
ccgacauggucacguccag

SEQ ID NO: 1838 - RNA, 19 nt, Artificial
  SYNTHETIC CONSTRUCT
gcuguaccagugcaggucc

SEQ ID NO: 1839 - RNA, 19 nt, Artificial
  SYNTHETIC CONSTRUCT
cgacauggucacguccagg

SEQ ID NO: 1840 - RNA, 19 nt, Artificial
  SYNTHETIC CONSTRUCT
uguaccagugcagguccuc

SEQ ID NO: 1841 - RNA, 19 nt, Artificial
  SYNTHETIC CONSTRUCT
acauggucacguccaggag

SEQ ID NO: 1842 - RNA, 19 nt, Artificial
  SYNTHETIC CONSTRUCT
ccauugcaucauuggccgc

SEQ ID NO: 1843 - RNA, 19 nt, Artificial
  SYNTHETIC CONSTRUCT
gguaacguaguaaccggcg

SEQ ID NO: 1844 - RNA, 19 nt, Artificial
  SYNTHETIC CONSTRUCT
auuggccgcacacuggugg

SEQ ID NO: 1845 - RNA, 19 nt, Artificial
  SYNTHETIC CONSTRUCT
uaaccggcgugugaccacc

SEQ ID NO: 1846 - RNA, 19 nt, Artificial
  SYNTHETIC CONSTRUCT
uggccgcacacuggugguc

SEQ ID NO: 1847 - RNA, 19 nt, Artificial
  SYNTHETIC CONSTRUCT
accggcgugugaccaccag

SEQ ID NO: 1848 - RNA, 19 nt, Artificial
  SYNTHETIC CONSTRUCT
cgcacacuggugguccaug

SEQ ID NO: 1849 - RNA, 19 nt, Artificial
  SYNTHETIC CONSTRUCT
gcgugugaccaccagguac

SEQ ID NO: 1850 - RNA, 19 nt, Artificial
  SYNTHETIC CONSTRUCT
caggaaacgcuggaagucg

SEQ ID NO: 1851 - RNA, 19 nt, Artificial
  SYNTHETIC CONSTRUCT
guccuuugcgaccuucagc

SEQ ID NO: 1852 - RNA, 19 nt, Artificial
  SYNTHETIC CONSTRUCT
acgcuggaagucguuuggc

SEQ ID NO: 1853 - RNA, 19 nt, Artificial
  SYNTHETIC CONSTRUCT
ugcgaccuucagcaaaccg

SEQ ID NO: 1854 - RNA, 19 nt, Artificial
  SYNTHETIC CONSTRUCT
ggauguagucugaggcccc

SEQ ID NO: 1855 - RNA, 19 nt, Artificial
  SYNTHETIC CONSTRUCT
ccuacaucagacuccgggg

SEQ ID NO: 1856 - RNA, 19 nt, Artificial
  SYNTHETIC CONSTRUCT
gccuauaaaguagucgcgg

SEQ ID NO: 1857 - RNA, 19 nt, Artificial
  SYNTHETIC CONSTRUCT
cggauauuucaucagcgcc

SEQ ID NO: 1858 - RNA, 19 nt, Artificial
  SYNTHETIC CONSTRUCT
uggggccagagugggcgag

SEQ ID NO: 1859 - RNA, 19 nt, Artificial
  SYNTHETIC CONSTRUCT
accccggucucacccgcuc

SEQ ID NO: 1860 - RNA, 19 nt, Artificial
  SYNTHETIC CONSTRUCT
ggggccagagugggcgagg

SEQ ID NO: 1861 - RNA, 19 nt, Artificial
  SYNTHETIC CONSTRUCT
ccccggucucacccgcucc

SEQ ID NO: 1862 - RNA, 19 nt, Artificial
  SYNTHETIC CONSTRUCT
agugggcgaggcgcggagg

SEQ ID NO: 1863 - RNA, 19 nt, Artificial
  SYNTHETIC CONSTRUCT
ucacccgcuccgcgccucc

SEQ ID NO: 1864 - RNA, 19 nt, Artificial
  SYNTHETIC CONSTRUCT
ucagcgccucugccccacg

SEQ ID NO: 1865 - RNA, 19 nt, Artificial
  SYNTHETIC CONSTRUCT
ucagcgccucugccccacg

SEQ ID NO: 1866 - RNA, 19 nt, Artificial
  SYNTHETIC CONSTRUCT
cgcggagacggggugcugg

SEQ ID NO: 1867 - RNA, 19 nt, Artificial
  SYNTHETIC CONSTRUCT
gcgccucugccccacgacc

SEQ ID NO: 1868 - RNA, 19 nt, Artificial
  SYNTHETIC CONSTRUCT
gucuccugcagcgucuggg

SEQ ID NO: 1869 - RNA, 19 nt, Artificial
  SYNTHETIC CONSTRUCT
cagaggacgucgcagaccc

SEQ ID NO: 1870 - RNA, 19 nt, Artificial
  SYNTHETIC CONSTRUCT
guuuccguugcaguccucg

SEQ ID NO: 1871 - RNA, 19 nt, Artificial
  SYNTHETIC CONSTRUCT
caaaggcaacgucaggagc

SEQ ID NO: 1872 - RNA, 19 nt, Artificial
  SYNTHETIC CONSTRUCT
ccucggaaccaggaccucg

SEQ ID NO: 1873 - RNA, 19 nt, Artificial
  SYNTHETIC CONSTRUCT
ggagccuugguccuggagc

SEQ ID NO: 1874 - RNA, 19 nt, Artificial
  SYNTHETIC CONSTRUCT
cggaaccaggaccucggcg

SEQ ID NO: 1875 - RNA, 19 nt, Artificial
  SYNTHETIC CONSTRUCT
gccuugguccuggagccgc

SEQ ID NO: 1876 - RNA, 19 nt, Artificial
  SYNTHETIC CONSTRUCT
aaccaggaccucggcgugg

SEQ ID NO: 1877 - RNA, 19 nt, Artificial
  SYNTHETIC CONSTRUCT
uugguccuggagccgcacc

SEQ ID NO: 1878 - RNA, 19 nt, Artificial
  SYNTHETIC CONSTRUCT
aguuauggcgacgaaggcc

SEQ ID NO: 1879 - RNA, 19 nt, Artificial
  SYNTHETIC CONSTRUCT
ucaauaccgcugcuuccgg

SEQ ID NO: 1880 - RNA, 19 nt, Artificial
  SYNTHETIC CONSTRUCT
guuauggcgacgaaggccg

SEQ ID NO: 1881 - RNA, 19 nt, Artificial
  SYNTHETIC CONSTRUCT
caauaccgcugcuuccggc

SEQ ID NO: 1882 - RNA, 19 nt, Artificial
  SYNTHETIC CONSTRUCT
uauggcgacgaaggccgug

SEQ ID NO: 1883 - RNA, 19 nt, Artificial
  SYNTHETIC CONSTRUCT
auaccgcugcuuccggcac

SEQ ID NO: 1884 - RNA, 19 nt, Artificial
  SYNTHETIC CONSTRUCT
gcgacgaaggccgugugcg

SEQ ID NO: 1885 - RNA, 19 nt, Artificial
  SYNTHETIC CONSTRUCT
cgcugcuuccggcacacgc

SEQ ID NO: 1886 - RNA, 19 nt, Artificial
  SYNTHETIC CONSTRUCT
gacgaaggccgugugcgug

SEQ ID NO: 1887 - RNA, 19 nt, Artificial
  SYNTHETIC CONSTRUCT
cugcuuccggcacacgcac

SEQ ID NO: 1888 - RNA, 19 nt, Artificial
  SYNTHETIC CONSTRUCT
cgugugcgugcugaagggc

SEQ ID NO: 1889 - RNA, 19 nt, Artificial
  SYNTHETIC CONSTRUCT
gcacacgcacgacuucccg

SEQ ID NO: 1890 - RNA, 19 nt, Artificial
  SYNTHETIC CONSTRUCT
gugugcgugcugaagggcg

SEQ ID NO: 1891 - RNA, 19 nt, Artificial
  SYNTHETIC CONSTRUCT
cacacgcacgacuucccgc

SEQ ID NO: 1892 - RNA, 19 nt, Artificial
  SYNTHETIC CONSTRUCT
gugcgugcugaagggcgac

SEQ ID NO: 1893 - RNA, 19 nt, Artificial
  SYNTHETIC CONSTRUCT
cacgcacgacuucccgcug

SEQ ID NO: 1894 - RNA, 19 nt, Artificial
  SYNTHETIC CONSTRUCT
ugcgugcugaagggcgacg

SEQ ID NO: 1895 - RNA, 19 nt, Artificial
  SYNTHETIC CONSTRUCT
acgcacgacuucccgcugc

SEQ ID NO: 1896 - RNA, 19 nt, Artificial
  SYNTHETIC CONSTRUCT
gcgugcugaagggcgacgg

SEQ ID NO: 1897 - RNA, 19 nt, Artificial
  SYNTHETIC CONSTRUCT
cgcacgacuucccgcugcc

SEQ ID NO: 1898 - RNA, 19 nt, Artificial
  SYNTHETIC CONSTRUCT
ugcugaagggcgacggccc

SEQ ID NO: 1899 - RNA, 19 nt, Artificial
  SYNTHETIC CONSTRUCT
acgacuucccgcugccggg

SEQ ID NO: 1900 - RNA, 19 nt, Artificial
  SYNTHETIC CONSTRUCT
cugaagggcgacggcccag

SEQ ID NO: 1901 - RNA, 19 nt, Artificial
  SYNTHETIC CONSTRUCT
gacuucccgcugccggguc

SEQ ID NO: 1902 - RNA, 19 nt, Artificial
  SYNTHETIC CONSTRUCT
ggcgacggcccagugcagg

SEQ ID NO: 1903 - RNA, 19 nt, Artificial
  SYNTHETIC CONSTRUCT
ccgcugccgggucacgucc

SEQ ID NO: 1904 - RNA, 19 nt, Artificial
  SYNTHETIC CONSTRUCT
caggcuguaccagugcagg

SEQ ID NO: 1905 - RNA, 19 nt, Artificial
  SYNTHETIC CONSTRUCT
guccgacauggucacgucc

SEQ ID NO: 1906 - RNA, 19 nt, Artificial
  SYNTHETIC CONSTRUCT
uccagaaaacacggugggc

SEQ ID NO: 1907 - RNA, 19 nt, Artificial
  SYNTHETIC CONSTRUCT
aggucuuuugugccacccg

SEQ ID NO: 1908 - RNA, 19 nt, Artificial
  SYNTHETIC CONSTRUCT
ccagaaaacacggugggcc

SEQ ID NO: 1909 - RNA, 19 nt, Artificial
  SYNTHETIC CONSTRUCT
ggucuuuugugccacccgg

SEQ ID NO: 1910 - RNA, 19 nt, Artificial
  SYNTHETIC CONSTRUCT
ggccgcacacugguggucc

SEQ ID NO: 1911 - RNA, 19 nt, Artificial
  SYNTHETIC CONSTRUCT
ccggcgugugaccaccagg

SEQ ID NO: 1912 - RNA, 19 nt, Artificial
  SYNTHETIC CONSTRUCT
gguguaauugggaucgccc

SEQ ID NO: 1913 - RNA, 19 nt, Artificial
  SYNTHETIC CONSTRUCT
ccacauuaacccuagcggg

SEQ ID NO: 1914 - RNA, 19 nt, Artificial
  SYNTHETIC CONSTRUCT
uggauguagucugaggccc

SEQ ID NO: 1915 - RNA, 19 nt, Artificial
  SYNTHETIC CONSTRUCT
accuacaucagacuccggg

SEQ ID NO: 1916 - RNA, 19 nt, Artificial
  SYNTHETIC CONSTRUCT
uuuggggccagagugggcg

SEQ ID NO: 1917 - RNA, 19 nt, Artificial
  SYNTHETIC CONSTRUCT
aaaccccggucucacccgc

SEQ ID NO: 1918 - RNA, 19 nt, Artificial
  SYNTHETIC CONSTRUCT
gccagagugggcgaggcgc

SEQ ID NO: 1919 - RNA, 19 nt, Artificial
  SYNTHETIC CONSTRUCT
cggucucacccgcuccgcg

SEQ ID NO: 1920 - RNA, 19 nt, Artificial
  SYNTHETIC CONSTRUCT
cagagugggcgaggcgcgg

SEQ ID NO: 1921 - RNA, 19 nt, Artificial
  SYNTHETIC CONSTRUCT
gucucacccgcuccgcgcc

SEQ ID NO: 1922 - RNA, 19 nt, Artificial
  SYNTHETIC CONSTRUCT
aguagucgcggagacgggg

SEQ ID NO: 1923 - RNA, 19 nt, Artificial
  SYNTHETIC CONSTRUCT
ucaucagcgccucugcccc

SEQ ID NO: 1924 - RNA, 19 nt, Artificial
  SYNTHETIC CONSTRUCT
gugcugaagggcgacggcc

SEQ ID NO: 1925 - RNA, 19 nt, Artificial
  SYNTHETIC CONSTRUCT
cacgacuucccgcugccgg

SEQ ID NO: 1926 - RNA, 19 nt, Artificial
  SYNTHETIC CONSTRUCT
gacaaagaugguguggccg

SEQ ID NO: 1927 - RNA, 19 nt, Artificial
  SYNTHETIC CONSTRUCT
cuguuucuaccacaccggc